(19) United States
(12) Patent Application Publication
(10) Pub. No.: US 2022/0275389 A1
(43) Pub. Date: Sep. 1, 2022

(54) MODIFIED EXOPOLYSACCHARIDE RECEPTORS FOR RECOGNIZING AND STRUCTURING MICROBIOTA

(71) Applicant: Aarhus Universitet, Aarhus C (DK)

(72) Inventors: Kasper Røjkjær ANDERSEN, Aarhus C (DK); Simon KELLY, Aarhus C (DK); Mei Mei Jaslyn Elizabeth WONG, Aarhus C (DK); Kira GYSEL, Aarhus C (DK); Simona RADUTOIU, Aarhus C (DK); Ke TAO, Aarhus C (DK); Simon Boje HANSEN, Aarhus C (DK); Jens STOUGAARD, Aarhus C (DK); Sha ZHANG, Aarhus C (DK)

(73) Assignee: Aarhus Universitet, Aarhus C (DK)

(21) Appl. No.: 17/631,692

(22) PCT Filed: Aug. 19, 2020
(86) PCT No.: PCT/EP2020/073164

§ 371 Date: Jan. 31, 2022

Related U.S. Application Data
(60) Provisional application No. 62/888,944, filed on Aug. 19, 2019.

(51) Int. Cl. C12N 15/82; C07K 14/415

(52) U.S. Cl. CPC C12N 15/8261; C07K 14/415

(57) ABSTRACT

Aspects of the present disclosure relate to genetically altered plants having a heterologous EPR3 or EPR3-like polypeptide or a modified EPR3 or EPR3-like polypeptide and/or having a heterologous EPR3a or EPR3a-like polypeptide or a modified EPR3a or EPR3a-like polypeptide, wherein the EPR3 or EPR3-like polypeptide and/or the EPR3a or EPR3a-like polypeptide provide increased selectivity for a beneficial commensal microbe as compared to a wild-type plant under the same conditions. Other aspects of the present disclosure relate to methods of making such plants as well as cultivating these genetically altered plants. Additional aspects of the present disclosure relate to methods of identifying a beneficial commensal microbe capable of interacting with a plant root microbiota.

### CROSS-REFERENCE TO RELATED APPLICATION

This application claims the benefit of U.S. Provisional Application No. 62/888,944, filed Aug. 19, 2019, which is hereby incorporated by reference in its entirety.

### SUBMISSION OF SEQUENCE LISTING AS ASCII TEXT FILE

The content of the following submission on ASCII text file is incorporated herein by reference in its entirety: a computer readable form (CRF) of the Sequence Listing (file name: 794542000940SEQLIST.TXT, date recorded: Aug. 13, 2020, size: 723 KB).

### TECHNICAL FIELD

The present disclosure relates to genetically altered plants. In particular, the present disclosure relates to genetically altered plants with a heterologous EPR3 or EPR3-like polypeptide or a modified EPR3 or EPR3-like polypeptide and/or with a heterologous EPR3a or EPR3a-like polypeptide or a modified EPR3a or EPR3a-like polypeptide, wherein the EPR3 or EPR3-like polypeptide and/or the EPR3a or EPR3a-like polypeptide provide increased selectivity for a beneficial commensal microbe as compared to a wild-type plant under the same conditions.

### BACKGROUND

Microbes produce extracellular polysaccharides, such as lipopolysaccharides and exopolysaccharides (EPS), which can be displayed on their surface or secreted into their environment. These polysaccharides are characteristic of the microbial species that produces them and can therefore be used as microbial associated molecular patterns for receptor-mediated recognition by mammals and plants.

One example of this recognition is found in legumes, which monitor the molecular composition of EPS of the surrounding soil to determine whether symbiosis pathways in the plant for rhizobial bacteria should be blocked or promoted. Symbiosis between legumes and rhizobia that fix nitrogen is governed by a two-step receptor-mediated recognition system. In the first step, rhizobial lipo-chitooligosaccharides (LCOs, also referred to as Nod factors) are perceived by plant LCO receptors. This perception induces the development of root nodule primordia, the entrapment of rhizobia in root hair curls, and triggers the plant program for bacterial infection. In the second step, rhizobial EPS are perceived, and this controls subsequent progression of nodule infection. In the legume Lotus japonicus, the single-pass transmembrane receptor-kinase EPR3 recognizes the R7A EPS produced by the Lotus symbiont Mesorhizobium loti. Studies of rhizobia and host plant mutants have showed that EPS perception, and subsequent EPR3 signaling, promote infection of the epidermal and cortical tissues of Lotus roots (Kawaharada, Y. et al. Nature 2015 523: 308-312; Kawaharada, Y. et al. Nat Commun 2017 8: 14534). In the absence of the correct R7A EPS, rhizobial infection and colonization are blocked in an EPR3-dependent manner (Kawaharada, Y. et al. Nature 2015 523: 308-312; Kelly, S. J. et al. Mol. Plant Microbe Interact 2013 26: 319-329), suggesting that EPS perception determines compatibility in legume-rhizobia interactions. These results, however, characterize the role of EPS perception in a limited context and primarily as a secondary gate on the symbiotic process with nitrogen fixing rhizobia. Many plant species, including most crop plants, do not establish nitrogen-fixing symbiosis, but instead have less well characterized plant-microbial interactions in the form of complex plant-associated microbial communities (microbiota).

Beyond just nitrogen-fixation symbiosis, soil-borne microbes can improve plant fitness by increasing nutrient availability, conferring pathogen resistance, and improving resilience to abiotic stresses. While recent studies have improved the understanding of the plant microbiota, the principles guiding if and how plants select for microbiota and encourage a healthy microbiota in the local soil space are largely unknown. Moreover, the role of EPS perception in the selection of microbiota has remained unknown. The microbiota that associate with healthy plants in nature have great potential for use in sustainable agriculture. Without a better understanding of the mechanisms used by plants to select microbiota, these promising resources will remain untapped.

### BRIEF SUMMARY

In order to better understand the role of EPR3 in EPS perception during nitrogen-fixation symbiosis, a crystal structure of EPR3 was determined. Surprisingly, this crystal structure identified EPR3 as a member of a new class of EPS receptors that shared the same overall architecture and was conserved in dicots (legumes and non-legumes) as well as in monocots (e.g., cereals) demonstrating that this new class of receptors must have a larger role than merely as a second gate in symbiosis with nitrogen-fixing bacteria. Further, an EPR3 receptor homolog, EPR3a, was identified in L. japonicus, and surprisingly shown to also be important for the bacterial infection process in root nodulation. In addition, it was also surprisingly found that epr3 mutant L. japonicus plants had significantly reduced fitness when grown in natural soil, and that these mutant plants assembled a bacterial community distinct from that of wild-type plants. These results indicated that EPS signaling through this new class of EPR3 receptors was crucial for plant growth in a microbe-rich environment, and that the larger role for this new class of EPR3 receptors was for selection of beneficial commensal microbes to organize a healthy microbiota around the roots and rhizosphere of plants generally. The surprising identification of a new class of EPS receptors conserved across dicots and monocots, the identification of the EPR3 receptor homolog EPR3a, and the role of EPR3-mediated EPS signaling in selecting microbiota identified by the inventors serves as the basis for many of the aspects and their various embodiments of the present disclosure.

This determination of the role of the EPR3 and EPR3a receptors will allow persons of skill in the art to genetically alter plants to allow the plant to recognize and select for different beneficial commensal microbes, for example by adding a heterologous EPR3 or EPR3a receptor from a plant that recognizes and selects for the different beneficial commensal microbes or by modification of the ectodomain of the endogenous EPR3 or EPR3a receptors to alter the selectivity for a select beneficial commensal microbe. In addition, this determination allows one of skill in the art to identify beneficial commensal microbes that can interact with a plant by screening beneficial commensal microbes or samples of their exopolysaccharides for the ability to bind to the EPR3 or EPR3a receptor ectodomain or to induce signaling. These beneficial commensal microbes can then be used to enhance cultivation of the plant through seed treatments and the like.

An aspect of the disclosure includes a genetically altered plant or part thereof including a first nucleic acid sequence encoding a heterologous EPR3 or EPR3-like polypeptide or a modified EPR3 or EPR3-like polypeptide, wherein the heterologous EPR3 or EPR3-like polypeptide or the modified EPR3 or EPR3-like polypeptide provides increased selectivity for a beneficial commensal microbe as compared to a wild-type plant under the same conditions. An additional embodiment of this aspect includes the plant or part thereof further including a second nucleic acid sequence encoding a heterologous EPR3a or EPR3a-like polypeptide or a modified EPR3a or EPR3a-like polypeptide. In a further embodiment of this aspect, which may be combined with any of the preceding embodiments, the heterologous EPR3 or EPR3-like polypeptide is selected from the group of a first polypeptide with at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 1 [L. japonicus (BAI79269.1)], a second polypeptide with at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 2 [Chickpea (XP_004489790.1)], a third polypeptide with at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 3 [Medicago (XP_003613165.1)], a fourth polypeptide with at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 4 [Soybean (XP_003517716.1)], a fifth polypeptide with at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 5 [Phaseolus (XP_007157313.1)], a sixth polypeptide with at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 6 [Populus (XP_002322185.1)], a seventh polypeptide with at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 7 [Malus (XP_008340354.1)], an eighth polypeptide with at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 8 [Vitis (XP_002272814.2)], a ninth polypeptide with at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 9 [Theobroma (XP_007036352.1)], a tenth polypeptide with at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 10 [Ricinus (XP_002527912.1)], an eleventh polypeptide with at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 11 [Fragaria (XP_004300916.1)], a twelfth polypeptide with at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 12 [Maize (XP_008657477.1)], a thirteenth polypeptide with at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 13 [Rice (XP_015628733.1)], a fourteenth polypeptide with at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 14 [Wheat (CDM80098.1)], or a fifteenth polypeptide with at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 15 [Barley (MLOC_5489.2)]. Yet another embodiment of this aspect includes the heterologous EPR3 or EPR3-like polypeptide being selected from the group of SEQ ID NO: 1 [L. japonicus (EPR3)], SEQ ID NO: 2 [Chickpea (XP_004489790.1)], SEQ ID NO: 3 [Medicago (XP_003613165.1)], SEQ ID NO: 4 [Soybean (XP_003517716.1)], SEQ ID NO: 5 [Phaseolus (XP_007157313.1)], SEQ ID NO: 6 [Populus (XP_002322185.1)], SEQ ID NO: 7 [Malus (XP_008340354.1)], SEQ ID NO: 8 [Vitis (XP_002272814.2)], SEQ ID NO: 9 [Theobroma (XP_007036352.1)], SEQ ID NO: 10 [Ricinus (XP_002527912.1)], SEQ ID NO: 11 [Fragaria (XP_004300916.1)], SEQ ID NO: 12 [Maize (XP_008657477.1)], SEQ ID NO: 13 [Rice (XP_015628733.1)], SEQ ID NO: 14 [Wheat (CDM80098.1)], or SEQ ID NO: 15 [Barley (MLOC_5489.2)]. Still another embodiment of this aspect, which may be combined with any of the preceding embodiments that has a heterologous EPR3a or EPR3a-like polypeptide, the heterologous EPR3a or EPR3a-like polypeptide is selected from the group of a polypeptide with at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 62 [L. japonicus (EPR3a)], SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, or SEQ ID NO: 92. A further embodiment of this aspect includes the heterologous EPR3a or EPR3a-like polypeptide being SEQ ID NO: 62 [L. japonicus (EPR3a)], SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, or SEQ ID NO: 92.

Still another embodiment of this aspect, which may be combined with any of the preceding embodiments, the modified EPR3 or EPR3-like polypeptide comprises a modified ectodomain that has been replaced with all or a portion of an ectodomain of the heterologous EPR3 or EPR3-like polypeptide, optionally all or a part of the M1 domain, the M2 domain, the LysM3 domain, or all three. In an additional embodiment of this aspect, the portion replaced is at least 10%, at least 20%, at least 30%, at least 40%, at least 50%, at least 60%, at least 70%, at least 80%, at least 90%, less than 10%, less than 20%, less than 30%, less than 40%, less than 50%, less than 60%, less than 70%, less than 80%, or less than 90%, of the ectodomain or, optionally all or a part of the M1 domain, the M2 domain, the LysM3 domain, or all three. In yet another embodiment of this aspect, which may be combined with any of the preceding embodiments that have an EPR3a or EPR3a-like polypeptide, the modified EPR3a or EPR3a-like polypeptide includes a modified ectodomain that has been replaced with all or a portion of an ectodomain of the heterologous EPR3a or EPR3a-like polypeptide, optionally all or a part of the M1 domain, the M2 domain, the LysM3 domain, or all three. In an additional embodiment of this aspect, the portion replaced is at least 10%, at least 20%, at least 30%, at least 40%, at least 50%, at least 60%, at least 70%, at least 80%, at least 90%, less than 10%, less than 20%, less than 30%, less than 40%, less than 50%, less than 60%, less than 70%, less than 80%, or less than 90%, of the ectodomain or, optionally all or a part of the M1 domain, the M2 domain, the LysM3 domain, or all three. A further embodiment of this aspect, which may be combined with any of the preceding embodiments that have an EPR3a or EPR3a-like polypeptide, includes the heterologous EPR3 or EPR3-like polypeptide and the heterologous EPR3a or EPR3a-like polypeptide being from the same plant species or the same plant variety.

Yet another embodiment of this aspect, which may be combined with any of the preceding embodiments, includes the expression of the heterologous EPR3 or EPR3-like polypeptide or the modified EPR3 or EPR3-like polypeptide allowing the plant or part thereof to recognize an exopolysaccharide (EPS), a beta-glucan, a cyclic beta-glucan, a LPS, or a surface carbohydrate produced by the microbe. Still another embodiment of this aspect, which may be combined with any of the preceding embodiments that have an EPR3a or EPR3a-like polypeptide, includes the expression of the heterologous EPR3a or EPR3a-like polypeptide or the modified EPR3a or EPR3a-like polypeptide allowing the plant or part thereof to recognize an EPS, a beta-glucan, a cyclic beta-glucan, a LPS, or a surface carbohydrate produced by the microbe. In a further embodiment of this aspect, the expression of the heterologous EPR3 or EPR3-like polypeptide or the modified EPR3 or EPR3-like polypeptide and the expression of the heterologous EPR3a or EPR3a-like polypeptide or the modified EPR3a or EPR3a-like polypeptide allows the plant or part thereof to recognize an EPS, a beta-glucan, a cyclic beta-glucan, a LPS, or a surface carbohydrate produced by the microbe. In an additional embodiment of this aspect that can be combined with any preceding embodiments including an EPS produced by the microbe, the microbe being a commensal bacteria, optionally a nitrogen-fixing bacteria, or a mycorrhizal fungi. A further embodiment of this aspect includes the nitrogen-fixing bacteria being selected from the group of Mesorhizobium loti, Mesorhizobium huakuii, Mesorhizobium mediterraneum, Mesorhizobium ciceri, Mesorhizobium spp., Rhizobium mongolense, Rhizobium tropici, Rhizobium etli phaseoli, Rhizobium giardinii, Rhizobium leguminosarum optionally R. leguminosarum trifolii, R. leguminosarum viciae, and R. leguminosarum phaseoli, Burkholderiales optionally symbionts of Mimosa, Sinorhizobium meliloti, Sinorhizobium medicae, Sinorhizobium fredii, Sinorhizobium fredii NGR234, Azorhizobium caulinodans, Bradyrhizobium japonicum, Bradyrhizobium elkanii, Bradyrhizobium liaonginense, Rhizobium spp., Mesorhizobium spp., Sinorhizobium spp., Azorhizobium spp. Frankia spp., or any combination thereof, or the mycorrhizal fungi being selected from the group of Acaulosporaceae spp., Diversisporaceae spp., Gigasporaceae spp., Pacisporaceae spp., Funneliformis spp., Glomus spp., Rhizophagus spp., Sclerocystis spp., Septoglomus spp., Claroideoglomus spp., Ambispora spp., Archaeospora spp., Geosiphon pyriformis, Paraglomus spp., other species in the division Glomeromycota, or any combination thereof. Still another embodiment of this aspect, which may be combined with any preceding embodiments, includes the heterologous EPR3 or EPR3-like polypeptide or the modified EPR3 or EPR3-like polypeptide being localized to a plant cell plasma membrane. Yet another embodiment of this aspect, which may be combined with any of the preceding embodiments that have an EPR3a or EPR3a-like polypeptide, includes the heterologous EPR3a or EPR3a-like polypeptide or the modified EPR3a or EPR3a-like polypeptide being localized to a plant cell plasma membrane. A further embodiment of this aspect that can be combined with any of the preceding embodiments that have localization to a plant cell plasma membrane includes the plant cell being a root cell. An additional embodiment of this aspect includes the root cell being a root epidermal cell or a root cortex cell. In a further embodiment of this aspect that can be combined with any of the preceding embodiments, the heterologous EPR3 or EPR3-like polypeptide or the modified EPR3 or EPR3-like polypeptide is expressed in a developing plant root system. In an additional embodiment of this aspect that can be combined with any of the preceding embodiments that has an EPR3a or EPR3a-like polypeptide, the heterologous EPR3a or EPR3a-like polypeptide or the modified EPR3a or EPR3a-like polypeptide is expressed in a developing plant root system.

In still another embodiment of this aspect, which may be combined with any of the preceding embodiments, the first nucleic acid sequence is operably linked to a first promoter. In an additional embodiment of this aspect, the first promoter is a root specific promoter, and the root specific promoter is optionally selected from the group of a NFR1 or NFR5/NFP promoter, an EPR3 or an EPR3a promoter, a Lotus NFR5 promoter, a Lotus NFR1 promoter, a maize allothioneine promoter, a chitinase promoter, a maize ZRP2 promoter, a tomato LeExt1 promoter, a glutamine synthetase soybean root promoter, a RCC3 promoter, a rice antiquitine promoter, a LRR receptor kinase promoter, or an Arabidopsis pCO2 promoter. In a further embodiment of this aspect, the first promoter is a constitutive promoter, and the constitutive promoter is optionally selected from the group of a CaMV35S promoter, a derivative of the CaMV35S promoter, a maize ubiquitin promoter, a trefoil promoter, a vein mosaic cassava virus promoter, or an Arabidopsis UBQ10 promoter. In yet another embodiment of this aspect, which may be combined with any of the preceding embodiments that has an EPR3a or EPR3a-like polypeptide, the second nucleic acid sequence is operably linked to a second promoter. In an additional embodiment of this aspect, the second promoter is a root specific promoter, and the root specific promoter is optionally selected from the group of a NFR1 or NFR5/NFP promoter, an EPR3 or an EPR3a promoter, a Lotus NFR5 promoter, a Lotus NFR1 promoter, a maize allothioneine promoter, a chitinase promoter, a maize ZRP2 promoter, a tomato LeExt1 promoter, a glutamine synthetase soybean root promoter, a RCC3 promoter, a rice antiquitine promoter, a LRR receptor kinase promoter, or an Arabidopsis pCO2 promoter. In a further embodiment of this aspect, the second promoter is a constitutive promoter, and the constitutive promoter is optionally selected from the group of a CaMV35S promoter, a derivative of the CaMV35S promoter, a maize ubiquitin promoter, a trefoil promoter, a vein mosaic cassava virus promoter, or an Arabidopsis UBQ10 promoter. In an additional embodiment of this aspect, which may be combined with any of the preceding embodiments, the plant is selected from the group of cassava, corn, cowpea, rice, barley, wheat, Trema spp., apple, pear, plum, apricot, peach, almond, walnut, strawberry, raspberry, blackberry, red currant, black currant, melon, cucumber, pumpkin, squash, grape, tomato, pepper, or hemp. In yet another embodiment of this aspect, which may be combined with any of the preceding embodiments, the plant lacks functional rhizobial Nod factor receptors. In still another embodiment of this aspect, which may be combined with any of the preceding embodiments, the plant is not a legume. In an additional embodiment of this aspect, which may be combined with any of the preceding embodiments, the plant is not A. thaliana, N. tabacum, L. japonicus, or M. truncatula. In a further embodiment of this aspect, which may be combined with any of the above embodiments, the plant part is a leaf, a stem, a root, a root primordia, a flower, a seed, a fruit, a kernel, a grain, a cell, or a portion thereof. An additional embodiment of this aspect includes the plant part being a fruit, a kernel, or a grain.

In some aspects, the present disclosure relates to a pollen grain or an ovule of the genetically altered plant of any of the above embodiments.

In some aspects, the present disclosure relates to a protoplast produced from the plant of any of the above embodiments.

In some aspects, the present disclosure relates to a tissue culture produced from protoplasts or cells from the plant of any of the above embodiments, wherein the cells or protoplasts are produced from a plant part selected from the group of leaf, anther, pistil, stem, petiole, root, root primordia, root tip, fruit, seed, flower, cotyledon, hypocotyl, embryo, or meristematic cell.

A further aspect of the present disclosure relates to methods of producing the genetically altered plant of any of the above embodiments, including introducing a genetic alteration to the plant comprising the first nucleic acid sequence encoding the heterologous EPR3 or EPR3-like polypeptide. An additional embodiment of this aspect includes the first nucleic acid sequence being operably linked to a first promoter. Yet another embodiment of this aspect includes the first promoter being a root specific promoter, and the root specific promoter being optionally selected from the group of a NFR1 or NFR5/NFP promoter, an EPR3 or an EPR3a promoter, a Lotus NFR5 promoter, a Lotus NFR1 promoter, a maize allothioneine promoter, a chitinase promoter, a maize ZRP2 promoter, a tomato LeExt1 promoter, a glutamine synthetase soybean root promoter, a RCC3 promoter, a rice antiquitine promoter, a LRR receptor kinase promoter, or an Arabidopsis pCO2 promoter. Still another embodiment of this aspect includes the first promoter being a constitutive promoter, and the constitutive promoter being optionally selected from the group of a CaMV35S promoter, a derivative of the CaMV35S promoter, a maize ubiquitin promoter, a trefoil promoter, a vein mosaic cassava virus promoter, or an Arabidopsis UBQ10 promoter. An additional embodiment of this aspect further includes introducing a genetic alteration to the plant including the second nucleic acid sequence encoding the heterologous EPR3a or EPR3a-like polypeptide. A further embodiment of this aspect includes the second nucleic acid sequence being operably linked to a second promoter. Yet another embodiment of this aspect includes the second promoter being a root specific promoter, and the root specific promoter being optionally selected from the group of a NFR1 or NFR5/NFP promoter, an EPR3 or an EPR3a promoter, a Lotus NFR5 promoter, a Lotus NFR1 promoter, a maize allothioneine promoter, a chitinase promoter, a maize ZRP2 promoter, a tomato LeExt1 promoter, a glutamine synthetase soybean root promoter, a RCC3 promoter, a rice antiquitine promoter, a LRR receptor kinase promoter, or an Arabidopsis pCO2 promoter. Still another embodiment of this aspect includes the second promoter being a constitutive promoter, and the constitutive promoter being optionally selected from the group consisting of a CaMV35S promoter, a derivative of the CaMV35S promoter, a maize ubiquitin promoter, a trefoil promoter, a vein mosaic cassava virus promoter, or an Arabidopsis UBQ10 promoter. In a further embodiment of this aspect, with may be combined with any of the preceding embodiments, the first nucleic acid sequence is inserted into the genome of the plant so that the nucleic acid sequence is operably linked to a first endogenous promoter. An additional embodiment of this aspect includes the first endogenous promoter being a root specific promoter. In yet another embodiment of this aspect, with may be combined with any of the preceding embodiments that has the second nucleic acid sequence, the second nucleic acid sequence is inserted into the genome of the plant so that the nucleic acid sequence is operably linked to a second endogenous promoter. A further embodiment of this aspect includes the second endogenous promoter being a root specific promoter.

An additional aspect of the present disclosure relates to methods of producing the genetically altered plant of any one of the preceding embodiments that have a modified polypeptide, including genetically editing a gene encoding an endogenous LysM receptor polypeptide in the plant to comprise the modified ectodomain. In a further embodiment of this aspect, the endogenous LysM receptor polypeptide is an endogenous EPR3 or EPR3-like polypeptide. In another embodiment of this aspect, which may be combined with any of the preceding embodiments, the modified EPR3 or EPR3-like polypeptide was generated by: (a) providing a heterologous EPR3 or EPR3-like polypeptide model including a structural model, a molecular model, a surface characteristics model, and/or an electrostatic potential model of a M1 domain, a M2 domain, a LysM3 domain, any combination thereof, or the ectodomain of the heterologous EPR3 or EPR3-like polypeptide having selectivity for the beneficial commensal microbe and an unmodified EPR3 or EPR3-like polypeptide; (b) identifying one or more amino acid residues for modification in the unmodified EPR3 or EPR3-like polypeptide by comparing amino acid residues of a oligosaccharide binding feature in the unmodified EPR3 or EPR3-like polypeptide with the corresponding amino acid residues in the heterologous EPR3 or EPR3-like polypeptide model; and (c) generating the unmodified EPR3 or EPR3-like polypeptide wherein the one or more amino acid residues in the oligosaccharide binding feature of the unmodified EPR3 or EPR3-like polypeptide have been substituted with corresponding amino acid residues from the heterologous EPR3 or EPR3-like polypeptide. Yet another embodiment of this aspect includes the heterologous EPR3 or EPR3-like polypeptide model being a protein crystal structure, a molecular model, a cryo-EM structure, or a NMR structure. In an additional embodiment of this aspect, the endogenous LysM receptor polypeptide is an endogenous EPR3a or EPR3a-like polypeptide. In another embodiment of this aspect, the modified EPR3a or EPR3a-like polypeptide was generated by: (a) providing a heterologous EPR3a or EPR3a-like polypeptide model including a structural model, a molecular model, a surface characteristics model, and/or an electrostatic potential model of a M1 domain, a M2 domain, a LysM3 domain, any combination thereof, or the ectodomain of the heterologous EPR3a or EPR3a-like polypeptide having selectivity for the beneficial commensal microbe and an unmodified EPR3a or EPR3a-like polypeptide; (b) identifying one or more amino acid residues for modification in the unmodified EPR3a or EPR3a-like polypeptide by comparing amino acid residues of a oligosaccharide binding feature in the unmodified EPR3a or EPR3a-like polypeptide with the corresponding amino acid residues in the heterologous EPR3a or EPR3a-like polypeptide model; and (c) generating the unmodified EPR3a or EPR3a-like polypeptide wherein the one or more amino acid residues in the oligosaccharide binding feature of the unmodified EPR3a or EPR3a-like polypeptide have been substituted with corresponding amino acid residues from the heterologous EPR3a or EPR3a-like polypeptide. Yet another embodiment of this aspect includes the heterologous EPR3a or EPR3a-like polypeptide model being a protein crystal structure, a molecular model, a cryo-EM structure, or a NMR structure. A further embodiment of this aspect that can be combined with any of the preceding embodiments includes a plant or plant part produced by the method of any one of the preceding embodiments.

An additional aspect of the disclosure includes a genetically altered plant or part thereof including a first nucleic acid sequence encoding a heterologous EPR3a or EPR3a-like polypeptide or a modified EPR3a or EPR3a-like polypeptide, wherein the heterologous EPR3a or EPR3a-like polypeptide or the modified EPR3a or EPR3a-like polypeptide provides increased selectivity for a beneficial commensal microbe as compared to a wild-type plant under the same conditions. An additional embodiment of this aspect includes the plant or part thereof further including a second nucleic acid sequence encoding a heterologous EPR3 or EPR3-like polypeptide or a modified EPR3 or EPR3-like polypeptide. In a further embodiment of this aspect, which may be combined with any of the preceding embodiments, the heterologous EPR3a or EPR3a-like polypeptide is selected from the group of a polypeptide with at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 62 [L. japonicus (EPR3a)], SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, or SEQ ID NO: 92. A further embodiment of this aspect includes the heterologous EPR3a or EPR3a-like polypeptide being SEQ ID NO: 62 [L. japonicus (EPR3a)], SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, or SEQ ID NO: 92. In still another embodiment of this aspect, which may be combined with any of the preceding embodiments having the heterologous EPR3 or EPR3-like polypeptide, the heterologous EPR3 or EPR3-like polypeptide is selected from the group of a polypeptide with at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 1 [L. japonicus (BAI79269.1)], a second polypeptide with at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 2 [Chickpea (XP_004489790.1)], a third polypeptide with at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 3 [Medicago (XP_003613165.1)], a fourth polypeptide with at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 4 [Soybean (XP_003517716.1)], a fifth polypeptide with at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 5 [Phaseolus (XP_007157313.1)], a sixth polypeptide with at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 6 [Populus (XP_002322185.1)], a seventh polypeptide with at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 7 [Malta (XP_008340354.1)], an eighth polypeptide with at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 8 [Vitis (XP_002272814.2)], a ninth polypeptide with at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 9 [Theobroma (XP_007036352.1)], a tenth polypeptide with at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 10 [Ricinus (XP_002527912.1)], an eleventh polypeptide with at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 11 [Fragaria (XP_004300916.1)], a twelfth polypeptide with at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 12 [Maize (XP_008657477.1)], a thirteenth polypeptide with at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 13 [Rice (XP_015628733.1)], a fourteenth polypeptide with at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 14 [Wheat (CDM80098.1)], or a fifteenth polypeptide with at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 15 [Barley (MLOC_5489.2)]. A further embodiment of this aspect includes the heterologous EPR3 or EPR3-like polypeptide being selected from the group of SEQ ID NO: 1 [L. japonicus (EPR3)], SEQ ID NO: 2 [Chickpea (XP_004489790.1)], SEQ ID NO: 3 [Medicago (XP_003613165.1)], SEQ ID NO: 4 [Soybean (XP_003517716.1)], SEQ ID NO: 5 [Phaseolus (XP_007157313.1)], SEQ ID NO: 6 [Populus (XP_002322185.1)], SEQ ID NO: 7 [Malus (XP_008340354.1)], SEQ ID NO: 8 [Vitis (XP_002272814.2)], SEQ ID NO: 9 [Theobroma (XP_007036352.1)], SEQ ID NO: 10 [Ricinus (XP_002527912.1)], SEQ ID NO: 11 [Fragaria (XP_004300916.1)], SEQ ID NO: 12 [Maize (XP_008657477.1)], SEQ ID NO: 13 [Rice (XP_015628733.1)], SEQ ID NO: 14 [Wheat (CDM80098.1)], or SEQ ID NO: 15 [Barley (MLOC_5489.2)].

Still another embodiment of this aspect, which may be combined with any of the preceding embodiments, the modified EPR3a or EPR3a-like polypeptide comprises a modified ectodomain that has been replaced with all or a portion of an ectodomain of the heterologous EPR3a or EPR3a-like polypeptide, optionally all or a part of the M1 domain, the M2 domain, the LysM3 domain, or all three. In an additional embodiment of this aspect, the portion replaced is at least 10%, at least 20%, at least 30%, at least 40%, at least 50%, at least 60%, at least 70%, at least 80%, at least 90%, less than 10%, less than 20%, less than 30%, less than 40%, less than 50%, less than 60%, less than 70%, less than 80%, or less than 90%, of the ectodomain or, optionally all or a part of the M1 domain, the M2 domain, the LysM3 domain, or all three. In yet another embodiment of this aspect, which may be combined with any of the preceding embodiments that have an EPR3 or EPR3-like polypeptide, the modified EPR3 or EPR3-like polypeptide includes a modified ectodomain that has been replaced with all or a portion of an ectodomain of the heterologous EPR3 or EPR3-like polypeptide, optionally all or a part of the M1 domain, the M2 domain, the LysM3 domain, or all three. In an additional embodiment of this aspect, the portion replaced is at least 10%, at least 20%, at least 30%, at least 40%, at least 50%, at least 60%, at least 70%, at least 80%, at least 90%, less than 10%, less than 20%, less than 30%, less than 40%, less than 50%, less than 60%, less than 70%, less than 80%, or less than 90%, of the ectodomain or, optionally all or a part of the M1 domain, the M2 domain, the LysM3 domain, or all three. A further embodiment of this aspect, which may be combined with any of the preceding embodiments that have an EPR3 or EPR3-like polypeptide, includes the heterologous EPR3a or EPR3a-like polypeptide and the heterologous EPR3 or EPR3-like polypeptide being from the same plant species or the same plant variety.

Yet another embodiment of this aspect, which may be combined with any of the preceding embodiments, includes the expression of the heterologous EPR3a or EPR3a-like polypeptide or the modified EPR3a or EPR3a-like polypeptide allowing the plant or part thereof to recognize an EPS, a beta-glucan, a cyclic beta-glucan, a LPS, or a surface carbohydrate produced by the microbe. Still another embodiment of this aspect, which may be combined with any of the preceding embodiments that have an EPR3 or EPR3-like polypeptide, includes the expression of the heterologous EPR3 or EPR3-like polypeptide or the modified EPR3 or EPR3-like polypeptide allowing the plant or part thereof to recognize an EPS, a beta-glucan, a cyclic beta-glucan, a LPS, or a surface carbohydrate produced by the microbe. In a further embodiment of this aspect, the expression of the heterologous EPR3a or EPR3a-like polypeptide or the modified EPR3a or EPR3a-like polypeptide and the expression of the heterologous EPR3 or EPR3-like polypeptide or the modified EPR3 or EPR3-like polypeptide allows the plant or part thereof to recognize an EPS, a beta-glucan, a cyclic beta-glucan, a LPS, or a surface carbohydrate produced by the microbe. In an additional embodiment of this aspect that can be combined with any preceding embodiments including an EPS produced by the microbe, the microbe is a commensal bacteria, optionally a nitrogen-fixing bacteria, or a mycorrhizal fungi. A further embodiment of this aspect includes the nitrogen-fixing bacteria being selected from the group of Mesorhizobium loti, Mesorhizobium huakuii, Mesorhizobium mediterraneum, Mesorhizobium ciceri, Mesorhizobium spp., Rhizobium mongolense, Rhizobium tropici, Rhizobium etli phaseoli, Rhizobium giardinii, Rhizobium leguminosarum optionally R. leguminosarum trifolii, R. leguminosarum viciae, and R. leguminosarum phaseoli, Burkholderiales optionally symbionts of Mimosa, Sinorhizobium meliloti, Sinorhizobium medicae, Sinorhizobium fredii, Sinorhizobium fredii NGR234, Azorhizobium caulinodans, Bradyrhizobium japonicum, Bradyrhizobium elkanii, Bradyrhizobium liaonginense, Rhizobium spp., Mesorhizobium spp., Sinorhizobium spp., Azorhizobium spp. Frankia spp., or any combination thereof, or the mycorrhizal fungi being selected from the group of Acaulosporaceae spp., Diversisporaceae spp., Gigasporaceae spp., Pacisporaceae spp., Funneliformis spp., Glomus spp., Rhizophagus spp., Sclerocystis spp., Septoglomus spp., Claroideoglomus spp., Ambispora spp., Archaeospora spp., Geosiphon pyriformis, Paraglomus spp., other species in the division Glomeromycota, or any combination thereof. Still another embodiment of this aspect, which may be combined with any preceding embodiments, includes the heterologous EPR3a or EPR3a-like polypeptide or the modified EPR3a or EPR3a-like polypeptide being localized to a plant cell plasma membrane. Yet another embodiment of this aspect, which may be combined with any of the preceding embodiments that have an EPR3 or EPR3-like polypeptide, includes the heterologous EPR3 or EPR3-like polypeptide or the modified EPR3 or EPR3-like polypeptide being localized to a plant cell plasma membrane. A further embodiment of this aspect that can be combined with any of the preceding embodiments that have localization to a plant cell plasma membrane includes the plant cell being a root cell. An additional embodiment of this aspect includes the root cell being a root epidermal cell or a root cortex cell. In a further embodiment of this aspect that can be combined with any of the preceding embodiments, the heterologous EPR3a or EPR3a-like polypeptide or the modified EPR3a or EPR3a-like polypeptide is expressed in a developing plant root system. In an additional embodiment of this aspect that can be combined with any of the preceding embodiments that has an EPR3 or EPR3-like polypeptide, the heterologous EPR3 or EPR3-like polypeptide or the modified EPR3 or EPR3-like polypeptide is expressed in a developing plant root system.

In still another embodiment of this aspect, which may be combined with any of the preceding embodiments, the first nucleic acid sequence is operably linked to a first promoter. In an additional embodiment of this aspect, the first promoter is a root specific promoter, and the root specific promoter is optionally selected from the group of a NFR1 or NFR5/NFP promoter, an EPR3 or an EPR3a promoter, a Lotus NFR5 promoter, a Lotus NFR1 promoter, a maize allothioneine promoter, a chitinase promoter, a maize ZRP2 promoter, a tomato LeExt1 promoter, a glutamine synthetase soybean root promoter, a RCC3 promoter, a rice antiquitine promoter, a LRR receptor kinase promoter, or an Arabidopsis pCO2 promoter. In a further embodiment of this aspect, the first promoter is a constitutive promoter, and the constitutive promoter is optionally selected from the group of a CaMV35S promoter, a derivative of the CaMV35S promoter, a maize ubiquitin promoter, a trefoil promoter, a vein mosaic cassava virus promoter, or an Arabidopsis UBQ10 promoter. In yet another embodiment of this aspect, which may be combined with any of the preceding embodiments that has an EPR3 or EPR3-like polypeptide, the second nucleic acid sequence is operably linked to a second promoter. In an additional embodiment of this aspect, the second promoter is a root specific promoter, and the root specific promoter is optionally selected from the group of a NFR1 or NFR5/NFP promoter, an EPR3 or an EPR3a promoter, a Lotus NFR5 promoter, a Lotus NFR1 promoter, a maize allothioneine promoter, a chitinase promoter, a maize ZRP2 promoter, a tomato LeExt1 promoter, a glutamine synthetase soybean root promoter, a RCC3 promoter, a rice antiquitine promoter, a LRR receptor kinase promoter, or an Arabidopsis pCO2 promoter. In a further embodiment of this aspect, the second promoter is a constitutive promoter, and the constitutive promoter is optionally selected from the group of a CaMV35S promoter, a derivative of the CaMV35S promoter, a maize ubiquitin promoter, a trefoil promoter, a vein mosaic cassava virus promoter, or an Arabidopsis UBQ10 promoter. In an additional embodiment of this aspect, which may be combined with any of the preceding embodiments, the plant is selected from the group of cassava, corn, cowpea, rice, barley, wheat, Trema spp., apple, pear, plum, apricot, peach, almond, walnut, strawberry, raspberry, blackberry, red currant, black currant, melon, cucumber, pumpkin, squash, grape, tomato, pepper, or hemp. In yet another embodiment of this aspect, which may be combined with any of the preceding embodiments, the plant lacks functional rhizobial Nod factor receptors. In still another embodiment of this aspect, which may be combined with any of the preceding embodiments, the plant is not a legume. In an additional embodiment of this aspect, which may be combined with any of the preceding embodiments, the plant is not A. thaliana, N. tabacum, L. japonicus, or/14. truncatula. In a further embodiment of this aspect, which may be combined with any of the above embodiments, the plant part is a leaf, a stem, a root, a root primordia, a flower, a seed, a fruit, a kernel, a grain, a cell, or a portion thereof. An additional embodiment of this aspect includes the plant part being a fruit, a kernel, or a grain.

In some aspects, the present disclosure relates to a pollen grain or an ovule of the genetically altered plant of any of the above embodiments.

In some aspects, the present disclosure relates to a protoplast produced from the plant of any of the above embodiments.

In some aspects, the present disclosure relates to a tissue culture produced from protoplasts or cells from the plant of any of the above embodiments, wherein the cells or protoplasts are produced from a plant part selected from the group of leaf, anther, pistil, stem, petiole, root, root primordia, root tip, fruit, seed, flower, cotyledon, hypocotyl, embryo, or meristematic cell.

A further aspect of the present disclosure relates to methods of producing the genetically altered plant of any of the above embodiments, including introducing a genetic alteration to the plant comprising the first nucleic acid sequence encoding the heterologous EPR3a or EPR3a-like polypeptide. An additional embodiment of this aspect includes the first nucleic acid sequence being operably linked to a first promoter. Yet another embodiment of this aspect includes the first promoter being a root specific promoter, and the root specific promoter being optionally selected from the group of a NFR1 or NFR5/NFP promoter, an EPR3 or an EPR3a promoter, a Lotus NFR5 promoter, a Lotus NFR1 promoter, a maize allothioneine promoter, a chitinase promoter, a maize ZRP2 promoter, a tomato LeExt1 promoter, a glutamine synthetase soybean root promoter, a RCC3 promoter, a rice antiquitine promoter, a LRR receptor kinase promoter, or an Arabidopsis pCO2 promoter. Still another embodiment of this aspect includes the first promoter being a constitutive promoter, and the constitutive promoter being optionally selected from the group of a CaMV35S promoter, a derivative of the CaMV35S promoter, a maize ubiquitin promoter, a trefoil promoter, a vein mosaic cassava virus promoter, or an Arabidopsis UBQ10 promoter. An additional embodiment of this aspect further includes introducing a genetic alteration to the plant including the second nucleic acid sequence encoding the heterologous EPR3 or EPR3-like polypeptide. A further embodiment of this aspect includes the second nucleic acid sequence being operably linked to a second promoter. Yet another embodiment of this aspect includes the second promoter being a root specific promoter, and the root specific promoter being optionally selected from the group of a NFR1 or NFR5/NFP promoter, an EPR3 or an EPR3a promoter, a Lotus NFR5 promoter, a Lotus NFR1 promoter, a maize allothioneine promoter, a chitinase promoter, a maize ZRP2 promoter, a tomato LeExt1 promoter, a glutamine synthetase soybean root promoter, a RCC3 promoter, a rice antiquitine promoter, a LRR receptor kinase promoter, or an Arabidopsis pCO2 promoter. Still another embodiment of this aspect includes the second promoter being a constitutive promoter, and the constitutive promoter being optionally selected from the group consisting of a CaMV35S promoter, a derivative of the CaMV35S promoter, a maize ubiquitin promoter, a trefoil promoter, a vein mosaic cassava virus promoter, or an Arabidopsis UBQ10 promoter. In a further embodiment of this aspect, with may be combined with any of the preceding embodiments, the first nucleic acid sequence is inserted into the genome of the plant so that the nucleic acid sequence is operably linked to a first endogenous promoter. An additional embodiment of this aspect includes the first endogenous promoter being a root specific promoter. In yet another embodiment of this aspect, with may be combined with any of the preceding embodiments that has the second nucleic acid sequence, the second nucleic acid sequence is inserted into the genome of the plant so that the nucleic acid sequence is operably linked to a second endogenous promoter. A further embodiment of this aspect includes the second endogenous promoter being a root specific promoter.

An additional aspect of the present disclosure relates to methods of producing the genetically altered plant of any one of the preceding embodiments that have a modified polypeptide, including genetically editing a gene encoding an endogenous LysM receptor polypeptide in the plant to comprise the modified ectodomain. In a further embodiment of this aspect, the endogenous LysM receptor polypeptide is an endogenous EPR3a or EPR3a-like polypeptide. In another embodiment of this aspect, which may be combined with any of the preceding embodiments, the modified EPR3a or EPR3a-like polypeptide was generated by: (a) providing a heterologous EPR3a or EPR3a-like polypeptide model including a structural model, a molecular model, a surface characteristics model, and/or an electrostatic potential model of a M1 domain, a M2 domain, a LysM3 domain, any combination thereof, or the ectodomain of the heterologous EPR3a or EPR3a-like polypeptide having selectivity for the beneficial commensal microbe and an unmodified EPR3a or EPR3a-like polypeptide; (b) identifying one or more amino acid residues for modification in the unmodified EPR3a or EPR3a-like polypeptide by comparing amino acid residues of a oligosaccharide binding feature in the unmodified EPR3a or EPR3a-like polypeptide with the corresponding amino acid residues in the heterologous EPR3a or EPR3a-like polypeptide model; and (c) generating the unmodified EPR3a or EPR3a-like polypeptide wherein the one or more amino acid residues in the oligosaccharide binding feature of the unmodified EPR3a or EPR3a-like polypeptide have been substituted with corresponding amino acid residues from the heterologous EPR3a or EPR3a-like polypeptide. Yet another embodiment of this aspect includes the heterologous EPR3a or EPR3a-like polypeptide model being a protein crystal structure, a molecular model, a cryo-EM structure, or a NMR structure. In an additional embodiment of this aspect, the endogenous LysM receptor polypeptide is an endogenous EPR3 or EPR3-like polypeptide. In another embodiment of this aspect, the modified EPR3 or EPR3-like polypeptide was generated by: (a) providing a heterologous EPR3 or EPR3-like polypeptide model including a structural model, a molecular model, a surface characteristics model, and/or an electrostatic potential model of a M1 domain, a M2 domain, a LysM3 domain, any combination thereof, or the ectodomain of the heterologous EPR3 or EPR3-like polypeptide having selectivity for the beneficial commensal microbe and an unmodified EPR3 or EPR3-like polypeptide; (b) identifying one or more amino acid residues for modification in the unmodified EPR3 or EPR3-like polypeptide by comparing amino acid residues of a oligosaccharide binding feature in the unmodified EPR3 or EPR3-like polypeptide with the corresponding amino acid residues in the heterologous EPR3 or EPR3-like polypeptide model; and (c) generating the unmodified EPR3 or EPR3-like polypeptide wherein the one or more amino acid residues in the oligosaccharide binding feature of the unmodified EPR3 or EPR3-like polypeptide have been substituted with corresponding amino acid residues from the heterologous EPR3 or EPR3-like polypeptide. Yet another embodiment of this aspect includes the heterologous EPR3 or EPR3-like polypeptide model being a protein crystal structure, a molecular model, a cryo-EM structure, or a NMR structure. A further embodiment of this aspect that can be combined with any of the preceding embodiments includes a plant or plant part produced by the method of any one of the preceding embodiments

Yet another aspect of the present disclosure relates to methods of identifying a beneficial commensal microbe capable of participating in a plant root microbiota including: a) providing a first polypeptide including an EPR3 or EPR3-like polypeptide, an ectodomain of an EPR3 or EPR3-like polypeptide, a M1 domain of an EPR3 or EPR3-like polypeptide, a M2 domain of an EPR3 or EPR3-like polypeptide, or a LysM3 domain of an EPR3 or EPR3-like polypeptide of the plant; b) contacting the first polypeptide with a sample including a microbe or an EPS, a beta-glucan, a cyclic beta-glucan, a LPS, or a surface carbohydrate produced by the microbe; and c) detecting binding of the EPS, the beta-glucan, the cyclic beta-glucan, the LPS, or the surface carbohydrate produced by the microbe to the polypeptide, wherein binding of the EPS, the beta-glucan, the cyclic beta-glucan, the LPS, or the surface carbohydrate to the polypeptide indicates that the microbe is a beneficial commensal bacteria capable of participating in the plant root microbiota; optionally, detecting enrichment of taxa in Burkholderiales and/or Rhizobiales in a plant rhizosphere or endosphere, wherein enrichment of taxa in Burkholderiales and/or Rhizobiales in the plant rhizosphere or endosphere indicates that the microbe is a beneficial commensal microbe capable of participating in a plant root microbiota; optionally, the detecting is by a functional assay optionally selected from (i) detecting enrichment of taxa in Burkholderiales and/or Rhizobiales in a plant rhizosphere or endosphere, wherein enrichment of taxa in Burkholderiales and/or Rhizobiales in the plant rhizosphere or endosphere indicates that the microbe is a beneficial commensal microbe capable of participating in a plant root microbiota; optionally, (ii) detecting nodulation in a plant root system, wherein nodulation indicates that the microbe is a beneficial commensal microbe capable of participating in a plant root microbiota; and/or (iii) detecting mycorrhization in a plant root system, wherein mycorrhization indicates that the microbe is a beneficial commensal microbe capable of participating in a plant root microbiota, or optionally the detecting is by a direct binding assay optionally selected from (1) a competition assay optionally with a known signaling saccharide, or (2) an affinity assay optionally wherein the detected affinity is compared to the affinity for the known signaling saccharide. A further embodiment of this aspect further includes providing a second polypeptide including an EPR3a or EPR3a-like polypeptide, an ectodomain of an EPR3a or EPR3a-like polypeptide, a M1 domain of an EPR3a or EPR3a-like polypeptide, a M2 domain of an EPR3a or EPR3a-like polypeptide, or a LysM3 domain of an EPR3a or EPR3a-like polypeptide of the plant of the plant in step (a), wherein the second polypeptide is in contact with the first polypeptide. An additional embodiment of this aspect further includes step (d) culturing the beneficial commensal microbe if binding is detected in step (c). Yet another embodiment of this aspect further includes step (e) applying the beneficial commensal microbe to the plant or a part thereof. A further embodiment of this aspect includes the plant part being a plant propagation material, optionally a seed, a tuber, or a plantlet, and the beneficial commensal microbe being applied to the plant propagation material, optionally to the seed as part of a seed coating, to the tuber, or to a root of the plantlet. An additional embodiment of this aspect includes the plant part being a plant vegetative or reproductive material, optionally a root, a shoot, a stem, a pollen grain, or an ovule, and the beneficial commensal microbe is applied to the plant vegetative or reproductive material of the plant, optionally as part of a coating, a solution, or a powder. Still another embodiment of this aspect further includes step (e) applying the beneficial commensal microbe, optionally in admixture with a soil-compatible carrier, a fungal carrier, or a growth medium, optionally soil, where the plant is growing or is to be grown. Yet another embodiment of this aspect, which may be combined with any of the preceding embodiments having an ectodomain of an EPR3 or EPR3-like polypeptide, includes the ectodomain of the EPR3 or EPR3-like polypeptide having at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to the ectodomain of SEQ ID NO: 1 [L. japonicus (EPR3)], SEQ ID NO: 2 [Chickpea (XP_004489790.1)], SEQ ID NO: 3 [Medicago (XP_003613165.1)], SEQ ID NO: 4 [Soybean (XP_003517716.1)], SEQ ID NO: 5 [Phaseolus (XP_007157313.1)], SEQ ID NO: 6 [Populus (XP_002322185.1)], SEQ ID NO: 7 [Malus (XP_008340354.1)], SEQ ID NO: 8 [Vitis (XP_002272814.2)], SEQ ID NO: 9 [Theobroma (XP_007036352.1)], SEQ ID NO: 10 [Ricinus (XP_002527912.1)], SEQ ID NO: 11 [Fragaria (XP_004300916.1)], SEQ ID NO: 12 [Maize (XP_008657477.1)], SEQ ID NO: 13 [Rice (XP_015628733.1)], SEQ ID NO: 14 [Wheat (CDM80098.1)], or SEQ ID NO: 15 [Barley (MLOC_5489.2)]. An additional embodiment of this aspect, which may be combined with any of the preceding embodiments having an ectodomain of an EPR3 or EPR3-like polypeptide, includes the ectodomain of the EPR3 or EPR3-like polypeptide being the ectodomain of SEQ ID NO: 1 [L. japonicus (EPR3)], SEQ ID NO: 2 [Chickpea (XP_004489790.1)], SEQ ID NO: 3 [Medicago (XP_003613165.1)], SEQ ID NO: 4 [Soybean (XP_003517716.1)], SEQ ID NO: 5 [Phaseolus (XP_007157313.1)], SEQ ID NO: 6 [Populus (XP_002322185.1)], SEQ ID NO: 7 [Malus (XP_008340354.1)], SEQ ID NO: 8 [Vitis (XP_002272814.2)], SEQ ID NO: 9 [Theobroma (XP_007036352.1)], SEQ ID NO: 10 [Ricinus (XP_002527912.1)], SEQ ID NO: 11 [Fragaria (XP_004300916.1)], SEQ ID NO: 12 [Maize (XP_008657477.1)], SEQ ID NO: 13 [Rice (XP_015628733.1)], SEQ ID NO: 14 [Wheat (CDM80098.1)], or SEQ ID NO: 15 [Barley (MLOC_5489.2)]. A further embodiment of this aspect, which may be combined with any of the preceding embodiments having an ectodomain of an EPR3a or EPR3a-like polypeptide, includes the ectodomain of the EPR3a or EPR3a-like polypeptide having at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to the ectodomain of SEQ ID NO: 62 [L. japonicus (EPR3a)], SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, or SEQ ID NO: 92. Yet another embodiment of this aspect, which may be combined with any of the preceding embodiments having an ectodomain of an EPR3a or EPR3a-like polypeptide, includes the ectodomain of the EPR3a or EPR3a-like polypeptide being the ectodomain of SEQ ID NO: 62 [L. japonicus (EPR3a)], SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, or SEQ ID NO: 92. Still another embodiment of this aspect includes the beneficial commensal microbe being a commensal bacteria, optionally a nitrogen-fixing bacteria, or a mycorrhizal fungi.

Still another aspect of the present disclosure relates to methods of identifying a beneficial commensal microbe capable of participating in a plant root microbiota including: a) providing a first polypeptide including an EPR3a or EPR3a-like polypeptide, an ectodomain of an EPR3a or EPR3a-like polypeptide, a M1 domain of an EPR3a or EPR3a-like polypeptide, a M2 domain of an EPR3a or EPR3a-like polypeptide, or a LysM3 domain of an EPR3a or EPR3a-like polypeptide of the plant; b) contacting the first polypeptide with a sample including a microbe or an EPS, a beta-glucan, a cyclic beta-glucan, a LPS, or a surface carbohydrate produced by the microbe; and c) detecting binding of the EPS, the beta-glucan, the cyclic beta-glucan, the LPS, or the surface carbohydrate produced by the microbe to the polypeptide, wherein binding of the EPS, the beta-glucan, the cyclic beta-glucan, the LPS, or the surface carbohydrate to the polypeptide indicates that the microbe is a beneficial commensal microbe capable of participating in a plant root microbiota; optionally, detecting enrichment of taxa in Burkholderiales and/or Rhizobiales in a plant rhizosphere or endosphere, wherein enrichment of taxa in Burkholderiales and/or Rhizobiales in the plant rhizosphere or endosphere indicates that the microbe is a beneficial commensal microbe capable of participating in a plant root microbiota; optionally, the detecting is by a functional assay optionally selected from (i) detecting enrichment of taxa in Burkholderiales and/or Rhizobiales in a plant rhizosphere or endosphere, wherein enrichment of taxa in Burkholderiales and/or Rhizobiales in the plant rhizosphere or endosphere indicates that the microbe is a beneficial commensal microbe capable of participating in a plant root microbiota; optionally, (ii) detecting nodulation in a plant root system, wherein nodulation indicates that the microbe is a beneficial commensal microbe capable of participating in a plant root microbiota; and/or (iii) detecting mycorrhization in a plant root system, wherein mycorrhization indicates that the microbe is a beneficial commensal microbe capable of participating in a plant root microbiota, or optionally the detecting is by a direct binding assay optionally selected from (1) a competition assay optionally with a known signaling saccharide, or (2) an affinity assay optionally wherein the detected affinity is compared to the affinity for the known signaling saccharide. A further embodiment of this aspect further includes providing a second polypeptide including an EPR3 or EPR3-like polypeptide, an ectodomain of an EPR3 or EPR3-like polypeptide, a M1 domain of an EPR3 or EPR3-like polypeptide, a M2 domain of an EPR3 or EPR3-like polypeptide, or a LysM3 domain of an EPR3 or EPR3-like polypeptide of the plant in step (a), wherein the second polypeptide is in contact with the first polypeptide. An additional embodiment of this aspect further includes step (d) culturing the beneficial commensal microbe if binding is detected in step (c). Yet another embodiment of this aspect further includes step (e) applying the beneficial commensal microbe to the plant or a part thereof. A further embodiment of this aspect includes the plant part being a plant propagation material, optionally a seed, a tuber, or a plantlet, and the beneficial commensal microbe being applied to the plant propagation material, optionally to the seed as part of a seed coating, to the tuber, or to a root of the plantlet. An additional embodiment of this aspect includes the plant part being a plant vegetative or reproductive material, optionally a root, a shoot, a stem, a pollen grain, or an ovule, and the beneficial commensal microbe is applied to the plant vegetative or reproductive material of the plant, optionally as part of a coating, a solution, or a powder. Still another embodiment of this aspect further includes step (e) applying the beneficial commensal microbe, optionally in admixture with a soil-compatible carrier, a fungal carrier, or a growth medium, optionally soil, where the plant is growing or is to be grown. A further embodiment of this aspect, which may be combined with any of the preceding embodiments having an ectodomain of an EPR3a or EPR3a-like polypeptide, includes the ectodomain of the EPR3a or EPR3a-like polypeptide having at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to the ectodomain of SEQ ID NO: 62 [L. japonicus (EPR3a)], SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, or SEQ ID NO: 92. Yet another embodiment of this aspect, which may be combined with any of the preceding embodiments having an ectodomain of an EPR3a or EPR3a-like polypeptide, includes the ectodomain of the EPR3a or EPR3a-like polypeptide being the ectodomain of SEQ ID NO: 62 [L. japonicus (EPR3a)], SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, or SEQ ID NO: 92. Still another embodiment of this aspect, which may be combined with any of the preceding embodiments having an ectodomain of an EPR3 or EPR3-like polypeptide, includes the ectodomain of the EPR3 or EPR3-like polypeptide having at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to the ectodomain of SEQ ID NO: 1 [L. japonicus (EPR3)], SEQ ID NO: 2 [Chickpea (XP_004489790.1)], SEQ ID NO: 3 [Medicago (XP_003613165.1)], SEQ ID NO: 4 [Soybean (XP_003517716.1)], SEQ ID NO: 5 [Phaseolus (XP_007157313.1)], SEQ ID NO: 6 [Populus (XP_002322185.1)], SEQ ID NO: 7 [Malus (XP_008340354.1)], SEQ ID NO: 8 [Vitis (XP_002272814.2)], SEQ ID NO: 9 [Theobroma (XP_007036352.1)], SEQ ID NO: 10 [Ricinus (XP_002527912.1)], SEQ ID NO: 11 [Fragaria (XP_004300916.1)], SEQ ID NO: 12 [Maize (XP_008657477.1)], SEQ ID NO: 13 [Rice (XP_015628733.1)], SEQ ID NO: 14 [Wheat (CDM80098.1)], or SEQ ID NO: 15 [Barley (MLOC_5489.2)]. An additional embodiment of this aspect, which may be combined with any of the preceding embodiments having an ectodomain of an EPR3 or EPR3-like polypeptide, includes the ectodomain of the EPR3 or EPR3-like polypeptide being the ectodomain of SEQ ID NO: 1 [L. japonicus (EPR3)], SEQ ID NO: 2 [Chickpea (XP_004489790.1)], SEQ ID NO: 3 [Medicago (XP_003613165.1)], SEQ ID NO: 4 [Soybean (XP_003517716.1)], SEQ ID NO: 5 [Phaseolus (XP_007157313.1)], SEQ ID NO: 6 [Populus (XP_002322185.1)], SEQ ID NO: 7 [Malus (XP_008340354.1)], SEQ ID NO: 8 [Vitis (XP_002272814.2)], SEQ ID NO: 9 [Theobroma (XP_007036352.1)], SEQ ID NO: 10 [Ricinus (XP_002527912.1)], SEQ ID NO: 11 [Fragaria (XP_004300916.1)], SEQ ID NO: 12 [Maize (XP_008657477.1)], SEQ ID NO: 13 [Rice (XP_015628733.1)], SEQ ID NO: 14 [Wheat (CDM80098.1)], or SEQ ID NO: 15 [Barley (MLOC_5489.2)]. Still another embodiment of this aspect includes the beneficial commensal microbe being a commensal bacteria, optionally a nitrogen-fixing bacteria, or a mycorrhizal fungi.

### Enumerated Embodiments

1. A genetically altered plant or part thereof comprising a first nucleic acid sequence encoding a heterologous EPR3a or EPR3a-like polypeptide or a modified EPR3a or EPR3a-like polypeptide, wherein the heterologous EPR3a or EPR3a-like polypeptide or the modified EPR3a or EPR3a-like polypeptide provides increased selectivity for a beneficial commensal microbe as compared to a wild-type plant under the same conditions.
2. The genetically altered plant or part thereof of embodiment 1, wherein the beneficial commensal microbe is a mycorrhizal fungi.
3. The genetically altered plant or part thereof of embodiment 2, wherein the plant or part thereof further comprises a second nucleic acid sequence encoding a heterologous EPR3 or EPR3-like polypeptide or a modified EPR3 or EPR3-like polypeptide, wherein the heterologous EPR3 or EPR3-like polypeptide or the modified EPR3 or EPR3-like polypeptide provides increased selectivity for a beneficial commensal microbe as compared to a wild-type plant under the same conditions.
4. The genetically altered plant or part thereof of embodiment 3, wherein the modified EPR3a or EPR3a-like polypeptide comprises a modified ectodomain that has been replaced with all or a portion of an ectodomain of the heterologous EPR3a or EPR3a-like polypeptide, optionally all or a part of the M1 domain, the M2 domain, the LysM3 domain, or all three; and wherein the modified EPR3 or EPR3-like polypeptide comprises a modified ectodomain that has been replaced with all or a portion of an ectodomain of the heterologous EPR3 or EPR3-like polypeptide, optionally all or a part of the M1 domain, the M2 domain, the LysM3 domain, or all three.
5. The genetically altered plant or part thereof of embodiment 3, wherein the expression of the heterologous EPR3a or EPR3a-like polypeptide, the modified EPR3a or EPR3a-like polypeptide, the heterologous EPR3 or EPR3-like polypeptide, the modified EPR3 or EPR3-like polypeptide, or a combination thereof allows the plant or part thereof to recognize an EPS, a beta-glucan, a cyclic beta-glucan, a LPS, or a surface carbohydrate produced by the microbe, and wherein the microbe is a commensal bacteria, optionally a nitrogen-fixing bacteria, or a mycorrhizal fungi.
6. The genetically altered plant of embodiment 5, wherein the heterologous EPR3a or EPR3a-like polypeptide, the modified EPR3a or EPR3a-like polypeptide, the heterologous EPR3 or EPR3-like polypeptide, or the modified EPR3 or EPR3-like polypeptide is localized to a plant cell plasma membrane, or both the EPR3 or EPR3-like polypeptide and the EPR3a or EPR3a-like polypeptide are localized to a plant cell plasma membrane, and wherein the plant cell is a root cell.
7. A method of producing the genetically altered plant of embodiment 3, comprising introducing a genetic alteration to the plant comprising the first nucleic acid sequence encoding the heterologous EPR3a or EPR3a-like polypeptide, and optionally further comprising introducing a genetic alteration to the plant comprising the second nucleic acid sequence encoding the heterologous EPR3 or EPR3-like polypeptide.
8. A method of producing the genetically altered plant of embodiment 3, comprising genetically editing a gene encoding an endogenous LysM receptor polypeptide in the plant to comprise the modified ectodomain, wherein the endogenous LysM receptor polypeptide is an endogenous EPR3a or EPR3a-like polypeptide, and wherein the modified EPR3a or EPR3a-like polypeptide was generated by:
(a) providing a heterologous EPR3a or EPR3a-like polypeptide model comprising a structural model, a molecular model, a surface characteristics model, and/or an electrostatic potential model of a M1 domain, a M2 domain, a LysM3 domain, any combination thereof, or the ectodomain of the heterologous EPR3a or EPR3a-like polypeptide having selectivity for the beneficial commensal microbe and an unmodified EPR3a or EPR3a-like polypeptide;
(b) identifying one or more amino acid residues for modification in the unmodified EPRa3 polypeptide by comparing amino acid residues of a oligosaccharide binding feature in the unmodified EPR3a or EPR3a-like polypeptide with the corresponding amino acid residues in the heterologous EPR3a or EPR3a-like polypeptide model; and
(c) generating the unmodified EPR3a or EPR3a-like polypeptide wherein the one or more amino acid residues in the oligosaccharide binding feature of the unmodified EPR3a or EPR3a-like polypeptide have been substituted with corresponding amino acid residues from the heterologous EPR3a or EPR3a-like polypeptide; or
   wherein the endogenous LysM receptor polypeptide is an endogenous EPR3 or EPR3-like polypeptide, and wherein the modified EPR3 or EPR3-like polypeptide was generated by:
(a) providing a heterologous EPR3 or EPR3-like polypeptide model comprising a structural model, a molecular model, a surface characteristics model, and/or an electrostatic potential model of a M1 domain, a M2 domain, a LysM3 domain, any combination thereof, or the ectodomain of the heterologous EPR3 or EPR3-like polypeptide having selectivity for the beneficial commensal microbe and an unmodified EPR3 or EPR3-like polypeptide;
(b) identifying one or more amino acid residues for modification in the unmodified EPR3 or EPR3-like polypeptide by comparing amino acid residues of a oligosaccharide binding feature in the unmodified EPR3 or EPR3-like polypeptide with the corresponding amino acid residues in the heterologous EPR3 or EPR3-like polypeptide model; and
(c) generating the unmodified EPR3 or EPR3-like polypeptide wherein the one or more amino acid residues in the oligosaccharide binding feature of the unmodified EPR3 or EPR3-like polypeptide have been substituted with corresponding amino acid residues from the heterologous EPR3 or EPR3-like polypeptide.
   9. A genetically altered plant or part thereof comprising a first nucleic acid sequence encoding a heterologous EPR3 or EPR3-like polypeptide or a modified EPR3 or EPR3-like polypeptide, wherein the heterologous EPR3 or EPR3-like polypeptide or the modified EPR3 or EPR3-like polypeptide provides increased selectivity for a beneficial commensal microbe as compared to a wild-type plant under the same conditions.
   10. The genetically altered plant or part thereof of embodiment 9, wherein the plant or part thereof further comprises a second nucleic acid sequence encoding a heterologous EPR3a or EPR3a-like polypeptide or a modified EPR3a or EPR3a-like polypeptide, wherein the heterologous EPR3a or EPR3a-like polypeptide or the modified EPR3a or EPR3a-like polypeptide provides increased selectivity for a beneficial commensal microbe as compared to a wild-type plant under the same conditions.
   11. The genetically altered plant or part thereof of embodiment 10, wherein the modified EPR3 or EPR3-like polypeptide comprises a modified ectodomain that has been replaced with all or a portion of an ectodomain of the heterologous EPR3 or EPR3-like polypeptide, optionally all or a part of the M1 domain, the M2 domain, the LysM3 domain, or all three; and wherein the modified EPR3a or EPR3a-like polypeptide comprises a modified ectodomain that has been replaced with all or a portion of an ectodomain of the heterologous EPR3a or EPR3a-like polypeptide, optionally all or a part of the M1 domain, the M2 domain, the LysM3 domain, or all three.
   12. The genetically altered plant or part thereof of embodiment 10, wherein the expression of the heterologous EPR3 or EPR3-like polypeptide, the modified EPR3 or EPR3-like polypeptide, the heterologous EPR3a or EPR3a-like polypeptide, the modified EPR3a or EPR3a-like polypeptide, or a combination thereof allows the plant or part thereof to recognize an exopolysaccharide (EPS), a beta-glucan, a cyclic beta-glucan, a LPS, or a surface carbohydrate produced by the microbe, and wherein the microbe is a commensal bacteria, optionally a nitrogen-fixing bacteria, or a mycorrhizal fungi.
   13. The genetically altered plant or part thereof of embodiment 12, wherein the heterologous EPR3 or EPR3-like polypeptide, the modified EPR3 or EPR3-like polypeptide, the heterologous EPR3a or EPR3a-like polypeptide, or the modified EPR3a or EPR3a-like polypeptide is localized to a plant cell plasma membrane, or both the EPR3 or EPR3-like polypeptide and the EPR3a or EPR3a-like polypeptide are localized to a plant cell plasma membrane, and wherein the plant cell is a root cell.
   14. A method of producing the genetically altered plant of embodiment 10, comprising introducing a genetic alteration to the plant comprising the first nucleic acid sequence encoding the heterologous EPR3 or EPR3-like polypeptide, and optionally further comprising introducing a genetic alteration to the plant comprising the second nucleic acid sequence encoding the heterologous EPR3a or EPR3a-like polypeptide.
   15. A method of producing the genetically altered plant of embodiment 10, comprising genetically editing a gene encoding an endogenous LysM receptor polypeptide in the plant to comprise the modified ectodomain, wherein the endogenous LysM receptor polypeptide is an endogenous EPR3 or EPR3-like polypeptide, and wherein the modified EPR3 or EPR3-like polypeptide was generated by:
(a) providing a heterologous EPR3 or EPR3-like polypeptide model comprising a structural model, a molecular model, a surface characteristics model, and/or an electrostatic potential model of a M1 domain, a M2 domain, a LysM3 domain, any combination thereof, or the ectodomain of the heterologous EPR3 or EPR3-like polypeptide having selectivity for the beneficial commensal microbe and an unmodified EPR3 or EPR3-like polypeptide;
(b) identifying one or more amino acid residues for modification in the unmodified EPR3 or EPR3-like polypeptide by comparing amino acid residues of a oligosaccharide binding feature in the unmodified EPR3 or EPR3-like polypeptide with the corresponding amino acid residues in the heterologous EPR3 or EPR3-like polypeptide model; and
(c) generating the unmodified EPR3 or EPR3-like polypeptide wherein the one or more amino acid residues in the oligosaccharide binding feature of the unmodified EPR3 or EPR3-like polypeptide have been substituted with corresponding amino acid residues from the heterologous EPR3 or EPR3-like polypeptide; or
   wherein the endogenous LysM receptor polypeptide is an endogenous EPR3a or EPR3a-like polypeptide, and wherein the modified EPR3a or EPR3a-like polypeptide was generated by:
(a) providing a heterologous EPR3a or EPR3a-like polypeptide model comprising a structural model, a molecular model, a surface characteristics model, and/or an electrostatic potential model of a M1 domain, a M2 domain, a LysM3 domain, any combination thereof, or the ectodomain of the heterologous EPR3a or EPR3a-like polypeptide having selectivity for the beneficial commensal microbe and an unmodified EPR3a or EPR3a-like polypeptide;
(b) identifying one or more amino acid residues for modification in the unmodified EPR3a or EPR3a-like polypeptide by comparing amino acid residues of a oligosaccharide binding feature in the unmodified EPR3a or EPR3a-like polypeptide with the corresponding amino acid residues in the heterologous EPR3a or EPR3a-like polypeptide model; and
(c) generating the unmodified EPR3a or EPR3a-like polypeptide wherein the one or more amino acid residues in the oligosaccharide binding feature of the unmodified EPR3a or EPR3a-like polypeptide have been substituted with corresponding amino acid residues from the heterologous EPR3a or EPR3a-like polypeptide.
   16. A method of identifying a beneficial commensal microbe capable of participating in a plant root microbiota comprising:
a) providing a first polypeptide comprising an EPR3a or EPR3a-like polypeptide, an ectodomain of an EPR3a or EPR3a-like polypeptide, a M1 domain of an EPR3a or EPR3a-like polypeptide, a M2 domain of an EPR3a or EPR3a-like polypeptide, or a LysM3 domain of an EPR3a or EPR3a-like polypeptide of the plant;
b) contacting the first polypeptide with a sample comprising a microbe or an EPS, a beta-glucan, a cyclic beta-glucan, a LPS, or a surface carbohydrate produced by the microbe;
c) detecting binding of the EPS, the beta-glucan, the cyclic beta-glucan, the LPS, or the surface carbohydrate produced by the microbe to the polypeptide, wherein binding of the EPS, the beta-glucan, the cyclic beta-glucan, the LPS, or the surface carbohydrate to the polypeptide indicates that the microbe is a beneficial commensal microbe capable of participating in the plant root microbiota; optionally, the detecting is by a functional assay optionally selected from (i) detecting enrichment of taxa in Burkholderiales and/or Rhizobiales in a plant rhizosphere or endosphere, wherein enrichment of taxa in Burkholderiales and/or Rhizobiales in the plant rhizosphere or endosphere indicates that the microbe is a beneficial commensal microbe capable of participating in a plant root microbiota; optionally, (ii) detecting nodulation in a plant root system, wherein nodulation indicates that the microbe is a beneficial commensal microbe capable of participating in a plant root microbiota; and/or (iii) detecting mycorrhization in a plant root system, wherein mycorrhization indicates that the microbe is a beneficial commensal microbe capable of participating in a plant root microbiota, or optionally the detecting is by a direct binding assay optionally selected from (1) a competition assay optionally with a known signaling saccharide, or (2) an affinity assay optionally wherein the detected affinity is compared to the affinity for the known signaling saccharide; and
   optionally further comprising:
d) culturing the beneficial commensal microbe if binding is detected in step (c); and
e) applying the beneficial commensal microbe to the plant or a part thereof or applying the beneficial commensal microbe, optionally in admixture with a soil-compatible carrier, a fungal carrier, or a growth medium, optionally soil, where the plant is growing or is to be grown.
   17. The method of embodiment 16, further comprising providing a second polypeptide comprising an EPR3 or EPR3-like polypeptide, an ectodomain of an EPR3 or EPR3-like polypeptide, a M1 domain of an EPR3 or EPR3-like polypeptide, a M2 domain of an EPR3 or EPR3-like polypeptide, or a LysM3 domain of an EPR3 or EPR3-like polypeptide of the plant in step (a), wherein the second polypeptide is in contact with the first polypeptide.
   18. A method of identifying a beneficial commensal microbe capable of participating in a plant root microbiota comprising:
a) providing a first polypeptide comprising an EPR3 or EPR3-like polypeptide, an ectodomain of an EPR3 or EPR3-like polypeptide, a M1 domain of an EPR3 or EPR3-like polypeptide, a M2 domain of an EPR3 or EPR3-like polypeptide, or a LysM3 domain of an EPR3 or EPR3-like polypeptide of the plant;
b) contacting the first polypeptide with a sample comprising a microbe or an EPS, a beta-glucan, a cyclic beta-glucan, a LPS, or a surface carbohydrate produced by the microbe;
c) detecting binding of the EPS, the beta-glucan, the cyclic beta-glucan, the LPS, or the surface carbohydrate produced by the microbe to the polypeptide, wherein binding of the EPS, the beta-glucan, the cyclic beta-glucan, the LPS, or the surface carbohydrate to the polypeptide indicates that the microbe is a beneficial commensal microbe capable of participating in a plant root microbiota; optionally, the detecting is by a functional assay optionally selected from (i) detecting enrichment of taxa in Burkholderiales and/or Rhizobiales in a plant rhizosphere or endosphere, wherein enrichment of taxa in Burkholderiales and/or Rhizobiales in the plant rhizosphere or endosphere indicates that the microbe is a beneficial commensal microbe capable of participating in a plant root microbiota; optionally, (ii) detecting nodulation in a plant root system, wherein nodulation indicates that the microbe is a beneficial commensal microbe capable of participating in a plant root microbiota; and/or (iii) detecting mycorrhization in a plant root system, wherein mycorrhization indicates that the microbe is a beneficial commensal microbe capable of participating in a plant root microbiota, or optionally the detecting is by a direct binding assay optionally selected from (1) a competition assay optionally with a known signaling saccharide, or (2) an affinity assay optionally wherein the detected affinity is compared to the affinity for the known signaling saccharide; and
   optionally further comprising:

d) culturing the beneficial commensal microbe if binding is detected in step (c); and

e) applying the beneficial commensal microbe to the plant or a part thereof or applying the beneficial commensal microbe, optionally in admixture with a soil-compatible carrier, a fungal carrier, or a growth medium, optionally soil, where the plant is growing or is to be grown.

19. The method of embodiment 18, further comprising providing a second polypeptide comprising an EPR3a or EPR3a-like polypeptide, an ectodomain of an EPR3a or EPR3a-like polypeptide, a M1 domain of an EPR3a or EPR3a-like polypeptide, a M2 domain of an EPR3a or EPR3a-like polypeptide, or a LysM3 domain of an EPR3a or EPR3a-like polypeptide of the plant in step (a), wherein the second polypeptide is in contact with the first polypeptide.
20. A genetically altered plant or part thereof comprising a first nucleic acid sequence encoding a heterologous EPR3 or EPR3-like polypeptide or a modified EPR3 or EPR3-like polypeptide, wherein the heterologous EPR3 or EPR3-like polypeptide or the modified EPR3 or EPR3-like polypeptide provides increased selectivity for a beneficial commensal microbe as compared to a wild-type plant under the same conditions.
21. The genetically altered plant or part thereof of embodiment 20, wherein the plant or part thereof further comprises a second nucleic acid sequence encoding a heterologous EPR3a or EPR3a-like polypeptide or a modified EPR3a or EPR3a-like polypeptide.
22. The genetically altered plant or part thereof of embodiment 20 or embodiment 21, wherein the heterologous EPR3 or EPR3-like polypeptide is selected from the group consisting of a first polypeptide with at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 1 [L. japonicus (BAI79269.1)], a second polypeptide with at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 2 [Chickpea (XP_004489790.1)], a third polypeptide with at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 3 [Medicago (XP_003613165.1)], a fourth polypeptide with at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 4 [Soybean (XP_003517716.1)], a fifth polypeptide with at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 5 [Phaseolus (XP_007157313.1)], a sixth polypeptide with at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 6 [Populus (XP_002322185.1)], a seventh polypeptide with at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 7 [Malus (XP_008340354.1)], an eighth polypeptide with at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 8 [Vitis (XP_002272814.2)], a ninth polypeptide with at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 9 [Theobroma (XP_007036352.1)], a tenth polypeptide with at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 10 [Ricinus (XP_002527912.1)], an eleventh polypeptide with at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 11 [Fragaria (XP_004300916.1)], a twelfth polypeptide with at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 12 [Maize (XP_008657477.1)], a thirteenth polypeptide with at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 13 [Rice (XP_015628733.1)], a fourteenth polypeptide with at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 14 [Wheat (CDM80098.1)], and a fifteenth polypeptide with at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 15 [Barley (MLOC 5489.2)].
23. The genetically altered plant or part thereof of embodiment 22, wherein the heterologous EPR3 or EPR3-like polypeptide is selected from the group consisting of SEQ ID NO: 1 [L. japonicus (EPR3)], SEQ ID NO: 2 [Chickpea (XP_004489790.1)], SEQ ID NO: 3 [Medicago (XP_003613165.1)], SEQ ID NO: 4 [Soybean (XP_003517716.1)], SEQ ID NO: 5 [Phaseolus (XP_007157313.1)], SEQ ID NO: 6 [Populus (XP_002322185.1)], SEQ ID NO: 7 [Malus (XP_008340354.1)], SEQ ID NO: 8 [Vitis (XP_002272814.2)], SEQ ID NO: 9 [Theobroma (XP_007036352.1)], SEQ ID NO: 10 [Ricinus (XP_002527912.1)], SEQ ID NO: 11 [Fragaria (XP_004300916.1)], SEQ ID NO: 12 [Maize (XP_008657477.1)], SEQ ID NO: 13 [Rice (XP_015628733.1)], SEQ ID NO: 14 [Wheat (CDM80098.1)], and SEQ ID NO: 15 [Barley (MLOC_5489.2)].
24. The genetically altered plant or part thereof of any one of embodiments 21-23, wherein the heterologous EPR3a or EPR3a-like polypeptide is selected from the group consisting of a polypeptide with at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 62 [L. japonicus (EPR3a)], SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, or SEQ ID NO: 92.
25. The genetically altered plant or part thereof of embodiment 24, wherein the heterologous EPR3a or EPR3a-like polypeptide is SEQ ID NO: 62 [L. japonicus (EPR3a)], SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, or SEQ ID NO: 92.
26. The genetically altered plant or part thereof of any one of embodiments 20-25, wherein the modified EPR3 or EPR3-like polypeptide comprises a modified ectodomain that has been replaced with all or a portion of an ectodomain of the heterologous EPR3 or EPR3-like polypeptide, optionally all or a part of the M1 domain, the M2 domain, the LysM3 domain, or all three.
27. The genetically altered plant or part thereof of embodiment 26, wherein the portion replaced is at least 10%, at least 20%, at least 30%, at least 40%, at least 50%, at least 60%, at least 70%, at least 80%, at least 90%, less than 10%, less than 20%, less than 30%, less than 40%, less than 50%, less than 60%, less than 70%, less than 80%, or less than 90%, of the ectodomain or, optionally all or a part of the M1 domain, the M2 domain, the LysM3 domain, or all three.
28. The genetically altered plant or part thereof of any one of embodiments 21-25, wherein the modified EPR3a or EPR3a-like polypeptide comprises a modified ectodomain that has been replaced with all or a portion of an ectodomain of the heterologous EPR3a or EPR3a-like polypeptide, optionally all or a part of the M1 domain, the M2 domain, the LysM3 domain, or all three.
29. The genetically altered plant or part thereof of embodiment 28, wherein the portion replaced is at least 10%, at least 20%, at least 30%, at least 40%, at least 50%, at least 60%, at least 70%, at least 80%, at least 90%, less than 10%, less than 20%, less than 30%, less than 40%, less than 50%, less than 60%, less than 70%, less than 80%, or less than 90%, of the ectodomain or, optionally all or a part of the M1 domain, the M2 domain, the LysM3 domain, or all three.
30. The genetically altered plant or part thereof of any one of embodiments 21-29, wherein the heterologous EPR3 or EPR3-like polypeptide and the heterologous EPR3a or EPR3a-like polypeptide are from the same plant species or the same plant variety.
31. The genetically altered plant or part thereof of any one of embodiments 20-30, wherein the expression of the heterologous EPR3 or EPR3-like polypeptide or the modified EPR3 or EPR3-like polypeptide allows the plant or part thereof to recognize an exopolysaccharide (EPS), a beta-glucan, a cyclic beta-glucan, a LPS, or a surface carbohydrate produced by the microbe.
32. The genetically altered plant or part thereof of any one of embodiments 21-31, wherein the expression of the heterologous EPR3a or EPR3a-like polypeptide or the modified EPR3a or EPR3a-like polypeptide allows the plant or part thereof to recognize an EPS, a beta-glucan, a cyclic beta-glucan, a LPS, or a surface carbohydrate produced by the microbe.
33. The genetically altered plant or part thereof of embodiment 32, wherein the expression of the heterologous EPR3 or EPR3-like polypeptide or the modified EPR3 or EPR3-like polypeptide and the expression of the heterologous EPR3a or EPR3a-like polypeptide or the modified EPR3a or EPR3a-like polypeptide allows the plant or part thereof to recognize an EPS, a beta-glucan, a cyclic beta-glucan, a LPS, or a surface carbohydrate produced by the microbe.
34. The genetically altered plant or part thereof of any one of embodiments 31-33, wherein the microbe is a commensal bacteria, optionally a nitrogen-fixing bacteria, or a mycorrhizal fungi.
35. The genetically altered plant or part thereof of embodiment 34, wherein the nitrogen-fixing bacteria is selected from the group consisting of Mesorhizobium loti, Mesorhizobium huakuii, Mesorhizobium mediterraneum, Mesorhizobium ciceri, Mesorhizobium spp., Rhizobium mongolense, Rhizobium tropici, Rhizobium etli phaseoli, Rhizobium giardinii, Rhizobium leguminosarum optionally R. leguminosarum trifolii, R. leguminosarum viciae, and R. leguminosarum phaseoli, Burkholderiales optionally symbionts of Mimosa, Sinorhizobium meliloti, Sinorhizobium medicae, Sinorhizobium fredii, Sinorhizobium fredii NGR234, Azorhizobium caulinodans, Bradyrhizobium japonicum, Bradyrhizobium elkanii, Bradyrhizobium liaonginense, Rhizobium spp., Mesorhizobium spp., Sinorhizobium spp., Azorhizobium spp. Frankia spp., and any combination thereof, or the mycorrhizal fungi is selected from the group consisting of Acaulosporaceae spp., Diversisporaceae spp., Gigasporaceae spp., Pacisporaceae spp., Funneliformis spp., Glomus spp., Rhizophagus spp., Sclerocystis spp., Septoglomus spp., Claroideoglomus spp., Ambispora spp., Archaeospora spp., Geosiphon pyriformis, Paraglomus spp., other species in the division Glomeromycota, and any combination thereof.
36. The genetically altered plant or part thereof of any one of embodiments 20-35, wherein the heterologous EPR3 or EPR3-like polypeptide or the modified EPR3 or EPR3-like polypeptide is localized to a plant cell plasma membrane.
37. The genetically altered plant or part thereof of any one of embodiments 21-36, wherein the heterologous EPR3a or EPR3a-like polypeptide or the modified EPR3a or EPR3a-like polypeptide is localized to a plant cell plasma membrane.
38. The genetically altered plant or part thereof of embodiment 36 or embodiment 37, wherein the plant cell is a root cell.
39. The genetically altered plant or part thereof of embodiment 38, wherein the root cell is a root epidermal cell or a root cortex cell.
40. The genetically altered plant or part thereof of any one of embodiments 20-39, wherein the heterologous EPR3 or EPR3-like polypeptide or the modified EPR3 or EPR3-like polypeptide is expressed in a developing plant root system.
41. The genetically altered plant or part thereof of any one of embodiments 21-40, wherein the heterologous EPR3a or EPR3a-like polypeptide or the modified EPR3a or EPR3a-like polypeptide is expressed in a developing plant root system.
42. The genetically altered plant or part thereof of any one of embodiments 20-41, wherein the first nucleic acid sequence is operably linked to a first promoter.
43. The genetically altered plant or part thereof of embodiment 42, wherein the first promoter is a root specific promoter, and wherein the root specific promoter is optionally selected from the group consisting of a NFR1 or NFR5/NFP promoter, an EPR3 or an EPR3a promoter, a Lotus NFR5 promoter, a Lotus NFR1 promoter, a maize allothioneine promoter, a chitinase promoter, a maize ZRP2 promoter, a tomato LeExt1 promoter, a glutamine synthetase soybean root promoter, a RCC3 promoter, a rice antiquitine promoter, a LRR receptor kinase promoter, and an Arabidopsis pCO2 promoter.
44. The genetically altered plant or part thereof of embodiment 42, wherein the first promoter is a constitutive promoter, and wherein the constitutive promoter is optionally selected from the group consisting of a CaMV35S promoter, a derivative of the CaMV35S promoter, a maize ubiquitin promoter, a trefoil promoter, a vein mosaic cassava virus promoter, and an Arabidopsis UBQ10 promoter.
45. The genetically altered plant or part thereof of any one of embodiments 21-44, wherein the second nucleic acid sequence is operably linked to a second promoter.
46. The genetically altered plant or part thereof of embodiment 45, wherein the second promoter is a root specific promoter, and wherein the root specific promoter is optionally selected from the group consisting of a NFR1 or NFR5/NFP promoter, an EPR3 or an EPR3a promoter, a Lotus NFR5 promoter, a Lotus NFR1 promoter, a maize allothioneine promoter, a chitinase promoter, a maize ZRP2 promoter, a tomato LeExt1 promoter, a glutamine synthetase soybean root promoter, a RCC3 promoter, a rice antiquitine promoter, a LRR receptor kinase promoter, and an Arabidopsis pCO2 promoter.
47. The genetically altered plant or part thereof of embodiment 45, wherein the second promoter is a constitutive promoter, and wherein the constitutive promoter is optionally selected from the group consisting of a CaMV35S promoter, a derivative of the CaMV35S promoter, a maize ubiquitin promoter, a trefoil promoter, a vein mosaic cassava virus promoter, and an Arabidopsis UBQ10 promoter.
48. The genetically altered plant or part thereof of any one of embodiments 20-47, wherein the plant is selected from the group consisting of cassava, corn, cowpea, rice, barley, wheat, Trema spp., apple, pear, plum, apricot, peach, almond, walnut, strawberry, raspberry, blackberry, red currant, black currant, melon, cucumber, pumpkin, squash, grape, tomato, pepper, and hemp.
49. The genetically altered plant part of any one of embodiments 20-48, wherein the plant part is a leaf, a stem, a root, a root primordia, a flower, a seed, a fruit, a kernel, a grain, a cell, or a portion thereof.
50. The genetically altered plant part of embodiment 49, wherein the plant part is a fruit, a kernel, or a grain.
51. A pollen grain or an ovule of the genetically altered plant of any one of embodiments 20-48.
52. A protoplast produced from the plant of any one of embodiments 20-48.
53. A tissue culture produced from protoplasts or cells from the plant of any one of embodiments 20-48, wherein the cells or protoplasts are produced from a plant part selected from the group consisting of leaf, anther, pistil, stem, petiole, root, root primordia, root tip, fruit, seed, flower, cotyledon, hypocotyl, embryo, and meristematic cell.

54. A method of producing the genetically altered plant of any one of embodiments 20-48, comprising introducing a genetic alteration to the plant comprising the first nucleic acid sequence encoding the heterologous EPR3 or EPR3-like polypeptide.

55. The method of embodiment 54, wherein the first nucleic acid sequence is operably linked to a first promoter.
56. The method of embodiment 55, wherein the first promoter is a root specific promoter, and wherein the root specific promoter is optionally selected from the group consisting of a NFR1 or NFR5/NFP promoter, an EPR3 or an EPR3a promoter, a Lotus NFR5 promoter, a Lotus NFR1 promoter, a maize allothioneine promoter, a chitinase promoter, a maize ZRP2 promoter, a tomato LeExt1 promoter, a glutamine synthetase soybean root promoter, a RCC3 promoter, a rice antiquitine promoter, a LRR receptor kinase promoter, and an Arabidopsis pCO2 promoter.
57. The method of embodiment 55, wherein the first promoter is a constitutive promoter, and wherein the constitutive promoter is optionally selected from the group consisting of a CaMV35S promoter, a derivative of the CaMV35S promoter, a maize ubiquitin promoter, a trefoil promoter, a vein mosaic cassava virus promoter, and an Arabidopsis UBQ10 promoter.
58. The method of any one of embodiments 54-57, further comprising introducing a genetic alteration to the plant comprising the second nucleic acid sequence encoding the heterologous EPR3a or EPR3a-like polypeptide.
59. The method of embodiment 58, wherein the second nucleic acid sequence is operably linked to a second promoter.
60. The method of embodiment 59, wherein the second promoter is a root specific promoter, and wherein the root specific promoter is optionally selected from the group consisting of a NFR1 or NFR5/NFP promoter, an EPR3 or an EPR3a promoter, a Lotus NFR5 promoter, a Lotus NFR1 promoter, a maize allothioneine promoter, a chitinase promoter, a maize ZRP2 promoter, a tomato LeExt1 promoter, a glutamine synthetase soybean root promoter, a RCC3 promoter, a rice antiquitine promoter, a LRR receptor kinase promoter, and an Arabidopsis pCO2 promoter.
61. The method of embodiment 59, wherein the second promoter is a constitutive promoter, and wherein the constitutive promoter is optionally selected from the group consisting of a CaMV35S promoter, a derivative of the CaMV35S promoter, a maize ubiquitin promoter, a trefoil promoter, a vein mosaic cassava virus promoter, and an Arabidopsis UBQ10 promoter.
62. The method of any one of embodiments 54-61, wherein the first nucleic acid sequence is inserted into the genome of the plant so that the nucleic acid sequence is operably linked to a first endogenous promoter.
63. The method of embodiment 62, wherein the first endogenous promoter is a root specific promoter.
64. The method of any one of embodiments 58-63, wherein the second nucleic acid sequence is inserted into the genome of the plant so that the nucleic acid sequence is operably linked to a second endogenous promoter.
65. The method of embodiment 64, wherein the second endogenous promoter is a root specific promoter.
66. A method of producing the genetically altered plant of any one of embodiments 26-65, comprising genetically editing a gene encoding an endogenous LysM receptor polypeptide in the plant to comprise the modified ectodomain.
67. The method of embodiment 66, wherein the endogenous LysM receptor polypeptide is an endogenous EPR3 or EPR3-like polypeptide.
68. The method of embodiment 66 or embodiment 67, wherein the modified EPR3 or EPR3-like polypeptide was generated by:
(a) providing a heterologous EPR3 or EPR3-like polypeptide model comprising a structural model, a molecular model, a surface characteristics model, and/or an electrostatic potential model of a M1 domain, a M2 domain, a LysM3 domain, any combination thereof, or the ectodomain of the heterologous EPR3 or EPR3-like polypeptide having selectivity for the beneficial commensal microbe and an unmodified EPR3 or EPR3-like polypeptide;
(b) identifying one or more amino acid residues for modification in the unmodified EPR3 or EPR3-like polypeptide by comparing amino acid residues of a oligosaccharide binding feature in the unmodified EPR3 or EPR3-like polypeptide with the corresponding amino acid residues in the heterologous EPR3 or EPR3-like polypeptide model; and
(c) generating the unmodified EPR3 or EPR3-like polypeptide wherein the one or more amino acid residues in the oligosaccharide binding feature of the unmodified EPR3 or EPR3-like polypeptide have been substituted with corresponding amino acid residues from the heterologous EPR3 or EPR3-like polypeptide.
   69. The method of embodiment 68, wherein the heterologous EPR3 or EPR3-like polypeptide model is a protein crystal structure, a molecular model, a cryo-EM structure, or a NMR structure.
   70. The method of embodiment 66, wherein the endogenous LysM receptor polypeptide is an endogenous EPR3a or EPR3a-like polypeptide.
   71. The method of embodiment 70, wherein the modified EPR3a or EPR3a-like polypeptide was generated by:
(a) providing a heterologous EPR3a or EPR3a-like polypeptide model comprising a structural model, a molecular model, a surface characteristics model, and/or an electrostatic potential model of a M1 domain, a M2 domain, a LysM3 domain, any combination thereof, or the ectodomain of the heterologous EPR3a or EPR3a-like polypeptide having selectivity for the beneficial commensal microbe and an unmodified EPR3a or EPR3a-like polypeptide;
(b) identifying one or more amino acid residues for modification in the unmodified EPR3a or EPR3a-like polypeptide by comparing amino acid residues of a oligosaccharide binding feature in the unmodified EPR3a or EPR3a-like polypeptide with the corresponding amino acid residues in the heterologous EPR3a or EPR3a-like polypeptide model; and
(c) generating the unmodified EPR3a or EPR3a-like polypeptide wherein the one or more amino acid residues in the oligosaccharide binding feature of the unmodified EPR3a or EPR3a-like polypeptide have been substituted with corresponding amino acid residues from the heterologous EPR3a or EPR3a-like polypeptide.
   72. The method of embodiment 71, wherein the heterologous EPR3a or EPR3a-like polypeptide model is a protein crystal structure, a molecular model, a cryo-EM structure, or a NMR structure.
   73. A plant or part thereof produced by the method of any one of embodiments 54-72.
   74. A genetically altered plant or part thereof comprising a first nucleic acid sequence encoding a heterologous EPR3a or EPR3a-like polypeptide or a modified EPR3a or EPR3a-like polypeptide, wherein the heterologous EPR3a or EPR3a-like polypeptide or the modified EPR3a or EPR3a-like polypeptide provides increased selectivity for a beneficial commensal microbe as compared to a wild-type plant under the same conditions.
   75. The genetically altered plant or part thereof of embodiment 74, wherein the plant or part thereof further comprises a second nucleic acid sequence encoding a heterologous EPR3 or EPR3-like polypeptide or a modified EPR3 or EPR3-like polypeptide.
   76. The genetically altered plant or part thereof of embodiment 74 or embodiment 75, wherein the heterologous EPR3a or EPR3a-like polypeptide is selected from the group consisting of a polypeptide with at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 62 [L. japonicus (EPR3a)], SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, or SEQ ID NO: 92.
   77. The genetically altered plant or part thereof of embodiment 76, wherein the heterologous EPR3a or EPR3a-like polypeptide is the heterologous EPR3a or EPR3a-like polypeptide is SEQ ID NO: 62 [L. japonicus (EPR3a)], SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, or SEQ ID NO: 92.
   78. The genetically altered plant or part thereof of any one of embodiments 75-77, wherein the heterologous EPR3 or EPR3-like polypeptide is selected from the group consisting of a first polypeptide with at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 1 [L. japonicus (BAI79269.1)], a second polypeptide with at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 2 [Chickpea (XP_004489790.1)], a third polypeptide with at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 3 [Medicago (XP_003613165.1)], a fourth polypeptide with at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 4 [Soybean (XP_003517716.1)], a fifth polypeptide with at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 5 [Phaseolus (XP_007157313.1)], a sixth polypeptide with at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 6 [Populus (XP_002322185.1)], a seventh polypeptide with at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 7 [Malus (XP_008340354.1)], an eighth polypeptide with at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 8 [Vitis (XP_002272814.2)], a ninth polypeptide with at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 9 [Theobroma (XP_007036352.1)], a tenth polypeptide with at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 10 [Ricinus (XP_002527912.1)], an eleventh polypeptide with at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 11 [Fragaria (XP_004300916.1)], a twelfth polypeptide with at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 12 [Maize (XP_008657477.1)], a thirteenth polypeptide with at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 13 [Rice (XP_015628733.1)], a fourteenth polypeptide with at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 14 [Wheat (CDM80098.1)], and a fifteenth polypeptide with at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 15 [Barley (MLOC_5489.2)].
   79. The genetically altered plant or part thereof of embodiment 78, wherein the heterologous EPR3 or EPR3-like polypeptide is selected from the group consisting of SEQ ID NO: 1 [L. japonicus (EPR3)], SEQ ID NO: 2 [Chickpea (XP_004489790.1)], SEQ ID NO: 3 [Medicago (XP_003613165.1)], SEQ ID NO: 4 [Soybean (XP_003517716.1)], SEQ ID NO: 5 [Phaseolus (XP_007157313.1)], SEQ ID NO: 6 [Populus (XP_002322185.1)], SEQ ID NO: 7 [Malta (XP_008340354.1)], SEQ ID NO: 8 [Vitis (XP_002272814.2)], SEQ ID NO: 9 [Theobroma (XP_007036352.1)], SEQ ID NO: 10 [Ricinus (XP_002527912.1)], SEQ ID NO: 11 [Fragaria (XP_004300916.1)], SEQ ID NO: 12 [Maize (XP_008657477.1)], SEQ ID NO: 13 [Rice (XP_015628733.1)], SEQ ID NO: 14 [Wheat (CDM80098.1)], and SEQ ID NO: 15 [Barley (MLOC_5489.2)].
   80. The genetically altered plant or part thereof of any one of embodiments 74-79, wherein the modified EPR3a or EPR3a-like polypeptide comprises a modified ectodomain that has been replaced with all or a portion of an ectodomain of the heterologous EPR3a or EPR3a-like polypeptide, optionally all or a part of the M1 domain, the M2 domain, the LysM3 domain, or all three.
   81. The genetically altered plant or part thereof of embodiment 80, wherein the portion replaced is at least 10%, at least 20%, at least 30%, at least 40%, at least 50%, at least 60%, at least 70%, at least 80%, at least 90%, less than 10%, less than 20%, less than 30%, less than 40%, less than 50%, less than 60%, less than 70%, less than 80%, or less than 90%, of the ectodomain or, optionally all or a part of the M1 domain, the M2 domain, the LysM3 domain, or all three.
   82. The genetically altered plant or part thereof of any one of embodiments 75-81, wherein the modified EPR3 or EPR3-like polypeptide comprises a modified ectodomain that has been replaced with all or a portion of an ectodomain of the heterologous EPR3 or EPR3-like polypeptide, optionally all or a part of the M1 domain, the M2 domain, the LysM3 domain, or all three.
   83. The genetically altered plant or part thereof of embodiment 82, wherein the portion replaced is at least 10%, at least 20%, at least 30%, at least 40%, at least 50%, at least 60%, at least 70%, at least 80%, at least 90%, less than 10%, less than 20%, less than 30%, less than 40%, less than 50%, less than 60%, less than 70%, less than 80%, or less than 90%, of the ectodomain or, optionally all or a part of the M1 domain, the M2 domain, the LysM3 domain, or all three.
   84. The genetically altered plant or part thereof of any one of embodiments 75-83, wherein the heterologous EPR3a or EPR3a-like polypeptide and the heterologous EPR3 or EPR3-like polypeptide are from the same plant species or the same plant variety.
   85. The genetically altered plant or part thereof of any one of embodiments 74-84, wherein the expression of the heterologous EPR3a or EPR3a-like polypeptide or the modified EPR3a or EPR3a-like polypeptide allows the plant or part thereof to recognize an EPS, a beta-glucan, a cyclic beta-glucan, a LPS, or a surface carbohydrate produced by the microbe.
   86. The genetically altered plant or part thereof of any one of embodiments 75-85, wherein the expression of the heterologous EPR3 or EPR3-like polypeptide or the modified EPR3 or EPR3-like polypeptide allows the plant or part thereof to recognize an EPS, a beta-glucan, a cyclic beta-glucan, a LPS, or a surface carbohydrate produced by the microbe.
   87. The genetically altered plant or part thereof of embodiment 86, wherein the expression of the heterologous EPR3a or EPR3a-like polypeptide or the modified EPR3a or EPR3a-like polypeptide and the expression of the heterologous EPR3 or EPR3-like polypeptide or the modified EPR3 or EPR3-like polypeptide allows the plant or part thereof to recognize an EPS, a beta-glucan, a cyclic beta-glucan, a LPS, or a surface carbohydrate produced by the microbe.
   88. The genetically altered plant or part thereof of any one of embodiments 85-87, wherein the microbe is a commensal bacteria, optionally a nitrogen-fixing bacteria, or a mycorrhizal fungi.
   89. The genetically altered plant or part thereof of embodiment 88, wherein the nitrogen-fixing bacteria is selected from the group consisting of Mesorhizobium loti, Mesorhizobium huakuii, Mesorhizobium mediterraneum, Mesorhizobium ciceri, Mesorhizobium spp., Rhizobium mongolense, Rhizobium tropici, Rhizobium etli phaseoli, Rhizobium giardinii, Rhizobium leguminosarum optionally R. leguminosarum trifolii, R. leguminosarum viciae, and R. leguminosarum phaseoli, Burkholderiales optionally symbionts of Mimosa, Sinorhizobium meliloti, Sinorhizobium medicae, Sinorhizobium fredii, Sinorhizobium fredii NGR234, Azorhizobium caulinodans, Bradyrhizobium japonicum, Bradyrhizobium elkanii, Bradyrhizobium liaonginense, Rhizobium spp., Mesorhizobium spp., Sinorhizobium spp., Azorhizobium spp. Frankia spp., and any combination thereof, or the mycorrhizal fungi is selected from the group consisting of Acaulosporaceae spp., Diversisporaceae spp., Gigasporaceae spp., Pacisporaceae spp., Funneliformis spp., Glomus spp., Rhizophagus spp., Sclerocystis spp., Septoglomus spp., Claroideoglomus spp., Ambispora spp., Archaeospora spp., Geosiphon pyriformis, Paraglomus spp., other species in the division Glomeromycota, and any combination thereof.
   90. The genetically altered plant or part thereof of any one of embodiments 74-89, wherein the heterologous EPR3a or EPR3a-like polypeptide or the modified EPR3a or EPR3a-like polypeptide is localized to a plant cell plasma membrane.
   91. The genetically altered plant or part thereof of any one of embodiments 75-90, wherein the heterologous EPR3 or EPR3-like polypeptide or the modified EPR3 or EPR3-like polypeptide is localized to a plant cell plasma membrane.
   92. The genetically altered plant or part thereof of embodiment 90 or embodiment 91, wherein the plant cell is a root cell.
   93. The genetically altered plant or part thereof of embodiment 92, wherein the root cell is a root epidermal cell or a root cortex cell.
   94. The genetically altered plant or part thereof of any one of embodiments 74-93, wherein the heterologous EPR3a or EPR3a-like polypeptide or the modified EPR3a or EPR3a-like polypeptide is expressed in a developing plant root system.
   95. The genetically altered plant or part thereof of any one of embodiments 75-94, wherein the heterologous EPR3 or EPR3-like polypeptide or the modified EPR3 or EPR3-like polypeptide is expressed in a developing plant root system.
   96. The genetically altered plant or part thereof of any one of embodiments 74-95, wherein the first nucleic acid sequence is operably linked to a first promoter.
   97. The genetically altered plant or part thereof of embodiment 96, wherein the first promoter is a root specific promoter, and wherein the root specific promoter is optionally selected from the group consisting of a NFR1 or NFR5/NFP promoter, an EPR3 or an EPR3a promoter, a Lotus NFR5 promoter, a Lotus NFR1 promoter, a maize allothioneine promoter, a chitinase promoter, a maize ZRP2 promoter, a tomato LeExt1 promoter, a glutamine synthetase soybean root promoter, a RCC3 promoter, a rice antiquitine promoter, a LRR receptor kinase promoter, and an Arabidopsis pCO2 promoter.
   98. The genetically altered plant or part thereof of embodiment 96, wherein the first promoter is a constitutive promoter, and wherein the constitutive promoter is optionally selected from the group consisting of a CaMV35S promoter, a derivative of the CaMV35S promoter, a maize ubiquitin promoter, a trefoil promoter, a vein mosaic cassava virus promoter, and a Arabidopsis UBQ10 promoter.
   99. The genetically altered plant or part thereof of any one of embodiments 75-98, wherein the second nucleic acid sequence is operably linked to a second promoter.
   100. The genetically altered plant or part thereof of embodiment 99, wherein the second promoter is a root specific promoter, and wherein the root specific promoter is optionally selected from the group consisting of a NFR1 or NFR5/NFP promoter, an EPR3 or an EPR3a promoter, a Lotus NFR5 promoter, a Lotus NFR1 promoter, a maize allothioneine promoter, a chitinase promoter, a maize ZRP2 promoter, a tomato LeExt1 promoter, a glutamine synthetase soybean root promoter, a RCC3 promoter, a rice antiquitine promoter, a LRR receptor kinase promoter, and an Arabidopsis pCO2 promoter.
   101. The genetically altered plant or part thereof of embodiment 99, wherein the second promoter is a constitutive promoter, and wherein the constitutive promoter is optionally selected from the group consisting of a CaMV35S promoter, a derivative of the CaMV35S promoter, a maize ubiquitin promoter, a trefoil promoter, a vein mosaic cassava virus promoter, and an Arabidopsis UBQ10 promoter.
   102. The genetically altered plant or part thereof of any one of embodiments 74-101, wherein the plant is selected from the group consisting of cassava, corn, cowpea, rice, barley, wheat, Trema spp., apple, pear, plum, apricot, peach, almond, walnut, strawberry, raspberry, blackberry, red currant, black currant, melon, cucumber, pumpkin, squash, grape, tomato, pepper, and hemp.
   103. The genetically altered plant part of any one of embodiments 74-102, wherein the plant part is a leaf, a stem, a root, a root primordia, a flower, a seed, a fruit, a kernel, a grain, a cell, or a portion thereof.
   104. The genetically altered plant part of embodiment 103, wherein the part is a fruit, a kernel, or a grain.
   105. A pollen grain or an ovule of the genetically altered plant of any one of embodiments 74-101.
   106. A protoplast produced from the plant of any one of embodiments 74-101.
   107. A tissue culture produced from protoplasts or cells from the plant of any one of embodiments 74-101, wherein the cells or protoplasts are produced from a plant part selected from the group consisting of leaf, anther, pistil, stem, petiole, root, root primordia, root tip, fruit, seed, flower, cotyledon, hypocotyl, embryo, and meristematic cell.
   108. A method of producing the genetically altered plant of any one of embodiments 74-101, comprising introducing a genetic alteration to the plant comprising the first nucleic acid sequence encoding the heterologous EPR3a or EPR3a-like polypeptide.
   109. The method of embodiment 108, wherein the first nucleic acid sequence is operably linked to a first promoter.
   110. The method of embodiment 109, wherein the first promoter is a root specific promoter, and wherein the root specific promoter is optionally selected from the group consisting of a NFR1 or NFR5/NFP promoter, an EPR3 or an EPR3a promoter, a Lotus NFR5 promoter, a Lotus NFR1 promoter, a maize allothioneine promoter, a chitinase promoter, a maize ZRP2 promoter, a tomato LeExt1 promoter, a glutamine synthetase soybean root promoter, a RCC3 promoter, a rice antiquitine promoter, a LRR receptor kinase promoter, and an Arabidopsis pCO2 promoter.
   111. The method of embodiment 109, wherein the first promoter is a constitutive promoter, and wherein the constitutive promoter is optionally selected from the group consisting of a CaMV35S promoter, a derivative of the CaMV35S promoter, a maize ubiquitin promoter, a trefoil promoter, a vein mosaic cassava virus promoter, and an Arabidopsis UBQ10 promoter.
   112. The method of any one of embodiments 75-111, further comprising introducing a genetic alteration to the plant comprising the second nucleic acid sequence encoding the heterologous EPR3 or EPR3-like polypeptide.
   113. The method of embodiment 112, wherein the second nucleic acid sequence is operably linked to a second promoter.
   114. The method of embodiment 113, wherein the second promoter is a root specific promoter, and wherein the root specific promoter is optionally selected from the group consisting of a NFR1 or NFR5/NFP promoter, an EPR3 or an EPR3a promoter, a Lotus NFR5 promoter, a Lotus NFR1 promoter, a maize allothioneine promoter, a chitinase promoter, a maize ZRP2 promoter, a tomato LeExt1 promoter, a glutamine synthetase soybean root promoter, a RCC3 promoter, a rice antiquitine promoter, a LRR receptor kinase promoter, and an Arabidopsis pCO2 promoter.
   115. The method of embodiment 113, wherein the second promoter is a constitutive promoter, and wherein the constitutive promoter is optionally selected from the group consisting of a CaMV35S promoter, a derivative of the CaMV35S promoter, a maize ubiquitin promoter, a trefoil promoter, a vein mosaic cassava virus promoter, and an Arabidopsis UBQ10 promoter.
   116. The method of any one of embodiments 108-115, wherein the first nucleic acid sequence is inserted into the genome of the plant so that the nucleic acid sequence is operably linked to a first endogenous promoter.
   117. The method of embodiment 116, wherein the first endogenous promoter is a root specific promoter.
   118. The method of any one of embodiments 112-117, wherein the second nucleic acid sequence is inserted into the genome of the plant so that the nucleic acid sequence is operably linked to a second endogenous promoter.
   119. The method of embodiment 118, wherein the second endogenous promoter is a root specific promoter.
   120. A method of producing the genetically altered plant of any one of embodiments 80-119, comprising genetically editing a gene encoding an endogenous LysM receptor polypeptide in the plant to comprise the modified ectodomain.
   121. The method of embodiment 120, wherein the endogenous LysM receptor polypeptide is an endogenous EPR3a or EPR3a-like polypeptide.
   122. The method of embodiment 120 or embodiment 121, wherein the modified EPR3a or EPR3a-like polypeptide was generated by:
(a) providing a heterologous EPR3a or EPR3a-like polypeptide model comprising a structural model, a molecular model, a surface characteristics model, and/or an electrostatic potential model of a M1 domain, a M2 domain, a LysM3 domain, any combination thereof, or the ectodomain of the heterologous EPR3a or EPR3a-like polypeptide having selectivity for the beneficial commensal microbe and an unmodified EPR3a or EPR3a-like polypeptide;
(b) identifying one or more amino acid residues for modification in the unmodified EPRa3 polypeptide by comparing amino acid residues of a oligosaccharide binding feature in the unmodified EPR3a or EPR3a-like polypeptide with the corresponding amino acid residues in the heterologous EPR3a or EPR3a-like polypeptide model; and
(c) generating the unmodified EPR3a or EPR3a-like polypeptide wherein the one or more amino acid residues in the oligosaccharide binding feature of the unmodified EPR3a or EPR3a-like polypeptide have been substituted with corresponding amino acid residues from the heterologous EPR3a or EPR3a-like polypeptide.
   123. The method of embodiment 122, wherein the heterologous EPR3a or EPR3a-like polypeptide model is a protein crystal structure, a molecular model, a cryo-EM structure, and a NMR structure.
   124. The method of embodiment 120, wherein the endogenous LysM receptor polypeptide is an endogenous EPR3 or EPR3-like polypeptide.
   125. The method of embodiment 124, wherein the modified EPR3 or EPR3-like polypeptide was generated by:
(a) providing a heterologous EPR3 or EPR3-like polypeptide model comprising a structural model, a molecular model, a surface characteristics model, and/or an electrostatic potential model of a M1 domain, a M2 domain, a LysM3 domain, any combination thereof, or the ectodomain of the heterologous EPR3 or EPR3-like polypeptide having selectivity for the beneficial commensal microbe and an unmodified EPR3 or EPR3-like polypeptide;
(b) identifying one or more amino acid residues for modification in the unmodified EPR3 or EPR3-like polypeptide by comparing amino acid residues of a oligosaccharide binding feature in the unmodified EPR3 or EPR3-like polypeptide with the corresponding amino acid residues in the heterologous EPR3 or EPR3-like polypeptide model; and
(c) generating the unmodified EPR3 or EPR3-like polypeptide wherein the one or more amino acid residues in the oligosaccharide binding feature of the unmodified EPR3 or EPR3-like polypeptide have been substituted with corresponding amino acid residues from the heterologous EPR3 or EPR3-like polypeptide.
   126. The method of embodiment 125, wherein the heterologous EPR3 or EPR3-like polypeptide model is a protein crystal structure, a molecular model, a cryo-EM structure, and a NMR structure.
   127. A plant or part thereof produced by the method of any one of embodiments 108-126.
   128. A method of identifying a beneficial commensal microbe capable of participating in a plant root microbiota comprising:
a) providing a first polypeptide comprising an EPR3 or EPR3-like polypeptide, an ectodomain of an EPR3 or EPR3-like polypeptide, a M1 domain of an EPR3 or EPR3-like polypeptide, a M2 domain of an EPR3 or EPR3-like polypeptide, or a LysM3 domain of an EPR3 or EPR3-like polypeptide of the plant;
b) contacting the first polypeptide with a sample comprising a microbe or an EPS, a beta-glucan, a cyclic beta-glucan, a LPS, or a surface carbohydrate produced by the microbe; and
c) detecting binding of the EPS, the beta-glucan, the cyclic beta-glucan, the LPS, or the surface carbohydrate produced by the microbe to the polypeptide, wherein binding of the EPS, the beta-glucan, the cyclic beta-glucan, the LPS, or the surface carbohydrate to the polypeptide indicates that the microbe is a beneficial commensal microbe capable of participating in a plant root microbiota; optionally, the detecting is by a functional assay optionally selected from (i) detecting enrichment of taxa in Burkholderiales and/or Rhizobiales in a plant rhizosphere or endosphere, wherein enrichment of taxa in Burkholderiales and/or Rhizobiales in the plant rhizosphere or endosphere indicates that the microbe is a beneficial commensal microbe capable of participating in a plant root microbiota; optionally, (ii) detecting nodulation in a plant root system, wherein nodulation indicates that the microbe is a beneficial commensal microbe capable of participating in a plant root microbiota; and/or (iii) detecting mycorrhization in a plant root system, wherein mycorrhization indicates that the microbe is a beneficial commensal microbe capable of participating in a plant root microbiota, or optionally the detecting is by a direct binding assay optionally selected from (1) a competition assay optionally with a known signaling saccharide, or (2) an affinity assay optionally wherein the detected affinity is compared to the affinity for the known signaling saccharide.
   129. The method of embodiment 128, further comprising providing a second polypeptide comprising an EPR3a or EPR3a-like polypeptide, an ectodomain of an EPR3a or EPR3a-like polypeptide, a M1 domain of an EPR3a or EPR3a-like polypeptide, a M2 domain of an EPR3a or EPR3a-like polypeptide, or a LysM3 domain of an EPR3a or EPR3a-like polypeptide of the plant in step (a), wherein the second polypeptide is in contact with the first polypeptide.
   130. The method of embodiment 128 or embodiment 129, further comprising step (d) culturing the beneficial commensal microbe if binding is detected in step (c).
   131. The method of embodiment 130, further comprising step (e) applying the beneficial commensal microbe to the plant or a part thereof.
   132. The method of embodiment 131, wherein the plant part is a plant propagation material, optionally a seed, a tuber, or a plantlet, and the beneficial commensal microbe is applied to the plant propagation material, optionally to the seed as part of a seed coating, to the tuber, or to a root of the plantlet.
   133. The method of embodiment 131, wherein the plant part is a plant vegetative or reproductive material, optionally a root, a shoot, a stem, a pollen grain, or an ovule, and the beneficial commensal microbe is applied to the plant vegetative or reproductive material of the plant, optionally as part of a coating, a solution, or a powder.
   134. The method of embodiment 130 further comprising step (e) applying the beneficial commensal microbe, optionally in admixture with a soil-compatible carrier, a fungal carrier, or a growth medium, optionally soil, where the plant is growing or is to be grown.
   135. The method of any one of embodiments 128-134, wherein the beneficial commensal microbe is a commensal bacteria, optionally a nitrogen-fixing bacteria, or a mycorrhizal fungi.
   136. A method of identifying a beneficial commensal microbe capable of participating in a plant root microbiota comprising:
a) providing a first polypeptide comprising an EPR3a or EPR3a-like polypeptide, an ectodomain of an EPR3a or EPR3a-like polypeptide, a M1 domain of an EPR3a or EPR3a-like polypeptide, a M2 domain of an EPR3a or EPR3a-like polypeptide, or a LysM3 domain of an EPR3a or EPR3a-like polypeptide of the plant;
b) contacting the first polypeptide with a sample comprising a microbe or an EPS, a beta-glucan, a cyclic beta-glucan, a LPS, or a surface carbohydrate produced by the microbe; and
c) detecting binding of the EPS, the beta-glucan, the cyclic beta-glucan, the LPS, or the surface carbohydrate produced by the microbe to the polypeptide, wherein binding of the EPS, the beta-glucan, the cyclic beta-glucan, the LPS, or the surface carbohydrate to the polypeptide indicates that the microbe is a beneficial commensal microbe capable of participating in the plant root microbiota; optionally, the detecting is by a functional assay optionally selected from (i) detecting enrichment of taxa in Burkholderiales and/or Rhizobiales in a plant rhizosphere or endosphere, wherein enrichment of taxa in Burkholderiales and/or Rhizobiales in the plant rhizosphere or endosphere indicates that the microbe is a beneficial commensal microbe capable of participating in a plant root microbiota; optionally, (ii) detecting nodulation in a plant root system, wherein nodulation indicates that the microbe is a beneficial commensal microbe capable of participating in a plant root microbiota; and/or (iii) detecting mycorrhization in a plant root system, wherein mycorrhization indicates that the microbe is a beneficial commensal microbe capable of participating in a plant root microbiota, or optionally the detecting is by a direct binding assay optionally selected from (1) a competition assay optionally with a known signaling saccharide, or (2) an affinity assay optionally wherein the detected affinity is compared to the affinity for the known signaling saccharide.
   137. The method of embodiment 136, further comprising providing a second polypeptide comprising an EPR3 or EPR3-like polypeptide, an ectodomain of an EPR3 or EPR3-like polypeptide, a M1 domain of an EPR3 or EPR3-like polypeptide, a M2 domain of an EPR3 or EPR3-like polypeptide, or a LysM3 domain of an EPR3 or EPR3-like polypeptide of the plant in step (a), wherein the second polypeptide is in contact with the first polypeptide.
   138. The method of embodiment 136 or embodiment 137, further comprising step (d) culturing the beneficial commensal microbe if binding is detected in step (c).
   139. The method of embodiment 138, further comprising step (e) applying the beneficial commensal microbe to the plant or a part thereof.
   140. The method of embodiment 139, wherein the plant part is a plant propagation material, optionally a seed, a tuber, or a plantlet, and the beneficial commensal microbe is applied to the plant propagation material, optionally to the seed as part of a seed coating, to the tuber, or to a root of the plantlet.
   141. The method of embodiment 139, wherein the plant part is a plant vegetative or reproductive material, optionally a root, a shoot, a stem, a pollen grain, or an ovule, and the beneficial commensal microbe is applied to the plant vegetative or reproductive material of the plant, optionally as part of a coating, a solution, or a powder.
   142. The method of embodiment 138 further comprising step (e) applying the beneficial commensal microbe, optionally in admixture with a soil-compatible carrier, a fungal carrier, or a growth medium, optionally soil, where the plant is growing or is to be grown.
   143. The method of any one of embodiments 136-142, wherein the beneficial commensal microbe is a commensal bacteria, optionally a nitrogen-fixing bacteria, or a mycorrhizal fungi.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1D show purification of the EPR3 ectodomain (ED)-Nb186 complex and the crystal structure of the EPR3 ED-Nb186 complex. FIG. 1A shows overlay of gel filtration runs with EPR3 ED (EPR3; light gray), Nb186 (dark gray), and the EPR3 ED-Nb186 complex (EPR3-Nb186; gray) (x-axis shows elution volume in ml; y-axis shows absorbance (Abs.) at 280 nm (mAU)). FIG. 1B shows SDS-PAGE of the EPR3 ED-Nb186 complex used for crystallization with the bands corresponding to EPR3 ED (EPR3) and Nb186 labeled on the right, and the molecular weights of the protein bands provided on the left in kilodaltons (kDa). FIG. 1C shows a representative image of a single EPR3 ED-Nb186 crystal. FIG. 1D shows the crystal structure of the EPR3 ED-Nb186 complex in two views (right view rotated 90° relative to left view), with Nb186 colored in dark gray (N-terminus (N) and C-terminus (C) are labeled) and EPR3 ED colored in shades of lighter gray (N-terminus (N), M1 domain (M1; gray), M2 domain (M2; light gray), LysM3 domain (LysM3; light gray), and C-terminus (C) are labeled).

FIGS. 2A-2D show the crystal structure of the EPR3 ED. FIG. 2A shows a cartoon representation of the EPR3 ED crystal structure in two views (right view rotated 90° relative to left view) with differently colored domains (M1 in gray; M2 in light gray; and LysM3 in light gray) having labels indicating the N and C termini, secondary structures (M1 α-helix is numbered α1, M1 β-sheets are numbered β1, β2, and β3; M2 α-helix is numbered α2, M2 β-sheets are numbered β4 and β5; LysM3 α-helices are numbered α3 and a4, and LysM3 β-sheets are numbered β6 and 137), and disulfide bridges indicated with arrows and the corresponding connected residues (C98-C155, C47-C100, and C54-C157). FIG. 2B shows the EPR3 ED carbohydrate-binding domain M1 with labels indicating the N and C termini and secondary structures (α1, β1, β2, and β3) (top), and M1 with labels indicating the N and C termini superimposed to the corresponding LysM1 domain in CERK6 (PDB-5LS2) colored in light gray (bottom). FIG. 2C shows the EPR3 ED carbohydrate-binding module M2 with labels indicating the N and C termini and secondary structures (α2, β4, and β5) (top), and M2 with labels indicating the N and C termini superimposed to the corresponding LysM2 domain in CERK6 (PDB-5LS2) colored in light gray (bottom). FIG. 2D shows the EPR3 ED carbohydrate-binding module LysM3 with labels indicating the N and C termini and secondary structures (α3, α4, β6, and β7) (top), and LysM3 with labels indicating the N and C termini superimposed to the corresponding LysM3 domain in CERK6 (PDB-5LS2) colored in light gray (bottom).

FIGS. 3A-3F show small-angle X-ray scattering (SAXS) analysis of the EPR3 ED and the stem region of EPR4 homologs alignment logo. FIG. 3A shows the EPR3 ED SAXS scattering curve with model fit (χ²=1.093; x-axis in s(Å⁻¹); y-axis in log(I)). FIG. 3B shows the EPR3 ED SAXS Guinier plot. FIG. 3C shows the EPR3 ED SAXS pair distance distribution (P(r)) plot with a Dₘₐₓ of 73.4 Å. FIG. 3D shows docking of the EPR3 ED crystal structure into the SAXS envelope showing an extended stem-like structure. The overall dimensions are shown in angstrom (Å; height ˜73 Å, width ˜45 Å, stem length ˜36 Å). FIG. 3E shows a model of the EPR3 receptor where the stem structure of the EPR3 ED positions the EPR3 ED with a distance to the plasma membrane (PM), and connects the EPR3 ED to the transmembrane domain (gray bar in PM) and the cell-internal kinase domain (kinase; gray oval). FIG. 3F shows the alignment logo of the stem region of EPR3 homologs with the sequence of the stem region of L. japonicus EPR3 shown below (SEQ ID NO: 188).

FIGS. 4A-4C show sequence alignments of the EPR3 ED M1, M2, and LysM3 domains from EPR3 homologs in dicot (legumes and non-legumes) and monocot species L. japonicus (BAI79269.1), Cicer arietinum (Chickpea; XP_004489790.1), Medicago truncatula (XP_003613165.1), Glycine max (Soybean XP_003517716.1), Phaseolus vulgaris (XP_007157313.1), Populus trichocarpa (XP_002322185.1), Malus domestica (XP_008340354.1), Vitis vinifera (XP_002272814.2), Theobroma cacao (XP_007036352.1), Ricinus communis (XP_002527912.1), Fragaria vesca subsp. vesca (XP_004300916.1), Zea mays (Maize; XP_008657477.1), Oryza sativa Japonica Group (Rice; XP_015628733.1), Triticum aestivum (Wheat; CDM80098.1), and Hordeum vulgare (Barley; MLOC_5489.2). FIG. 4A shows sequence alignment of EPR3 ED homologs showing the conserved secondary structure arrangement of the M1 domain (βαββ fold). The β-sheet β1″ is highlighted in gray with darker gray text, the α-helix “α1” is highlighted in gray with darker gray text, the β-sheet “β2” is highlighted in light gray with gray text, the β sheet “β3” is highlighted gray with darker gray text, and conserved cysteine residues are highlighted in dark gray with black text (Lotus=SEQ ID NO: 16, Chickpea=SEQ ID NO: 17, Medicago=SEQ ID NO: 18, Soybean=SEQ ID NO: 19, Phaseolus=SEQ ID NO: 20, Populus=SEQ ID NO: 21, Malus=SEQ ID NO: 22, Vitis=SEQ ID NO: 23, Theobroma=SEQ ID NO: 24, Ricinus=SEQ ID NO: 25, Fragaria=SEQ ID NO: 26, Maize=SEQ ID NO: 27, Rice=SEQ ID NO: 28, Wheat=SEQ ID NO: 29, Barley=SEQ ID NO: 30; Consensus=SEQ ID NO: 189). FIG. 4B shows sequence alignment of EPR3 ED homologs showing the conserved secondary structure arrangement of the M2 domain (βαβ fold). The β-sheets “β4” and “β5” are highlighted in light gray with gray text, the α-helix “α2” is highlighted in light gray with gray text, and conserved cysteine residues are highlighted in dark gray with black text (Lotus=SEQ ID NO: 31, Chickpea=SEQ ID NO: 32, Medicago=SEQ ID NO: 33, Soybean=SEQ ID NO: 34, Phaseolus=SEQ ID NO: 35, Populus=SEQ ID NO: 36, Malus=SEQ ID NO: 37, Vitis=SEQ ID NO: 38, Theobroma=SEQ ID NO: 39, Ricinus=SEQ ID NO: 40, Fragaria=SEQ ID NO: 41, Maize=SEQ ID NO: 42, Rice=SEQ ID NO: 43, Wheat=SEQ ID NO: 44, Barley=SEQ ID NO: 45; Consensus=SEQ ID NO: 190). FIG. 4C shows sequence alignment of EPR3 ED homologs showing the conserved secondary structure arrangement of the LysM3 domain (βααβ fold). The β-sheets “β6” and “β7” are highlighted in light gray with gray text, and the α-helices “α3” and “α4” are highlighted in light gray with dark gray text (Lotus=SEQ ID NO: 46, Chickpea=SEQ ID NO: 47, Medicago=SEQ ID NO: 48, Soybean=SEQ ID NO: 49, Phaseolus=SEQ ID NO: 50, Populus=SEQ ID NO: 51, Malus=SEQ ID NO: 52, Vitis=SEQ ID NO: 53, Theobroma=SEQ ID NO: 54, Ricinus=SEQ ID NO: 55, Fragaria=SEQ ID NO: 56, Maize=SEQ ID NO: 57, Rice=SEQ ID NO: 58, Wheat=SEQ ID NO: 59, Barley=SEQ ID NO: 60; Consensus=SEQ ID NO: 191).

FIGS. 5A-5C show structural modelling of the M1 domain from EPR3 homologs. FIG. 5A shows ab-initio models of the EPR3 M1 domain from receptor homologs revealing conserved βαββ structures. Molecular fits (root-mean-square deviation of atomic position, noted as RMSD values) based on superposition of these modelled M1 domains to the crystal structure of the EPR3 ED M1 domain are denoted in A (Angstrom). The N- and C-termini of the domains are labeled. FIG. 5B shows a side-by-side comparison of the L. japonicus EPR3 ED crystal structure of the M1 domain (Lotus EPR3 M1—crystal structure; left) and an ab-initio atomic-level force field model of L. japonicus EPR3 ED M1 (Lotus EPR3 M1—modelled; right). The β-sheets and α-helix secondary structures that make up the βαββ fold of the M1 domain (α1, β1, β2, and β3), and the N- and C-termini are labeled. FIG. 5C shows modelling of the M1 domain from EPR3 homologs, revealing the same overall βαββ arrangement. Models of the M1 domain from L. japonicus EPR3 and EPR3 homologs from Chickpea, Medicago, Soybean, Phaseolus, Populus, Malus, Vitis, Theobroma, Ricinus, Fragaria, Maize, and Wheat. The molecular fit (root-mean-square deviation of atomic positions, or RMSD) in A to the crystal structure of EPR3-M1 is noted for each model. The N- and C-termini of the domains are labeled.

FIG. 6 shows a structural comparison of plant receptors. Structural overviews of the exopolysaccharide (EPS) receptor EPR3 ED (left), the chitin receptor CERK6 ED (center), and the lipo-chitooligosaccharide (LCO) receptor NFP ED (right) are shown. The receptor EDs are colored such that the N-terminal domain (M1 or LysM1) is gray, the center domain (M2 or LysM2) is lighter gray, and the C-terminal domain (LysM3) is light gray, as indicated by the schematic model below the structures. The molecular fit (root-mean-square deviation of atomic positions, or RMSD) in Å between the receptors are indicated, with EPR3 ED to CERK6 ED RMSD=4.150 Å, EPR3 ED to NFP ED RMSD=5.096 Å, and CERK6 ED to NFP ED RMSD=2.059 Å.

FIGS. 7A-7F show the proposed structures of the exopolysaccharides (EPS) ligands and matrix-assisted laser desorption/ionization time of flight (MALDI-TOF) mass spectrometry spectra are shown. FIG. 7A shows the Mesorhizobium loti strain R7A EPS proposed structure and mass spectrometry spectrum. FIG. 7B shows the M. loti strain R7A deOAc-EPS proposed structure and mass spectrometry spectrum. FIG. 7C shows the M. loti strain R7A exoU EPS proposed structure and mass spectrometry spectrum. FIG. 7D shows the chitohexose (C06) proposed structure. FIG. 7E shows the R. leguminosarum EPS proposed structure and mass spectrometry spectrum. FIG. 7F shows the S. meliloti EPS proposed structure and mass spectrometry spectrum.

FIGS. 8A-8G show microscale thermophoresis (MST) experiments measuring the binding of the EPR3 ED to exopolysaccharides (EPS). FIG. 8A shows a MST binding experiment with EPR3 ED and M. loti strain R7A EPS. FIG. 8B shows a MST binding experiment with EPR3 ED and M. loti strain R7A exoU EPS. FIG. 8C shows a MST binding experiment with EPR3 ED and chitin (C06). FIG. 8D shows a MST binding experiment with EPR3 ED and M. loti strain R7A de-O-acetylated EPS (deOAc-EPS). FIG. 8E shows a MST binding experiment with EPR3 ED and EPS from R. leguminosarum. FIG. 8F shows a MST binding experiment with EPR3 ED and EPS from S. meliloti. FIG. 8G shows a table summarizing the equilibrium dissociation constants value (K_{d}) in the 95% confidence interval for the different ligands, and “NB” indicates no detectable binding. In FIGS. 8A-8F, the x-axis shows the molar concentration of the EPS ligand (M), the y-axis shows the percent change in normalized fluorescence (ΔFₙₒᵣₘ(%)), and the equilibrium dissociation constant (K_{d}) of each binding curve, the corresponding goodness of fit (R²), and number of replicates performed using independent protein preparations (n) are indicated. “NB” indicates no detectable binding, where applicable.

FIGS. 9A-9B show microscale thermophoresis (MST) experiments measuring the binding of de-glycosylated EPR3 ED or de-glycosylated EPR3 ED-Nb186 complex to EPS.

FIG. 9A shows a MST binding experiment with de-glycosylated EPR3 ED and M. loti strain R7A EPS. FIG. 9B shows a MST binding experiment with de-glycosylated EPR3 ED-Nb186 complex and M. loti strain R7A EPS. In FIGS. 9A-9B the x-axis shows the molar concentration of the EPS ligand (M), the y-axis shows the percent change in normalized fluorescence(ΔFₙₒᵣₘ(%)), and the equilibrium dissociation constants (K_{d}) in the 95% confidence interval, goodness of fit (R²), and number of independent protein preparations used for the measurements (n) are reported.

FIGS. 10A-10C show small-angle X-ray scattering (SAXS) data, fits and models of EPR ED alone, EPR ED in the presence of M. loti strain R7A EPS, or EPR ED in the presence of M. loti strain R7A exoU EPS. FIG. 10A shows SAXS data for EPR3 ED in the absence of ligand with SAXS scattering model fit χ²=1.093, Dₘₐₓ=73.4 Å, and overall dimensions of 73 Å by 45 Å. FIG. 10B shows SAXS data for EPR3 ED in the presence of M. loti strain R7A EPS with SAXS scattering model fit χ²=1.053, Dₘₐₓ=67.3 Å, and overall dimensions of 67 Å by 43 Å. FIG. 10C shows SAXS data for EPR3 ED in the presence of M. loti strain R7A exoU EPS with SAXS scattering model fit χ²=1.158, Dₘₐₓ=72.7 Å, and overall dimensions of 73 Å by 48 Å. In FIGS. 10A-10C, the SAXS scattering curve with model fit (χ²) is shown on left, Guinier plot is shown top middle, pair distance distribution (P(r)) plot with Dₘₐₓ indicated is shown bottom middle, and a model docking the EPR3 ED crystal structure into the SAXS envelope, with overall dimensions of the model shown in angstrom (A) is shown on right.

FIGS. 11A-11M show comparisons of the L. japonicus Epr3 and Epr3a genes and amino acid sequences, a microscale thermophoresis (MST) experiment measuring the binding of the L. japonicus EPR3a ED to EPS, and a kinase activity assay to measure the activity of the EPR3 and EPR3a kinase domains. FIG. 11A shows Epr3 and Epr3a gene models with the relative sizes and positions of 5′ UTRs shown as dark gray rectangles, exons shown as light gray rectangles, introns shown as black lines, and 3′ UTRs shown as darker gray rectangles. Relative positions of insertion of the Lotus retrotransposon 1 element (LORE1) in the mutant lines are indicated with black triangles and labeled according to the name of the mutant epr3 line (epr3-11 in Epr3; epr3a-1 and epr3a-2 in Epr3a). FIG. 11B shows a protein alignment of L. japonicus EPR3 (SEQ ID NO: 61) and L. japonicus EPR3a (SEQ ID NO: 62). Conserved residues are indicated by a ‘|’, highly similar residues by ‘:’, weakly similar residues by ‘.’ and gaps by ‘-’. FIG. 11C shows a MST binding experiment with L. japonicus EPR3a ED and M. loti EPS. The x-axis shows the molar concentration of the EPS ligand (M), the y-axis shows the percent change in normalized fluorescence (ΔFₙₒᵣₘ(%)); the equilibrium dissociation constant (K_{d}=25.6 μM), and the corresponding goodness of fit (R²=0.98) are indicated. FIG. 11D shows the result of purifying the ectodomain of Lotus EPR3a from insect cells. At left, FIG. 11D provides the results of gel filtration using a Superdex 75 10/300 column (x-axis shows elution volume (Ve) in ml; y-axis shows absorbance (Abs.) at 280 nm (mAU)), with peak absorbance at an elution volume of 11.45 ml. At right, FIG. 11D provides SDS-PAGE of the purified L. japonicus EPR3a ED with samples corresponding to the elution volumes shown at left labeled 8, and 12-16, and the molecular weights of the protein bands provided on the left in kilodaltons (kDa). The well labeled P contains PNGase F, and the well labeled G contains Glycosylated EPR3. The samples in wells 15 and 16 were used for the MST experiments shown in FIGS. 11E-11J. FIGS. 11E-11J show the results of MST experiments measuring binding of the L. japonicus EPR3a ED to polysaccharides. FIG. 11E shows binding of the L. japonicus EPR3a ED to M. loti EPS, with the equilibrium dissociation constant (K_{d}=44.4±11.2 μM), corresponding goodness of fit (R²=0.97), and sample size (n=3) indicated. FIG. 11F shows binding of the L. japonicus EPR3a ED to M. loti de-O-acetylated (deOAc) EPS, with the equilibrium dissociation constant (K_{d}=57.2±15.6 μM), corresponding goodness of fit (R²=0.96), and sample size (n=3) indicated. FIG. 11G shows binding of the L. japonicus EPR3a ED to S. meliloti EPS, with the equilibrium dissociation constant (K_{d}=936.7±389.4 μM), corresponding goodness of fit (R²=0.93), and sample size (n=3) indicated. FIG. 11H shows binding of the L. japonicus EPR3a ED to R. leguminosarum EPS, with the equilibrium dissociation constant (K_{d}=12.5±4.0 μM), corresponding goodness of fit (R²=0.95), and sample size (n=3) indicated. FIG. 11I shows the absence of binding of the L. japonicus EPR3a ED to chitotetraose (C04), with the sample size (n=3) indicated. FIG. 11J shows the absence of binding of the L. japonicus EPR3a ED to maltodextrin (MalDex), with the sample size (n=3) indicated. In each of FIGS. 11E-11J, the x-axis shows the molar concentration of the polysaccharide (M), the y-axis shows the percent change in normalized fluorescence (ΔFₙₒᵣₘ(%)), and the error bars show the 95% confidence interval. FIG. 11K shows kinase activity of L. japonicus EPR3 kinase domains purified from E. coli. FIG. 11L shows kinase activity of L. japonicus EPR3a kinase domains purified from E. coli. FIG. 11M shows kinase activity of L. japonicus EPR3a kinase domains purified from E. coli.

FIG. 12 shows RNA-seq data from L. japonicus Gifu showing expression of Epr3 (top row) and Epr3a (bottom row) across tissue types when treated with H₂O or the symbiotic bacteria M loti strain R7A. Relative expression is shown in root hairs, roots, nodules, and shoots each treated with either H₂O (mock) or M loci strain R7A (R7A), as indicated. For tissues treated with M. loti strain R7A, the number of days between the collection of the RNA and the treatment with M. loti strain R7A is indicated (dpi, days post inoculation). The normalized expression of Epr3 and Epr3a is indicated by the gray scale shown, with dark gray indicating the highest and lowest levels of relative expression.

FIGS. 13A-13B show the results of plate nodulation assays of L. japonicus of the indicated genotypes inoculated with different M. loti strains. FIG. 13A shows the results of plate nodulation assays of L. japonicus genotypes with M. loti strain R7A. FIG. 13B shows the results of plate nodulation assays of L. japonicus genotypes with M. loti strain R7AexoY/F. In FIGS. 13A-13B, the L. japonicus genotypes tested were wild type (WT) Gifu, epr3-11 single mutant, epr3a-1 single mutant, epr3a-2 single mutant, and epr3/epr3a double mutant. Both nitrogen-fixing (“Pink”; gray color) and uninfected (“White”; white color) nodules were counted periodically over 35 days. The x-axis shows the number of days post-treatment with M. loti (dpi), and the y-axis shows the number of nodules per plant (n=30/condition). Error bars represent SEM and statistical comparisons between genotypes for pink nodules are shown using ANOVA and Tukey post hoc testing with p-value (<0.05) and different letters indicating a statistically significant difference.

FIGS. 14A-14C show phenotypes of wild type (WT) L. japonicus Gifu compared to phenotypes of L. japonicus with mutations in epr3 and/or epr3a when inoculated with M. loti strain R7A. FIG. 14A shows pink nodules formed on the indicated genotypes inoculated with M. loti strain R7A at 4 weeks post inoculation. The x-axis indicates the genotype of the plant and the sample size per genotype (Gifu WT, n=25; epr3-11 single mutant, n=39; epr3a-1 single mutant, n=40; epr3a-2 single mutant, n=42; and epr3/epr3a double mutant, n=41) and the y-axis represents the number of nodules per plant. FIG. 14B shows fresh shoot weights of pot grown plants 4 weeks post inoculation with M. loti strain R7A. The x-axis indicates the genotype of the plant and the sample size per genotype (Gifu WT, n=5; epr3-11 single mutant, n=6; epr3a-1 single mutant, n=6; epr3a-2 single mutant, n=6; and epr3/epr3a double mutant, n=6) and the y-axis indicates the weight of the shoot (grams). FIG. 14C shows appearance of the pot-grown plants 4 weeks post inoculation with M. loti strain R7A. The plants are arranged according to genotype (in order: Gifu WT, epr3-11 single mutant, epr3a-1 single mutant, epr3a-2 single mutant, and epr3/epr3a double mutant). For FIGS. 14A-14B, box plots show the first quartile and third quartile with the line indicating the median (dots represent individual measurements); statistical comparisons between genotypes are shown using ANOVA and Tukey post hoc testing with p-value (<0.05), and different letters indicate a statistically significant difference.

FIGS. 15A-15C show phenotypes of wild-type L. japonicus Gifu compared to phenotypes of L. japonicus with mutations in epr3 and/or epr3a when inoculated with M. loti strain R7AexoY/F. FIG. 15A shows pink nodules formed on the indicated genotypes inoculated with M. loti strain R7AexoY/F at 4 weeks post inoculation. The x-axis indicates the genotype of the plant and the sample size per genotype (Gifu WT, n=42; epr3-11 single mutant, n=39; epr3a-1 single mutant, n=39; epr3a-2 single mutant, n=40; and epr3/epr3a double mutant, n=37) and the y-axis represents the number of nodules per plant. FIG. 15B shows fresh shoot weights of pot grown plants 4 weeks post inoculation with M. loti strain R7AexoY/F. The x-axis indicates the genotype of the plant and the sample size per genotype (Gifu WT, n=6; epr3-11 single mutant, n=6; epr3a-1 single mutant, n=6; epr3a-2 single mutant, n=6; and epr3/epr3a double mutant, n=6) and the y-axis represents shoot weight (grams). FIG. 15C shows the appearance of the pot-grown plants 4 weeks post inoculation with M. loti strain R7AexoY/F. The plants are arranged according to genotype (in order: Gifu WT, epr3-11 single mutant, epr3a-1 single mutant, epr3a-2 single mutant, and epr3/epr3a double mutant). For FIGS. 15A-15B, box plots show the first quartile and third quartile with the line indicating the median (dots represent individual measurements); statistical comparisons between genotypes are shown using ANOVA and Tukey post hoc testing with p-value (<0.05), and different letters indicate a statistically significant difference.

FIGS. 16A-16C show phenotypes of wild-type L. japonicus Gifu compared to phenotypes of L. japonicus with mutations in epr3 and/or epr3a when inoculated with M. loti strain R7AexoU. FIG. 16A shows pink nodules formed on the indicated genotypes inoculated with M. loti strain R7AexoU at 5 weeks post inoculation. The x-axis indicates the genotype of the plant and the sample size per genotype (Gifu WT, n=36; epr3-11 single mutant, n=32; epr3a-1 single mutant, n=38; epr3a-2 single mutant, n=35; and epr3/epr3a double mutant, n=36) and the y-axis represents the number of nodules per plant. FIG. 16B shows fresh shoot weights of pot grown plants 5 weeks post inoculation with M. loti strain R7AexoU. The x-axis indicates the genotype of the plant and the sample size per genotype (Gifu WT, n=6; epr3-11 single mutant, n=6; epr3a-1 single mutant, n=6; epr3a-2 single mutant, n=6; and epr3/epr3a double mutant, n=6) and the y-axis represents shoot weight (grams). FIG. 16C shows the appearance of the pot grown plants 5 weeks post inoculation with M. loti strain R7AexoU. The plants are arranged according to genotype (in order: Gifu WT, epr3-11 single mutant, epr3a-1 single mutant, epr3a-2 single mutant, and epr3/epr3a double mutant). For FIGS. 16A-16B, box plots show the first quartile and third quartile with the line indicating the median (dots represent individual measurements); statistical comparisons between genotypes are shown using ANOVA and Tukey post hoc testing with p-value (<0.05), and different letters indicate a statistically significant difference.

FIG. 17 shows the number of infection threads formed on wild-type L. japonicus Gifu compared to phenotypes of L. japonicus with mutations in epr3 and/or epr3a at 8 days post inoculation with M. loti strain R7A. The x-axis indicates the genotype of the plant and the sample size per genotype (Gifu WT, n=20; epr3-11 single mutant, n=20; epr3a-1 single mutant, n=20; epr3a-2 single mutant, n=20; and epr3/epr3a double mutant, n=10) and the y-axis represents the number of infection threads per plant (ITs/plant). The box plots show the first quartile and third quartile with the line indicating the median (dots represent individual measurements); statistical comparisons between genotypes are shown using ANOVA and Tukey post hoc testing with p-value (<0.05), and different letters indicate a statistically significant difference.

FIGS. 18A-18C show qRT-PCR of the symbiotic genes Nfyal, Npl, and Gh3.3 in wild-type L. japonicus (Gifu) or L. japonicus with mutations in epr3 and/or epr3a (labels below x-axis; epr3-11 single mutant, epr3a-1 single mutant, epr3a-2 single mutant, and epr3/epr3a double mutant). FIG. 18A shows qRT-PCR of the symbiotic gene Nfyal in wild-type L. japonicus (Gifu) or epr3-11 single mutant, epr3a-1 single mutant, epr3a-2 single mutant, and epr3/epr3a double mutant L. japonicus. FIG. 18B shows qRT-PCR of the symbiotic gene Npl in wild-type L. japonicus (Gifu) or epr3-11 single mutant, epr3a-1 single mutant, epr3a-2 single mutant, and epr3/epr3a double mutant L. japonicus. FIG. 18C shows qRT-PCR of the symbiotic gene Gh3.3 in wild-type L. japonicus (Gifu) or epr3-11 single mutant, epr3a-1 single mutant, epr3a-2 single mutant, and epr3/epr3a double mutant L. japonicus. In FIGS. 18A-18C, plants were treated with H₂O or M. loti strain R7A, and symbiotic gene expression was measured at 3 and 7 days post inoculation (dpi), as indicated on the x-axis. The y-axis represents the absolute expression of each transcript. The results represent the averages of three biological replicates with error bars indicating SEM.

FIGS. 19A-19B show models of symbiotic signaling output from the L. japonicus EPS receptors EPR3 and EPR3a in different genetic backgrounds (Gifu WT, epr3 single mutant, epr3a single mutant, epr3/epr3a double mutant). FIG. 19A shows a model of symbiotic signaling output from the L. japonicus EPS receptors in response to M. loti strain R7A wild-type EPS (large gray oval). FIG. 19B shows a model of symbiotic signaling output from the L. japonicus EPS receptors in response to M. loti strain R7AexoU truncated EPS (small gray oval). In FIGS. 19A-19B the relative strength of positive or negative symbiotic signaling in each of the plant genotypes is represented by the number of + or − symbols.

FIGS. 20A-20D show the phenotype of L. japonicus wild-type and epr-3 mutants when grown in natural soil. FIG. 20A shows shoot appearance after 7 weeks of growth in natural soil. Gifu represents wild-type L. japonicus, and all other plants are mutant in epr3 (epr3-11, epr3-10, and epr3-13). FIG. 20B shows shoot fresh weight (average and SEM are shown). The x-axis indicates the genotype of the plant (Gifu WT, epr3-11 mutant, epr3-10 mutant, and epr3-13 mutant) as well as the number of shoots measured in parentheses. The y-axis represents the shoot fresh weight of each plant in grams. FIG. 20C shows total nodule number (average and SEM are shown). The x-axis indicates the genotype of the plant (Gifu WT, epr3-11 mutant, epr3-10 mutant, and epr3-13 mutant) as well as the number of shoots measured in parentheses. The y-axis represents the total number of nodules per plant. FIG. 20D shows a schematic of the Epr3 gene with black boxes denoting the ten exons, dashed lines denoting the introns, and the positions of the start (labeled with ATG) and the end (labeled with TGA) as well as locations of the mutant alleles epr3-3, epr3-11, exo277 (epr3-10), epr3-12, epr3-13, and epr3-9 shown (from Kawaharada, Y et al. Nature 2015 523: 308-312).

FIGS. 21A-21D show α- and β-diversity analyses of L. japonicus wild-type (Gifu) and epr3-13 mutant microbiota. FIG. 21A shows Shannon indices of the diversity of bacteria in soil (dark gray), the rhizosphere (light gray), roots (gray), and nodules (black). The Shannon index of wild-type L. japonicus (Gifu) is shown as unfilled squares, the Shannon index of epr3-13 is shown as opaque circles, and soil is shown as Xs (box plots show the first quartile and third quartile with the line indicating the median). FIG. 21B shows a principal coordinates analysis (PCoA) plot of Bray-Curtis distances between samples from soil (dark gray), the rhizosphere (light gray), roots (gray), and nodules (black). L. japonicus (Gifu) is shown as opaque circles, epr3-13 mutant is shown as unfilled squares, and soil is shown as opaque triangles. The first principle component that explains 46.19% of the variance is plotted on the x-axis, and the second principle component that explains 20.17% of the variance is plotted on the y-axis. FIG. 21C shows a constrained PCoA plot of Bray-Curtis distances constrained by both genotype and compartment. Samples from the rhizosphere compartment are shown in lighter gray, and samples from roots are shown in gray. L. japonicus (Gifu) is shown as unfilled squares, epr3-13 mutant is shown as opaque circles. The figure shows microbiota dissimilarity (36.9% of variance explained by the two constrains, p<0.001, n=36). FIG. 21D shows a constrained PCoA plot of Bray-Curtis distances only constrained by genotype. Samples from the rhizosphere compartment are shown in lighter gray, and samples from roots are shown in gray. L. japonicus (Gifu) is shown as unfilled squares, epr3-13 is shown as opaque circles. In FIGS. 21B-21D the percentage in each axis indicates the fraction of total variance explained by projection.

FIGS. 22A-22B show the effects of mutations in Epr3 on the composition of the rhizosphere (FIG. 22A) and root (FIG. 22B) bacterial communities at distinct taxonomic level. FIG. 22A shows bacterial operational taxonomic units (OTUs) identified in the rhizosphere compartment grouped by taxa and arranged in Manhattan plots. FIG. 22B shows bacterial OTUs identified in the root compartment grouped by taxa and arranged in Manhattan plots. In FIGS. 22A-22B, OTUs are arranged on the x-axis, and the y-axis shows the significance of the difference in relative abundance of an OTU between L. japonicus wild-type and epr3-13 mutant plants (−log₁₀(p)). Each OTU is represented as a filled (statistically enriched in L. japonicus wild-type Gifu versus epr3-13) or empty (statistically not enriched in L. japonicus wild-type Gifu versus epr3-13) circle. The size of the circle is adjusted according to the relative abundance of the respective OTU in the analyzed compartment (see key at bottom of FIG. 22B). From left to right, the OTUs are grouped by the taxa Actinomycetales, Flavobacteriales, unknown, Caulobacterales, Rhizobiales, Rhodospirillales, Sphingomonadales, Burkholderiales, Pseudomonadales, and Xanthomonadales (labeled above the Manhattan plots).

FIGS. 23A-23D show the effect of Epr3 mutation on rhizosphere colonization of most abundant taxa. The average number of reads (FIGS. 23A-23B) and the relative abundance (FIGS. 23C-23D) of the top most abundant 100 OTUs identified in the rhizosphere compartment of wild-type L. japonicus Gifu are shown. FIG. 23A shows the average number of read counts for individual OTUs (column “OTUId”) of the taxa Betaproteobacteria and Burkholderiales in wild-type L. japonicus Gifu (column “G”) and epr3-13 (column “e”), represented as a heatmap (ranging from relatively higher read counts in dark gray, and relatively fewer read counts in light gray; key at bottom of FIG. 23B), the ratio between wild-type L. japonicus Gifu and epr3-13 average read count (column “G/e”), and the relative abundance (column “RA”) of the respective OTU in the indicated compartment of wild-type L. japonicus Gifu shown as horizontal bars. FIG. 23B shows the average number of read counts for individual OTUs (column “OTUId”) of the taxa Burkholderiales, Caulobacterales, Methylophilales, Pseudomonadales, Rhizobiales, Rhodospiralles, and Sphingomonadales in wild-type L. japonicus Gifu (column “G”) and epr3-13 (column “e”), represented as a heatmap (ranging from relatively higher read counts in dark gray, and relatively fewer read counts in light gray; key at bottom), the ratio between wild-type L. japonicus Gifu and epr3-13 average read count (column “G/e”), and the relative abundance (column “RA”) of the respective OTU in the indicated compartment of wild-type L. japonicus Gifu shown as horizontal bars. FIG. 23C shows the relative abundance of individual OTUs (column “OTUId”) grouped by taxa (column “Taxa”), order, and family (column “Family(f)/Order(o)”) in wild-type L. japonicus Gifu (column “G”) and epr3-13 (column “e”) represented as heatmap (ranging from relatively more abundant in light gray, and relatively less abundant in dark gray; key at bottom in FIG. 23D), and the relative abundance (column “RA”) of the respective OTU in wild-type L. japonicus Gifu, shown as horizontal bars (labeled dark gray if enriched in wild-type L. japonicus Gifu (p<0.05), labeled gray if enriched in epr3-13 (p<0.05), or labeled light gray if not significantly changed between the two genotypes). FIG. 23D shows the relative abundance of individual OTUs (column “OTUId”) grouped by taxa (column “Taxa”), order, and family (column “Family(f)/Order(o)”) in wild-type L. japonicus Gifu (column “G”) and epr3-13 (column “e”) represented as heatmap (ranging from relatively more abundant in light gray, and relatively less abundant in dark gray; key at bottom), and the relative abundance (column “RA”) of the respective OTU in wild-type L. japonicus Gifu, shown as horizontal bars (labeled dark gray if enriched in wild-type L. japonicus Gifu (p<0.05), labeled gray if enriched in epr3-13 (p<0.05), or labeled light gray if not significantly changed between the two genotypes).

FIG. 24 shows the effect of mutation of Epr3 on the abundance of Burkholderiales in the root compartment. Relative abundance of seven main phyla in the root compartment are arranged on the x-axis (in order: Actinomycetales, Burkholderiales, Caulobacteriales, Flavobacteriales, Rhizobiales, Sphingomonadales, and Xanthomonadales). On the y-axis, the relative abundance in the root for wild-type L. japonicus Gifu is shown in dark gray triangles, and the relative abundance in the root for epr3-13 is shown in light gray circles. The relative abundance of Burkholderiales is statistically decreased in the epr3-13 root, as indicated by the * label. Box plots show the first quartile and third quartile with the line indicating the median.

FIGS. 25A-25W show an alignment of the L. japonicus EPR3a polypeptide (EPR3A; SEQ ID NO: 62) and EPR3a-like polypeptide sequences from Prunus persica (XP_020410580_Prunus persica, SEQ ID NO: 63), Rosa chinensis (XP 024197374_Rosa chinensis, SEQ ID NO: 64), Vitis vinifera (RVW43308_Vitis vinifera, SEQ ID NO: 65), Ziziphus jujuba (XP_015894630_Ziziphus jujuba, SEQ ID NO: 66), Coffea arabica (XP_02 70993 33_Coffea arabica, SEQ ID NO: 67), Solanum pennellii (XP_015078544_Solanum pennellii, SEQ ID NO: 68), Solanum lycopersicum (XP_019069864_Solanum lycopersicum, SEQ ID NO: 69), Nicotiana attenuata (OIT29820_Nicotiana attenuata, SEQ ID NO: 70), Trema orientale (PON99018_Trema orientale, SEQ ID NO: 71), Trema levigatum (A5M47254_Trema levigatum, SEQ ID NO: 71), Juglans regia (XP_018805207_Juglans regia, SEQ ID NO: 72), Datisca glomerata (AZL41251_Datisca glomerata, SEQ ID NO: 73), Theobroma cacao (XP_017970654_Theobroma cacao, SEQ ID NO: 75), Durio zibethinus (XP_022775846_Durio zibethinus, SEQ ID NO: 76), Populus euphratica (XP_011034253_Populus euphratica, SEQ ID NO: 77; XP_011046250_Populus euphratica, SEQ ID NO: 90), Ricinus communis (XP_015580083_Ricinus communis, SEQ ID NO: 78), Manihot esculenta (OAY32540.1_Manihot esculenta, SEQ ID NO: 79), Daucus carota subsp. sativus (XP_017221830_Daucus carota subsp. sativus, SEQ ID NO: 80), Citrus sinensis (XP_006484322_Citrus sinensis, SEQ ID NO: 81), Citrus unshiu (GAY36258_Citrus unshiu, SEQ ID NO: 82), Vigna unguiculata (QCD82032 Vigna unguiculata, SEQ ID NO: 83), Vigna radiata var. radiata (XP_022632827 Vigna radiata var. radiata, SEQ ID NO: 84), Phaseolus vulgaris (XP 007153771_Phaseolus vulgaris, SEQ ID NO: 85), Glycine max (XP_003530632_Glycine max, SEQ ID NO: 86), Glycine soja (XP_028247343_Glycine soja, SEQ ID NO: 87), Lupinus angustifolius (XP_019423264 Lupinus angustifolius, SEQ ID NO: 88), Arachis ipaensis (XP_016192876_Arachis ipaensis, SEQ ID NO: 89), Zea mays (ZM8_Zea mays, SEQ ID NO: 91), and Hordeum vulgare (LysM-RLK8_Hordeum vulgare, SEQ ID NO: 92). Conservation is shown as black bars (scale: 0% to 100%) at the bottom of the alignment with similar residue shown with gray shading. FIG. 25A shows the first portion of the alignment. FIG. 25B shows the second portion of the alignment. FIG. 25C shows the third portion of the alignment. FIG. 25D shows the fourth portion of the alignment. FIG. 25E shows the fifth portion of the alignment. FIG. 25F shows the sixth portion of the alignment. FIG. 25G shows the seventh portion of the alignment. FIG. 25H shows the eighth portion of the alignment. FIG. 25I shows the ninth portion of the alignment. FIG. 25J shows the tenth portion of the alignment. FIG. 25K shows the eleventh portion of the alignment. FIG. 25L shows the twelfth portion of the alignment. FIG. 25M shows the thirteenth portion of the alignment. FIG. 25N shows the fourteenth portion of the alignment. FIG. 25O shows the fifteenth portion of the alignment. FIG. 25P shows the sixteenth portion of the alignment. FIG. 25Q shows the seventeenth portion of the alignment. FIG. 25R shows the eighteenth portion of the alignment. FIG. 25S shows the nineteenth portion of the alignment. FIG. 25T shows the twentieth portion of the alignment. FIG. 25U shows the twenty-first portion of the alignment. FIG. 25V shows the twenty-second portion of the alignment. FIG. 25W shows the twenty-third portion of the alignment.

FIGS. 26A-26L show an alignment of the L. japonicus EPR3 polypeptide (EPR3_Lj, SEQ ID NO: 61), the L. japonicus EPR3a polypeptide (EPR3A_Lj, SEQ ID NO: 62) and EPR3-like and EPR3a-like polypeptides from Lablab purpureus (Labpur_Labpu000468g0017.1, SEQ ID NO: 93; Labpur_Labpu000087g0009.1, SEQ ID NO: 108), Phaseolus vulgaris (Phavul_Phvul.002G059500.1, SEQ ID NO: 94; Phavul_Phvul.003G063700.1, SEQ ID NO: 107), Vigna unguiculata (Vigung_Vigun02g080500.1, SEQ ID NO: 95; Vigung_Vigun03g232900.1, SEQ ID NO: 105), Vigna subterranea (Vigsub_Vigsu002202g0039.1, SEQ ID NO: 96; Vigsub_Vigsul08716g0012.1, SEQ ID NO: 104), Vigna angularis (Vigang_vigan.Vang0057s00600.1, SEQ ID NO: 97; Vigang_vigan.Vang0033ss01040.1, SEQ ID NO: 109), Glycine max (Glymax_Glyma.01G027100.1, SEQ ID NO: 98; Glymax_Glyma.08G283300.1, SEQ ID NO: 110), Medicago truncatula (Medtru_MtrunA17_Chr5g0413071, SEQ ID NO: 99), Trifolium pratense (Tripra_Tp57577_TGAC_v2_mRNA40096, SEQ ID NO: 100), Castanospermum australe (Casaus_Castanospermum06838-PA, SEQ ID NO: 101), Nissolia schottii (Nissch_Nissc2308S18277, SEQ ID NO: 102; Nissch_Nissc255S07357, SEQ ID NO: 116), Cercis canadensis (Cercan_Cerca76S29361, SEQ ID NO: 103), Vigna radiata (Vigrad_Vradi0208s00120.1, SEQ ID NO: 106), Cajanus cajan (Cajcaj_Ccajan_03483, SEQ ID NO: 111), Arachis hypogaea (Arahyp_arahy.Tifrunner.gnm1.ann1.REE5L6.1, SEQ ID NO: 112; Arahyp_arahy.Tifrunner.gnm1.ann1.P7CPN3.1, SEQ ID NO: 113), Arachis ipaensis (Araipa_Araip.19T2H, SEQ ID NO: 114), Arachis duranensis (Aradur_Aradu.JOSGA, SEQ ID NO: 115), Lupinus angustifolius (Lupang_Lup020722.1, SEQ ID NO: 117), Faidherbia albida (Faialb_Faial00730g0027.1, SEQ ID NO: 118), Mimosa pudica (Mimpud_Mimpu35782S22845, SEQ ID NO: 119), Chamaecrista fasciculata (Chafas_Chafa3673S21651, SEQ ID NO: 120), and Prunus dulcis (Prudul_Prudul26A003177P1, SEQ ID NO: 121). The alignment is a CLUSTAL O(1.2.4) multiple sequence alignment, where an asterisk (*) indicates a fully conserved single residue, a colon (:) indicates conservation between residues with strongly similar properties (scoring >0.5 in the Gonnet PAM 250 matrix), and a period (.) indicates conservation between residues with weakly similar properties (scoring ≤0.5 in the Gonnet PAM 250 matrix). FIG. 26A shows the first portion of the alignment. FIG. 26B shows the second portion of the alignment. FIG. 26C shows the third portion of the alignment. FIG. 26D shows the fourth portion of the alignment. FIG. 26E shows the fifth portion of the alignment. FIG. 26F shows the sixth portion of the alignment. FIG. 26G shows the seventh portion of the alignment. FIG. 26H shows the eighth portion of the alignment. FIG. 26I shows the ninth portion of the alignment. FIG. 26J shows the tenth portion of the alignment. FIG. 26K shows the eleventh portion of the alignment. FIG. 26L shows the twelfth portion of the alignment.

FIGS. 27A-27L show an alignment of the L. japonicus EPR3 polypeptide (EPR3_Lj, SEQ ID NO: 61), and EPR3-like and EPR3a-like polypeptides from Prunus persica (Pruper_Prupe.1G247900.1, SEQ ID NO: 122), Prunus mume (Prumum_lcl_NC_024127.1_XP_016647040.1_6745, SEQ ID NO: 123), Rubus occidentalis (Rubocc_Bras_G02801, SEQ ID NO: 124), Theobroma cacao (Thecac_Thecc1EG010473t1, SEQ ID NO: 125), Gossypium raimondii (Gosrai_Gorai.005G179900.1, SEQ ID NO: 126), Sclerocarya birrea (Sclbir_Sclbi00092g0330.1, SEQ ID NO: 127), Ziziphus jujuba (Zizjuj_lcl_NC_029688.1_XP_015894630.2_26923, SEQ ID NO: 128), Discaria trinervis (Distri_Distr1293S13435, SEQ ID NO: 129), Trema orientalis (Treori_lcl_JXTC01000021.1_PON99018.1_5256, SEQ ID NO: 130), Parasponia andersonii (Parasponia_JXTB01000342.1_26365, SEQ ID NO: 131), Morus notabilis (Mornot_L484_027691, SEQ ID NO: 132), Citrus clementina (Citcle_Ciclev10033617m, SEQ ID NO: 133), Citrus sinensis (Citsin_orange 1.1g044997m, SEQ ID NO: 134), Datisca glomerata (Datglo_Datgl1333S01924, SEQ ID NO: 135), Manihot esculenta (Manesc_Manes.13G026000.1, SEQ ID NO: 136), Ricinus communis (Riccom_27504.m000627, SEQ ID NO: 137), Begoniafuchsioides (Begfuc_Begful064S15671, SEQ ID NO: 138), Alnus glutinosa (Alnglu_Alngl25086S21770, SEQ ID NO: 139), Casuarina glauca (Casgla_Casgl955S23425, SEQ ID NO: 140), Cajanus cajan (Cajcaj_Ccajan_12042, SEQ ID NO: 141), Fraxinus excelsior (Fraexc_FRAEX38873_v2_000332560.1, SEQ ID NO: 142), Solanum lycopersicum (Sollyc_Solyc06g069610.1.1, SEQ ID NO: 143), Solanum pennellii (Solpen_Sopen06g026910.1, SEQ ID NO: 144), Nicotiana benthamiana (Nicben_Niben101Scf02172g05003.1, SEQ ID NO: 145), Petunia axillaris (Petaxi_Peaxil62Scf00013g00053.1, SEQ ID NO: 146), Mimosa pudica (Mimpud_Mimpu5325S25442, SEQ ID NO: 147), Malus domestica (Maldom_MDP0000182108, SEQ ID NO: 148; Maldom_MDP0000137744, SEQ ID NO: 149), Pyrus communis (Pyrcom_PCP002145.1, SEQ ID NO: 150), and Pyrus x bretschneideri (Pyrbre_lcl_NW_008988137.1_XP_009363045.1_15483, SEQ ID NO: 151). The alignment is a CLUSTAL O(1.2.4) multiple sequence alignment, where an asterisk (*) indicates a fully conserved single residue, a colon (:) indicates conservation between residues with strongly similar properties (scoring >0.5 in the Gonnet PAM 250 matrix), and a period (.) indicates conservation between residues with weakly similar properties (scoring ≤0.5 in the Gonnet PAM 250 matrix). FIG. 27A shows the first portion of the alignment. FIG. 27B shows the second portion of the alignment. FIG. 27C shows the third portion of the alignment. FIG. 27D shows the fourth portion of the alignment. FIG. 27E shows the fifth portion of the alignment. FIG. 27F shows the sixth portion of the alignment. FIG. 27G shows the seventh portion of the alignment. FIG. 27H shows the eighth portion of the alignment. FIG. 27I shows the ninth portion of the alignment. FIG. 27J shows the tenth portion of the alignment. FIG. 27K shows the eleventh portion of the alignment. FIG. 27L shows the twelfth portion of the alignment.

FIGS. 28A-28M show an alignment of the L. japonicus EPR3 polypeptide (EPR3_Lj, SEQ ID NO: 61), and EPR3-like and EPR3a-like polypeptides from Prunus dulcis (Prudul_Prudul26A001224P1, SEQ ID NO: 152), Prunus persica (Pruper_Prupe.5G168000.1, SEQ ID NO: 153), Prunus mume (Prumum_lcl_NC_024132.1_XP_008239575.2_23116, SEQ ID NO: 154), Fragaria vesca (Fraves_FvH4_5g05950.1, SEQ ID NO: 155), Rubus occidentalis (Rubocc_Bras_G14455, SEQ ID NO: 156), Ziziphus jujuba (Zizjuj_lcl_NC_029683.1_XP_024929906.1_14394, SEQ ID NO: 157; Zizjuj_lcl_NC_029683.1_XP_024929890.1_14201; SEQ ID NO: 158), Populus trichocarpa (Poptri_Potri.015G082000.1, SEQ ID NO: 159), Sclerocarya birrea (Sclbir_Sclbi00184g0300.1, SEQ ID NO: 160), Manihot esculenta (Manesc_Manes.06G108500.1, SEQ ID NO: 161), Theobroma cacao (Thecac_Thecc1EG012192t1, SEQ ID NO: 162), Cucumis melo (Cucmel_MELO3C011965.2.1, SEQ ID NO: 163), Cucumis sativus (Cucsat_evm.model.Chr5.2205, SEQ ID NO: 164), Cucurbita pepo (Cucpep_Cp4.1LG01g15590.1, SEQ ID NO: 165), Cucurbita moschata (Cucmos_CmoCh04G018410.1, SEQ ID NO: 166), Helianthus annuus (Helann_HanXRQChr06g0174451, SEQ ID NO: 167; Helann_HanXRQChr05g0154581, SEQ ID NO: 168), Setaria italica (Setita Seita.5G192600.1, SEQ ID NO: 169), Sorghum bicolor (Sorbic_Sobic.003G184000.1, SEQ ID NO: 170), Zea mays (Zm00001d044536, SEQ ID NO: 171; Zm00001d009376, SEQ ID NO: 172), Brachypodium distachyon (Bradis_Bradi2g40627.1, SEQ ID NO: 173), Hordeum vulgare (Hv_RLK9, SEQ ID NO: 174), Oryza sativa (Orysat_LOC_Os01g36550.1, SEQ ID NO: 175), Musa acuminata (Musacu_GSMUA_AchrUn_randomP00190_001, SEQ ID NO: 176), Populus trichocarpa (Poptri_Potri.008G187500.2, SEQ ID NO: 177), Dendrobium catenatum (Dencat_lcl_NW_021394673.1_XP_020705880.2_31308, SEQ ID NO: 178), Apostasia shenzhenica (Aposhe_lcl_KZ451895.1_PKA65282.1_1767, SEQ ID NO: 179), Carica papaya (Carpap_evm.model.supercontig_33.2, SEQ ID NO: 180), and Quercus robur (Querob_Qrob_P0255210.2, SEQ ID NO: 181). The alignment is a CLUSTAL O(1.2.4) multiple sequence alignment, where an asterisk (*) indicates a fully conserved single residue, a colon (:) indicates conservation between residues with strongly similar properties (scoring >0.5 in the Gonnet PAM 250 matrix), and a period (.) indicates conservation between residues with weakly similar properties (scoring ≤0.5 in the Gonnet PAM 250 matrix). FIG. 28A shows the first portion of the alignment. FIG. 28B shows the second portion of the alignment. FIG. 28C shows the third portion of the alignment. FIG. 28D shows the fourth portion of the alignment. FIG. 28E shows the fifth portion of the alignment. FIG. 28F shows the sixth portion of the alignment. FIG. 28G shows the seventh portion of the alignment.

FIG. 28H shows the eighth portion of the alignment. FIG. 28I shows the ninth portion of the alignment. FIG. 28J shows the tenth portion of the alignment. FIG. 28K shows the eleventh portion of the alignment. FIG. 28L shows the twelfth portion of the alignment. FIG. 28M shows the thirteenth portion of the alignment.

FIGS. 29A-29D show comparisons of Epr3 and Epr3a genes, amino acid sequences, and protein structures across plant species. FIG. 29A shows a chart summarizing whether different plant have homologs of (from top to bottom) EPR3a or EPR3, and whether they form mutualistic associations with rhizobia (RNS), arbuscular mycorrhizal fungi (AMS), or ectomycorrhizal fungi (ECMS). The genus, species, or type of plant is indicated including, from left to right: Lotus, Medicago, Soybean, Parasponia, Trema, Populus, Malus, Fragaria, Maize, Rice, Wheat, Barley, Datisca, Lupinus, Arabidopsis, Brassica rapa, and Brassica napus. Filled-in boxes indicate that the plant does have a homolog of the indicated gene, empty boxes indicate that the plant does not have a homolog of the indicated gene, and the plus (+) and minus (−) signs indicate whether the plant does (+) or does not (−) form mutualistic associations with (from top to bottom) RNS, AMS, or ECMS. FIG. 29B shows an alignment of the amino acid sequences of the M1 domain of Lotus EPR3 (amino acid positions 54-98, top row) and EPR3a (amino acid positions 46-90, bottom row). The positions of the conserved secondary structures of the βαββ fold of the M1 domain are labeled (α1, β1, β2, and β3) above the alignment. FIG. 29C shows a predicted model of the structure of the L. japonicus EPR3a ED based on a homology model to the crystal structure of L. japonicus EPR3 (PDB code 6QUP). The positions of the M1, M2, and LysM3 domains are indicated, as well as the N- and C-termini. FIG. 29D shows a side-by-side comparison of a force field model of the structure of the M1 domain of L. japonicus EPR3a (top) with a crystal structure of the M1 domain of L. japonicus EPR3 (bottom, PDB code 6QUP). In both structures, the β-sheets and α-helix secondary structures that make up the βαββ fold of the M1 domain are labeled (α 1, β1, β2, and β3), and the position of the N- and C-termini are indicated.

FIGS. 30A-30B show analyses of the expression of EPR3a and EPR3. FIG. 30A shows qRT-PCR data showing expression of PT4 (top), Epr3a (center), and Epr3 (bottom) in L. japonicus during the establishment of symbiosis with arbuscular mycorrhizae. For each transcript, the number of days post inoculation (dpi) is shown on the x-axis, and the level of absolute expression is shown on the y-axis. The expression level of each transcript is shown under inoculation with arbuscular mycorrhizae (shaded box-and-whisker plots), and under a mock inoculation (white box-and-whisker plots). Results shown are from three biological replicates. Box plots show the first quartile and third quartile with the line indicating the median. FIG. 30B shows Epr3a promoter activity in roots during arbuscular mycorrhizal colonization. The Epr3a promoter was placed upstream of the marker GUS, and blue staining (seen as dark patches; indicated by arrows labeled “Epr3a promoter activity”) indicates GUS activity. Green fluorescence (seen as white structures; indicated by arrows labeled “Fungal structure”) indicates fungal (arbuscular mycorrhizae) structures.

FIG. 31 shows phenotypes of wild type L. japonicus Gifu (Gifu) compared to phenotypes of L. japonicus with mutations in epr3 and/or epr3a when inoculated with arbuscular mycorrhizae, 6 weeks post inoculation. The x-axis indicates the genotype of the plant (Gifu WT, epr3-11 single mutant, epr3a-1 single mutant, epr3a-2 single mutant, and epr3/epr3a double mutant) and the y-axis indicates the % occurrence of the presence of fungal infection (left box-and-whisker for each genotype, dark gray), arbuscules (center box- and whisker for each genotype, gray), and vesicles (right box-and-whisker for each genotype, light gray). Box plots show the first quartile and third quartile with the line indicating the median. Statistical comparisons between genotypes for each infection event are shown using ANOVA and Tukey post hoc testing with p-values (<0.05), as indicated by different letters, and the sample size for each genotype is indicated beneath the x-axis (n).

FIG. 32 shows expression of the M. truncatula A17 EPR3/EPR3a-like gene MtrunA17_Chr5g0413071 (Lyk10) during arbuscular mycorrhizal symbiosis. The number of days post inoculation (dpi) is shown on the x-axis, and the normalized number of counts from the MtrunA17_Chr5g0413071 transcript is shown on the y-axis. The expression level is shown under inoculation with arbuscular mycorrhizae (left bar for each number of days post infection), and under a mock inoculation (right bar for each number of days post infection). The expression data shown in FIG. 32 is RNA-seq data from (Gobbato, E. et al., Curr Biol 2012 22(23):2236-41) that was mined and analyzed. Error bars indicate standard error of the mean.

FIGS. 33A-33B show models of symbiotic signaling output from the L. japonicus EPS receptors EPR3 and EPR3a. FIG. 33A shows a model of symbiotic signaling output from the L. japonicus EPS receptors EPR3 and EPR3a in root nodule symbioses with bacteria (RNS). FIG. 33B shows a model of symbiotic signaling output from the L. japonicus EPS receptors EPR3 and EPR3a in arbuscular mycorrhizal symbioses with fungi (AMS). In FIGS. 33A-33B, EPR3 is shown at left, EPR3a is shown at right, and the relative strength of positive symbiotic signaling in each type of symbiosis is represented by the number of + symbols (increased number=increased relative strength).

FIGS. 34A-34B show the relative abundance of bacterial colonization of wild-type L. japonicus Gifu (Gifu) compared to L. japonicus with mutations in epr3 and/or epr3a. Plants were co-inoculated with the symbiotic bacteria M. loti R7A exoU, and non-symbiotic isolates of Burkholderiales bacteria. FIG. 34A shows the relative abundance of bacterial colonization of L. japonicus Gifu (Gifu) compared to L. japonicus with mutations in epr3 and/or epr3a with M. loti R7A exoU (R7AexoU) and total Burkholderiales (total Burk), representing the value of total Burkholderiales calculated based on the relative abundance of all Burkholderiales isolates used in this experiment (Burkholderiales isolates LjRoot223, LjRoot280, LjRoot194, LjRoot230, LjRoot241, LjRoot70, LjRoot29, LjRoot1, LjRoot131, LjRoot296, LjRoot122, LjRoot39, LjRoot294). FIG. 34B shows the relative abundance of bacterial colonization of L. japonicus Gifu (Gifu) compared to L. japonicus with mutations in epr3 and/or epr3a with the bacteria measured including, from left to right on the x-axis, Burkholderiales isolates LjRoot223, LjRoot280, LjRoot194, LjRoot230, LjRoot241, and LjRoot70. In FIGS. 34A-34B, the y-axis indicates the relative abundance of each type of bacteria as a percentage. For each type of bacteria, abundance is shown as a boxplot for, from left to right, wild-type L. japonicus (Gifu), epr3a-1 (e3a-1), epr3a-2 (e3a-2), or the epr3/epr3a double mutant (DM) plants. In each boxplot, the line indicates the median value, and the edges of the box illustrate the first and third quartile. Dots represent values from individual plants.

### DETAILED DESCRIPTION

The following description sets forth exemplary methods, parameters, and the like. It should be recognized, however, that such description is not intended as a limitation on the scope of the present disclosure but is instead provided as a description of exemplary embodiments.

### Genetically Altered Plants

An aspect of the disclosure includes a genetically altered plant or part thereof including a first nucleic acid sequence encoding a heterologous EPR3 or EPR3-like polypeptide or a modified EPR3 or EPR3-like polypeptide, wherein the heterologous EPR3 or EPR3-like polypeptide or the modified EPR3 or EPR3-like polypeptide provides increased selectivity for a beneficial commensal microbe as compared to a wild-type plant under the same conditions. Selectivity may mean positive selection of the beneficial commensal microbe, negative selection of other microbes that are not the beneficial commensal, or a combination thereof. An additional embodiment of this aspect includes the plant or part thereof further including a second nucleic acid sequence encoding a heterologous EPR3a or EPR3a-like polypeptide or a modified EPR3a or EPR3a-like polypeptide. In a further embodiment of this aspect, which may be combined with any of the preceding embodiments, the heterologous EPR3 or EPR3-like polypeptide is selected from the group of a first polypeptide with at least 70% sequence identity, at least 71% sequence identity, at least 72% sequence identity, at least 73% sequence identity, at least 74% sequence identity, at least 75% sequence identity, at least 76% sequence identity, at least 77% sequence identity, at least 78% sequence identity, at least 79% sequence identity, at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 1 [L. japonicus (BAI79269.1)], a second polypeptide with at least 70% sequence identity, at least 71% sequence identity, at least 72% sequence identity, at least 73% sequence identity, at least 74% sequence identity, at least 75% sequence identity, at least 76% sequence identity, at least 77% sequence identity, at least 78% sequence identity, at least 79% sequence identity, at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 2 [Chickpea (XP_004489790.1)], a third polypeptide with at least 70% sequence identity, at least 71% sequence identity, at least 72% sequence identity, at least 73% sequence identity, at least 74% sequence identity, at least 75% sequence identity, at least 76% sequence identity, at least 77% sequence identity, at least 78% sequence identity, at least 79% sequence identity, at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 3 [Medicago (XP_003613165.1)], a fourth polypeptide with at least 70% sequence identity, at least 71% sequence identity, at least 72% sequence identity, at least 73% sequence identity, at least 74% sequence identity, at least 75% sequence identity, at least 76% sequence identity, at least 77% sequence identity, at least 78% sequence identity, at least 79% sequence identity, at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 4 [Soybean (XP_003517716.1)], a fifth polypeptide with at least 70% sequence identity, at least 71% sequence identity, at least 72% sequence identity, at least 73% sequence identity, at least 74% sequence identity, at least 75% sequence identity, at least 76% sequence identity, at least 77% sequence identity, at least 78% sequence identity, at least 79% sequence identity, at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 5 [Phaseolus (XP_007157313.1)], a sixth polypeptide with at least 70% sequence identity, at least 71% sequence identity, at least 72% sequence identity, at least 73% sequence identity, at least 74% sequence identity, at least 75% sequence identity, at least 76% sequence identity, at least 77% sequence identity, at least 78% sequence identity, at least 79% sequence identity, at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 6 [Populus (XP_002322185.1)], a seventh polypeptide with at least 70% sequence identity, at least 71% sequence identity, at least 72% sequence identity, at least 73% sequence identity, at least 74% sequence identity, at least 75% sequence identity, at least 76% sequence identity, at least 77% sequence identity, at least 78% sequence identity, at least 79% sequence identity, at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 7 [Mains (XP_008340354.1)], an eighth polypeptide with at least 70% sequence identity, at least 71% sequence identity, at least 72% sequence identity, at least 73% sequence identity, at least 74% sequence identity, at least 75% sequence identity, at least 76% sequence identity, at least 77% sequence identity, at least 78% sequence identity, at least 79% sequence identity, at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 8 [Vitis (XP_002272814.2)], a ninth polypeptide with at least 70% sequence identity, at least 71% sequence identity, at least 72% sequence identity, at least 73% sequence identity, at least 74% sequence identity, at least 75% sequence identity, at least 76% sequence identity, at least 77% sequence identity, at least 78% sequence identity, at least 79% sequence identity, at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 9 [Theobroma (XP_007036352.1)], a tenth polypeptide with at least 70% sequence identity, at least 71% sequence identity, at least 72% sequence identity, at least 73% sequence identity, at least 74% sequence identity, at least 75% sequence identity, at least 76% sequence identity, at least 77% sequence identity, at least 78% sequence identity, at least 79% sequence identity, at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 10 [Ricinus (XP_002527912.1)], an eleventh polypeptide with at least 70% sequence identity, at least 71% sequence identity, at least 72% sequence identity, at least 73% sequence identity, at least 74% sequence identity, at least 75% sequence identity, at least 76% sequence identity, at least 77% sequence identity, at least 78% sequence identity, at least 79% sequence identity, at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 11 [Fragaria (XP_004300916.1)], a twelfth polypeptide with at least 70% sequence identity, at least 71% sequence identity, at least 72% sequence identity, at least 73% sequence identity, at least 74% sequence identity, at least 75% sequence identity, at least 76% sequence identity, at least 77% sequence identity, at least 78% sequence identity, at least 79% sequence identity, at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 12 [Maize (XP_008657477.1)], a thirteenth polypeptide with at least 70% sequence identity, at least 71% sequence identity, at least 72% sequence identity, at least 73% sequence identity, at least 74% sequence identity, at least 75% sequence identity, at least 76% sequence identity, at least 77% sequence identity, at least 78% sequence identity, at least 79% sequence identity, at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 13 [Rice (XP_015628733.1)], a fourteenth polypeptide with at least 70% sequence identity, at least 71% sequence identity, at least 72% sequence identity, at least 73% sequence identity, at least 74% sequence identity, at least 75% sequence identity, at least 76% sequence identity, at least 77% sequence identity, at least 78% sequence identity, at least 79% sequence identity, at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 14 [Wheat (CDM80098.1)], or a fifteenth polypeptide with at least 70% sequence identity, at least 71% sequence identity, at least 72% sequence identity, at least 73% sequence identity, at least 74% sequence identity, at least 75% sequence identity, at least 76% sequence identity, at least 77% sequence identity, at least 78% sequence identity, at least 79% sequence identity, at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 15 [Barley (MLOC_5489.2)]. Yet another embodiment of this aspect includes the heterologous EPR3 or EPR3-like polypeptide being selected from the group of SEQ ID NO: 1 [L. japonicus (EPR3)], SEQ ID NO: 2 [Chickpea (XP_004489790.1)], SEQ ID NO: 3 [Medicago (XP_003613165.1)], SEQ ID NO: 4 [Soybean (XP_003517716.1)], SEQ ID NO: 5 [Phaseolus (XP_007157313.1)], SEQ ID NO: 6 [Populus (XP_002322185.1)], SEQ ID NO: 7 [Malus (XP_008340354.1)], SEQ ID NO: 8 [Vitis (XP_002272814.2)], SEQ ID NO: 9 [Theobroma (XP_007036352.1)], SEQ ID NO: 10 [Ricinus (XP_002527912.1)], SEQ ID NO: 11 [Fragaria (XP_004300916.1)], SEQ ID NO: 12 [Maize (XP_008657477.1)], SEQ ID NO: 13 [Rice (XP_015628733.1)], SEQ ID NO: 14 [Wheat (CDM80098.1)], or SEQ ID NO: 15 [Barley (MLOC_5489.2)]. Still another embodiment of this aspect, which may be combined with any of the preceding embodiments that has a heterologous EPR3a or EPR3a-like polypeptide, the heterologous EPR3a or EPR3a-like polypeptide is selected from the group of a polypeptide with at least 70% sequence identity, at least 71% sequence identity, at least 72% sequence identity, at least 73% sequence identity, at least 74% sequence identity, at least 75% sequence identity, at least 76% sequence identity, at least 77% sequence identity, at least 78% sequence identity, at least 79% sequence identity, at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 62 [L. japonicus (EPR3a)], SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, or SEQ ID NO: 92. A further embodiment of this aspect includes the heterologous EPR3a or EPR3a-like polypeptide being SEQ ID NO: 62 [L. japonicus (EPR3a)], SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, or SEQ ID NO: 92.

Still another embodiment of this aspect, which may be combined with any of the preceding embodiments, the modified EPR3 or EPR3-like polypeptide comprises a modified ectodomain that has been replaced with all or a portion of an ectodomain of the heterologous EPR3 or EPR3-like polypeptide, optionally all or a part of the M1 domain, the M2 domain, the LysM3 domain, or all three. In an additional embodiment of this aspect, the portion replaced is at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, less than 10%, less than 11%, less than 12%, less than 13%, less than 14%, less than 15%, less than 16%, less than 17%, less than 18%, less than 19%, less than 20%, less than 21%, less than 22%, less than 23%, less than 24%, less than 25%, less than 26%, less than 27%, less than 28%, less than 29%, less than 30%, less than 31%, less than 32%, less than 33%, less than 34%, less than 35%, less than 36%, less than 37%, less than 38%, less than 39%, less than 40%, less than 41%, less than 42%, less than 43%, less than 44%, less than 45%, less than 46%, less than 47%, less than 48%, less than 49%, less than 50%, less than 51%, less than 52%, less than 53%, less than 54%, less than 55%, less than 56%, less than 57%, less than 58%, less than 59%, less than 60%, less than 61%, less than 62%, less than 63%, less than 64%, less than 65%, less than 66%, less than 67%, less than 68%, less than 69%, less than 70%, less than 71%, less than 72%, less than 73%, less than 74%, less than 75%, less than 76%, less than 77%, less than 78%, less than 79%, less than 80%, less than 81%, less than 82%, less than 83%, less than 84%, less than 85%, less than 86%, less than 87%, less than 88%, less than 89%, or less than 90%, of the ectodomain or, optionally all or a part of the M1 domain, the M2 domain, the LysM3 domain, or all three. In yet another embodiment of this aspect, which may be combined with any of the preceding embodiments that have an EPR3a or EPR3a-like polypeptide, the modified EPR3a or EPR3a-like polypeptide includes a modified ectodomain that has been replaced with all or a portion of an ectodomain of the heterologous EPR3a or EPR3a-like polypeptide, optionally all or a part of the M1 domain, the M2 domain, the LysM3 domain, or all three. In an additional embodiment of this aspect, the portion replaced is at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, less than 10%, less than 11%, less than 12%, less than 13%, less than 14%, less than 15%, less than 16%, less than 17%, less than 18%, less than 19%, less than 20%, less than 21%, less than 22%, less than 23%, less than 24%, less than 25%, less than 26%, less than 27%, less than 28%, less than 29%, less than 30%, less than 31%, less than 32%, less than 33%, less than 34%, less than 35%, less than 36%, less than 37%, less than 38%, less than 39%, less than 40%, less than 41%, less than 42%, less than 43%, less than 44%, less than 45%, less than 46%, less than 47%, less than 48%, less than 49%, less than 50%, less than 51%, less than 52%, less than 53%, less than 54%, less than 55%, less than 56%, less than 57%, less than 58%, less than 59%, less than 60%, less than 61%, less than 62%, less than 63%, less than 64%, less than 65%, less than 66%, less than 67%, less than 68%, less than 69%, less than 70%, less than 71%, less than 72%, less than 73%, less than 74%, less than 75%, less than 76%, less than 77%, less than 78%, less than 79%, less than 80%, less than 81%, less than 82%, less than 83%, less than 84%, less than 85%, less than 86%, less than 87%, less than 88%, less than 89%, or less than 90%, of the ectodomain or, optionally all or a part of the M1 domain, the M2 domain, the LysM3 domain, or all three. A further embodiment of this aspect, which may be combined with any of the preceding embodiments that have an EPR3a or EPR3a-like polypeptide, includes the heterologous EPR3 or EPR3-like polypeptide and the heterologous EPR3a or EPR3a-like polypeptide being from the same plant species or the same plant variety.

Yet another embodiment of this aspect, which may be combined with any of the preceding embodiments, includes the expression of the heterologous EPR3 or EPR3-like polypeptide or the modified EPR3 or EPR3-like polypeptide allowing the plant or part thereof to recognize an EPS, a beta-glucan, a cyclic beta-glucan, a LPS, or a surface carbohydrate produced by the microbe. Still another embodiment of this aspect, which may be combined with any of the preceding embodiments that have an EPR3a or EPR3a-like polypeptide, includes the expression of the heterologous EPR3a or EPR3a-like polypeptide or the modified EPR3a or EPR3a-like polypeptide allowing the plant or part thereof to recognize an EPS, a beta-glucan, a cyclic beta-glucan, a LPS, or a surface carbohydrate produced by the microbe. In a further embodiment of this aspect, the expression of the heterologous EPR3 or EPR3-like polypeptide or the modified EPR3 or EPR3-like polypeptide and the expression of the heterologous EPR3a or EPR3a-like polypeptide or the modified EPR3a or EPR3a-like polypeptide allows the plant or part thereof to recognize an EPS, a beta-glucan, a cyclic beta-glucan, a LPS, or a surface carbohydrate produced by the microbe. In an additional embodiment of this aspect that can be combined with any preceding embodiments including an EPS produced by the microbe, the microbe is a commensal bacteria, optionally a nitrogen-fixing bacteria, or a mycorrhizal fungi. A further embodiment of this aspect includes the nitrogen-fixing bacteria being selected from the group of Mesorhizobium loti, Mesorhizobium huakuii, Mesorhizobium mediterraneum, Mesorhizobium ciceri, Mesorhizobium spp., Rhizobium mongolense, Rhizobium tropici, Rhizobium etli phaseoli, Rhizobium giardinii, Rhizobium leguminosarum optionally R. leguminosarum trifolii, R. leguminosarum viciae, and R. leguminosarum phaseoli, Burkholderiales optionally symbionts of Mimosa, Sinorhizobium meliloti, Sinorhizobium medicae, Sinorhizobium fredii, Sinorhizobium fredii NGR234, Azorhizobium caulinodans, Bradyrhizobium japonicum, Bradyrhizobium elkanii, Bradyrhizobium liaonginense, Rhizobium spp., Mesorhizobium spp., Sinorhizobium spp., Azorhizobium spp. Frankia spp., or any combination thereof, or the mycorrhizal fungi being selected from the group of Acaulosporaceae spp., Diversisporaceae spp., Gigasporaceae spp., Pacisporaceae spp., Funneliformis spp., Glomus spp., Rhizophagus spp., Sclerocystis spp., Septoglomus spp., Claroideoglomus spp., Ambispora spp., Archaeospora spp., Geosiphon pyriformis, Paraglomus spp., other species in the division Glomeromycota, or any combination thereof. Still another embodiment of this aspect, which may be combined with any preceding embodiments, includes the heterologous EPR3 or EPR3-like polypeptide or the modified EPR3 or EPR3-like polypeptide being localized to a plant cell plasma membrane. Yet another embodiment of this aspect, which may be combined with any of the preceding embodiments that have an EPR3a or EPR3a-like polypeptide, includes the heterologous EPR3a or EPR3a-like polypeptide or the modified EPR3a or EPR3a-like polypeptide being localized to a plant cell plasma membrane. A further embodiment of this aspect that can be combined with any of the preceding embodiments that have localization to a plant cell plasma membrane includes the plant cell being a root cell. An additional embodiment of this aspect includes the root cell being a root epidermal cell or a root cortex cell. In a further embodiment of this aspect that can be combined with any of the preceding embodiments, the heterologous EPR3 or EPR3-like polypeptide or the modified EPR3 or EPR3-like polypeptide is expressed in a developing plant root system. In an additional embodiment of this aspect that can be combined with any of the preceding embodiments that has an EPR3a or EPR3a-like polypeptide, the heterologous EPR3a or EPR3a-like polypeptide or the modified EPR3a or EPR3a-like polypeptide is expressed in a developing plant root system.

In still another embodiment of this aspect, which may be combined with any of the preceding embodiments, the first nucleic acid sequence is operably linked to a first promoter. In an additional embodiment of this aspect, the first promoter is a root specific promoter, and the root specific promoter is optionally selected from the group of a NFR1 or NFR5/NFP promoter, an EPR3 or an EPR3a promoter, a Lotus NFR5 promoter, a Lotus NFR1 promoter, a maize allothioneine promoter, a chitinase promoter, a maize ZRP2 promoter, a tomato LeExt1 promoter, a glutamine synthetase soybean root promoter, a RCC3 promoter, a rice antiquitine promoter, a LRR receptor kinase promoter, or an Arabidopsis pCO2 promoter. In a further embodiment of this aspect, the first promoter is a constitutive promoter, and the constitutive promoter is optionally selected from the group of a CaMV35S promoter, a derivative of the CaMV35S promoter, a maize ubiquitin promoter, a trefoil promoter, a vein mosaic cassava virus promoter, or an Arabidopsis UBQ10 promoter. In yet another embodiment of this aspect, which may be combined with any of the preceding embodiments that has an EPR3a or an EPR3a-like polypeptide, the second nucleic acid sequence is operably linked to a second promoter. In an additional embodiment of this aspect, the second promoter is a root specific promoter, and the root specific promoter is optionally selected from the group of a NFR1 or NFR5/NFP promoter, an EPR3 or an EPR3a promoter, a Lotus NFR5 promoter, a Lotus NFR1 promoter, a maize allothioneine promoter, a chitinase promoter, a maize ZRP2 promoter, a tomato LeExt1 promoter, a glutamine synthetase soybean root promoter, a RCC3 promoter, a rice antiquitine promoter, a LRR receptor kinase promoter, or an Arabidopsis pCO2 promoter. In a further embodiment of this aspect, the second promoter is a constitutive promoter, and the constitutive promoter is optionally selected from the group of a CaMV35S promoter, a derivative of the CaMV35S promoter, a maize ubiquitin promoter, a trefoil promoter, a vein mosaic cassava virus promoter, or an Arabidopsis UBQ10 promoter. In an additional embodiment of this aspect, which may be combined with any of the preceding embodiments, the plant is selected from the group of corn (e.g., maize, Zea mays), rice (e.g., indica rice, japonica rice, aromatic rice, glutinous rice, Oryza sat/va, Oryza glaberrima), wild rice (e.g., Zizania spp., Porteresia spp.), wheat (e.g., common wheat, spelt, durum, einkorn, emmer, kamut, Triticum aestivum, Triticum spelta, Triticum durum, Triticum urartu, Triticum monococcum, Triticum turanicum, Triticum spp.), barley (e.g., Hordeum vulgare), sorghum (e.g., Sorghum bicolor), millet (e.g., finger millet, fonio millet, foxtail millet, pearl millet, barnyard millets, Eleusine coracana, Panicum sumatrense, Panicum milaceum, Setariaitalica, Pennisetum glaucum, Digitaria spp., Echinocloa spp.), teff (e.g., Eragrostis tef), oat (e.g., Avena sativa), triticale (e.g., X Triticosecale Wittmack, Triticosecale schlanstedtense Wittm., Triticosecale neoblaringhemii A. Camus, Triticosecale neoblaringhemii A. Camus), rye (e.g., Secale cereale, Secale cereanum), sugar cane (e.g., Saccharum officinarum, Saccharum spp.), apple (e.g., Malus pumila, Malus x domestica, Pyrus malus), pear (e.g., Pyrus communis, Pyrus x bretschneideri, Pyrus pyrifolia, Pyrus sinkiangensis, Pyrus pashia, Pyrus spp.), plum (e.g., Mirabelle, greengage, damson, Prunus domestica, Prunus salicina, Prunus mume), apricot (e.g., Prunus armeniaca, Prunus brigantine, Prunus mandshurica), peach (e.g., Prunus persica), almond (e.g., Prunus dulcis, Prunus amygdalus), walnut (e.g., Persian walnut, English walnut, black walnut, Juglans regia, Juglans nigra, Juglans cinerea, Juglans californica), cherry (e.g., Prunus avium, Prunus cerasus, Prunus yedoensis var. nudiflora), strawberry (e.g., Fragaria x ananassa, Fragaria chiloensis, Fragaria virginiana, Fragaria vesca), raspberry (e.g., European red raspberry, black raspberry, Rubus idaeus L., Rubus occidentalis, Rubus strigosus), blackberry (e.g., evergreen blackberry, Himalayan blackberry, Rubus fruticosus, Rubus ursinus, Rubus laciniatus, Rubus argutus, Rubus armeniacus, Rubus plicatus, Rubus ulmifolius, Rubus allegheniensis, Rubus subgenus Eubatus sect. Moriferi & Ursini), red currant (e.g., white currant, Ribes rubrum), black currant (e.g., cassis, Ribes nigrum), gooseberry (e.g., Ribes uva-crispa, Ribes grossulari, Ribes hirtellum), cowpea (e.g., Vigna unguiculata), melon (e.g., watermelon, winter melon, casabas, cantaloupe, honeydew, muskmelon, Citrullus lanatus, Benincasa hispida, Cucumis melo, Cucumis melo cantalupensis, Cucumis melo inodorus, Cucumis melo reticulatus), cucumber (e.g., slicing cucumbers, pickling cucumbers, English cucumber, Cucumis sativus), pumpkin (e.g., Cucurbita pepo, Cucurbita maxima), squash (e.g., gourd, Cucurbita argyrosperma, Cucurbita ficifolia, Cucurbita maxima, Cucurbita moschata), grape (e.g., Vitis vinifera, Vitis amurensis, Vitis labrusca, Vitis mustangensis, Vitis riparia, Vitis rotundifolia), hemp (e.g., cannabis, Cannabis sativa), hops (e.g., Humulus lupulus), birch (e.g., Betula spp.), beech (e.g., Fagus sylvatica, Fagus grandifolia, Fagus spp.), jujube (e.g., red date, Ziziphus jujube), cassava (e.g., manioc, yucca, Manihot esculenta), poplar (e.g., hybrid poplar, Populus trichocarpa, Populus tremula, Populus alba, Populus spp.), chestnut (e.g., Castanea mollissima, Castanea crenata, Castanea dentata, Castanea spp.), swamp oak (e.g., Casuarina glauca), rose gum (e.g., Eucalyptus grandis), oak (e.g., cork oak, Quercus suber, Quercus spp.), citrus (e.g., lemon, lime, orange, grapefruit, pomelo, citron, trifoliate orange, bergamot orange, bitter orange, blood orange, satsuma, clementine, mandarin, yuzu, finger lime, kaffir lime, kumquat, Citrus clementina, Citrus sinensis, Citrus trifoliata, Citrus japonica, Citrus maxima, Citrus australasica, Citrus reticulata, Citus aurantifolia, Citrus hystrix, Citrus x paradisi, Citrus x clementina, Citrus spp.), potato (e.g., russet potatoes, yellow potatoes, red potatoes, Solanum tuberosum), tomato (e.g., Solanum lycopersicum), pepper (e.g., sweet pepper, bell pepper, hot pepper, chili pepper, Capsicum L.), sweet potato (e.g., Ipomoea batatas), yam (e.g., Diascorea spp., Oxalis tuberosa), Trema spp. (e.g., Trema cannabina, Trema cubense, Trema discolor, Trema domingensis, Trema integerrima, Trema lamarckiana, Trema micrantha, Trema orientalis, Trema philippinensis, Trema strigilosa, Trema tomentosa, Trema levigata), or Jatropha spp. (e.g., Jatropha curcas). In yet another embodiment of this aspect, which may be combined with any of the preceding embodiments, the plant lacks functional rhizobial Nod factor receptors. In still another embodiment of this aspect, which may be combined with any of the preceding embodiments, the plant is not a legume. In an additional embodiment of this aspect, which may be combined with any of the preceding embodiments, the plant is not A. thaliana, N. tabacum, L. japonicus, or M. truncatula. In a further embodiment of this aspect, which may be combined with any of the above embodiments, the plant part is a leaf, a stem, a root, a root primordia, a flower, a seed, a fruit, a kernel, a grain, a cell, or a portion thereof. An additional embodiment of this aspect includes the plant part being a fruit, a kernel, or a grain.

In some aspects, the present disclosure relates to a pollen grain or an ovule of the genetically altered plant of any of the above embodiments.

In some aspects, the present disclosure relates to a protoplast produced from the plant of any of the above embodiments.

In some aspects, the present disclosure relates to a tissue culture produced from protoplasts or cells from the plant of any of the above embodiments, wherein the cells or protoplasts are produced from a plant part selected from the group of leaf, anther, pistil, stem, petiole, root, root primordia, root tip, fruit, seed, flower, cotyledon, hypocotyl, embryo, or meristematic cell.

An additional aspect of the disclosure includes a genetically altered plant or part thereof including a first nucleic acid sequence encoding a heterologous EPR3a or EPR3a-like polypeptide or a modified EPR3a or EPR3a-like polypeptide, wherein the heterologous EPR3a or EPR3a-like polypeptide or the modified EPR3a or EPR3a-like polypeptide provides increased selectivity for a beneficial commensal microbe as compared to a wild-type plant under the same conditions. Selectivity may mean positive selection of the beneficial commensal microbe, negative selection of other microbes that are not the beneficial commensal, or a combination thereof. An additional embodiment of this aspect includes the plant or part thereof further including a second nucleic acid sequence encoding a heterologous EPR3 or EPR3-like polypeptide or a modified EPR3 or EPR3-like polypeptide. In a further embodiment of this aspect, which may be combined with any of the preceding embodiments, the heterologous EPR3a or EPR3a-like polypeptide is selected from the group of a polypeptide with at least 70% sequence identity, at least 71% sequence identity, at least 72% sequence identity, at least 73% sequence identity, at least 74% sequence identity, at least 75% sequence identity, at least 76% sequence identity, at least 77% sequence identity, at least 78% sequence identity, at least 79% sequence identity, at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 62 [L. japonicus (EPR3a)], SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, or SEQ ID NO: 92. Yet another embodiment of this aspect includes the heterologous EPR3a or EPR3a-like polypeptide being SEQ ID NO: 62 [L. japonicus (EPR3a)], SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, or SEQ ID NO: 92. Still another embodiment of this aspect, which may be combined with any of the preceding embodiments that has a heterologous EPR3 or EPR3-like polypeptide, the heterologous EPR3 or EPR3-like polypeptide is selected from the group of a first polypeptide with at least 70% sequence identity, at least 71% sequence identity, at least 72% sequence identity, at least 73% sequence identity, at least 74% sequence identity, at least 75% sequence identity, at least 76% sequence identity, at least 77% sequence identity, at least 78% sequence identity, at least 79% sequence identity, at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 1 [L. japonicus (BAI79269.1)], a second polypeptide with at least 70% sequence identity, at least 71% sequence identity, at least 72% sequence identity, at least 73% sequence identity, at least 74% sequence identity, at least 75% sequence identity, at least 76% sequence identity, at least 77% sequence identity, at least 78% sequence identity, at least 79% sequence identity, at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 2 [Chickpea (XP_004489790.1)], a third polypeptide with at least 70% sequence identity, at least 71% sequence identity, at least 72% sequence identity, at least 73% sequence identity, at least 74% sequence identity, at least 75% sequence identity, at least 76% sequence identity, at least 77% sequence identity, at least 78% sequence identity, at least 79% sequence identity, at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 3 [Medicago (XP_003613165.1)], a fourth polypeptide with at least 70% sequence identity, at least 71% sequence identity, at least 72% sequence identity, at least 73% sequence identity, at least 74% sequence identity, at least 75% sequence identity, at least 76% sequence identity, at least 77% sequence identity, at least 78% sequence identity, at least 79% sequence identity, at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 4 [Soybean (XP_003517716.1)], a fifth polypeptide with at least 70% sequence identity, at least 71% sequence identity, at least 72% sequence identity, at least 73% sequence identity, at least 74% sequence identity, at least 75% sequence identity, at least 76% sequence identity, at least 77% sequence identity, at least 78% sequence identity, at least 79% sequence identity, at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 5 [Phaseolus (XP_007157313.1)], a sixth polypeptide with at least 70% sequence identity, at least 71% sequence identity, at least 72% sequence identity, at least 73% sequence identity, at least 74% sequence identity, at least 75% sequence identity, at least 76% sequence identity, at least 77% sequence identity, at least 78% sequence identity, at least 79% sequence identity, at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 6 [Populus (XP_002322185.1)], a seventh polypeptide with at least 70% sequence identity, at least 71% sequence identity, at least 72% sequence identity, at least 73% sequence identity, at least 74% sequence identity, at least 75% sequence identity, at least 76% sequence identity, at least 77% sequence identity, at least 78% sequence identity, at least 79% sequence identity, at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 7 [Malta (XP_008340354.1)], an eighth polypeptide with at least 70% sequence identity, at least 71% sequence identity, at least 72% sequence identity, at least 73% sequence identity, at least 74% sequence identity, at least 75% sequence identity, at least 76% sequence identity, at least 77% sequence identity, at least 78% sequence identity, at least 79% sequence identity, at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 8 [Vitis (XP_002272814.2)], a ninth polypeptide with at least 70% sequence identity, at least 71% sequence identity, at least 72% sequence identity, at least 73% sequence identity, at least 74% sequence identity, at least 75% sequence identity, at least 76% sequence identity, at least 77% sequence identity, at least 78% sequence identity, at least 79% sequence identity, at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 9 [Theobroma (XP_007036352.1)], a tenth polypeptide with at least 70% sequence identity, at least 71% sequence identity, at least 72% sequence identity, at least 73% sequence identity, at least 74% sequence identity, at least 75% sequence identity, at least 76% sequence identity, at least 77% sequence identity, at least 78% sequence identity, at least 79% sequence identity, at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 10 [Ricinus (XP_002527912.1)], an eleventh polypeptide with at least 70% sequence identity, at least 71% sequence identity, at least 72% sequence identity, at least 73% sequence identity, at least 74% sequence identity, at least 75% sequence identity, at least 76% sequence identity, at least 77% sequence identity, at least 78% sequence identity, at least 79% sequence identity, at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 11 [Fragaria (XP_004300916.1)], a twelfth polypeptide with at least 70% sequence identity, at least 71% sequence identity, at least 72% sequence identity, at least 73% sequence identity, at least 74% sequence identity, at least 75% sequence identity, at least 76% sequence identity, at least 77% sequence identity, at least 78% sequence identity, at least 79% sequence identity, at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 12 [Maize (XP_008657477.1)], a thirteenth polypeptide with at least 70% sequence identity, at least 71% sequence identity, at least 72% sequence identity, at least 73% sequence identity, at least 74% sequence identity, at least 75% sequence identity, at least 76% sequence identity, at least 77% sequence identity, at least 78% sequence identity, at least 79% sequence identity, at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 13 [Rice (XP_015628733.1)], a fourteenth polypeptide with at least 70% sequence identity, at least 71% sequence identity, at least 72% sequence identity, at least 73% sequence identity, at least 74% sequence identity, at least 75% sequence identity, at least 76% sequence identity, at least 77% sequence identity, at least 78% sequence identity, at least 79% sequence identity, at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 14 [Wheat (CDM80098.1)], or a fifteenth polypeptide with at least 70% sequence identity, at least 71% sequence identity, at least 72% sequence identity, at least 73% sequence identity, at least 74% sequence identity, at least 75% sequence identity, at least 76% sequence identity, at least 77% sequence identity, at least 78% sequence identity, at least 79% sequence identity, at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 15 [Barley (MLOC_5489.2)]. A further embodiment of this aspect includes the heterologous EPR3 or EPR3-like polypeptide being selected from the group of SEQ ID NO: 1 [L. japonicus (EPR3)], SEQ ID NO: 2 [Chickpea (XP_004489790.1)], SEQ ID NO: 3 [Medicago (XP_003613165.1)], SEQ ID NO: 4 [Soybean (XP_003517716.1)], SEQ ID NO: 5 [Phaseolus (XP_007157313.1)], SEQ ID NO: 6 [Populus (XP_002322185.1)], SEQ ID NO: 7 [Malus (XP_008340354.1)], SEQ ID NO: 8 [Vitis (XP_002272814.2)], SEQ ID NO: 9 [Theobroma (XP_007036352.1)], SEQ ID NO: 10 [Ricinus (XP_002527912.1)], SEQ ID NO: 11 [Fragaria (XP_004300916.1)], SEQ ID NO: 12 [Maize (XP_008657477.1)], SEQ ID NO: 13 [Rice (XP_015628733.1)], SEQ ID NO: 14 [Wheat (CDM80098.1)], or SEQ ID NO: 15 [Barley (MLOC_5489.2)].

Still another embodiment of this aspect, which may be combined with any of the preceding embodiments, the modified EPR3a or EPR3a-like polypeptide comprises a modified ectodomain that has been replaced with all or a portion of an ectodomain of the heterologous EPR3a or EPR3a-like polypeptide, optionally all or a part of the M1 domain, the M2 domain, the LysM3 domain, or all three. In an additional embodiment of this aspect, the portion replaced is at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, less than 10%, less than 11%, less than 12%, less than 13%, less than 14%, less than 15%, less than 16%, less than 17%, less than 18%, less than 19%, less than 20%, less than 21%, less than 22%, less than 23%, less than 24%, less than 25%, less than 26%, less than 27%, less than 28%, less than 29%, less than 30%, less than 31%, less than 32%, less than 33%, less than 34%, less than 35%, less than 36%, less than 37%, less than 38%, less than 39%, less than 40%, less than 41%, less than 42%, less than 43%, less than 44%, less than 45%, less than 46%, less than 47%, less than 48%, less than 49%, less than 50%, less than 51%, less than 52%, less than 53%, less than 54%, less than 55%, less than 56%, less than 57%, less than 58%, less than 59%, less than 60%, less than 61%, less than 62%, less than 63%, less than 64%, less than 65%, less than 66%, less than 67%, less than 68%, less than 69%, less than 70%, less than 71%, less than 72%, less than 73%, less than 74%, less than 75%, less than 76%, less than 77%, less than 78%, less than 79%, less than 80%, less than 81%, less than 82%, less than 83%, less than 84%, less than 85%, less than 86%, less than 87%, less than 88%, less than 89%, or less than 90%, of the ectodomain or, optionally all or a part of the M1 domain, the M2 domain, the LysM3 domain, or all three. In yet another embodiment of this aspect, which may be combined with any of the preceding embodiments that have an EPR3 or EPR3-like polypeptide, the modified EPR3 or EPR3-like polypeptide includes a modified ectodomain that has been replaced with all or a portion of an ectodomain of the heterologous EPR3 or EPR3-like polypeptide, optionally all or a part of the M1 domain, the M2 domain, the LysM3 domain, or all three. In an additional embodiment of this aspect, the portion replaced is at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, less than 10%, less than 11%, less than 12%, less than 13%, less than 14%, less than 15%, less than 16%, less than 17%, less than 18%, less than 19%, less than 20%, less than 21%, less than 22%, less than 23%, less than 24%, less than 25%, less than 26%, less than 27%, less than 28%, less than 29%, less than 30%, less than 31%, less than 32%, less than 33%, less than 34%, less than 35%, less than 36%, less than 37%, less than 38%, less than 39%, less than 40%, less than 41%, less than 42%, less than 43%, less than 44%, less than 45%, less than 46%, less than 47%, less than 48%, less than 49%, less than 50%, less than 51%, less than 52%, less than 53%, less than 54%, less than 55%, less than 56%, less than 57%, less than 58%, less than 59%, less than 60%, less than 61%, less than 62%, less than 63%, less than 64%, less than 65%, less than 66%, less than 67%, less than 68%, less than 69%, less than 70%, less than 71%, less than 72%, less than 73%, less than 74%, less than 75%, less than 76%, less than 77%, less than 78%, less than 79%, less than 80%, less than 81%, less than 82%, less than 83%, less than 84%, less than 85%, less than 86%, less than 87%, less than 88%, less than 89%, or less than 90%, of the ectodomain or, optionally all or a part of the M1 domain, the M2 domain, the LysM3 domain, or all three. A further embodiment of this aspect, which may be combined with any of the preceding embodiments that have an EPR3 or EPR3-like polypeptide, includes the heterologous EPR3a or EPR3a-like polypeptide and the heterologous EPR3 or EPR3-like polypeptide being from the same plant species or the same plant variety.

Yet another embodiment of this aspect, which may be combined with any of the preceding embodiments, includes the expression of the heterologous EPR3a or EPR3a-like polypeptide or the modified EPR3a or EPR3a-like polypeptide allowing the plant or part thereof to recognize an EPS, a beta-glucan, a cyclic beta-glucan, a LPS, or a surface carbohydrate produced by the microbe. Still another embodiment of this aspect, which may be combined with any of the preceding embodiments that have an EPR3 or EPR3-like polypeptide, includes the expression of the heterologous EPR3 or EPR3-like polypeptide or the modified EPR3a or EPR3a-like polypeptide allowing the plant or part thereof to recognize an EPS, a beta-glucan, a cyclic beta-glucan, a LPS, or a surface carbohydrate produced by the microbe. In a further embodiment of this aspect, the expression of the heterologous EPR3a or EPR3a-like polypeptide or the modified EPR3a or EPR3a-like polypeptide and the expression of the heterologous EPR3 or EPR3-like polypeptide or the modified EPR3 or EPR3-like polypeptide allows the plant or part thereof to recognize an EPS, a beta-glucan, a cyclic beta-glucan, a LPS, or a surface carbohydrate produced by the microbe. In an additional embodiment of this aspect that can be combined with any preceding embodiments including an EPS produced by the microbe, the microbe is a commensal bacteria, optionally a nitrogen-fixing bacteria, or a mycorrhizal fungi. A further embodiment of this aspect includes the nitrogen-fixing bacteria being selected from the group of Mesorhizobium loti, Mesorhizobium huakuii, Mesorhizobium mediterraneum, Mesorhizobium ciceri, Mesorhizobium spp., Rhizobium mongolense, Rhizobium tropici, Rhizobium etli phaseoli, Rhizobium giardinii, Rhizobium leguminosarum optionally R. leguminosarum trifolii, R. leguminosarum viciae, and R. leguminosarum phaseoli, Burkholderiales optionally symbionts of Mimosa, Sinorhizobium meliloti, Sinorhizobium medicae, Sinorhizobium fredii, Sinorhizobium fredii NGR234, Azorhizobium caulinodans, Bradyrhizobium japonicum, Bradyrhizobium elkanii, Bradyrhizobium liaonginense, Rhizobium spp., Mesorhizobium spp., Sinorhizobium spp., Azorhizobium spp. Frankia spp., or any combination thereof, or the mycorrhizal fungi being selected from the group of Acaulosporaceae spp., Diversisporaceae spp., Gigasporaceae spp., Pacisporaceae spp., Funneliformis spp., Glomus spp., Rhizophagus spp., Sclerocystis spp., Septoglomus spp., Claroideoglomus spp., Ambispora spp., Archaeospora spp., Geosiphon pyriformis, Paraglomus spp., other species in the division Glomeromycota, or any combination thereof. Still another embodiment of this aspect, which may be combined with any preceding embodiments, includes the heterologous EPR3a or EPR3a-like polypeptide or the modified EPR3a or EPR3a-like polypeptide being localized to a plant cell plasma membrane. Yet another embodiment of this aspect, which may be combined with any of the preceding embodiments that have an EPR3 or EPR3-like polypeptide, includes the heterologous EPR3 or EPR3-like polypeptide or the modified EPR3 or EPR3-like polypeptide being localized to a plant cell plasma membrane. A further embodiment of this aspect that can be combined with any of the preceding embodiments that have localization to a plant cell plasma membrane includes the plant cell being a root cell. An additional embodiment of this aspect includes the root cell being a root epidermal cell or a root cortex cell. In a further embodiment of this aspect that can be combined with any of the preceding embodiments, the heterologous EPR3a or EPR3a-like polypeptide or the modified EPR3a or EPR3a-like polypeptide is expressed in a developing plant root system. In an additional embodiment of this aspect that can be combined with any of the preceding embodiments that has an EPR3 or EPR3-like polypeptide, the heterologous EPR3 or EPR3-like polypeptide or the modified EPR3 or EPR3-like polypeptide is expressed in a developing plant root system.

In still another embodiment of this aspect, which may be combined with any of the preceding embodiments, the first nucleic acid sequence is operably linked to a first promoter. In an additional embodiment of this aspect, the first promoter is a root specific promoter, and the root specific promoter is optionally selected from the group of a NFR1 or NFR5/NFP promoter, an EPR3 or an EPR3a promoter, a Lotus NFR5 promoter, a Lotus NFR1 promoter, a maize allothioneine promoter, a chitinase promoter, a maize ZRP2 promoter, a tomato LeExt1 promoter, a glutamine synthetase soybean root promoter, a RCC3 promoter, a rice antiquitine promoter, a LRR receptor kinase promoter, or an Arabidopsis pCO2 promoter. In a further embodiment of this aspect, the first promoter is a constitutive promoter, and the constitutive promoter is optionally selected from the group of a CaMV35S promoter, a derivative of the CaMV35S promoter, a maize ubiquitin promoter, a trefoil promoter, a vein mosaic cassava virus promoter, or an Arabidopsis UBQ10 promoter. In yet another embodiment of this aspect, which may be combined with any of the preceding embodiments that has an EPR3 or an EPR3-like polypeptide, the second nucleic acid sequence is operably linked to a second promoter. In an additional embodiment of this aspect, the second promoter is a root specific promoter, and the root specific promoter is optionally selected from the group of a NFR1 or NFR5/NFP promoter, an EPR3 or an EPR3a promoter, a Lotus NFR5 promoter, a Lotus NFR1 promoter, a maize allothioneine promoter, a chitinase promoter, a maize ZRP2 promoter, a tomato LeExt1 promoter, a glutamine synthetase soybean root promoter, a RCC3 promoter, a rice antiquitine promoter, a LRR receptor kinase promoter, or an Arabidopsis pCO2 promoter. In a further embodiment of this aspect, the second promoter is a constitutive promoter, and the constitutive promoter is optionally selected from the group of a CaMV35S promoter, a derivative of the CaMV35S promoter, a maize ubiquitin promoter, a trefoil promoter, a vein mosaic cassava virus promoter, or an Arabidopsis UBQ10 promoter. In an additional embodiment of this aspect, which may be combined with any of the preceding embodiments, the plant is selected from the group of group of corn (e.g., maize, Zea mays), rice (e.g., indica rice, japonica rice, aromatic rice, glutinous rice, Oryza sativa, Oryza glaberrima), wild rice (e.g., Zizania spp., Porteresia spp.), wheat (e.g., common wheat, spelt, durum, einkorn, emmer, kamut, Triticum aestivum, Triticum spelta, Triticum durum, Triticum urartu, Triticum monococcum, Triticum turanicum, Triticum spp.), barley (e.g., Hordeum vulgare), sorghum (e.g., Sorghum bicolor), millet (e.g., finger millet, fonio millet, foxtail millet, pearl millet, barnyard millets, Eleusine coracana, Panicum sumatrense, Panicum milaceum, Setaria italica, Pennisetum glaucum, Digitaria spp., Echinocloa spp.), teff (e.g., Eragrostis tef), oat (e.g., Avena sativa), triticale (e.g., X Triticosecale Wittmack, Triticosecale schlanstedtense Wittm., Triticosecale neoblaringhemii A. Camus, Triticosecale neoblaringhemii A. Camus), rye (e.g., Secale cereale, Secale cereanum), sugar cane (e.g., Saccharum officinarum, Saccharum spp.), apple (e.g., Malus pumila, Malus x domestica, Pyrus malus), pear (e.g., Pyrus communis, Pyrus x bretschneideri, Pyrus pyrifolia, Pyrus sinkiangensis, Pyrus pashia, Pyrus spp.), plum (e.g., Mirabelle, greengage, damson, Prunus domestica, Prunus salicina, Prunus mume), apricot (e.g., Prunus armeniaca, Prunus brigantine, Prunus mandshurica), peach (e.g., Prunus persica), almond (e.g., Prunus dulcis, Prunus amygdalus), walnut (e.g., Persian walnut, English walnut, black walnut, Juglans regia, Juglans nigra, Juglans cinerea, Juglans californica), cherry (e.g., Prunus avium, Prunus cerasus, Prunus yedoensis var. nudiflora), strawberry (e.g., Fragaria x ananassa, Fragaria chiloensis, Fragaria virginiana, Fragaria vesca), raspberry (e.g., European red raspberry, black raspberry, Rubus idaeus L., Rubus occidentalis, Rubus strigosus), blackberry (e.g., evergreen blackberry, Himalayan blackberry, Rubus fruticosus, Rubus ursinus, Rubus laciniatus, Rubus argutus, Rubus armeniacus, Rubus plicatus, Rubus ulmifolius, Rubus allegheniensis, Rubus subgenus Eubatus sect. Moriferi & Ursini), red currant (e.g., white currant, Ribes rubrum), black currant (e.g., cassis, Ribes nigrum), gooseberry (e.g., Ribes uva-crispa, Ribes grossulari, Ribes hirtellum), cowpea (e.g., Vigna unguiculata), melon (e.g., watermelon, winter melon, casabas, cantaloupe, honeydew, muskmelon, Citrullus lanatus, Benincasa hispida, Cucumis melo, Cucumis melo cantalupensis, Cucumis melo inodorus, Cucumis melo reticulatus), cucumber (e.g., slicing cucumbers, pickling cucumbers, English cucumber, Cucumis sativus), pumpkin (e.g., Cucurbita pepo, Cucurbita maxima), squash (e.g., gourd, Cucurbita argyrosperma, Cucurbita ficifolia, Cucurbita maxima, Cucurbita moschata), grape (e.g., Vitis vinifera, Vitis amurensis, Vitis labrusca, Vitis mustangensis, Vitis riparia, Vitis rotundifolia), hemp (e.g., cannabis, Cannabis sativa), hops (e.g., Humulus lupulus), birch (e.g., Betula spp.), beech (e.g., Fagus sylvatica, Fagus grandifolia, Fagus spp.), jujube (e.g., red date, Ziziphus jujube), cassava (e.g., manioc, yucca, Manihot esculenta), poplar (e.g., hybrid poplar, Populus trichocarpa, Populus tremula, Populus alba, Populus spp.), chestnut (e.g., Castanea mollissima, Castanea crenata, Castanea dentata, Castanea spp.), swamp oak (e.g., Casuarina glauca), rose gum (e.g., Eucalyptus grandis), oak (e.g., cork oak, Quercus suber, Quercus spp.), citrus (e.g., lemon, lime, orange, grapefruit, pomelo, citron, trifoliate orange, bergamot orange, bitter orange, blood orange, satsuma, clementine, mandarin, yuzu, finger lime, kaffir lime, kumquat, Citrus clementina, Citrus sinensis, Citrus trifoliata, Citrus japonica, Citrus maxima, Citrus australasica, Citrus reticulata, Citus aurantifolia, Citrus hystrix, Citrus x paradisi, Citrus x clementina, Citrus spp.), potato (e.g., russet potatoes, yellow potatoes, red potatoes, Solanum tuberosum), tomato (e.g., Solanum lycopersicum), pepper (e.g., sweet pepper, bell pepper, hot pepper, chili pepper, Capsicum L.), sweet potato (e.g., Ipomoea batatas), yam (e.g., Diascorea spp., Oxalis tuberosa), Trema spp. (e.g., Trema cannabina, Trema cubense, Trema discolor, Trema domingensis, Trema integerrima, Trema lamarckiana, Trema micrantha, Trema orientalis, Trema philippinensis, Trema strigilosa, Trema tomentosa, Trema levigata), and Jatropha spp. (e.g., Jatropha curcas). In yet another embodiment of this aspect, which may be combined with any of the preceding embodiments, the plant lacks functional rhizobial Nod factor receptors. In still another embodiment of this aspect, which may be combined with any of the preceding embodiments, the plant is not a legume. In an additional embodiment of this aspect, which may be combined with any of the preceding embodiments, the plant is not A. thaliana, N. tabacum, L. japonicus, or M. truncatula. In a further embodiment of this aspect, which may be combined with any of the above embodiments, the plant part is a leaf, a stem, a root, a root primordia, a flower, a seed, a fruit, a kernel, a grain, a cell, or a portion thereof. An additional embodiment of this aspect includes the plant part being a fruit, a kernel, or a grain.

In some aspects, the present disclosure relates to a pollen grain or an ovule of the genetically altered plant of any of the above embodiments.

In some aspects, the present disclosure relates to a protoplast produced from the plant of any of the above embodiments.

In some aspects, the present disclosure relates to a tissue culture produced from protoplasts or cells from the plant of any of the above embodiments, wherein the cells or protoplasts are produced from a plant part selected from the group of leaf, anther, pistil, stem, petiole, root, root primordia, root tip, fruit, seed, flower, cotyledon, hypocotyl, embryo, or meristematic cell.

### Methods of Producing and Cultivating Genetically Altered Plants

Another aspect of the disclosure includes methods of producing the genetically altered plant of any of the above embodiments, including introducing a genetic alteration to the plant comprising the first nucleic acid sequence encoding the heterologous EPR3 or EPR3-like polypeptide. An additional embodiment of this aspect includes the first nucleic acid sequence being operably linked to a first promoter. Yet another embodiment of this aspect includes the first promoter being a root specific promoter, and the root specific promoter being optionally selected from the group of a NFR1 or NFR5/NFP promoter, an EPR3 or an EPR3a promoter, a Lotus NFR5 promoter, a Lotus NFR1 promoter, a maize allothioneine promoter, a chitinase promoter, a maize ZRP2 promoter, a tomato LeExt1 promoter, a glutamine synthetase soybean root promoter, a RCC3 promoter, a rice antiquitine promoter, a LRR receptor kinase promoter, or an Arabidopsis pCO2 promoter. Still another embodiment of this aspect includes the first promoter being a constitutive promoter, and the constitutive promoter being optionally selected from the group of a CaMV35S promoter, a derivative of the CaMV35S promoter, a maize ubiquitin promoter, a trefoil promoter, a vein mosaic cassava virus promoter, or an Arabidopsis UBQ10 promoter. An additional embodiment of this aspect further includes introducing a genetic alteration to the plant including the second nucleic acid sequence encoding the heterologous EPR3a or EPR3a-like polypeptide. A further embodiment of this aspect includes the second nucleic acid sequence being operably linked to a second promoter. Yet another embodiment of this aspect includes the second promoter being a root specific promoter, and the root specific promoter being optionally selected from the group of a NFR1 or NFR5/NFP promoter, an EPR3 or an EPR3a promoter, a Lotus NFR5 promoter, a Lotus NFR1 promoter, a maize allothioneine promoter, a chitinase promoter, a maize ZRP2 promoter, a tomato LeExt1 promoter, a glutamine synthetase soybean root promoter, a RCC3 promoter, a rice antiquitine promoter, a LRR receptor kinase promoter, or an Arabidopsis pCO2 promoter. Still another embodiment of this aspect includes the second promoter being a constitutive promoter, and the constitutive promoter being optionally selected from the group consisting of a CaMV35S promoter, a derivative of the CaMV35S promoter, a maize ubiquitin promoter, a trefoil promoter, a vein mosaic cassava virus promoter, or an Arabidopsis UBQ10 promoter. In a further embodiment of this aspect, with may be combined with any of the preceding embodiments, the first nucleic acid sequence is inserted into the genome of the plant so that the nucleic acid sequence is operably linked to a first endogenous promoter. An additional embodiment of this aspect includes the first endogenous promoter being a root specific promoter. In yet another embodiment of this aspect, with may be combined with any of the preceding embodiments that has the second nucleic acid sequence, the second nucleic acid sequence is inserted into the genome of the plant so that the nucleic acid sequence is operably linked to a second endogenous promoter. A further embodiment of this aspect includes the second endogenous promoter being a root specific promoter. Yet another embodiment of this aspect, which may be combined with any preceding embodiment that has the first nucleic acid sequence being inserted into the genome of the plant or the second nucleic acid sequence being inserted into the genome of the plant includes insertion resulting from the use of one or more gene editing components that target a nuclear genome sequence operably linked to an endogenous promoter. Still another embodiment of this aspect includes one or more gene editing components being selected from the group of a ribonucleoprotein complex that targets the nuclear genome sequence; a vector including a TALEN protein encoding sequence, wherein the TALEN protein targets the nuclear genome sequence; a vector including a ZFN protein encoding sequence, wherein the ZFN protein targets the nuclear genome sequence; an oligonucleotide donor (ODN), wherein the ODN targets the nuclear genome sequence; or a vector including a CRISPR/Cas enzyme encoding sequence and a targeting sequence, wherein the targeting sequence targets the nuclear genome sequence.

An additional aspect of the present disclosure relates to methods of producing the genetically altered plant of any one of the preceding embodiments that have a modified polypeptide, including genetically editing a gene encoding an endogenous LysM receptor polypeptide in the plant to comprise the modified ectodomain. In a further embodiment of this aspect, the endogenous LysM receptor polypeptide is an endogenous EPR3 or EPR3-like polypeptide. In another embodiment of this aspect, which may be combined with any of the preceding embodiments, the modified EPR3 or EPR3-like polypeptide was generated by: (a) providing a heterologous EPR3 or EPR3-like polypeptide model including a structural model, a molecular model, a surface characteristics model, and/or an electrostatic potential model of a M1 domain, a M2 domain, a LysM3 domain, any combination thereof, or the ectodomain of the heterologous EPR3 or EPR3-like polypeptide having selectivity for the beneficial commensal microbe and an unmodified EPR3 or EPR3-like polypeptide; (b) identifying one or more amino acid residues for modification in the unmodified EPR3 or EPR3-like polypeptide by comparing amino acid residues of a oligosaccharide binding feature in the unmodified EPR3 or EPR3-like polypeptide with the corresponding amino acid residues in the heterologous EPR3 or EPR3-like polypeptide model; and (c) generating the unmodified EPR3 or EPR3-like polypeptide wherein the one or more amino acid residues in the oligosaccharide binding feature of the unmodified EPR3 or EPR3-like polypeptide have been substituted with corresponding amino acid residues from the heterologous EPR3 or EPR3-like polypeptide. Selectivity may mean positive selection of the beneficial commensal microbe, negative selection of other microbes that are not the beneficial commensal, or a combination thereof. Yet another embodiment of this aspect includes the heterologous EPR3 or EPR3-like polypeptide model being a protein crystal structure, a molecular model, a cryo-EM structure, or a NMR structure. In an additional embodiment of this aspect, the endogenous LysM receptor polypeptide is an endogenous EPR3a or EPR3a-like polypeptide. In another embodiment of this aspect, the modified EPR3a or EPR3a-like polypeptide was generated by: (a) providing a heterologous EPR3a or EPR3a-like polypeptide model including a structural model, a molecular model, a surface characteristics model, and/or an electrostatic potential model of a M1 domain, a M2 domain, a LysM3 domain, any combination thereof, or the ectodomain of the heterologous EPR3a or EPR3a-like polypeptide having selectivity for the beneficial commensal microbe and an unmodified EPR3a or EPR3a-like polypeptide; (b) identifying one or more amino acid residues for modification in the unmodified EPR3a or EPR3a-like polypeptide by comparing amino acid residues of a oligosaccharide binding feature in the unmodified EPR3a or EPR3a-like polypeptide with the corresponding amino acid residues in the heterologous EPR3a or EPR3a-like polypeptide model; and (c) generating the unmodified EPR3a or EPR3a-like polypeptide wherein the one or more amino acid residues in the oligosaccharide binding feature of the unmodified EPR3a or EPR3a-like polypeptide have been substituted with corresponding amino acid residues from the heterologous EPR3a or EPR3a-like polypeptide. Selectivity may mean positive selection of the beneficial commensal microbe, negative selection of other microbes that are not the beneficial commensal, or a combination thereof. Yet another embodiment of this aspect includes the heterologous EPR3a or EPR3a-like polypeptide model being a protein crystal structure, a molecular model, a cryo-EM structure, or a NMR structure. A further embodiment of this aspect that can be combined with any of the preceding embodiments includes a plant or plant part produced by the method of any one of the preceding embodiments.

A further aspect of the present disclosure relates to methods of producing the genetically altered plant of any of the above embodiments, including introducing a genetic alteration to the plant comprising the first nucleic acid sequence encoding the heterologous EPR3a or EPR3a-like polypeptide. An additional embodiment of this aspect includes the first nucleic acid sequence being operably linked to a first promoter. Yet another embodiment of this aspect includes the first promoter being a root specific promoter, and the root specific promoter being optionally selected from the group of a NFR1 or NFR5/NFP promoter, an EPR3 or an EPR3a promoter, a Lotus NFR5 promoter, a Lotus NFR1 promoter, a maize allothioneine promoter, a chitinase promoter, a maize ZRP2 promoter, a tomato LeExt1 promoter, a glutamine synthetase soybean root promoter, a RCC3 promoter, a rice antiquitine promoter, a LRR receptor kinase promoter, or an Arabidopsis pCO2 promoter. Still another embodiment of this aspect includes the first promoter being a constitutive promoter, and the constitutive promoter being optionally selected from the group of a CaMV35S promoter, a derivative of the CaMV35S promoter, a maize ubiquitin promoter, a trefoil promoter, a vein mosaic cassava virus promoter, or an Arabidopsis UBQ10 promoter. An additional embodiment of this aspect further includes introducing a genetic alteration to the plant including the second nucleic acid sequence encoding the heterologous EPR3 or EPR3-like polypeptide. A further embodiment of this aspect includes the second nucleic acid sequence being operably linked to a second promoter. Yet another embodiment of this aspect includes the second promoter being a root specific promoter, and the root specific promoter being optionally selected from the group of a NFR1 or NFR5/NFP promoter, an EPR3 or an EPR3a promoter, a Lotus NFR5 promoter, a Lotus NFR1 promoter, a maize allothioneine promoter, a chitinase promoter, a maize ZRP2 promoter, a tomato LeExt1 promoter, a glutamine synthetase soybean root promoter, a RCC3 promoter, a rice antiquitine promoter, a LRR receptor kinase promoter, or an Arabidopsis pCO2 promoter. Still another embodiment of this aspect includes the second promoter being a constitutive promoter, and the constitutive promoter being optionally selected from the group consisting of a CaMV35S promoter, a derivative of the CaMV35S promoter, a maize ubiquitin promoter, a trefoil promoter, a vein mosaic cassava virus promoter, or an Arabidopsis UBQ10 promoter. In a further embodiment of this aspect, with may be combined with any of the preceding embodiments, the first nucleic acid sequence is inserted into the genome of the plant so that the nucleic acid sequence is operably linked to a first endogenous promoter. An additional embodiment of this aspect includes the first endogenous promoter being a root specific promoter. In yet another embodiment of this aspect, with may be combined with any of the preceding embodiments that has the second nucleic acid sequence, the second nucleic acid sequence is inserted into the genome of the plant so that the nucleic acid sequence is operably linked to a second endogenous promoter. A further embodiment of this aspect includes the second endogenous promoter being a root specific promoter. Yet another embodiment of this aspect, which may be combined with any preceding embodiment that has the first nucleic acid sequence being inserted into the genome of the plant or the second nucleic acid sequence being inserted into the genome of the plant includes insertion resulting from the use of one or more gene editing components that target a nuclear genome sequence operably linked to an endogenous promoter. Still another embodiment of this aspect includes one or more gene editing components being selected from the group of a ribonucleoprotein complex that targets the nuclear genome sequence; a vector including a TALEN protein encoding sequence, wherein the TALEN protein targets the nuclear genome sequence; a vector including a ZFN protein encoding sequence, wherein the ZFN protein targets the nuclear genome sequence; an oligonucleotide donor (ODN), wherein the ODN targets the nuclear genome sequence; or a vector including a CRISPR/Cas enzyme encoding sequence and a targeting sequence, wherein the targeting sequence targets the nuclear genome sequence.

An additional aspect of the present disclosure relates to methods of producing the genetically altered plant of any one of the preceding embodiments that have a modified polypeptide, including genetically editing a gene encoding an endogenous LysM receptor polypeptide in the plant to comprise the modified ectodomain. In a further embodiment of this aspect, the endogenous LysM receptor polypeptide is an endogenous EPR3a or an EPR3a-like polypeptide. In another embodiment of this aspect, which may be combined with any of the preceding embodiments, the modified EPR3a or EPR3a-like polypeptide was generated by: (a) providing a heterologous EPR3a or EPR3a-like polypeptide model including a structural model, a molecular model, a surface characteristics model, and/or an electrostatic potential model of a M1 domain, a M2 domain, a LysM3 domain, any combination thereof, or the ectodomain of the heterologous EPR3a or EPR3a-like polypeptide having selectivity for the beneficial commensal microbe and an unmodified EPR3a or EPR3a-like polypeptide; (b) identifying one or more amino acid residues for modification in the unmodified EPR3a or EPR3a-like polypeptide by comparing amino acid residues of a oligosaccharide binding feature in the unmodified EPR3a or EPR3a-like polypeptide with the corresponding amino acid residues in the heterologous EPR3a or EPR3a-like polypeptide model; and (c) generating the unmodified EPR3a or EPR3a-like polypeptide wherein the one or more amino acid residues in the oligosaccharide binding feature of the unmodified EPR3a or EPR3a-like polypeptide have been substituted with corresponding amino acid residues from the heterologous EPR3a or EPR3a-like polypeptide. Selectivity may mean positive selection of the beneficial commensal microbe, negative selection of other microbes that are not the beneficial commensal, or a combination thereof. Yet another embodiment of this aspect includes the heterologous EPR3a or EPR3a-like polypeptide model being a protein crystal structure, a molecular model, a cryo-EM structure, or a NMR structure. In an additional embodiment of this aspect, the endogenous LysM receptor polypeptide is an endogenous EPR3 or EPR3-like polypeptide. In another embodiment of this aspect, the modified EPR3 or EPR3-like polypeptide was generated by: (a) providing a heterologous EPR3 or EPR3-like polypeptide model including a structural model, a molecular model, a surface characteristics model, and/or an electrostatic potential model of a M1 domain, a M2 domain, a LysM3 domain, any combination thereof, or the ectodomain of the heterologous EPR3 or EPR3-like polypeptide having selectivity for the beneficial commensal microbe and an unmodified EPR3 or EPR3-like polypeptide; (b) identifying one or more amino acid residues for modification in the unmodified EPR3 or EPR3-like polypeptide by comparing amino acid residues of a oligosaccharide binding feature in the unmodified EPR3 or EPR3-like polypeptide with the corresponding amino acid residues in the heterologous EPR3 or EPR3-like polypeptide model; and (c) generating the unmodified EPR3 or EPR3-like polypeptide wherein the one or more amino acid residues in the oligosaccharide binding feature of the unmodified EPR3 or EPR3-like polypeptide have been substituted with corresponding amino acid residues from the heterologous EPR3 or EPR3-like polypeptide. Selectivity may mean positive selection of the beneficial commensal microbe, negative selection of other microbes that are not the beneficial commensal, or a combination thereof. Yet another embodiment of this aspect includes the heterologous EPR3 or EPR3-like polypeptide model being a protein crystal structure, a molecular model, a cryo-EM structure, or a NMR structure. A further embodiment of this aspect that can be combined with any of the preceding embodiments includes a plant or plant part produced by the method of any one of the preceding embodiments.

Yet another aspect of the disclosure includes methods of cultivating the genetically altered plant of any of the preceding embodiments that has a genetically altered plant, including the steps of: a) planting a genetically altered seedling, a genetically altered plantlet, a genetically altered cutting, a genetically altered tuber, a genetically altered root, or a genetically altered seed in soil to produce the genetically altered plant or grafting the genetically altered seedling, the genetically altered plantlet, or the genetically altered cutting to a root stock or a second plant grown in soil to produce the genetically altered plant; b) cultivating the plant to produce harvestable seed, harvestable leaves, harvestable roots, harvestable cuttings, harvestable wood, harvestable fruit, harvestable kernels, harvestable tubers, and/or harvestable grain; and harvesting the harvestable seed, harvestable leaves, harvestable roots, harvestable cuttings, harvestable wood, harvestable fruit, harvestable kernels, harvestable tubers, and/or harvestable grain; and c) harvesting the harvestable seed, harvestable leaves, harvestable roots, harvestable cuttings, harvestable wood, harvestable fruit, harvestable kernels, harvestable tubers, and/or harvestable grain.

Methods of Identifying a Beneficial Commensal Microbe Yet another aspect of the present disclosure relates to methods of identifying a beneficial commensal microbe capable of participating in a plant root microbiota including: a) providing a first polypeptide including an EPR3 or EPR3-like polypeptide, an ectodomain of an EPR3 or EPR3-like polypeptide, a M1 domain of an EPR3 or EPR3-like polypeptide, a M2 domain of an EPR3 or EPR3-like polypeptide, or a LysM3 domain of an EPR3 or EPR3-like polypeptide of the plant; b) contacting the first polypeptide with a sample including a microbe or an EPS, a beta-glucan, a cyclic beta-glucan, a LPS, or a surface carbohydrate produced by the microbe; and c) detecting binding of the EPS, the beta-glucan, the cyclic beta-glucan, the LPS, or the surface carbohydrate produced by the microbe to the polypeptide, wherein binding of the EPS, the beta-glucan, the cyclic beta-glucan, the LPS, or the surface carbohydrate to the polypeptide indicates that the microbe is a beneficial commensal microbe capable of participating in a plant root microbiota; optionally, detecting enrichment of taxa in Burkholderiales and/or Rhizobiales in a plant rhizosphere or endosphere, wherein enrichment of taxa in Burkholderiales and/or Rhizobiales in the plant rhizosphere or endosphere indicates that the microbe is a beneficial commensal microbe capable of participating in a plant root microbiota; optionally, the detecting is by a functional assay optionally selected from (i) detecting enrichment of taxa in Burkholderiales and/or Rhizobiales in a plant rhizosphere or endosphere, wherein enrichment of taxa in Burkholderiales and/or Rhizobiales in the plant rhizosphere or endosphere indicates that the microbe is a beneficial commensal microbe capable of participating in a plant root microbiota; optionally, (ii) detecting nodulation in a plant root system, wherein nodulation indicates that the microbe is a beneficial commensal microbe capable of participating in a plant root microbiota; and/or (iii) detecting mycorrhization in a plant root system, wherein mycorrhization indicates that the microbe is a beneficial commensal microbe capable of participating in a plant root microbiota, or optionally the detecting is by a direct binding assay optionally selected from (1) a competition assay optionally with a known signaling saccharide, or (2) an affinity assay optionally wherein the detected affinity is compared to the affinity for the known signaling saccharide. A further embodiment of this aspect further includes providing a second polypeptide including an EPR3a or EPR3a-like polypeptide, an ectodomain of an EPR3a or EPR3a-like polypeptide, a M1 domain of an EPR3a or EPR3a-like polypeptide, a M2 domain of an EPR3a or EPR3a-like polypeptide, or a LysM3 domain of an EPR3a or EPR3a-like polypeptide of the plant of the plant in step (a), wherein the second polypeptide is in contact with the first polypeptide. An additional embodiment of this aspect further includes step (d) culturing the beneficial commensal microbe if binding is detected in step (c). Yet another embodiment of this aspect further includes step (e) applying the beneficial commensal microbe to the plant or a part thereof. A further embodiment of this aspect includes the plant part being a plant propagation material, optionally a seed, a tuber, or a plantlet, and the beneficial commensal microbe being applied to the plant propagation material, optionally to the seed as part of a seed coating, to the tuber, or to a root of the plantlet. An additional embodiment of this aspect includes the plant part being a plant vegetative or reproductive material, optionally a root, a shoot, a stem, a pollen grain, or an ovule, and the beneficial commensal microbe is applied to the plant vegetative or reproductive material of the plant, optionally as part of a coating, a solution, or a powder. Still another embodiment of this aspect further includes step (e) applying the beneficial commensal microbe, optionally in admixture with a soil-compatible carrier, a fungal carrier, or a growth medium, optionally soil, where the plant is growing or is to be grown. Yet another embodiment of this aspect, which may be combined with any of the preceding embodiments having an ectodomain of an EPR3 or EPR3-like polypeptide, includes the ectodomain of the EPR3 or EPR3-like polypeptide having at least 70% sequence identity, at least 71% sequence identity, at least 72% sequence identity, at least 73% sequence identity, at least 74% sequence identity, at least 75% sequence identity, at least 76% sequence identity, at least 77% sequence identity, at least 78% sequence identity, at least 79% sequence identity, at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to the ectodomain of SEQ ID NO: 1 [L. japonicus (EPR3)], SEQ ID NO: 2 [Chickpea (XP_004489790.1)], SEQ ID NO: 3 [Medicago (XP_003613165.1)], SEQ ID NO: 4 [Soybean (XP_003517716.1)], SEQ ID NO: 5 [Phaseolus (XP_007157313.1)], SEQ ID NO: 6 [Populus (XP_002322185.1)], SEQ ID NO: 7 [Malus (XP_008340354.1)], SEQ ID NO: 8 [Vitis (XP_002272814.2)], SEQ ID NO: 9 [Theobroma (XP_007036352.1)], SEQ ID NO: 10 [Ricinus (XP_002527912.1)], SEQ ID NO: 11 [Fragaria (XP_004300916.1)], SEQ ID NO: 12 [Maize (XP_008657477.1)], SEQ ID NO: 13 [Rice (XP_015628733.1)], SEQ ID NO: 14 [Wheat (CDM80098.1)], or SEQ ID NO: 15 [Barley (MLOC_5489.2)]. An additional embodiment of this aspect, which may be combined with any of the preceding embodiments having an ectodomain of an EPR3 or EPR3-like polypeptide, includes the ectodomain of the EPR3 or EPR3-like polypeptide being the ectodomain of SEQ ID NO: 1 [L. japonicus (EPR3)], SEQ ID NO: 2 [Chickpea (XP_004489790.1)], SEQ ID NO: 3 [Medicago (XP_003613165.1)], SEQ ID NO: 4 [Soybean (XP_003517716.1)], SEQ ID NO: 5 [Phaseolus (XP_007157313.1)], SEQ ID NO: 6 [Populus (XP_002322185.1)], SEQ ID NO: 7 [Malus (XP_008340354.1)], SEQ ID NO: 8 [Vitis (XP_002272814.2)], SEQ ID NO: 9 [Theobroma (XP_007036352.1)], SEQ ID NO: 10 [Ricinus (XP_002527912.1)], SEQ ID NO: 11 [Fragaria (XP_004300916.1)], SEQ ID NO: 12 [Maize (XP_008657477.1)], SEQ ID NO: 13 [Rice (XP_015628733.1)], SEQ ID NO: 14 [Wheat (CDM80098.1)], or SEQ ID NO: 15 [Barley (MLOC_5489.2)]. A further embodiment of this aspect, which may be combined with any of the preceding embodiments having an ectodomain of an EPR3a or EPR3a-like polypeptide, includes the ectodomain of the EPR3a or EPR3a-like polypeptide having at least 70% sequence identity, at least 71% sequence identity, at least 72% sequence identity, at least 73% sequence identity, at least 74% sequence identity, at least 75% sequence identity, at least 76% sequence identity, at least 77% sequence identity, at least 78% sequence identity, at least 79% sequence identity, at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to the ectodomain of SEQ ID NO: 62 [L. japonicus (EPR3a)], SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, or SEQ ID NO: 92. Yet another embodiment of this aspect, which may be combined with any of the preceding embodiments having an ectodomain of an EPR3a or EPR3a-like polypeptide, includes the ectodomain of the EPR3a or EPR3a-like polypeptide being the ectodomain of SEQ ID NO: 62 [L. japonicus (EPR3a)], SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, or SEQ ID NO: 92. Still another embodiment of this aspect includes beneficial commensal microbe being a commensal bacteria, optionally a nitrogen-fixing bacteria, or a mycorrhizal fungi.

Still another aspect of the present disclosure relates to methods of identifying a beneficial commensal microbe capable of participating in a plant root microbiota including: a) providing a first polypeptide including an EPR3a or EPR3a-like polypeptide, an ectodomain of an EPR3a or EPR3a-like polypeptide, a M1 domain of an EPR3a or EPR3a-like polypeptide, a M2 domain of an EPR3a or EPR3a-like polypeptide, or a LysM3 domain of an EPR3a or EPR3a-like polypeptide of the plant; b) contacting the first polypeptide with a sample including a microbe or an EPS, a beta-glucan, a cyclic beta-glucan, a LPS, or a surface carbohydrate produced by the microbe; and c) detecting binding of the EPS, the beta-glucan, the cyclic beta-glucan, the LPS, or the surface carbohydrate produced by the microbe to the polypeptide, wherein binding of the EPS, the beta-glucan, the cyclic beta-glucan, the LPS, or the surface carbohydrate to the polypeptide indicates that the microbe is a beneficial commensal microbe capable of participating in a plant root microbiota; optionally, detecting enrichment of taxa in Burkholderiales and/or Rhizobiales in a plant rhizosphere or endosphere, wherein enrichment of taxa in Burkholderiales and/or Rhizobiales in the plant rhizosphere or endosphere indicates that the microbe is a beneficial commensal microbe capable of participating in a plant root microbiota; optionally, the detecting is by a functional assay optionally selected from (i) detecting enrichment of taxa in Burkholderiales and/or Rhizobiales in a plant rhizosphere or endosphere, wherein enrichment of taxa in Burkholderiales and/or Rhizobiales in the plant rhizosphere or endosphere indicates that the microbe is a beneficial commensal microbe capable of participating in a plant root microbiota; optionally, (ii) detecting nodulation in a plant root system, wherein nodulation indicates that the microbe is a beneficial commensal microbe capable of participating in a plant root microbiota; and/or (iii) detecting mycorrhization in a plant root system, wherein mycorrhization indicates that the microbe is a beneficial commensal microbe capable of participating in a plant root microbiota, or optionally the detecting is by a direct binding assay optionally selected from (1) a competition assay optionally with a known signaling saccharide, or (2) an affinity assay optionally wherein the detected affinity is compared to the affinity for the known signaling saccharide. A further embodiment of this aspect further includes providing a second polypeptide including an EPR3 or EPR3-like polypeptide, an ectodomain of an EPR3 or EPR3-like polypeptide, a M1 domain of an EPR3 or EPR3-like polypeptide, a M2 domain of an EPR3 or EPR3-like polypeptide, or a LysM3 domain of an EPR3 or EPR3-like polypeptide of the plant in step (a), wherein the second polypeptide is in contact with the first polypeptide. An additional embodiment of this aspect further includes step (d) culturing the beneficial commensal microbe if binding is detected in step (c). Yet another embodiment of this aspect further includes step (e) applying the beneficial commensal microbe to the plant or a part thereof. A further embodiment of this aspect includes the plant part being a plant propagation material, optionally a seed, a tuber, or a plantlet, and the beneficial commensal microbe being applied to the plant propagation material, optionally to the seed as part of a seed coating, to the tuber, or to a root of the plantlet. An additional embodiment of this aspect includes the plant part being a plant vegetative or reproductive material, optionally a root, a shoot, a stem, a pollen grain, or an ovule, and the beneficial commensal microbe is applied to the plant vegetative or reproductive material of the plant, optionally as part of a coating, a solution, or a powder. Still another embodiment of this aspect further includes step (e) applying the beneficial commensal microbe, optionally in admixture with a soil-compatible carrier, a fungal carrier, or a growth medium, optionally soil, where the plant is growing or is to be grown. A further embodiment of this aspect, which may be combined with any of the preceding embodiments having an ectodomain of an EPR3a or EPR3a-like polypeptide, includes the ectodomain of the EPR3a or EPR3a-like polypeptide having at least 70% sequence identity, at least 71% sequence identity, at least 72% sequence identity, at least 73% sequence identity, at least 74% sequence identity, at least 75% sequence identity, at least 76% sequence identity, at least 77% sequence identity, at least 78% sequence identity, at least 79% sequence identity, at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to the ectodomain of SEQ ID NO: 62 [L. japonicus (EPR3a)], SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, or SEQ ID NO: 92. Yet another embodiment of this aspect, which may be combined with any of the preceding embodiments having an ectodomain of an EPR3a or EPR3a-like polypeptide, includes the ectodomain of the EPR3a or EPR3a-like polypeptide being the ectodomain of SEQ ID NO: 62 [L. japonicus (EPR3a)], SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, or SEQ ID NO: 92. Yet another embodiment of this aspect, which may be combined with any of the preceding embodiments having an ectodomain of an EPR3 or EPR3-like polypeptide, includes the ectodomain of the EPR3 or EPR3-like polypeptide having at least 70% sequence identity, at least 71% sequence identity, at least 72% sequence identity, at least 73% sequence identity, at least 74% sequence identity, at least 75% sequence identity, at least 76% sequence identity, at least 77% sequence identity, at least 78% sequence identity, at least 79% sequence identity, at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to the ectodomain of SEQ ID NO: 1 [L. japonicus (EPR3)], SEQ ID NO: 2 [Chickpea (XP_004489790.1)], SEQ ID NO: 3 [Medicago (XP_003613165.1)], SEQ ID NO: 4 [Soybean (XP_003517716.1)], SEQ ID NO: 5 [Phaseolus (XP_007157313.1)], SEQ ID NO: 6 [Populus (XP_002322185.1)], SEQ ID NO: 7 [Malus (XP_008340354.1)], SEQ ID NO: 8 [Vitis (XP_002272814.2)], SEQ ID NO: 9 [Theobroma (XP_007036352.1)], SEQ ID NO: 10 [Ricinus (XP_002527912.1)], SEQ ID NO: 11 [Fragaria (XP_004300916.1)], SEQ ID NO: 12 [Maize (XP_008657477.1)], SEQ ID NO: 13 [Rice (XP_015628733.1)], SEQ ID NO: 14 [Wheat (CDM80098.1)], or SEQ ID NO: 15 [Barley (MLOC_5489.2)]. An additional embodiment of this aspect, which may be combined with any of the preceding embodiments having an ectodomain of an EPR3 or EPR3-like polypeptide, includes the ectodomain of the EPR3 or EPR3-like polypeptide being the ectodomain of SEQ ID NO: 1 [L. japonicus (EPR3)], SEQ ID NO: 2 [Chickpea (XP_004489790.1)], SEQ ID NO: 3 [Medicago (XP_003613165.1)], SEQ ID NO: 4 [Soybean (XP_003517716.1)], SEQ ID NO: 5 [Phaseolus (XP_007157313.1)], SEQ ID NO: 6 [Populus (XP_002322185.1)], SEQ ID NO: 7 [Malus (XP_008340354.1)], SEQ ID NO: 8 [Vitis (XP_002272814.2)], SEQ ID NO: 9 [Theobroma (XP_007036352.1)], SEQ ID NO: 10 [Ricinus (XP_002527912.1)], SEQ ID NO: 11 [Fragaria (XP_004300916.1)], SEQ ID NO: 12 [Maize (XP_008657477.1)], SEQ ID NO: 13 [Rice (XP_015628733.1)], SEQ ID NO: 14 [Wheat (CDM80098.1)], or SEQ ID NO: 15 [Barley (MLOC_5489.2)]. Still another embodiment of this aspect includes the beneficial commensal microbe being commensal bacteria, optionally a nitrogen-fixing bacteria, or a mycorrhizal fungi.

### Molecular Biological Methods to Produce Genetically Altered Plants and Plant Cells

One embodiment of the present invention provides a genetically altered plant or plant cell containing a first nucleic acid sequence encoding a heterologous EPR3 or EPR3-like polypeptide or a modified EPR3 or EPR3-like polypeptide, and optionally containing a second nucleic acid sequence encoding a heterologous EPR3a or EPR3a-like polypeptide or a modified EPR3a or EPR3a-like polypeptide, for increased selectivity for a beneficial commensal microbe as compared to a wild-type plant under the same conditions. Another embodiment of the present invention provides a genetically altered plant or plant cell containing a first nucleic acid sequence encoding a heterologous EPR3a or EPR3a-like polypeptide or a modified EPR3a or EPR3a-like polypeptide, and optionally containing a second nucleic acid sequence encoding a heterologous EPR3 or EPR3-like polypeptide or a modified EPR3 or EPR3-like polypeptide, for increased selectivity for a beneficial commensal microbe as compared to a wild-type plant under the same conditions. Selectivity may mean positive selection of the beneficial commensal microbe, negative selection of other microbes that are not the beneficial commensal, or a combination thereof.

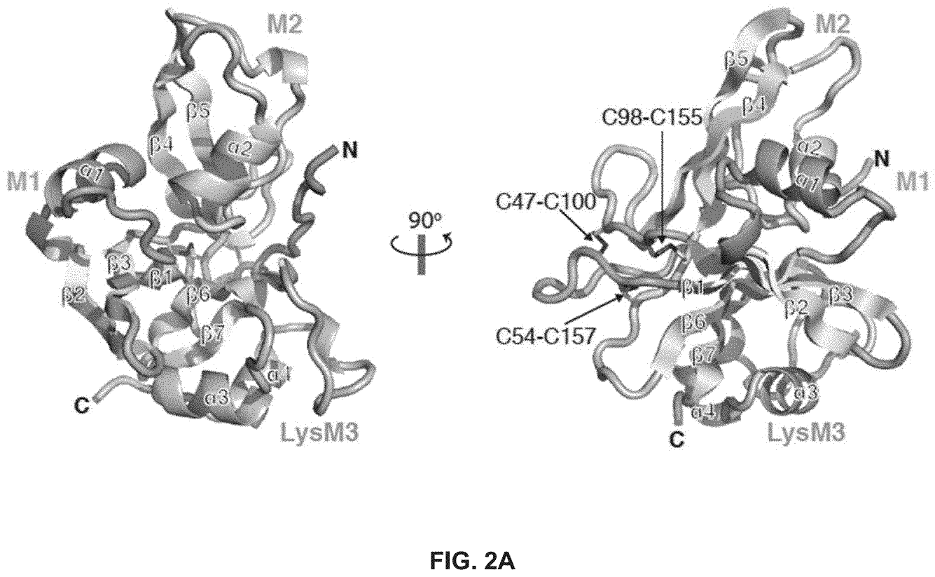

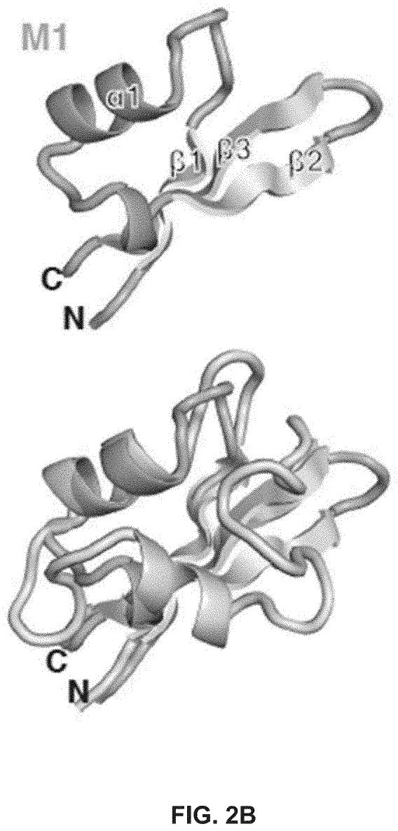

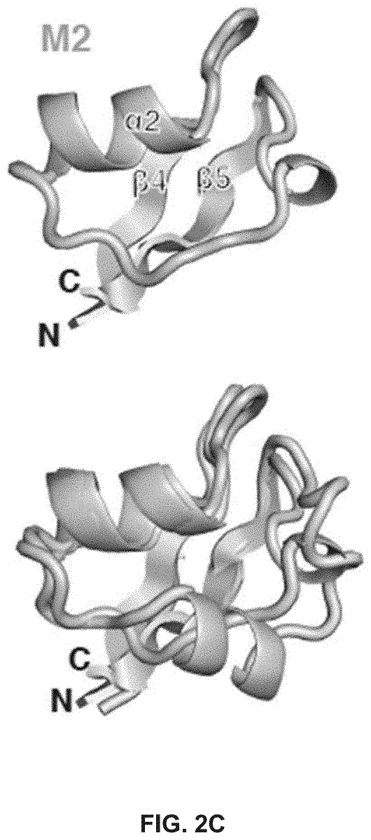

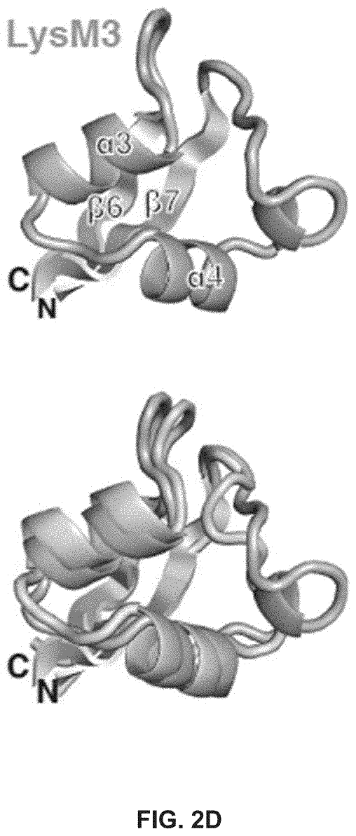

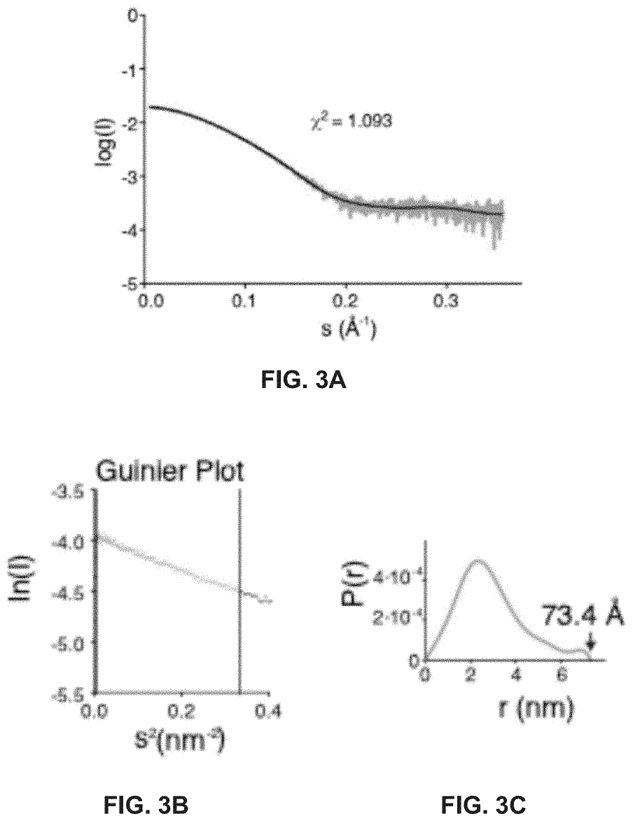

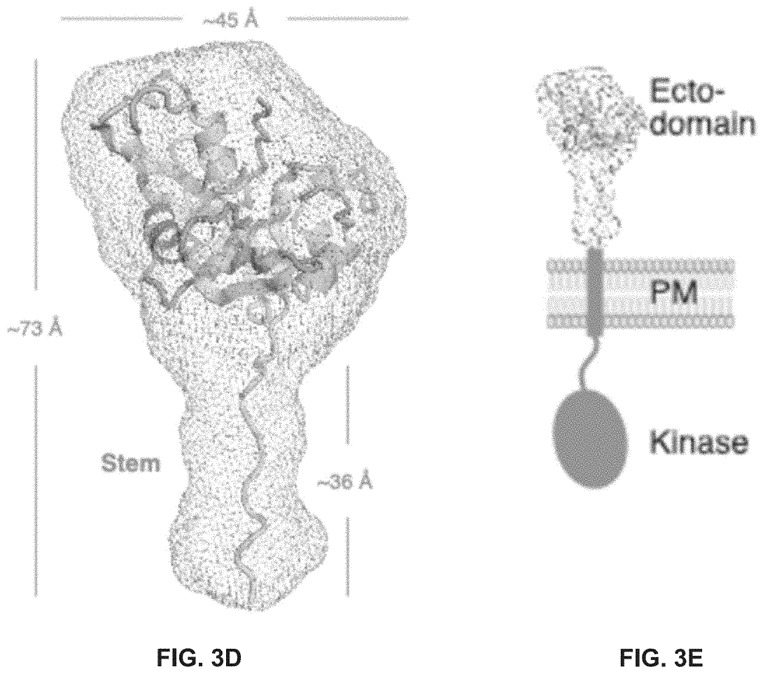

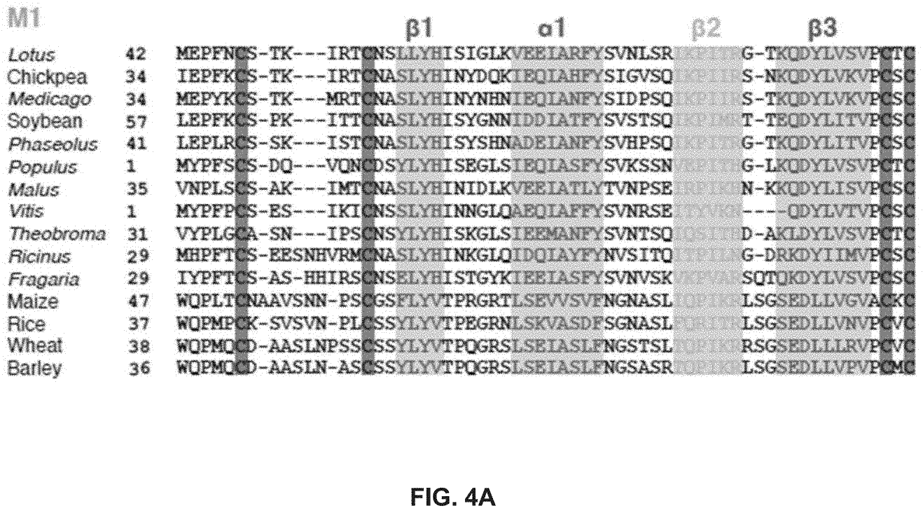

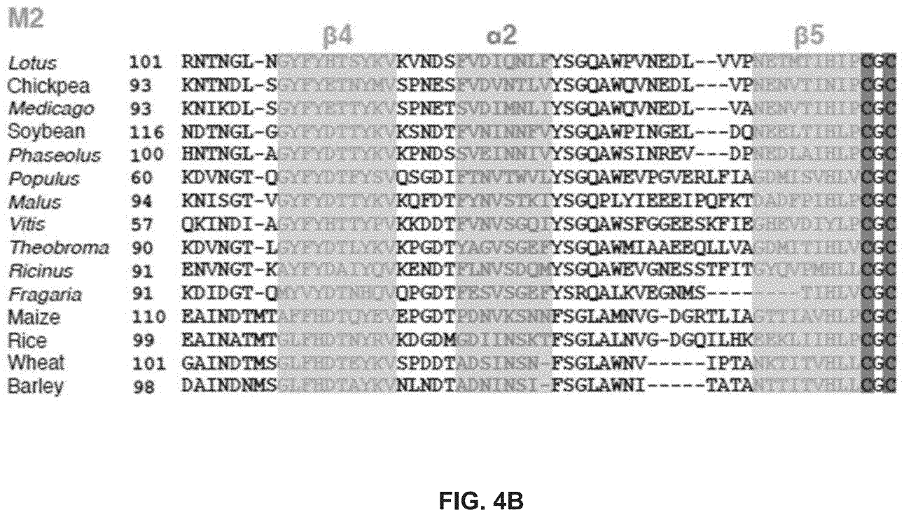

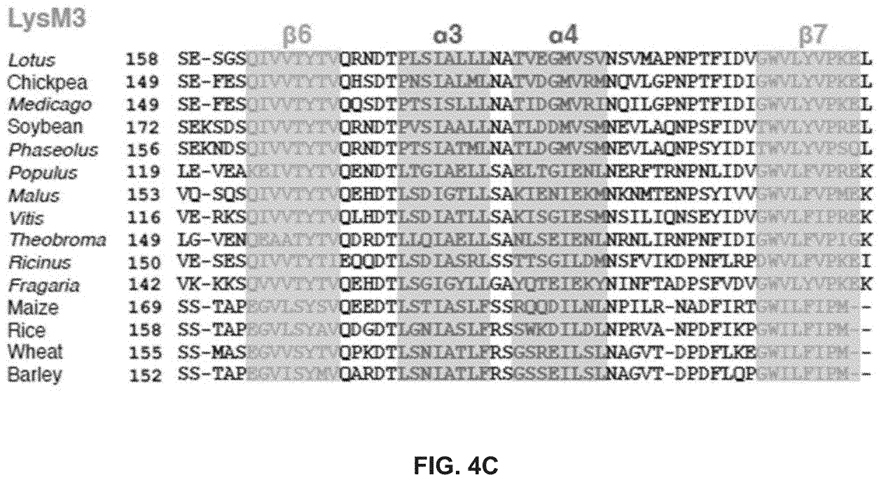

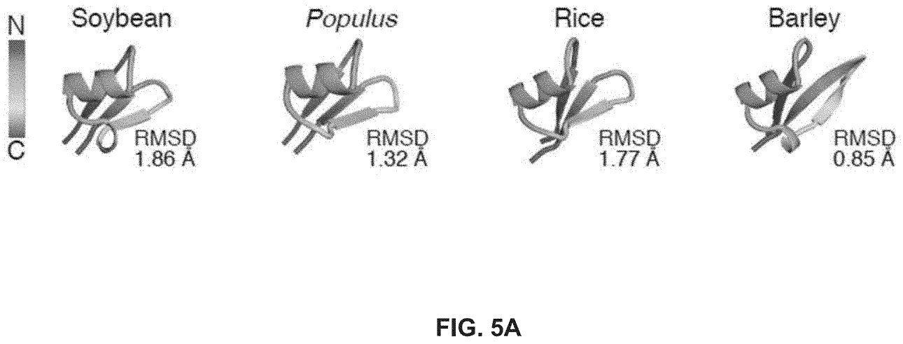

Certain aspects of the present invention relate to the L. japonicus protein EPR3 (SEQ ID NO: 61). EPR3 is a single-pass transmembrane receptor kinase that has an ectodomain with a globular portion and a stalk portion (FIG. 3D), a transmembrane domain, and a kinase domain (FIG. 3E). The EPR3 ectodomain has three domains, M1, M2, and LysM3, each of which contain specific α-helix and β-sheet secondary structures (FIG. 2A). Further aspects of the present disclosure relate to homologs or orthologs of EPR3 (e.g., EPR3-like proteins). In some embodiments, a homolog or ortholog is structurally similar to L. japonicus EPR3. As shown in FIGS. 4A-4C, other plant species have proteins homologous to L. japonicus EPR3 with the same M1, M2 and LysM3 regions containing specific α-helix and β-sheet secondary structures.

A heterologous EPR3 or EPR3-like polypeptide of the present disclosure includes an EPR3 or EPR3-like polypeptide from a dicot (legume or non-legume) plant species or a monocot plant species. An additional embodiment of this aspect includes the heterologous EPR3 or EPR3-like polypeptide being selected from the group of a first polypeptide with at least 70% sequence identity, at least 71% sequence identity, at least 72% sequence identity, at least 73% sequence identity, at least 74% sequence identity, at least 75% sequence identity, at least 76% sequence identity, at least 77% sequence identity, at least 78% sequence identity, at least 79% sequence identity, at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 1 [L. japonicus (BAI79269.1)], a second polypeptide with at least 70% sequence identity, at least 71% sequence identity, at least 72% sequence identity, at least 73% sequence identity, at least 74% sequence identity, at least 75% sequence identity, at least 76% sequence identity, at least 77% sequence identity, at least 78% sequence identity, at least 79% sequence identity, at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 2 [Chickpea (XP_004489790.1)], a third polypeptide with at least 70% sequence identity, at least 71% sequence identity, at least 72% sequence identity, at least 73% sequence identity, at least 74% sequence identity, at least 75% sequence identity, at least 76% sequence identity, at least 77% sequence identity, at least 78% sequence identity, at least 79% sequence identity, at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 3 [Medicago (XP_003613165.1)], a fourth polypeptide with at least 70% sequence identity, at least 71% sequence identity, at least 72% sequence identity, at least 73% sequence identity, at least 74% sequence identity, at least 75% sequence identity, at least 76% sequence identity, at least 77% sequence identity, at least 78% sequence identity, at least 79% sequence identity, at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 4 [Soybean (XP_003517716.1)], a fifth polypeptide with at least 70% sequence identity, at least 71% sequence identity, at least 72% sequence identity, at least 73% sequence identity, at least 74% sequence identity, at least 75% sequence identity, at least 76% sequence identity, at least 77% sequence identity, at least 78% sequence identity, at least 79% sequence identity, at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 5 [Phaseolus (XP_007157313.1)], a sixth polypeptide with at least 70% sequence identity, at least 71% sequence identity, at least 72% sequence identity, at least 73% sequence identity, at least 74% sequence identity, at least 75% sequence identity, at least 76% sequence identity, at least 77% sequence identity, at least 78% sequence identity, at least 79% sequence identity, at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 6 [Populus (XP_002322185.1)], a seventh polypeptide with at least 70% sequence identity, at least 71% sequence identity, at least 72% sequence identity, at least 73% sequence identity, at least 74% sequence identity, at least 75% sequence identity, at least 76% sequence identity, at least 77% sequence identity, at least 78% sequence identity, at least 79% sequence identity, at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 7 [Malta (XP_008340354.1)], an eighth polypeptide with at least 70% sequence identity, at least 71% sequence identity, at least 72% sequence identity, at least 73% sequence identity, at least 74% sequence identity, at least 75% sequence identity, at least 76% sequence identity, at least 77% sequence identity, at least 78% sequence identity, at least 79% sequence identity, at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 8 [Vitis (XP_002272814.2)], a ninth polypeptide with at least 70% sequence identity, at least 71% sequence identity, at least 72% sequence identity, at least 73% sequence identity, at least 74% sequence identity, at least 75% sequence identity, at least 76% sequence identity, at least 77% sequence identity, at least 78% sequence identity, at least 79% sequence identity, at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 9 [Theobroma (XP_007036352.1)], a tenth polypeptide with at least 70% sequence identity, at least 71% sequence identity, at least 72% sequence identity, at least 73% sequence identity, at least 74% sequence identity, at least 75% sequence identity, at least 76% sequence identity, at least 77% sequence identity, at least 78% sequence identity, at least 79% sequence identity, at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 10 [Ricinus (XP_002527912.1)], an eleventh polypeptide with at least 70% sequence identity, at least 71% sequence identity, at least 72% sequence identity, at least 73% sequence identity, at least 74% sequence identity, at least 75% sequence identity, at least 76% sequence identity, at least 77% sequence identity, at least 78% sequence identity, at least 79% sequence identity, at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 11 [Fragaria (XP_004300916.1)], a twelfth polypeptide with at least 70% sequence identity, at least 71% sequence identity, at least 72% sequence identity, at least 73% sequence identity, at least 74% sequence identity, at least 75% sequence identity, at least 76% sequence identity, at least 77% sequence identity, at least 78% sequence identity, at least 79% sequence identity, at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 12 [Maize (XP_008657477.1)], a thirteenth polypeptide with at least 70% sequence identity, at least 71% sequence identity, at least 72% sequence identity, at least 73% sequence identity, at least 74% sequence identity, at least 75% sequence identity, at least 76% sequence identity, at least 77% sequence identity, at least 78% sequence identity, at least 79% sequence identity, at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 13 [Rice (XP_015628733.1)], a fourteenth polypeptide with at least 70% sequence identity, at least 71% sequence identity, at least 72% sequence identity, at least 73% sequence identity, at least 74% sequence identity, at least 75% sequence identity, at least 76% sequence identity, at least 77% sequence identity, at least 78% sequence identity, at least 79% sequence identity, at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 14 [Wheat (CDM80098.1)], or a fifteenth polypeptide with at least 70% sequence identity, at least 71% sequence identity, at least 72% sequence identity, at least 73% sequence identity, at least 74% sequence identity, at least 75% sequence identity, at least 76% sequence identity, at least 77% sequence identity, at least 78% sequence identity, at least 79% sequence identity, at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 15 [Barley (MLOC_5489.2)]. A further embodiment of this aspect includes the heterologous EPR3 or EPR3-like polypeptide being selected from the group of SEQ ID NO: 1 [L. japonicus (EPR3)], SEQ ID NO: 2 [Chickpea (XP_004489790.1)], SEQ ID NO: 3 [Medicago (XP_003613165.1)], SEQ ID NO: 4 [Soybean (XP_003517716.1)], SEQ ID NO: 5 [Phaseolus (XP_007157313.1)], SEQ ID NO: 6 [Populus (XP_002322185.1)], SEQ ID NO: 7 [Malus (XP_008340354.1)], SEQ ID NO: 8 [Vitis (XP_002272814.2)], SEQ ID NO: 9 [Theobroma (XP_007036352.1)], SEQ ID NO: 10 [Ricinus (XP_002527912.1)], SEQ ID NO: 11 [Fragaria (XP_004300916.1)], SEQ ID NO: 12 [Maize (XP_008657477.1)], SEQ ID NO: 13 [Rice (XP_015628733.1)], SEQ ID NO: 14 [Wheat (CDM80098.1)], or SEQ ID NO: 15 [Barley (MLOC_5489.2)]. An additional embodiment of this aspect includes the heterologous EPR3 or EPR3-like polypeptide having at least 70% sequence identity, at least 71% sequence identity, at least 72% sequence identity, at least 73% sequence identity, at least 74% sequence identity, at least 75% sequence identity, at least 76% sequence identity, at least 77% sequence identity, at least 78% sequence identity, at least 79% sequence identity, at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, or SEQ ID NO: 187. A further embodiment of this aspect includes the heterologous EPR3 or EPR3-like polypeptide being SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, or SEQ ID NO: 187.

A modified EPR3 or EPR3-like polypeptide of the present disclosure includes an EPR3 or EPR3-like polypeptide including a modified ectodomain that has been replaced with all or a portion of an ectodomain of the heterologous EPR3 or EPR3-like polypeptide, optionally all or a part of the M1 domain, the M2 domain, the LysM3 domain, or all three. In an additional embodiment of this aspect, the portion replaced is at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, less than 10%, less than 11%, less than 12%, less than 13%, less than 14%, less than 15%, less than 16%, less than 17%, less than 18%, less than 19%, less than 20%, less than 21%, less than 22%, less than 23%, less than 24%, less than 25%, less than 26%, less than 27%, less than 28%, less than 29%, less than 30%, less than 31%, less than 32%, less than 33%, less than 34%, less than 35%, less than 36%, less than 37%, less than 38%, less than 39%, less than 40%, less than 41%, less than 42%, less than 43%, less than 44%, less than 45%, less than 46%, less than 47%, less than 48%, less than 49%, less than 50%, less than 51%, less than 52%, less than 53%, less than 54%, less than 55%, less than 56%, less than 57%, less than 58%, less than 59%, less than 60%, less than 61%, less than 62%, less than 63%, less than 64%, less than 65%, less than 66%, less than 67%, less than 68%, less than 69%, less than 70%, less than 71%, less than 72%, less than 73%, less than 74%, less than 75%, less than 76%, less than 77%, less than 78%, less than 79%, less than 80%, less than 81%, less than 82%, less than 83%, less than 84%, less than 85%, less than 86%, less than 87%, less than 88%, less than 89%, or less than 90%, of the ectodomain or, optionally all or a part of the M1 domain, the M2 domain, the LysM3 domain, or all three. A further embodiment of this aspect includes the EPR3 or EPR3-like polypeptide being an endogenous EPR3 or EPR3-like polypeptide.

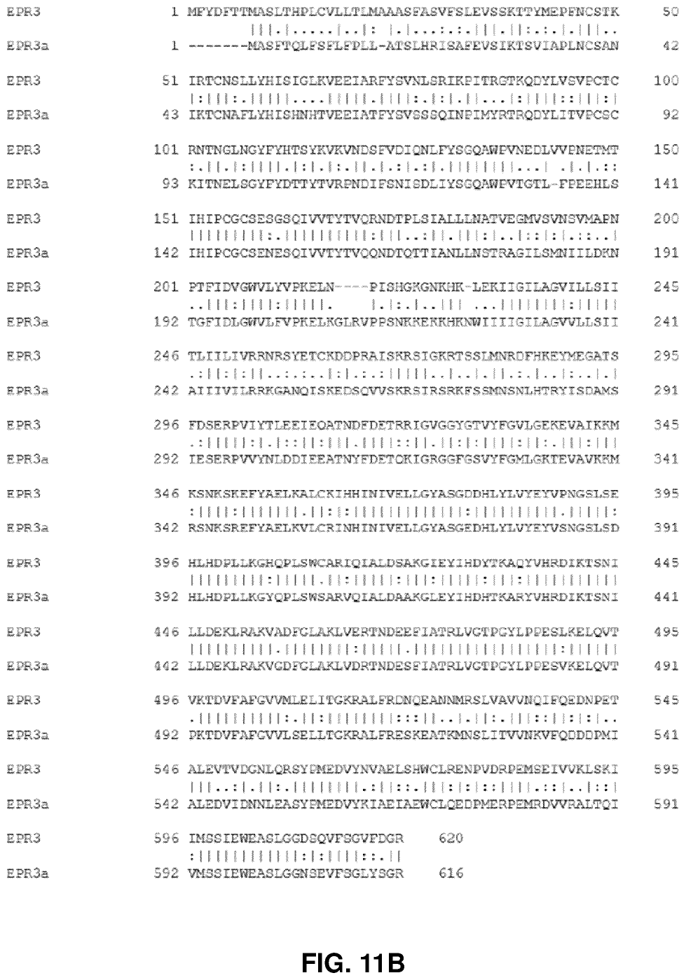

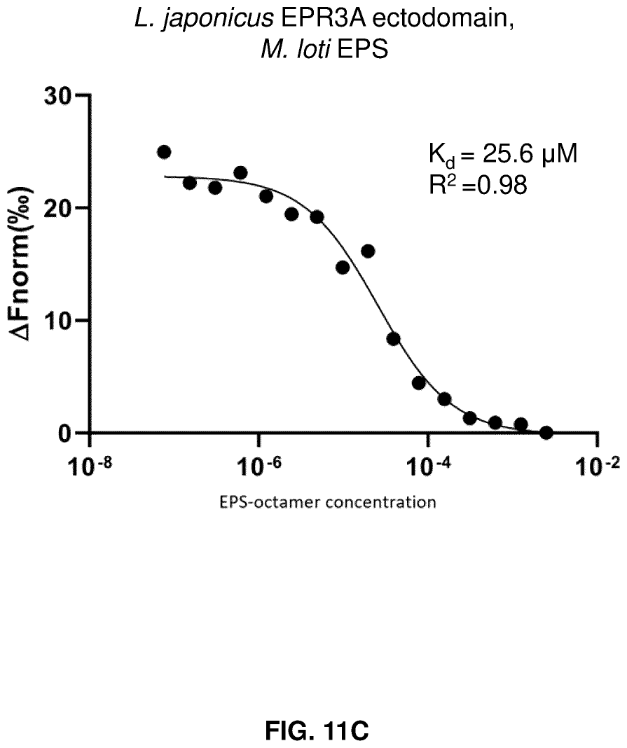

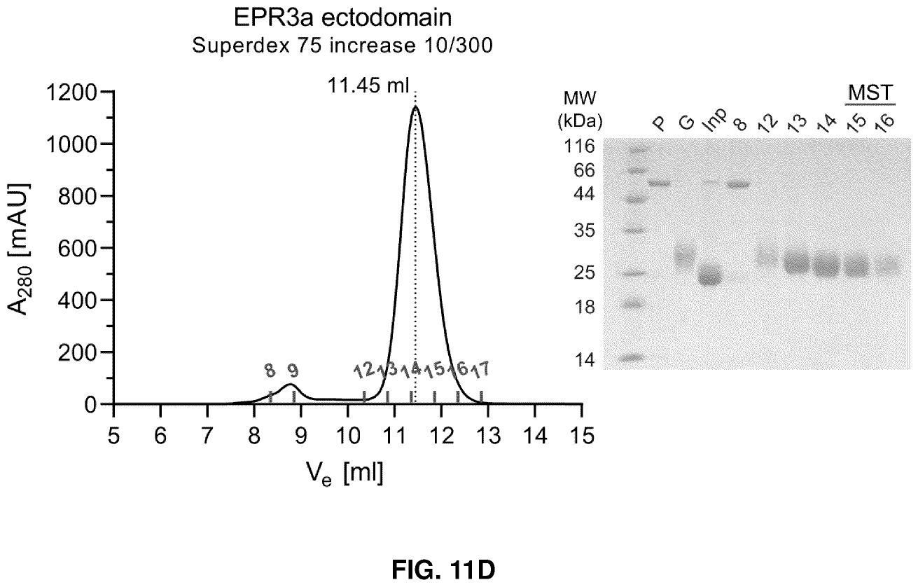

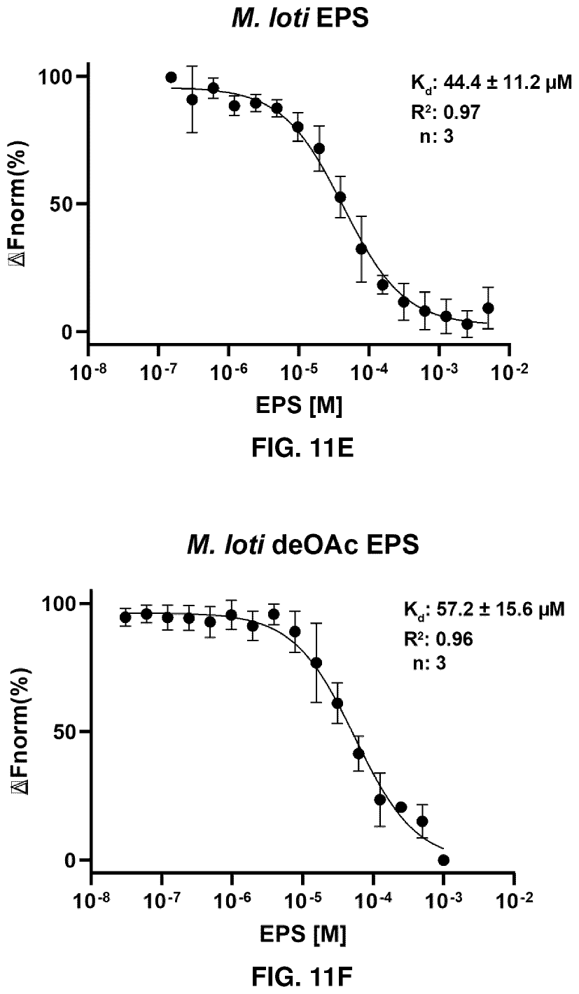

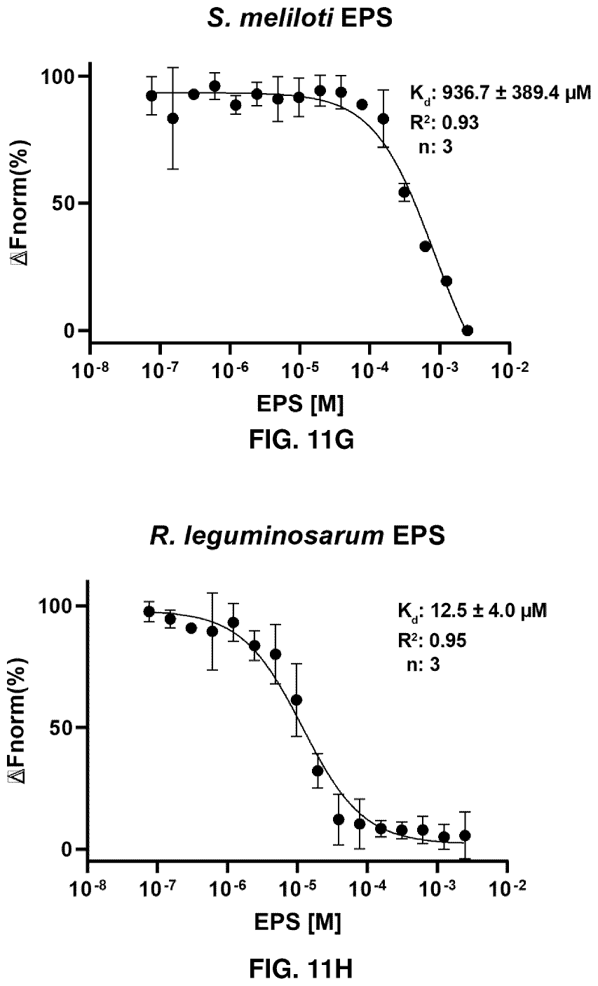

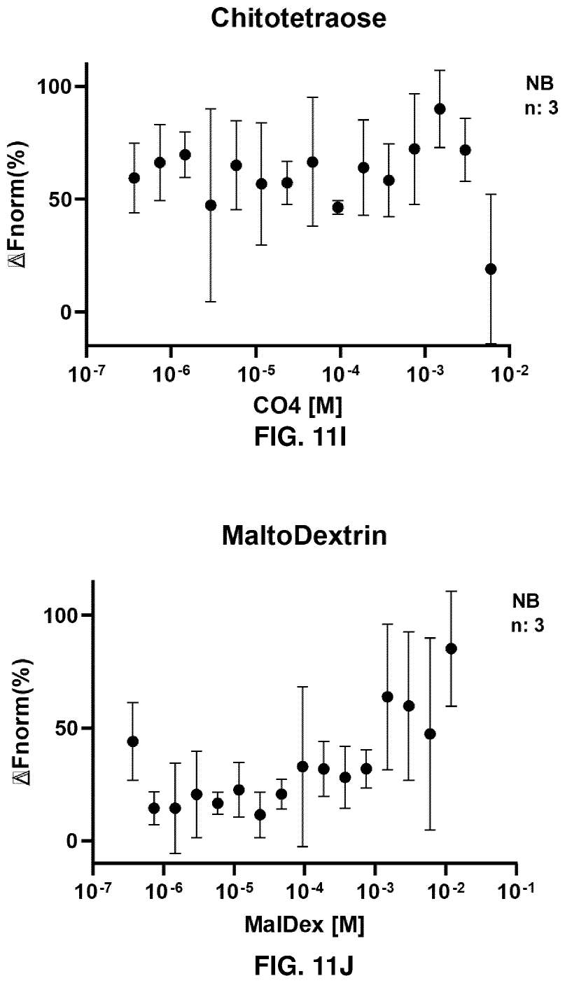

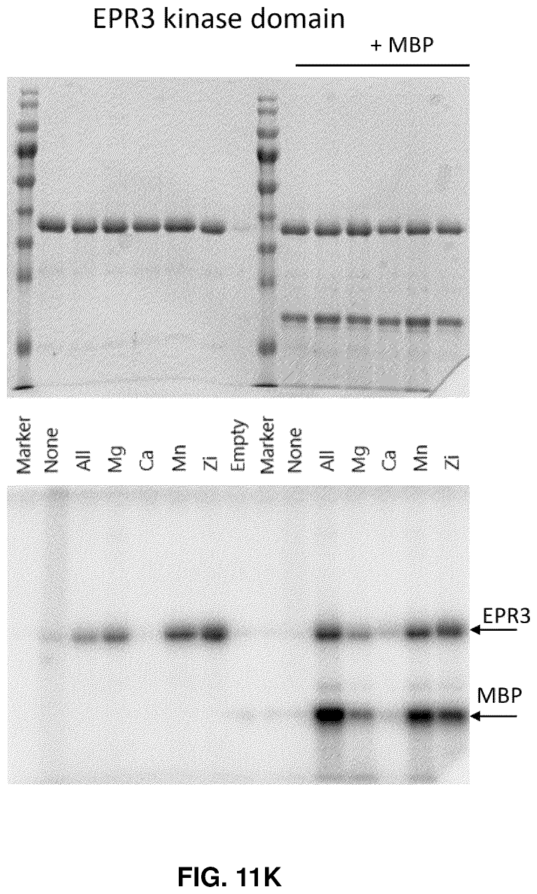

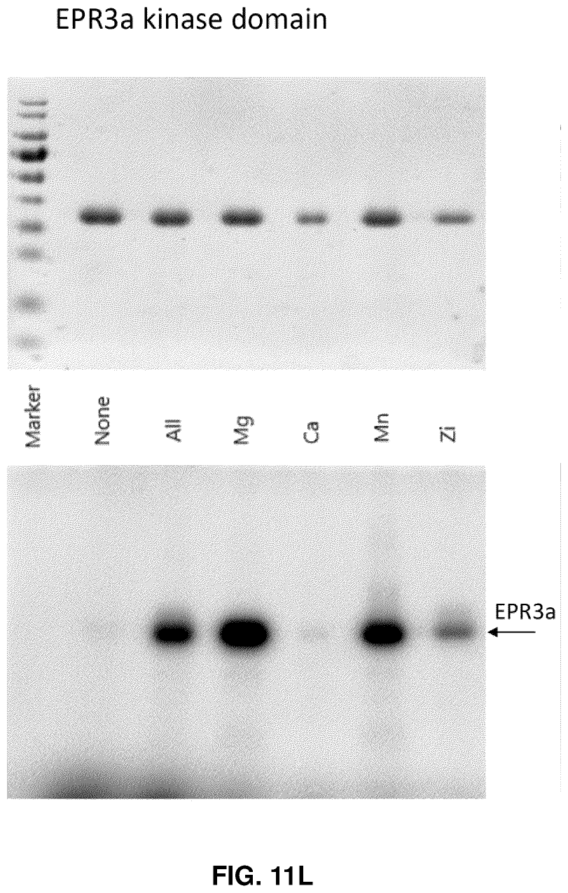

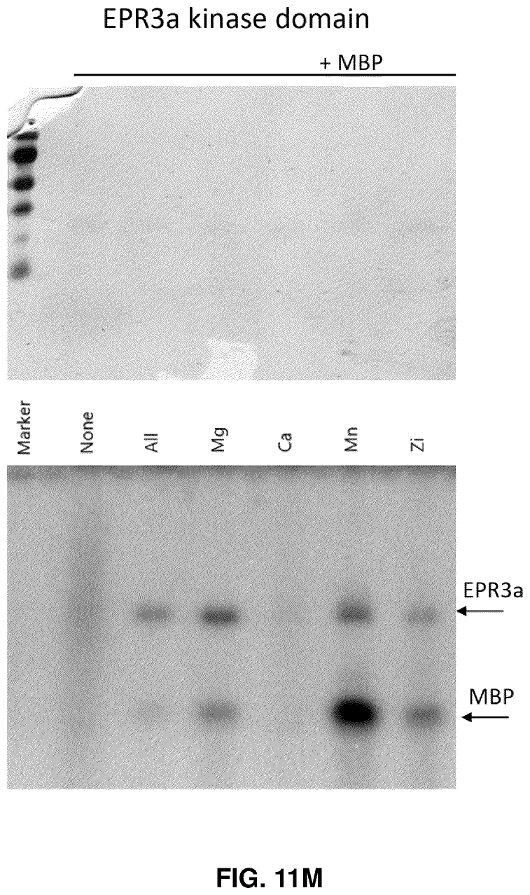

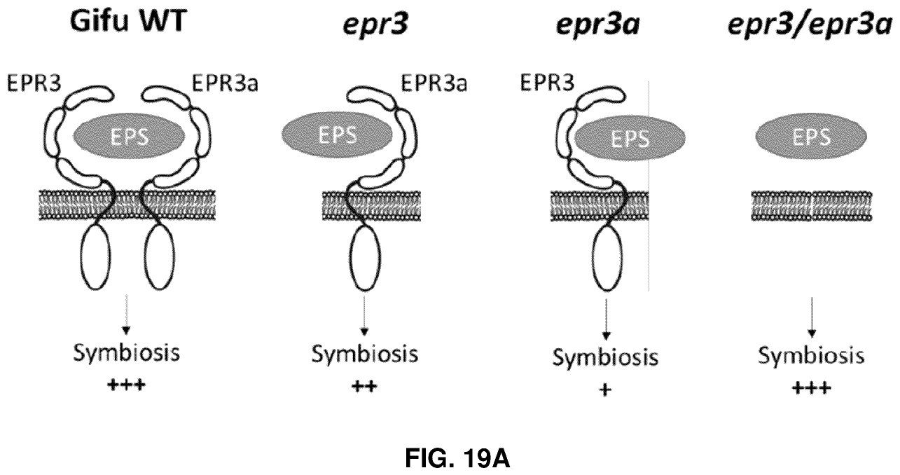

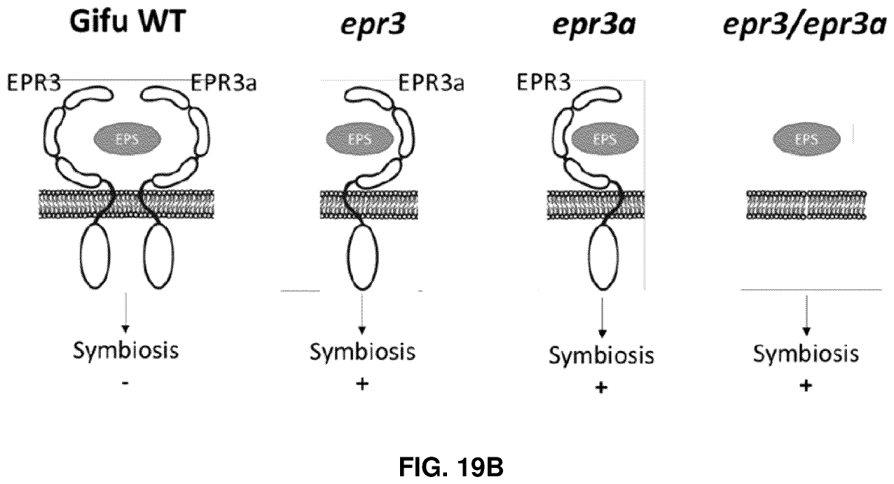

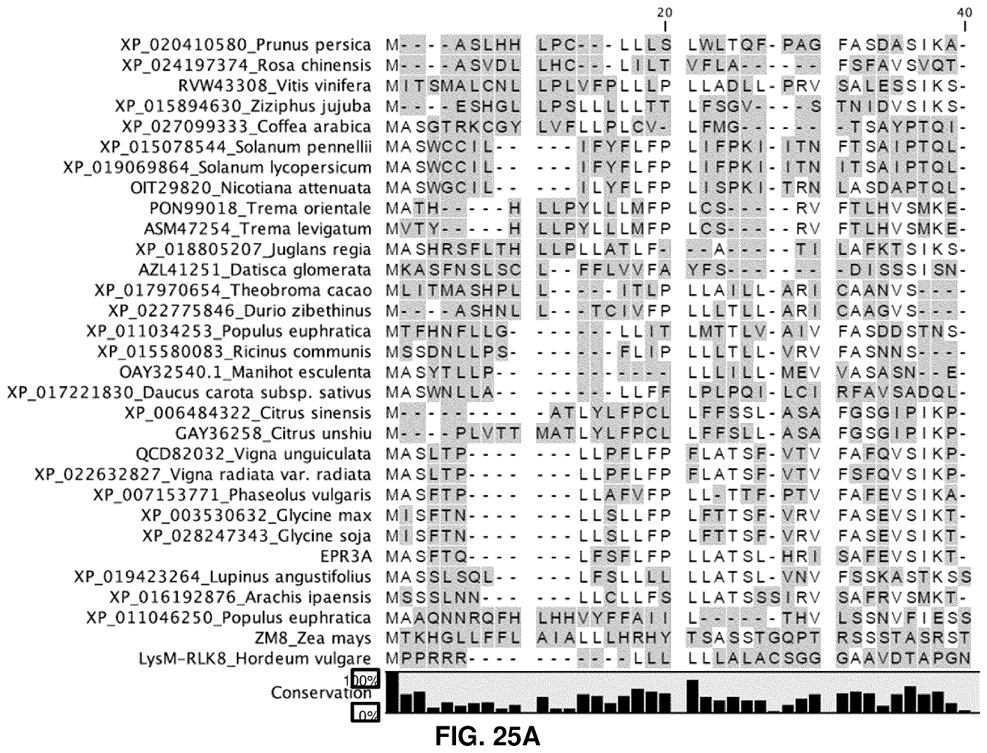

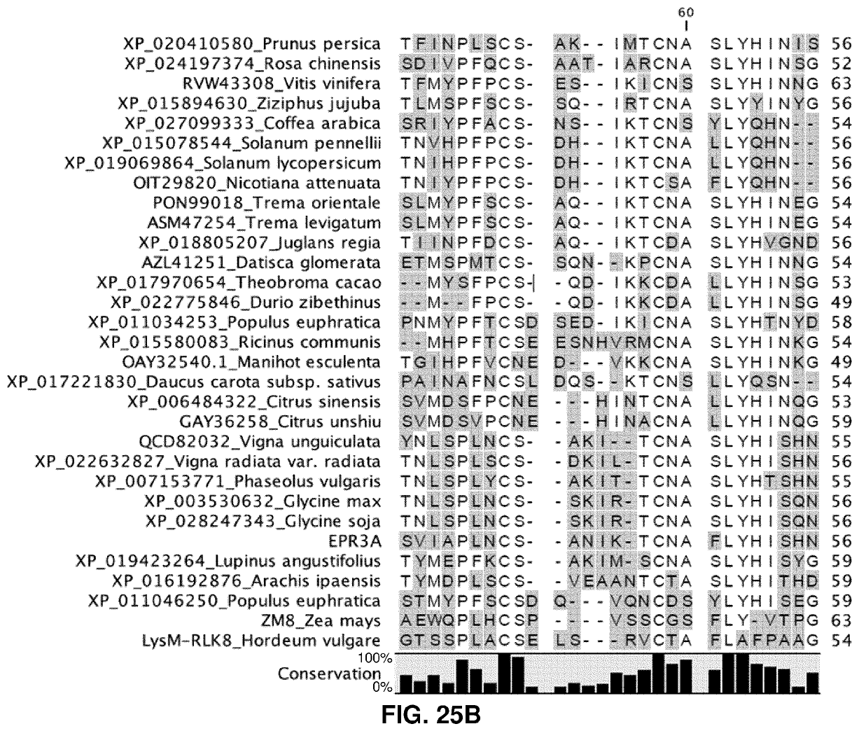

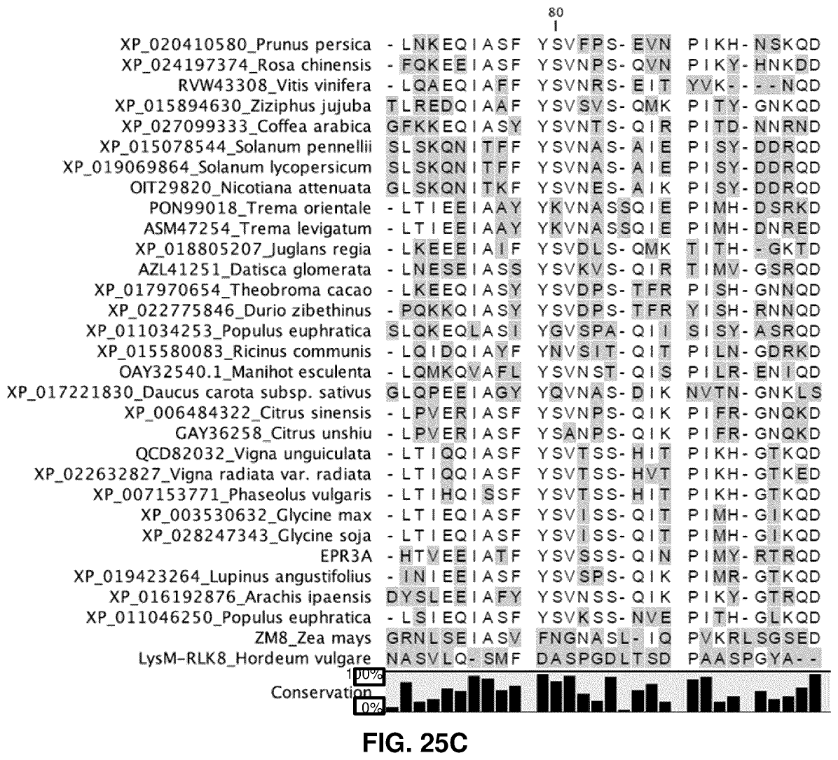

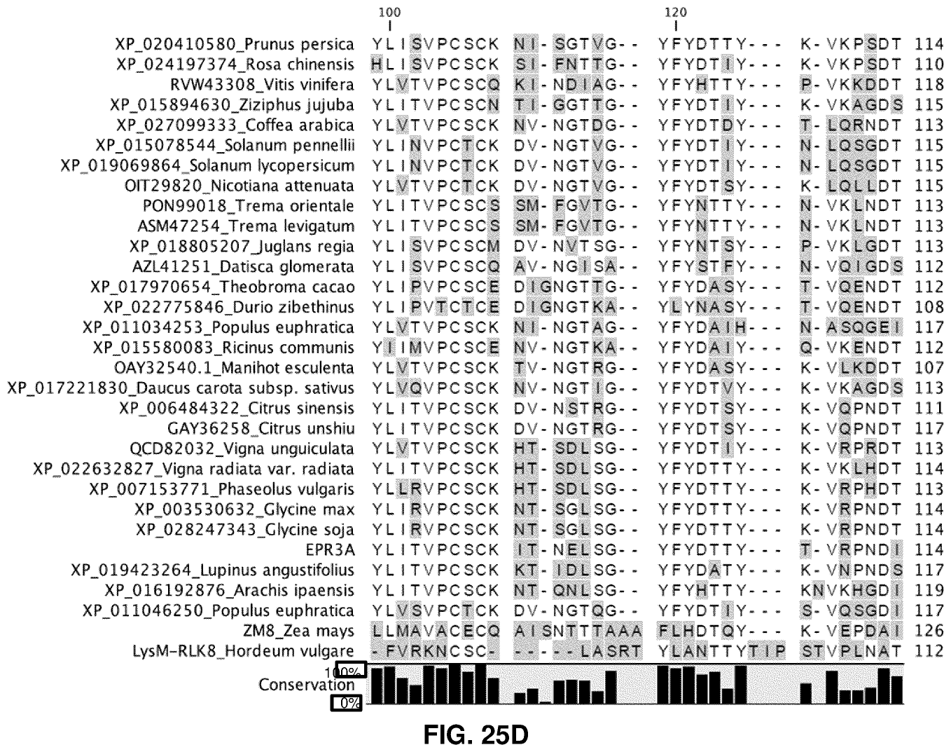

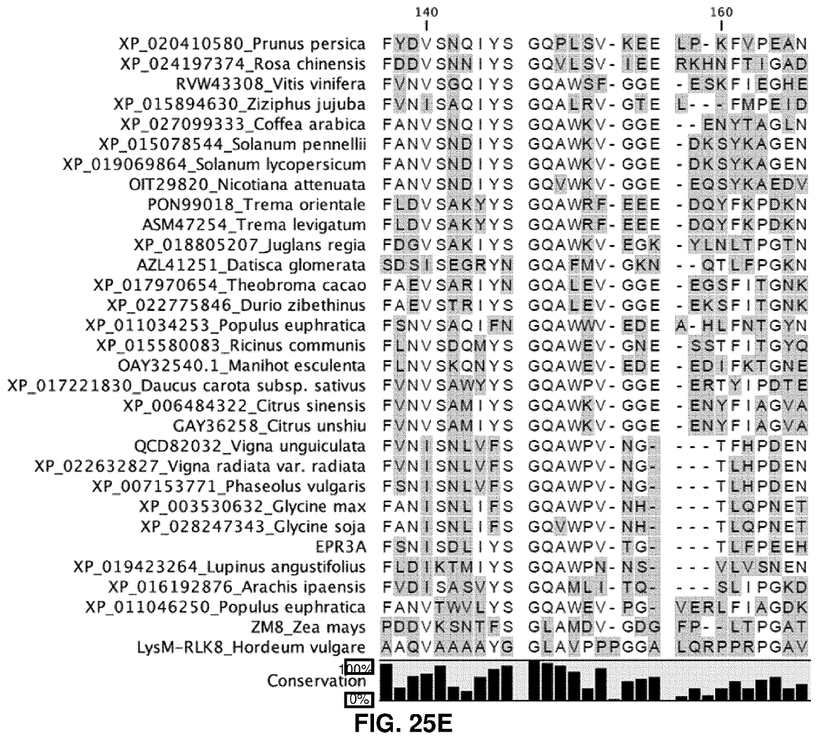

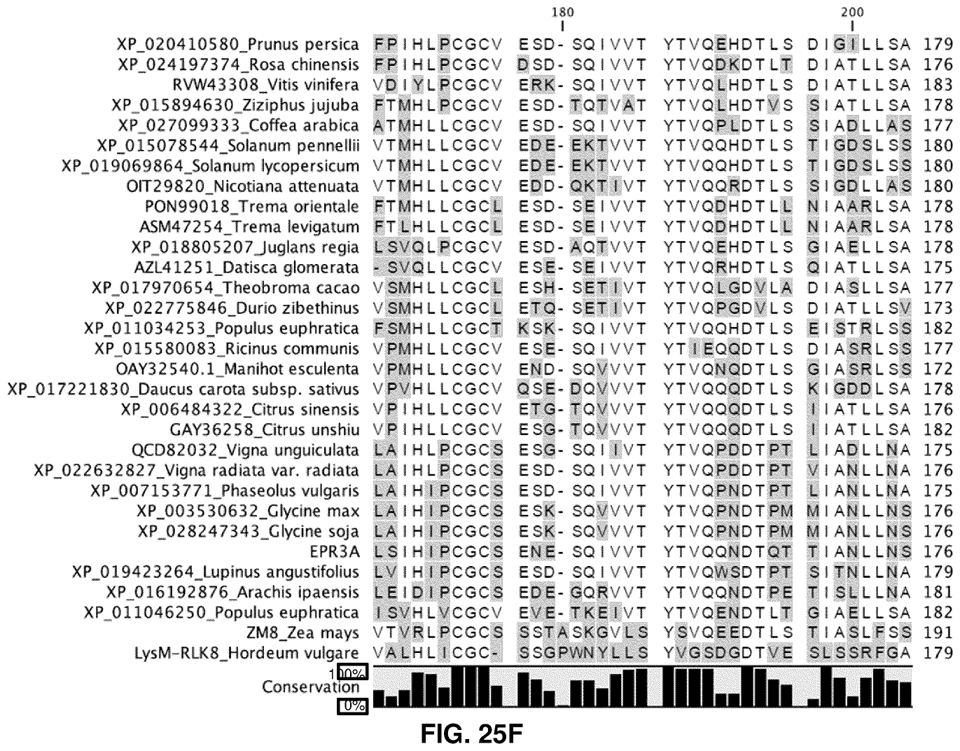

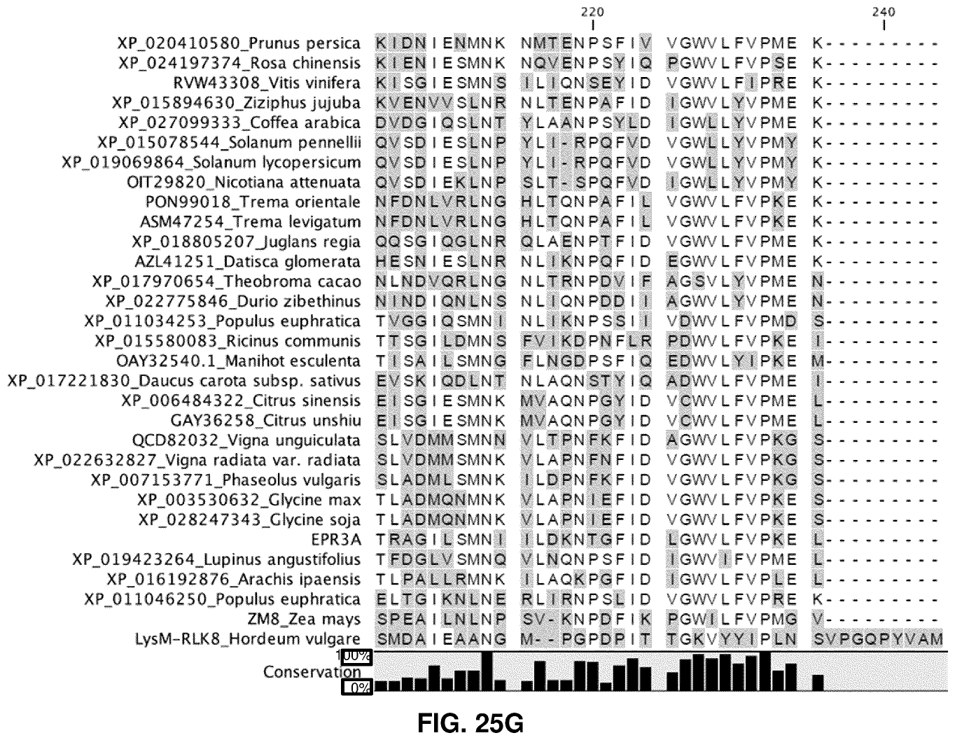

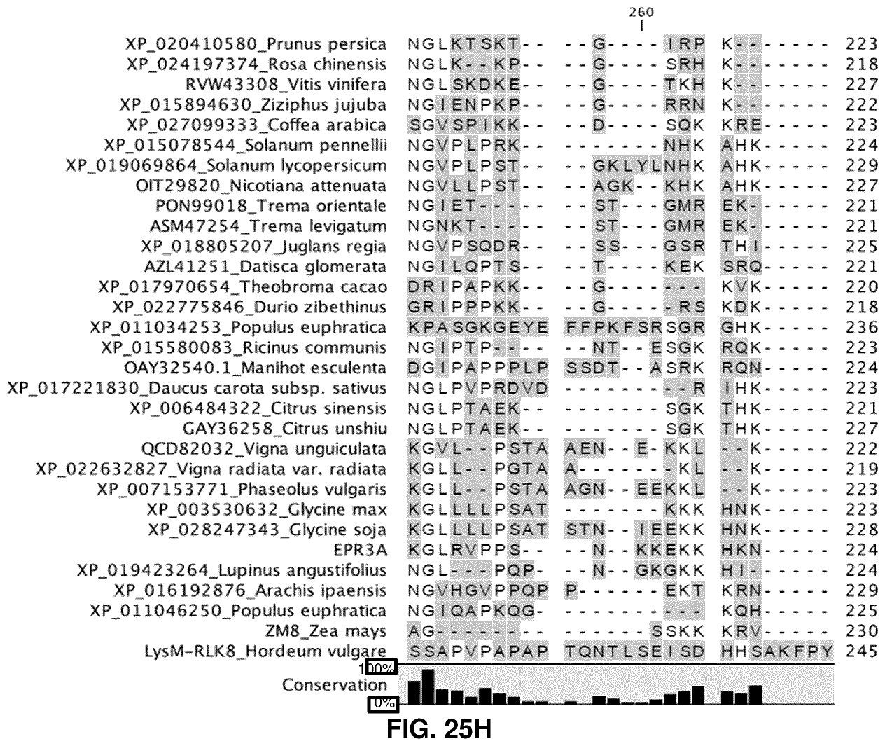

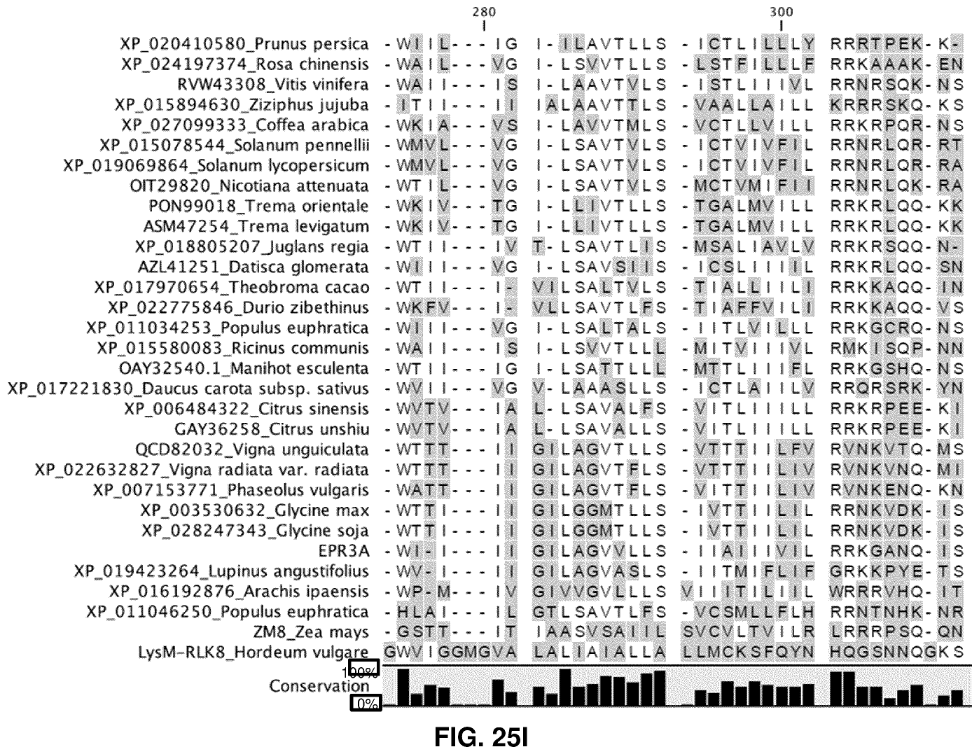

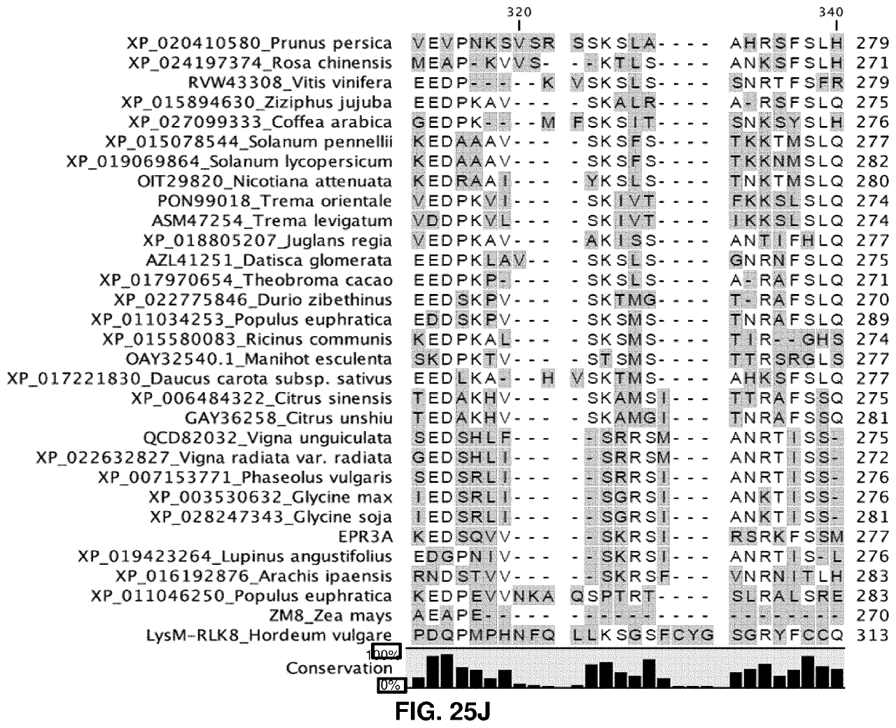

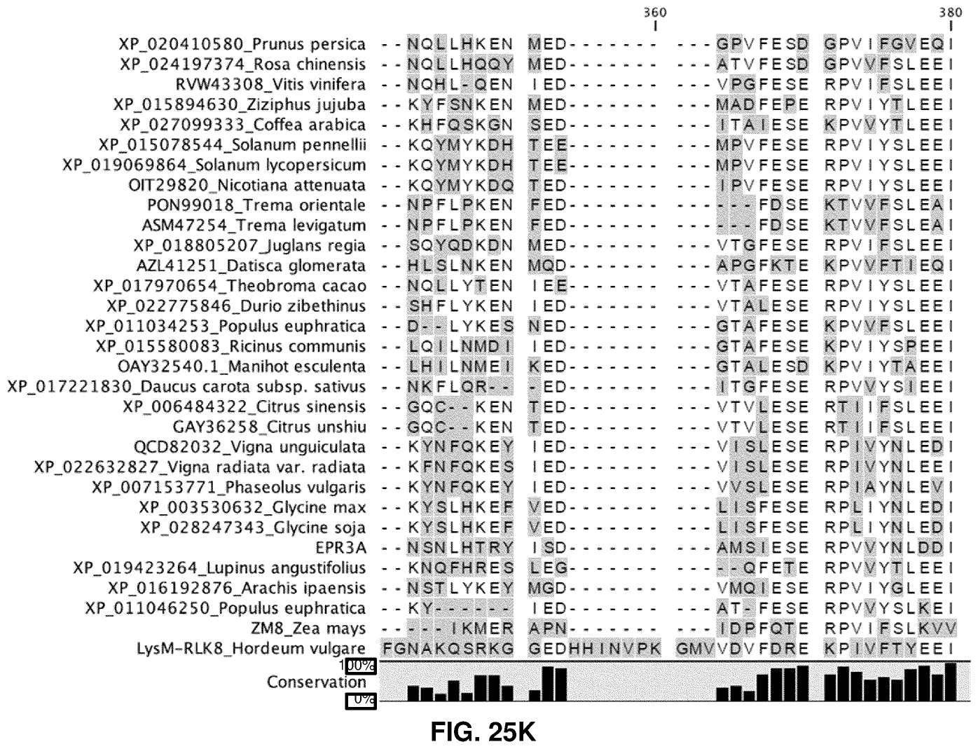

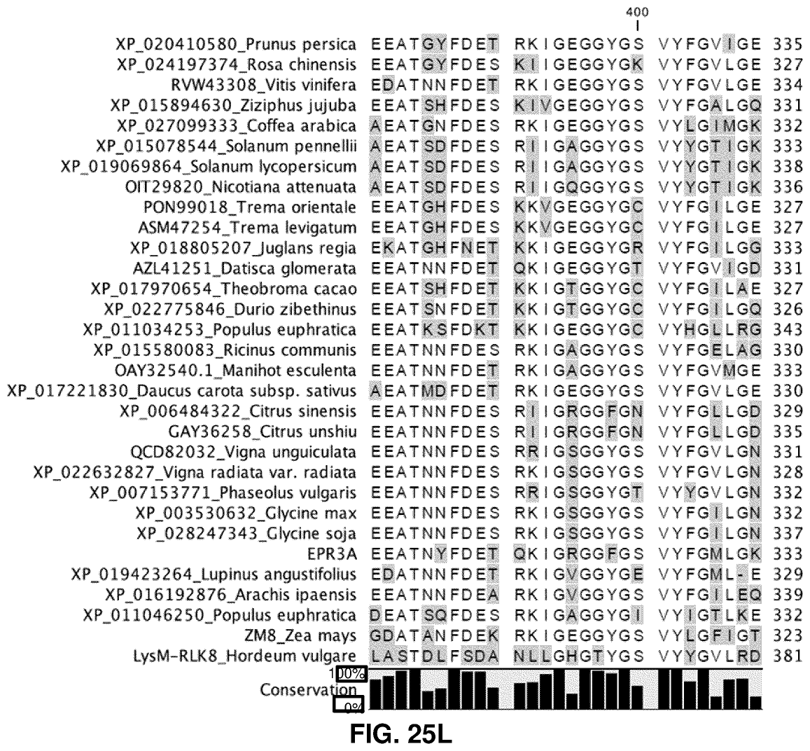

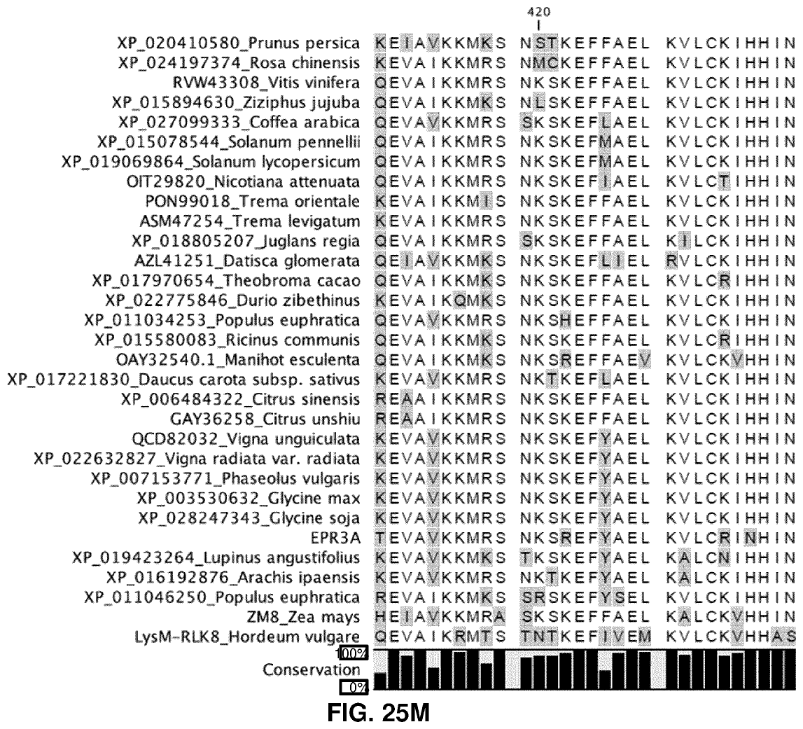

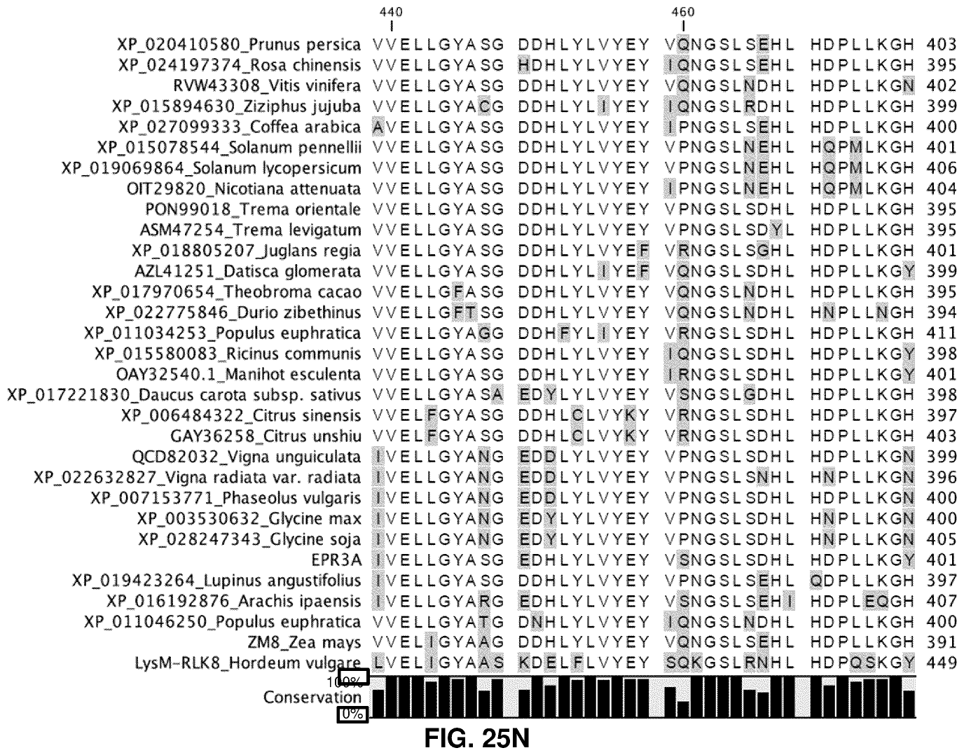

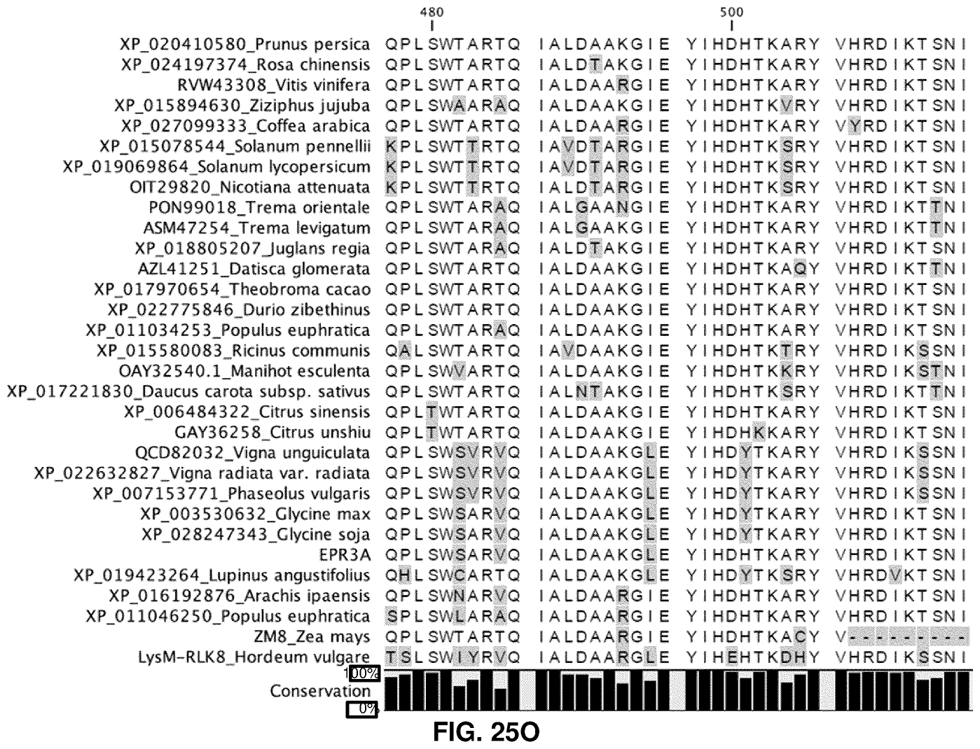

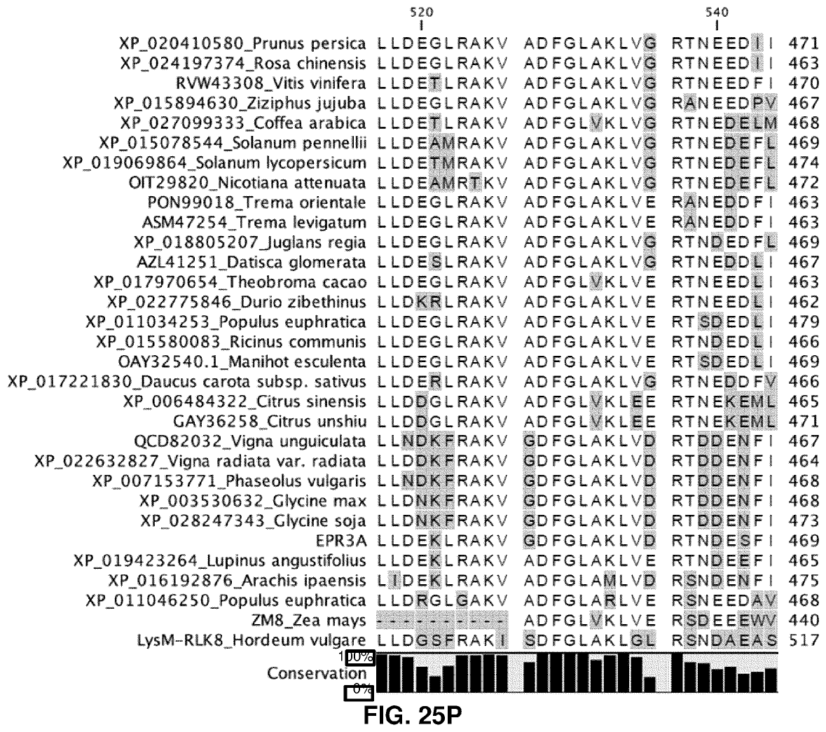

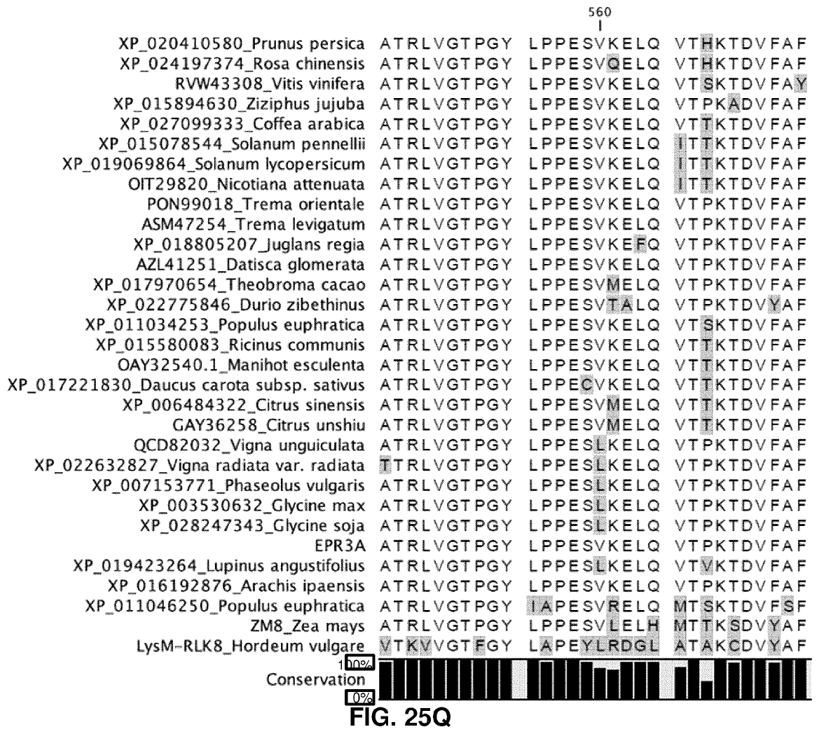

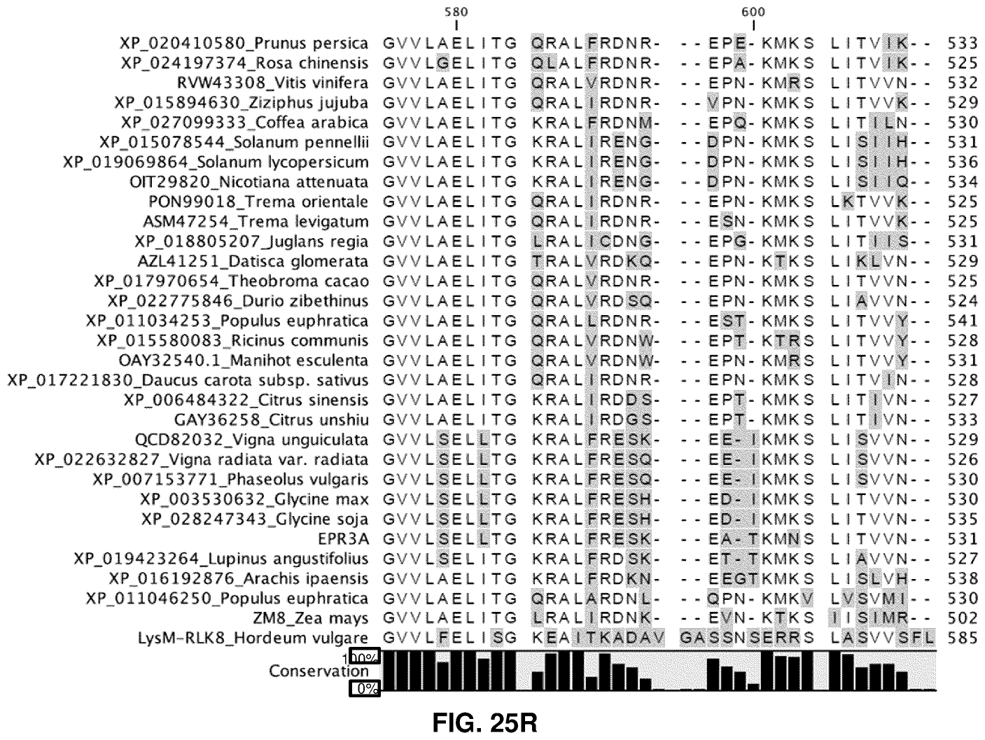

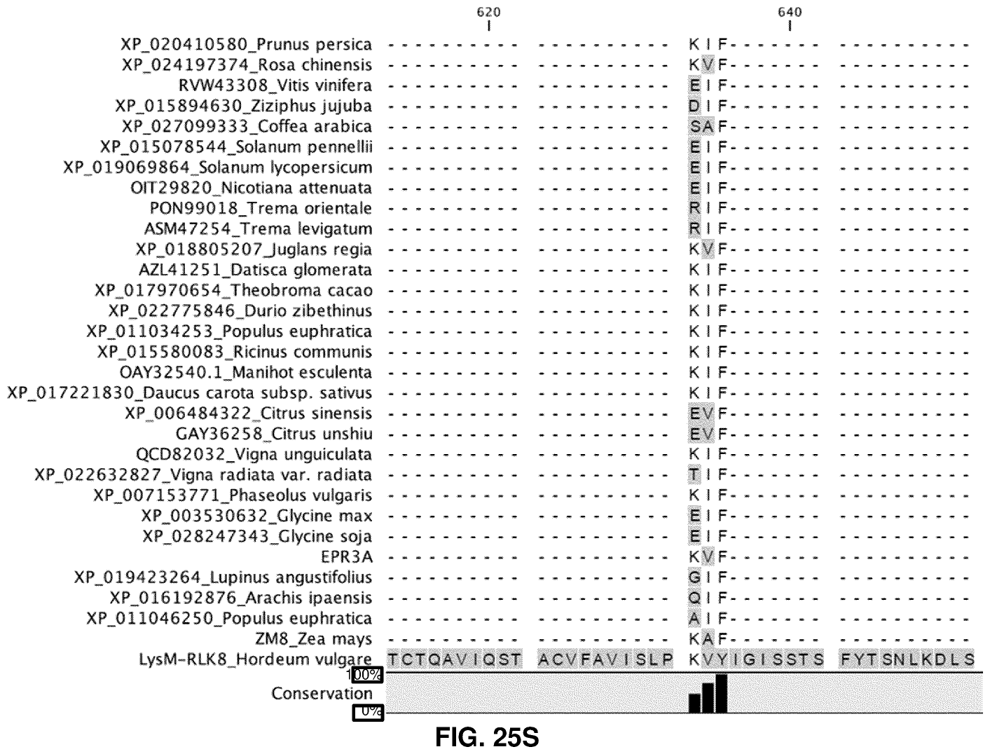

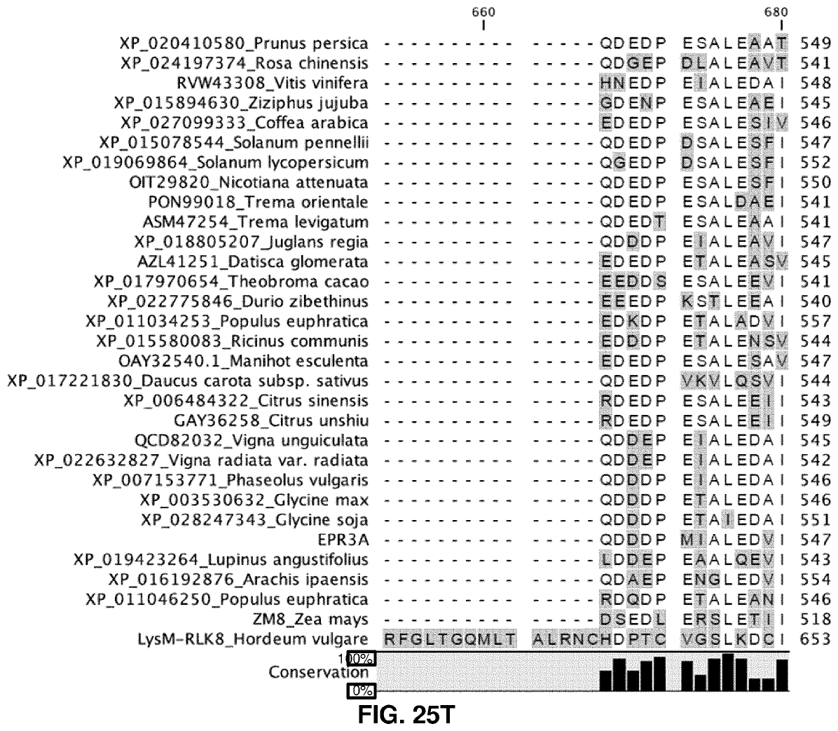

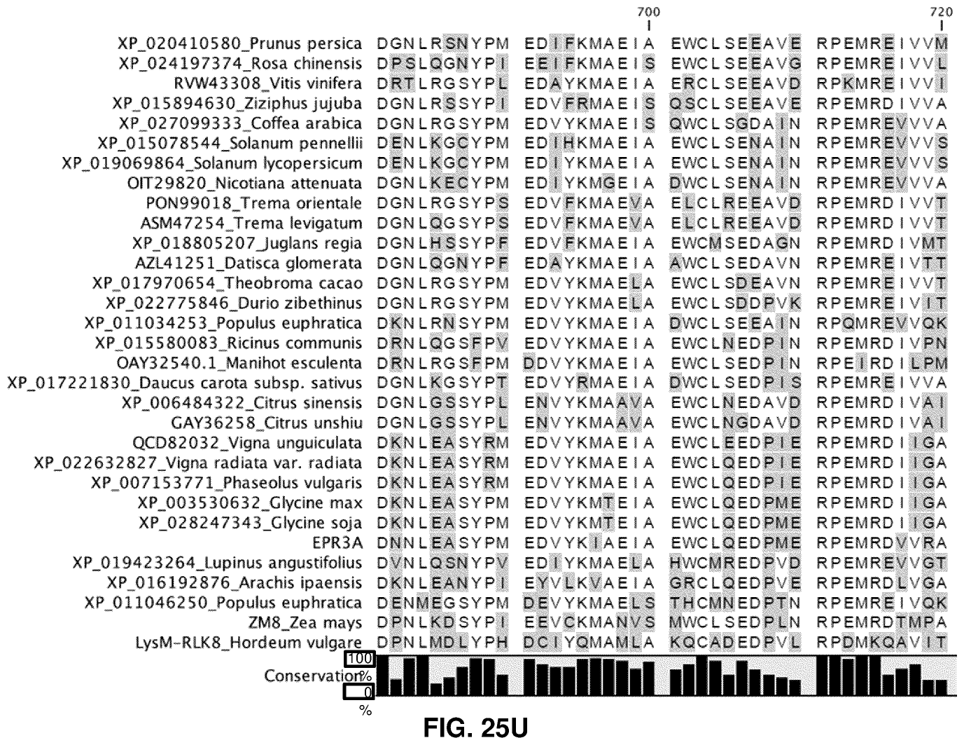

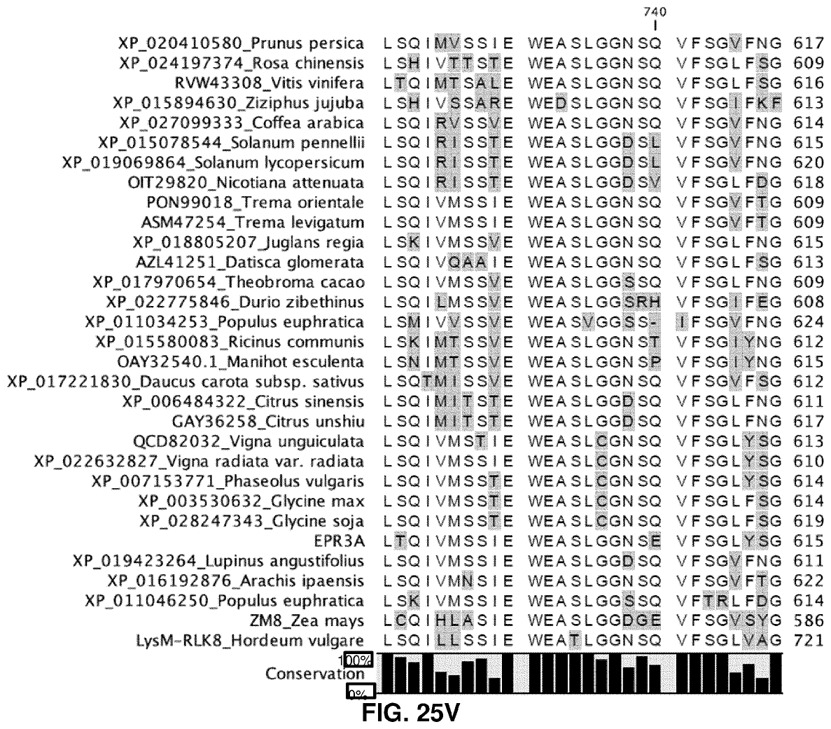

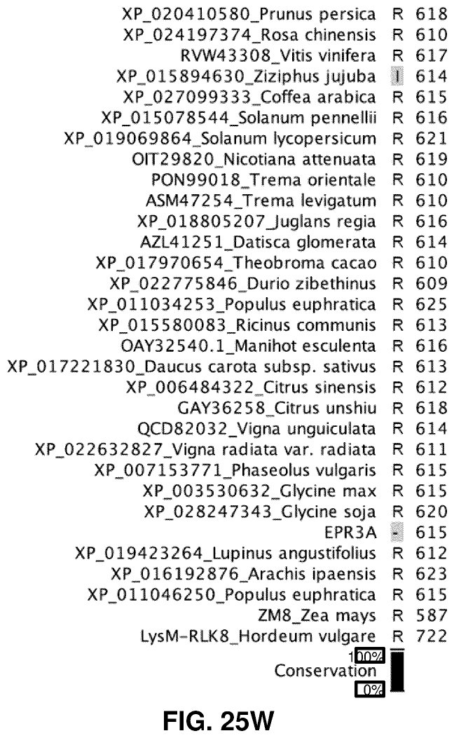

Certain aspects of the present invention relate to the L. japonicus protein EPR3a (SEQ ID NO: 62). EPR3a has 65% amino acid identity to EPR3 (FIG. 11B), and, based upon homology with EPR3, is a single-pass transmembrane receptor kinase that has an ectodomain with a globular portion and a stalk portion, a transmembrane domain, and a kinase domain (FIGS. 19A-19B). Further aspects of the present disclosure relate to homologs or orthologs of EPR3a (e.g., EPR3a-like proteins). In some embodiments, a homolog or ortholog is structurally similar to L. japonicus EPR3a. FIGS. 25A-25W show an alignment of L. japonicus EPR3a with EPR3a-like proteins from other plant species.

A heterologous EPR3a or EPR3a-like polypeptide of the present disclosure includes an EPR3a or EPR3a-like polypeptide from a dicot (legume or non-legume) plant species or a monocot plant species. An additional embodiment of this aspect includes the heterologous EPR3a or EPR3a-like polypeptide being selected from the group of a polypeptide with at least 70% sequence identity, at least 71% sequence identity, at least 72% sequence identity, at least 73% sequence identity, at least 74% sequence identity, at least 75% sequence identity, at least 76% sequence identity, at least 77% sequence identity, at least 78% sequence identity, at least 79% sequence identity, at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, or SEQ ID NO: 92. A further embodiment of this aspect includes the heterologous EPR3a or EPR3a-like polypeptide being selected from the group of SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, or SEQ ID NO: 92.

A modified EPR3a or EPR3a-like polypeptide of the present disclosure includes an EPR3a or EPR3a-like polypeptide including a modified ectodomain that has been replaced with all or a portion of an ectodomain of the heterologous EPR3a or EPR3a-like polypeptide, optionally all or a part of the M1 domain, the M2 domain, the LysM3 domain, or all three. In an additional embodiment of this aspect, the portion replaced is at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, less than 10%, less than 11%, less than 12%, less than 13%, less than 14%, less than 15%, less than 16%, less than 17%, less than 18%, less than 19%, less than 20%, less than 21%, less than 22%, less than 23%, less than 24%, less than 25%, less than 26%, less than 27%, less than 28%, less than 29%, less than 30%, less than 31%, less than 32%, less than 33%, less than 34%, less than 35%, less than 36%, less than 37%, less than 38%, less than 39%, less than 40%, less than 41%, less than 42%, less than 43%, less than 44%, less than 45%, less than 46%, less than 47%, less than 48%, less than 49%, less than 50%, less than 51%, less than 52%, less than 53%, less than 54%, less than 55%, less than 56%, less than 57%, less than 58%, less than 59%, less than 60%, less than 61%, less than 62%, less than 63%, less than 64%, less than 65%, less than 66%, less than 67%, less than 68%, less than 69%, less than 70%, less than 71%, less than 72%, less than 73%, less than 74%, less than 75%, less than 76%, less than 77%, less than 78%, less than 79%, less than 80%, less than 81%, less than 82%, less than 83%, less than 84%, less than 85%, less than 86%, less than 87%, less than 88%, less than 89%, or less than 90%, of the ectodomain or, optionally all or a part of the M1 domain, the M2 domain, the LysM3 domain, or all three. A further embodiment of this aspect includes the EPR3a or EPR3a-like polypeptide being an endogenous EPR3a or EPR3a-like polypeptide.

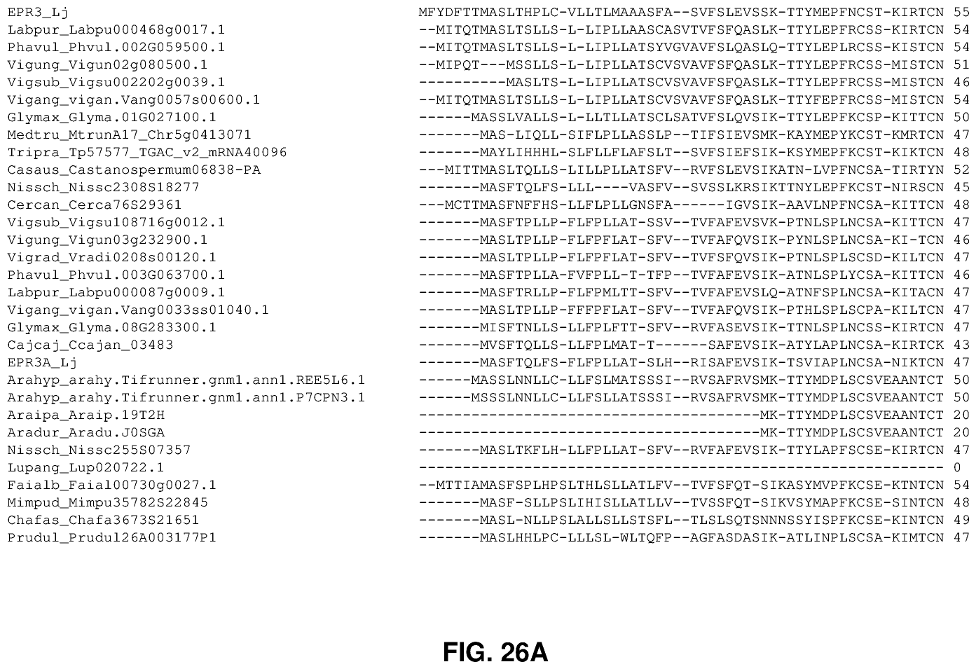

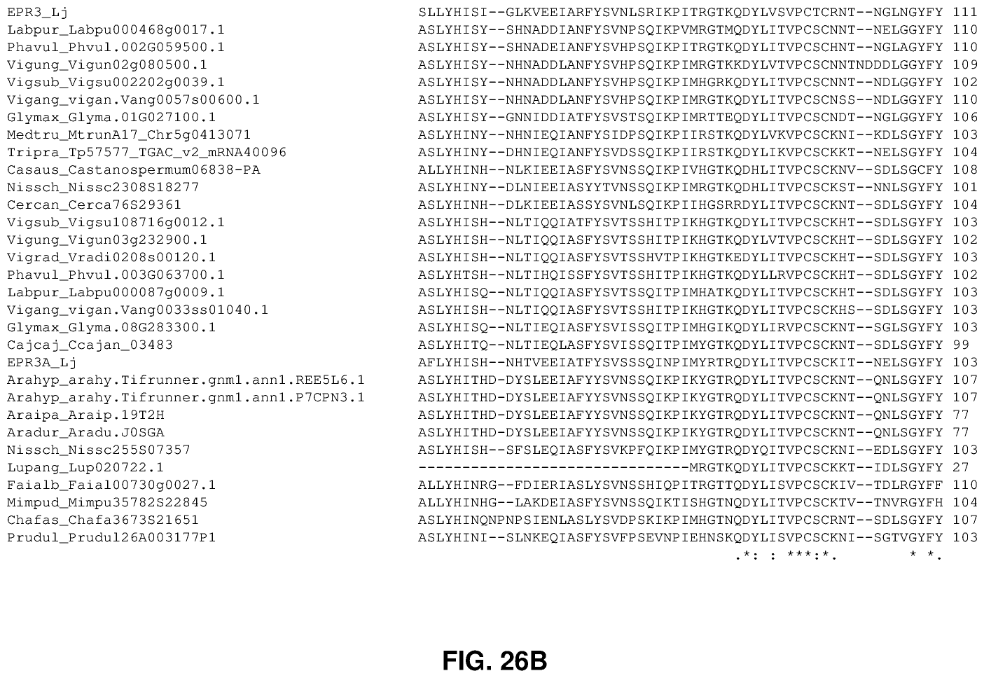

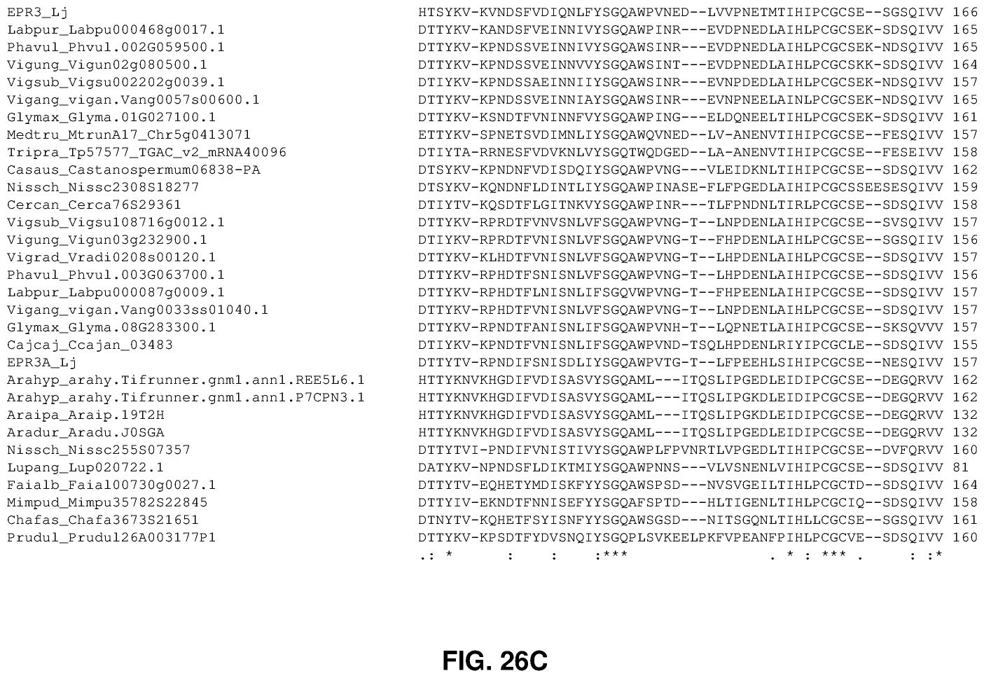

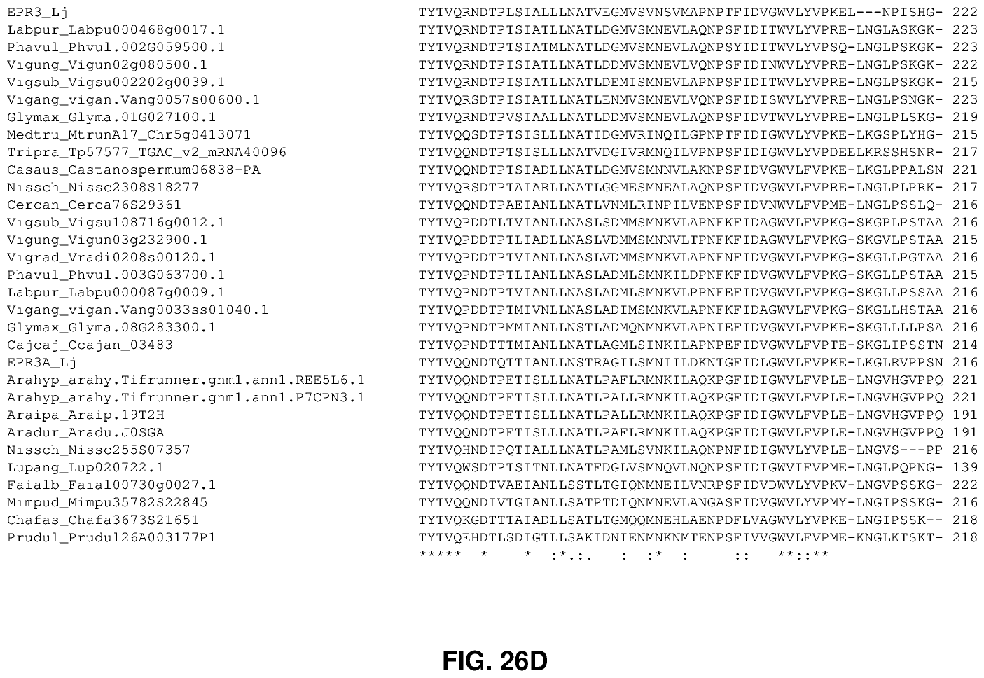

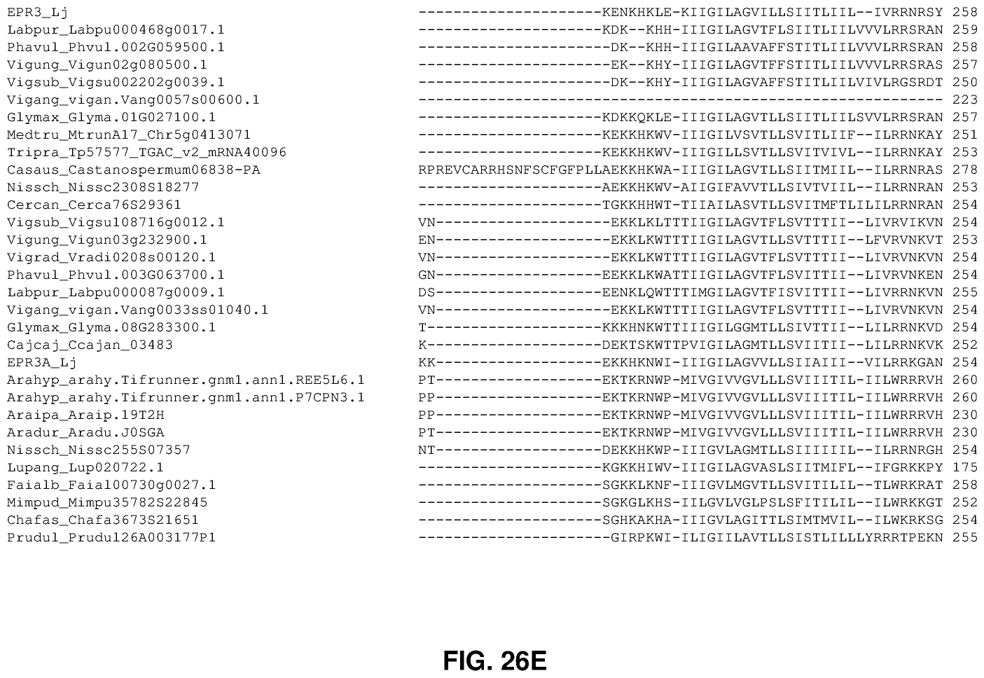

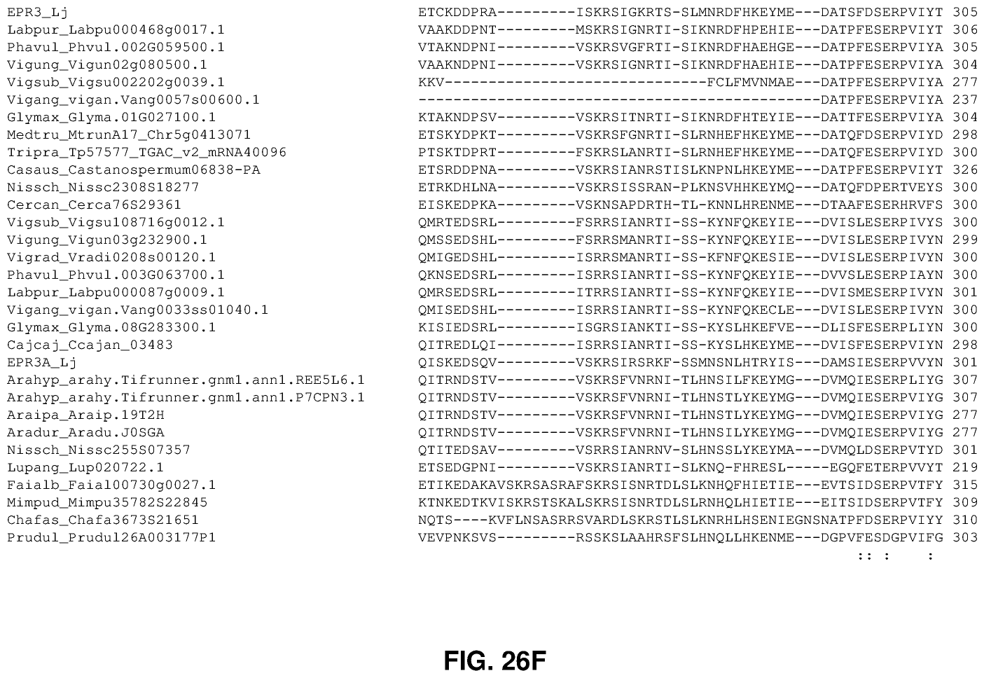

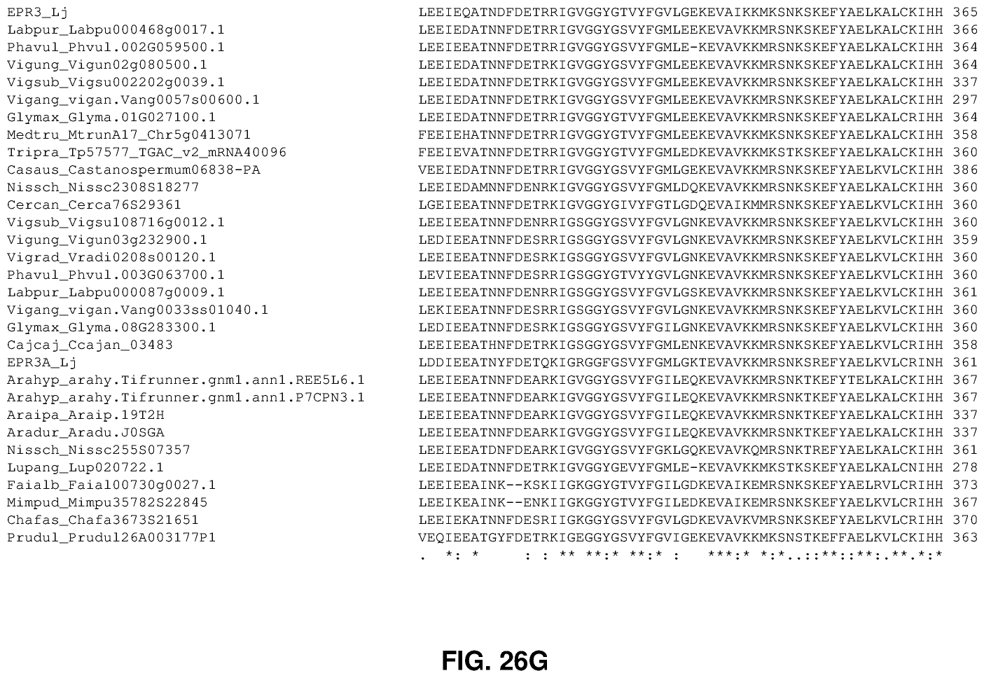

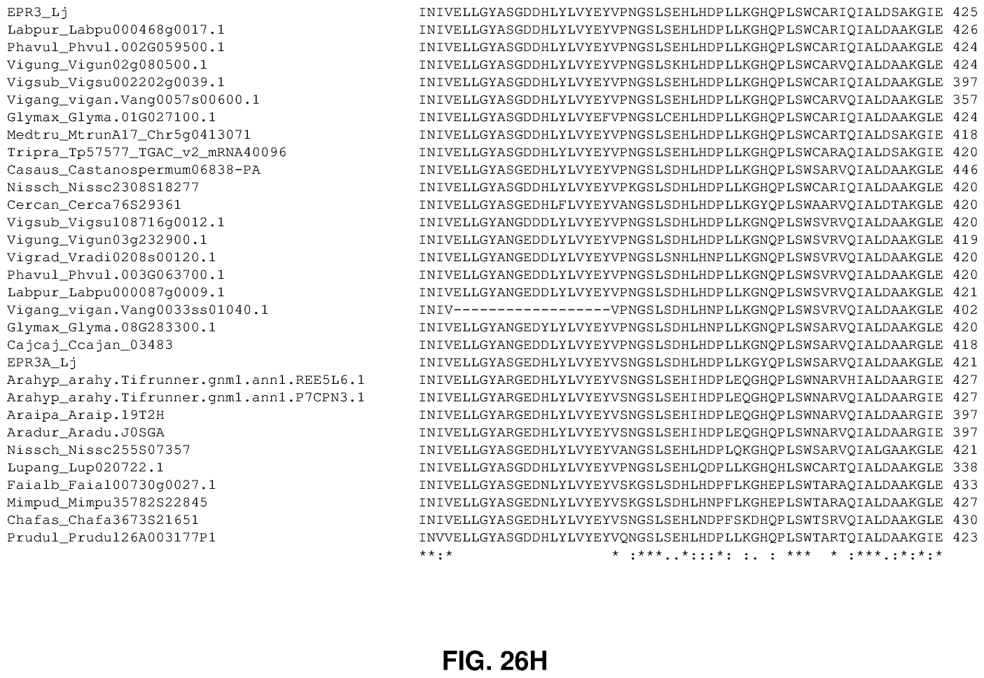

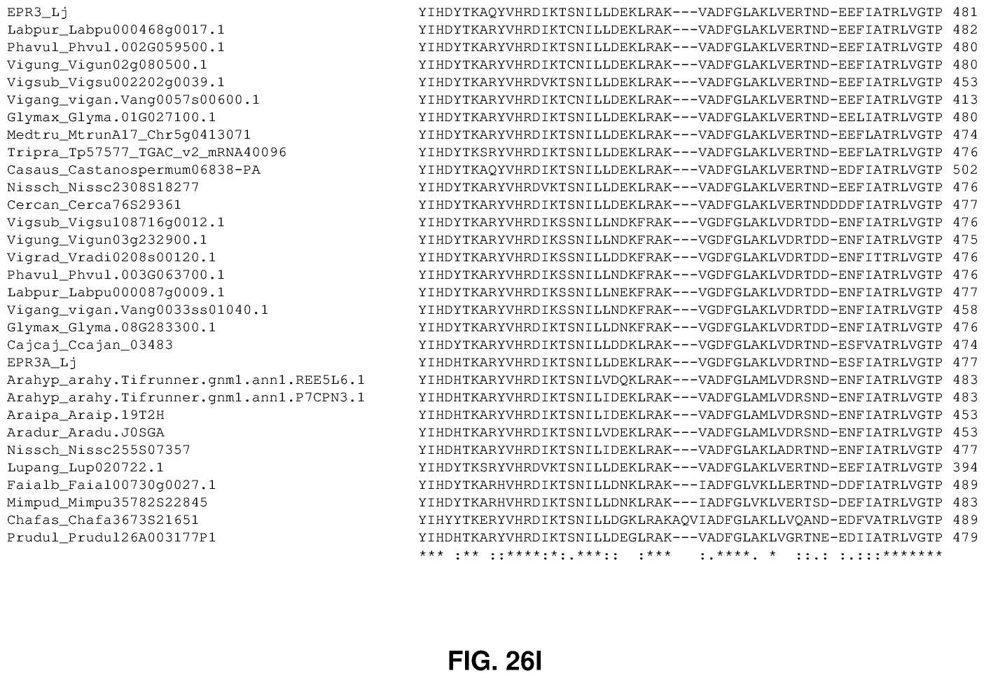

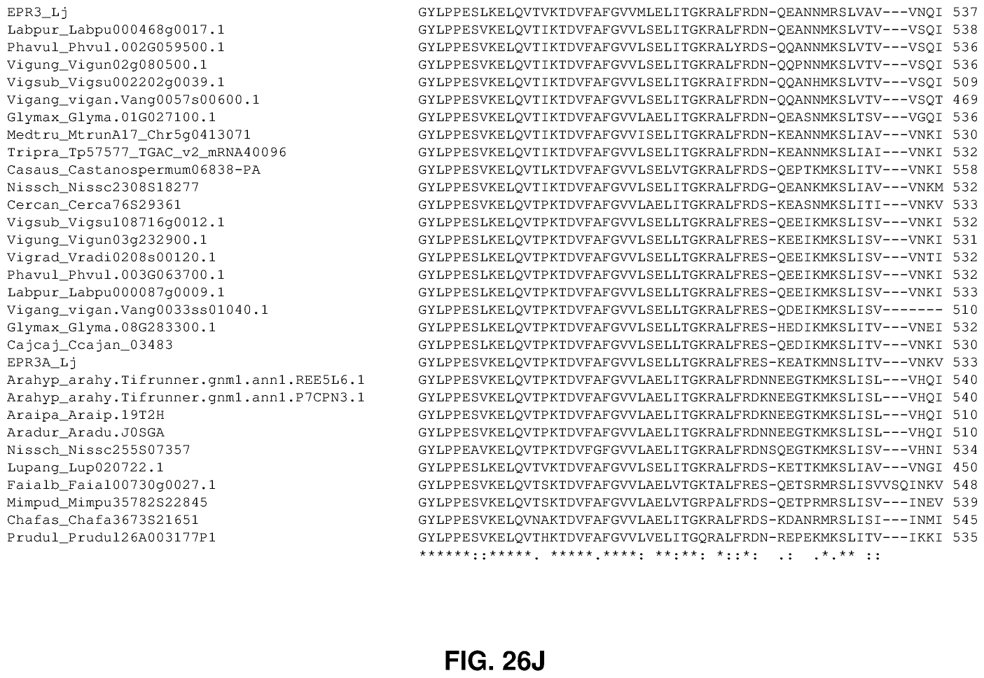

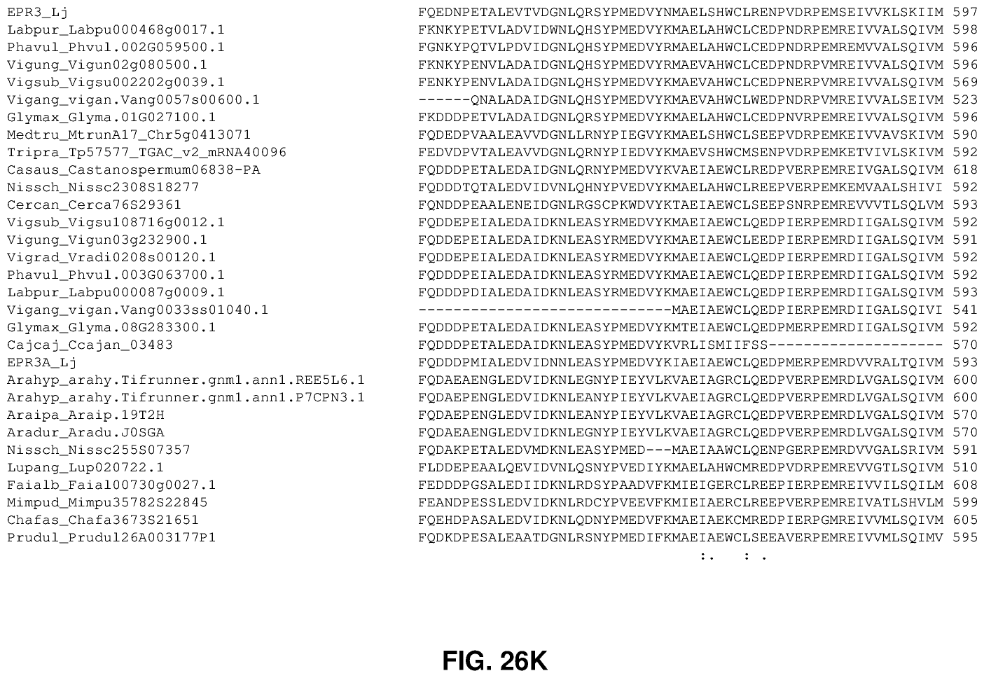

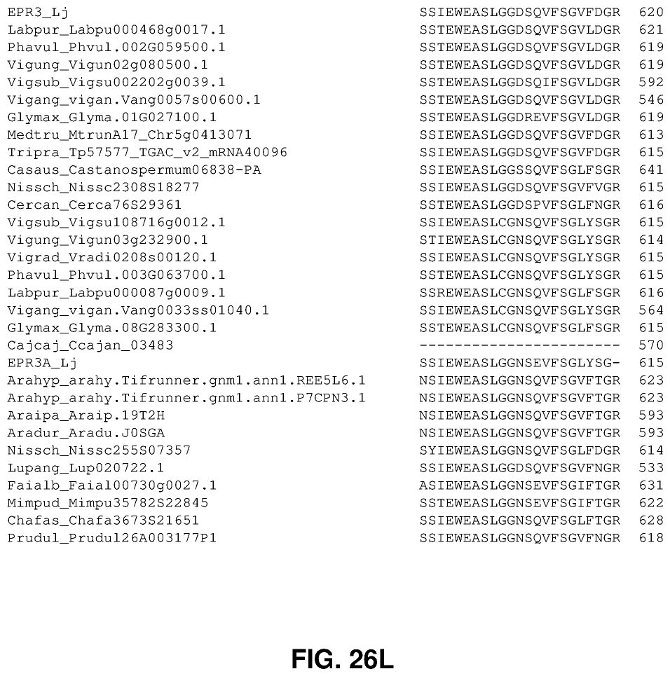

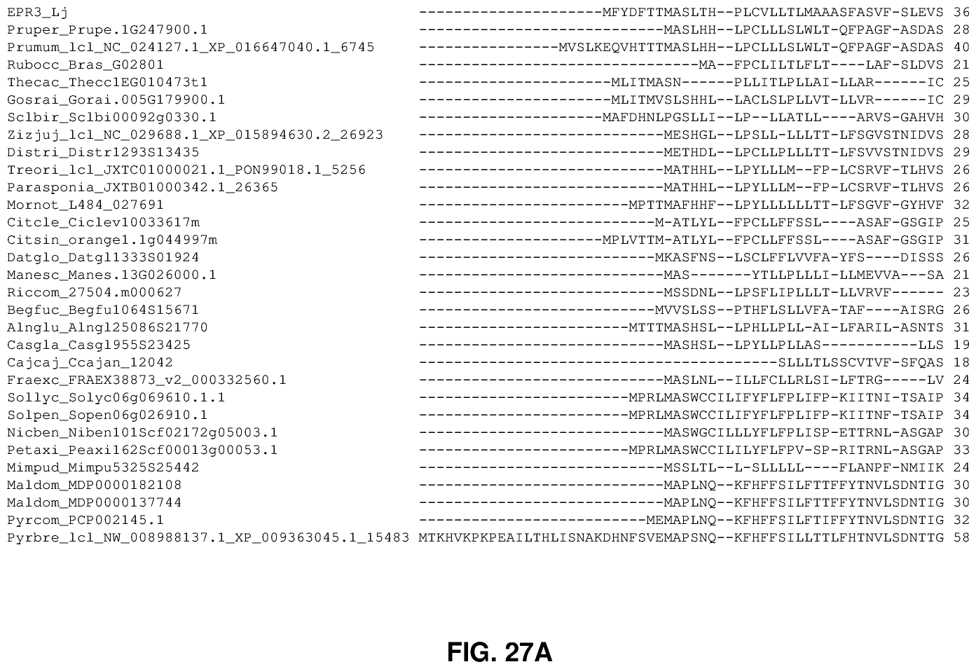

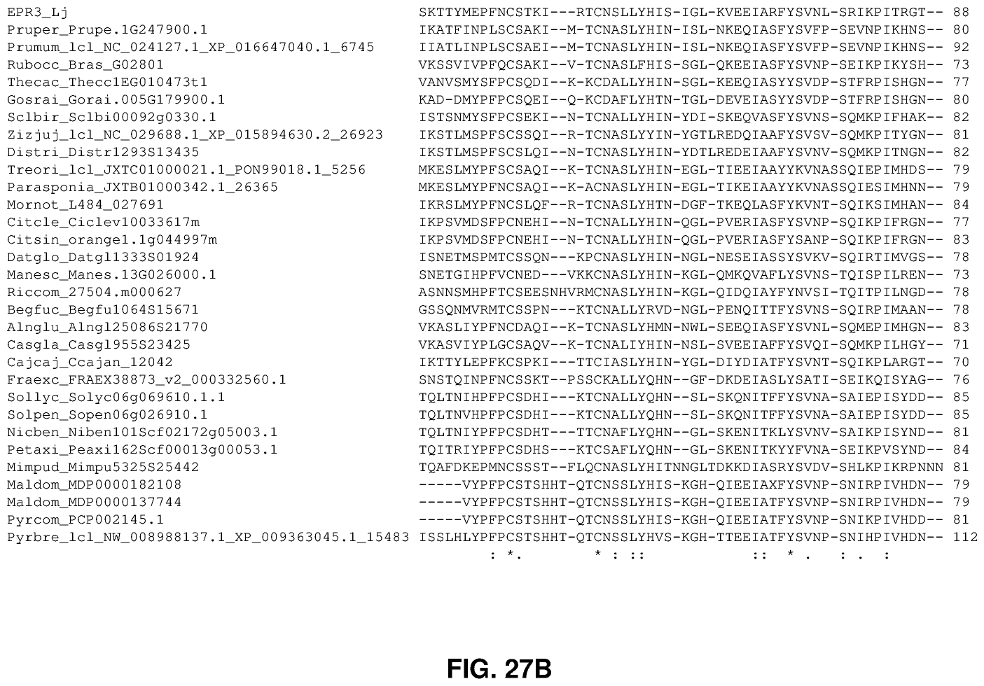

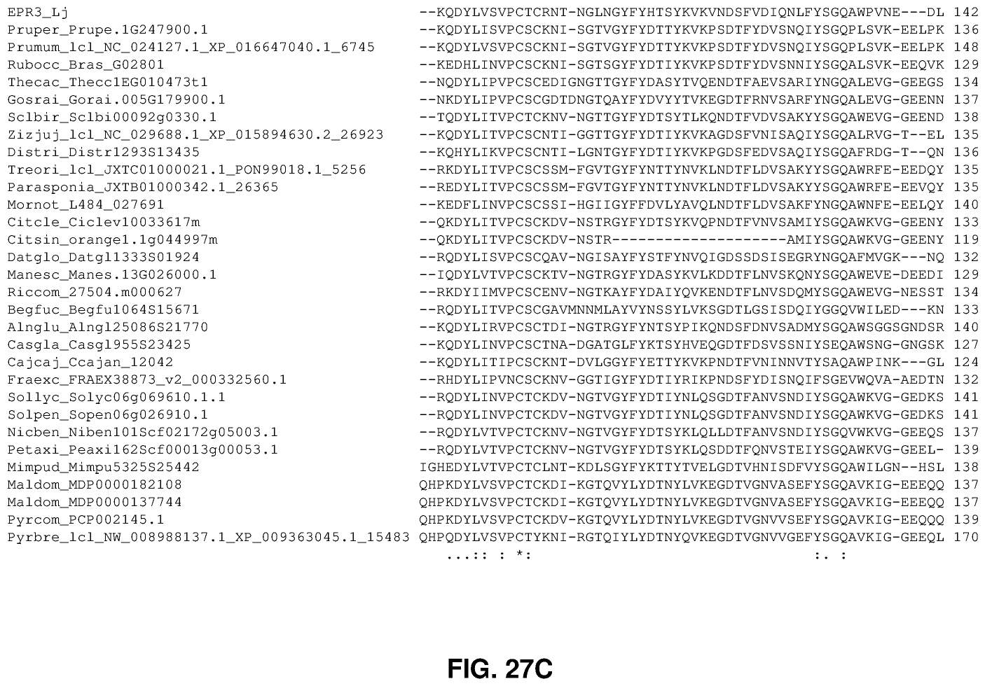

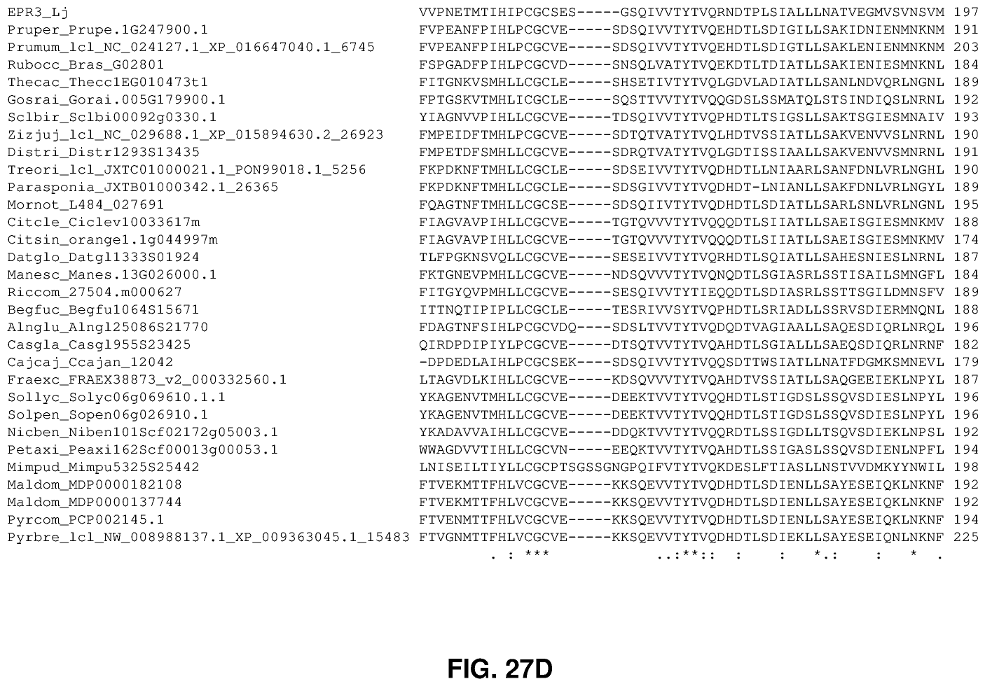

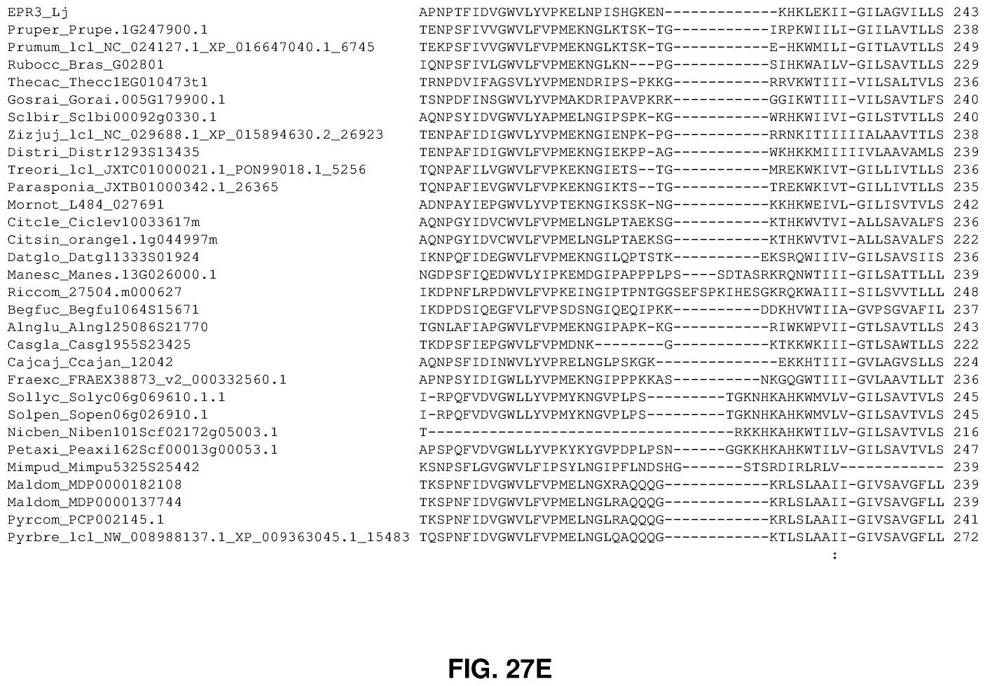

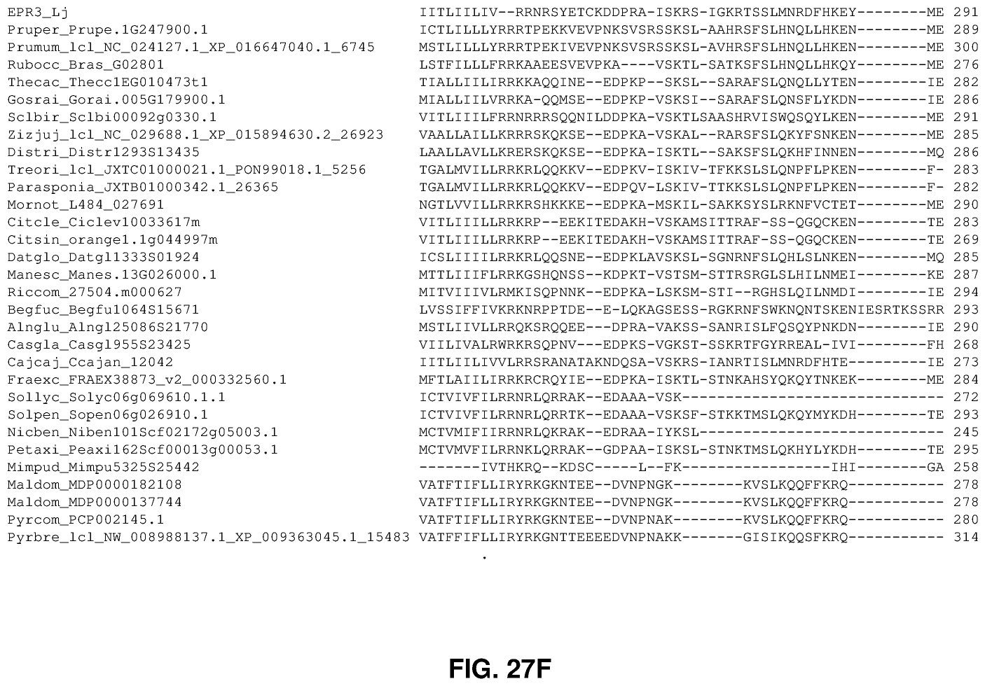

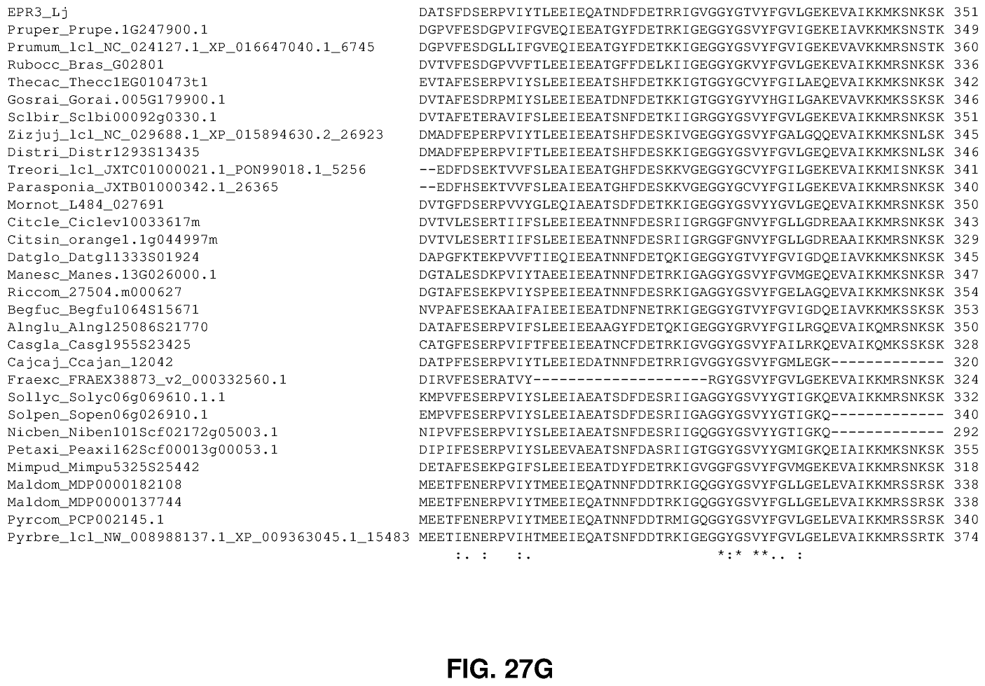

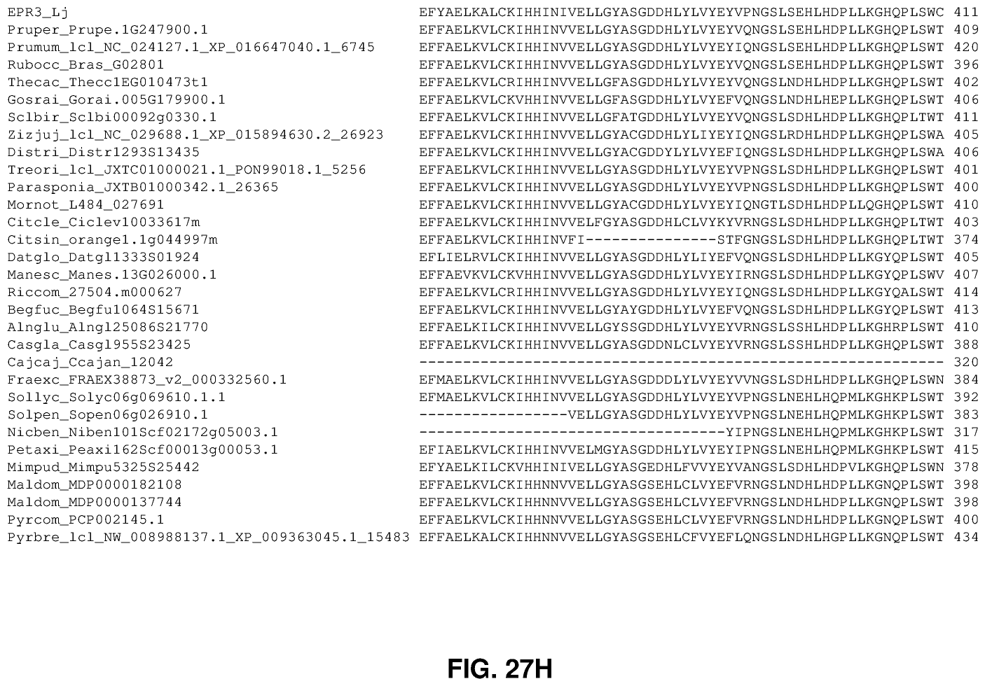

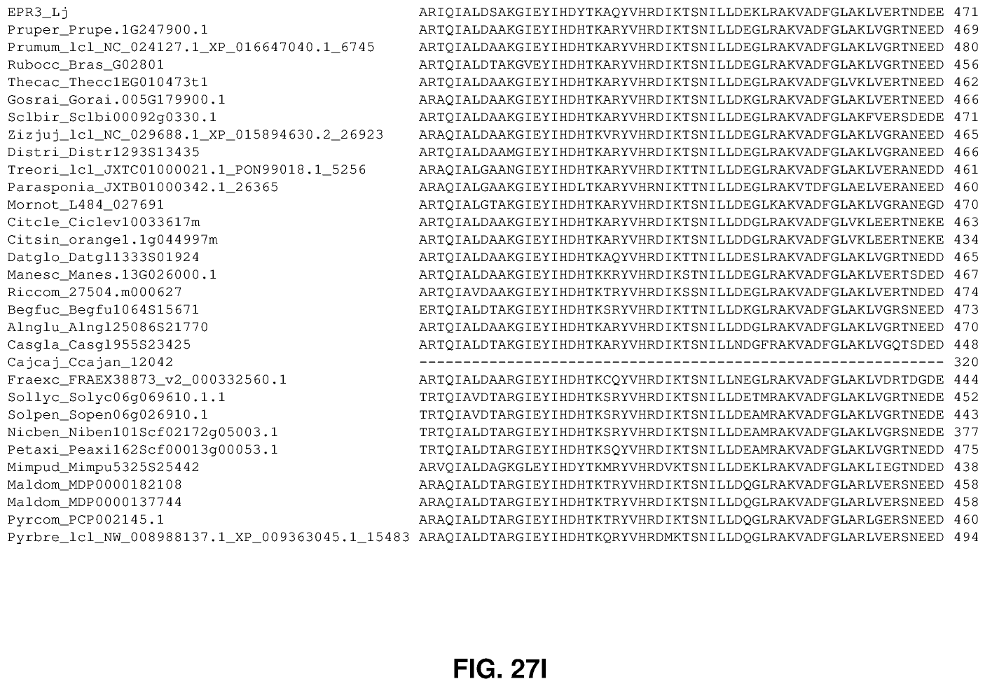

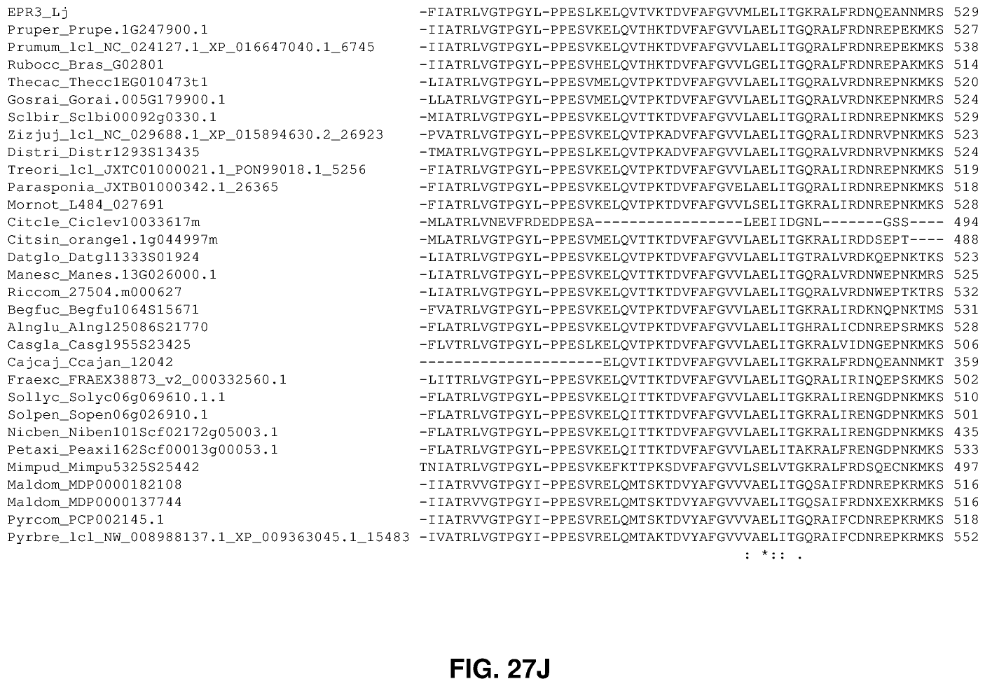

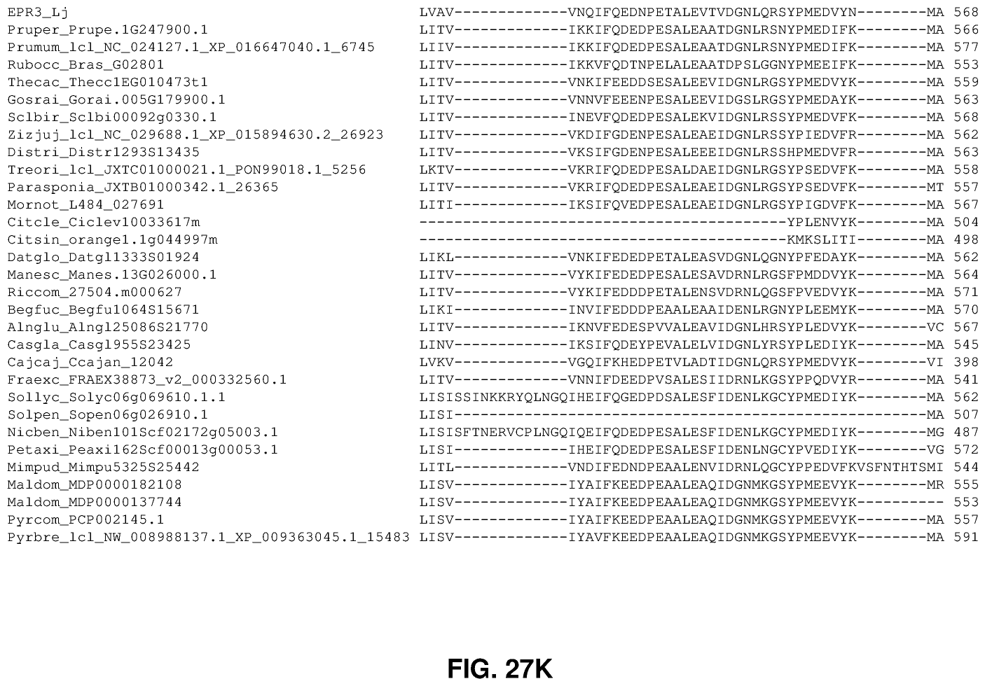

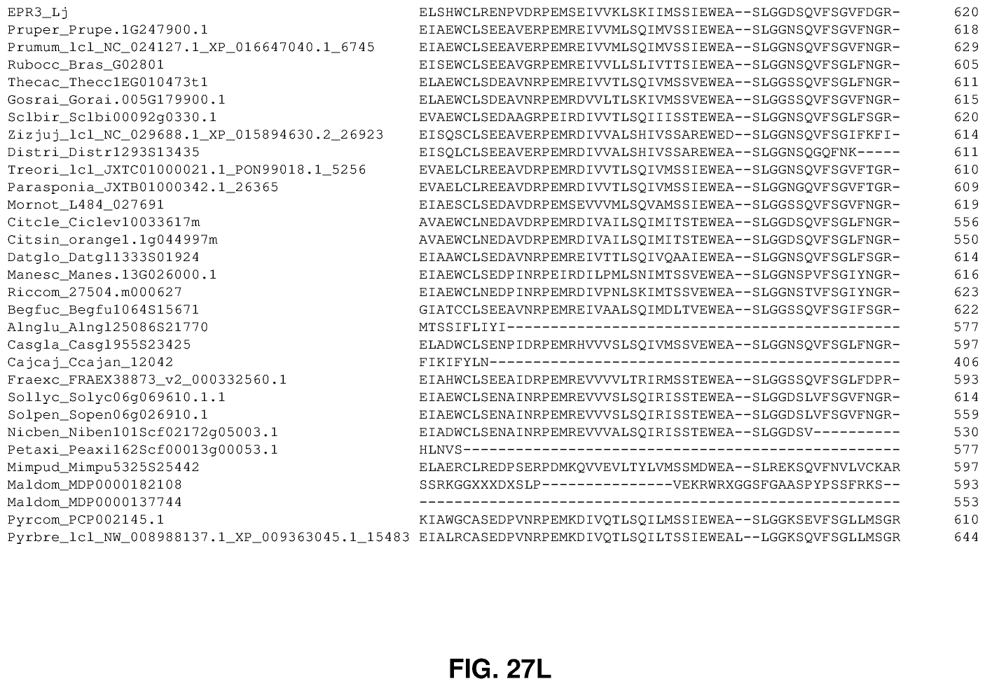

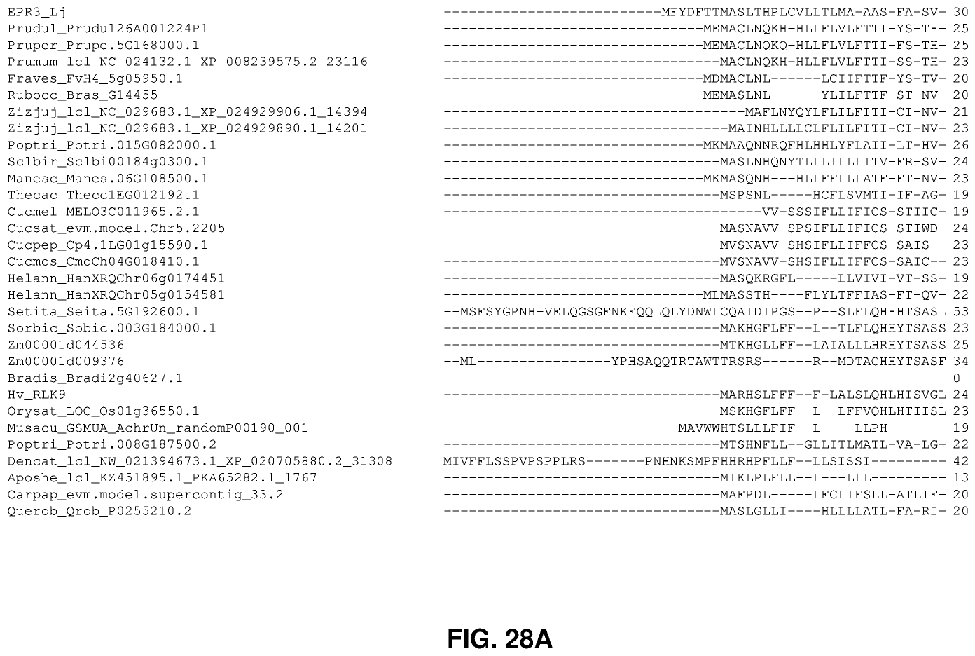

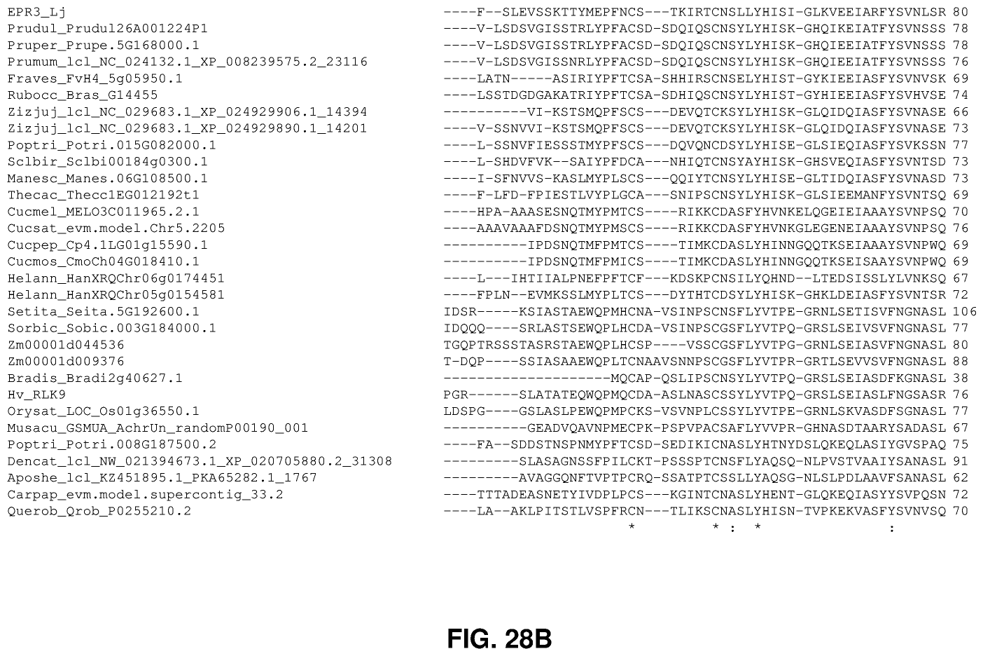

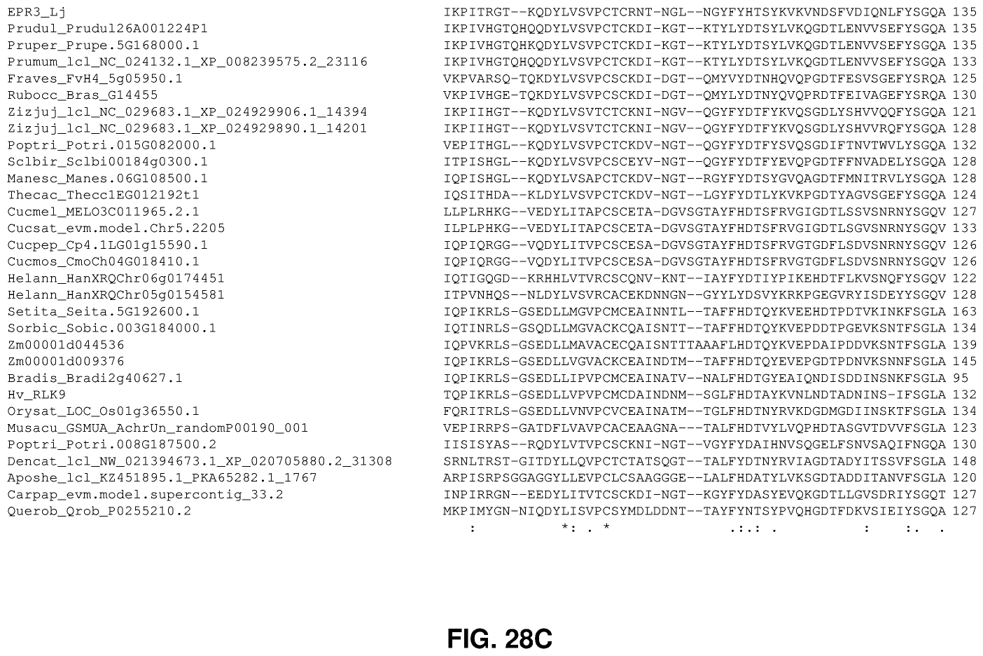

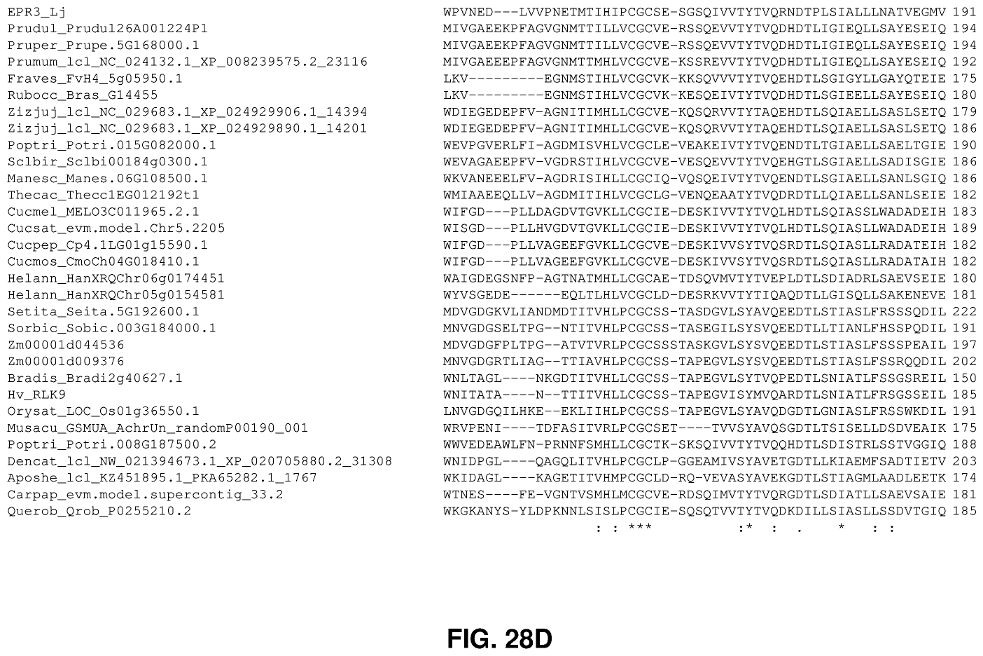

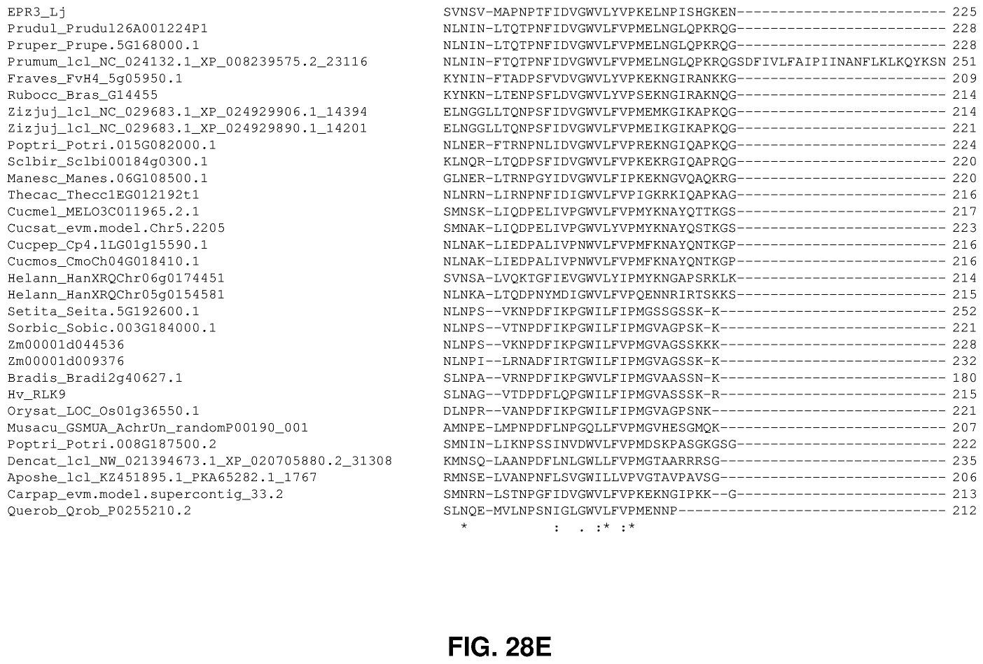

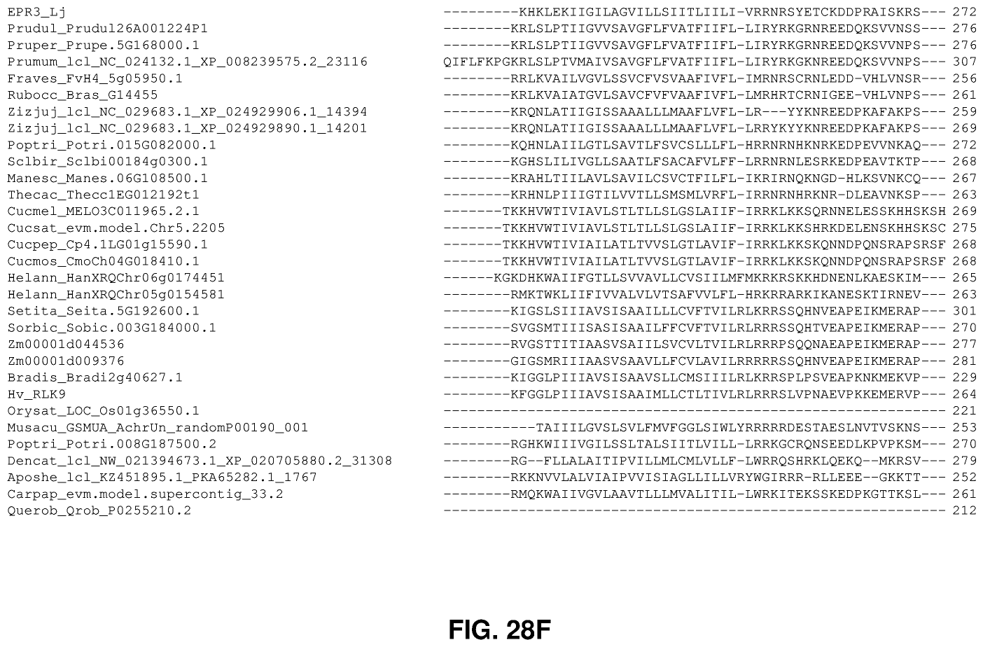

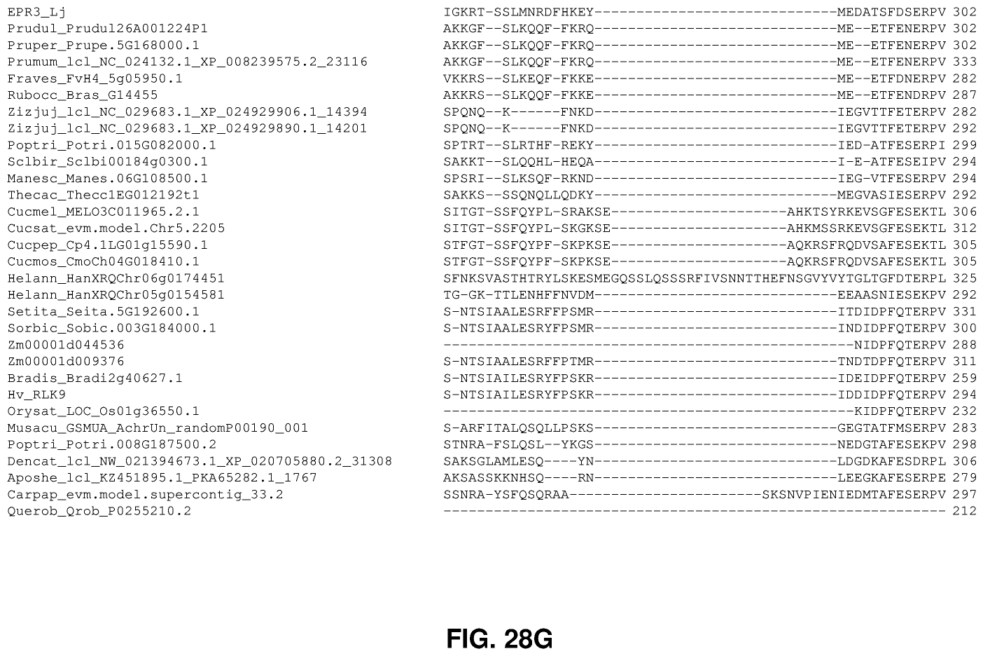

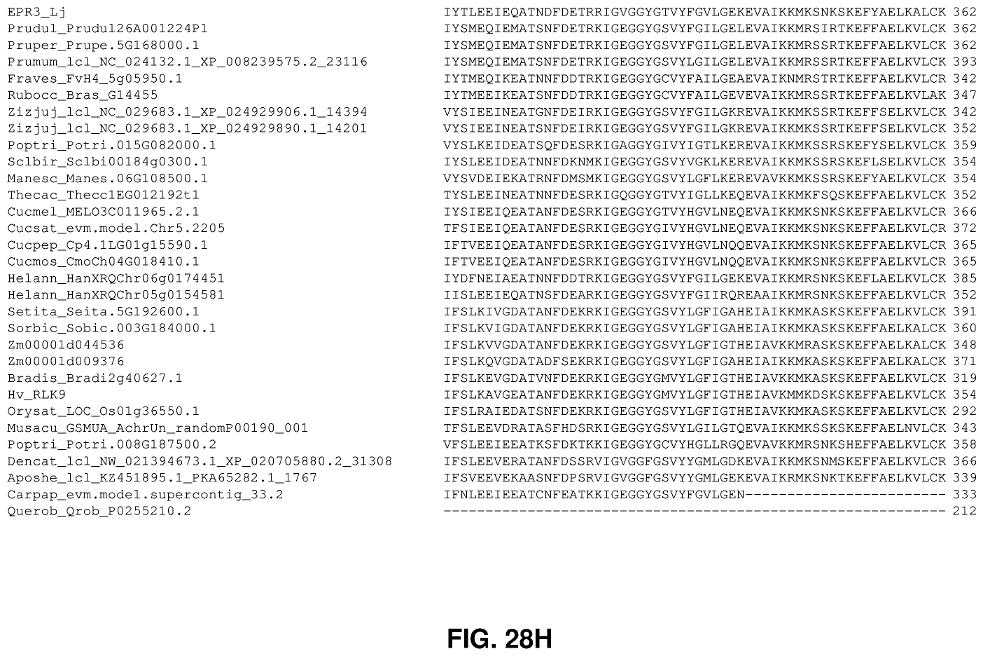

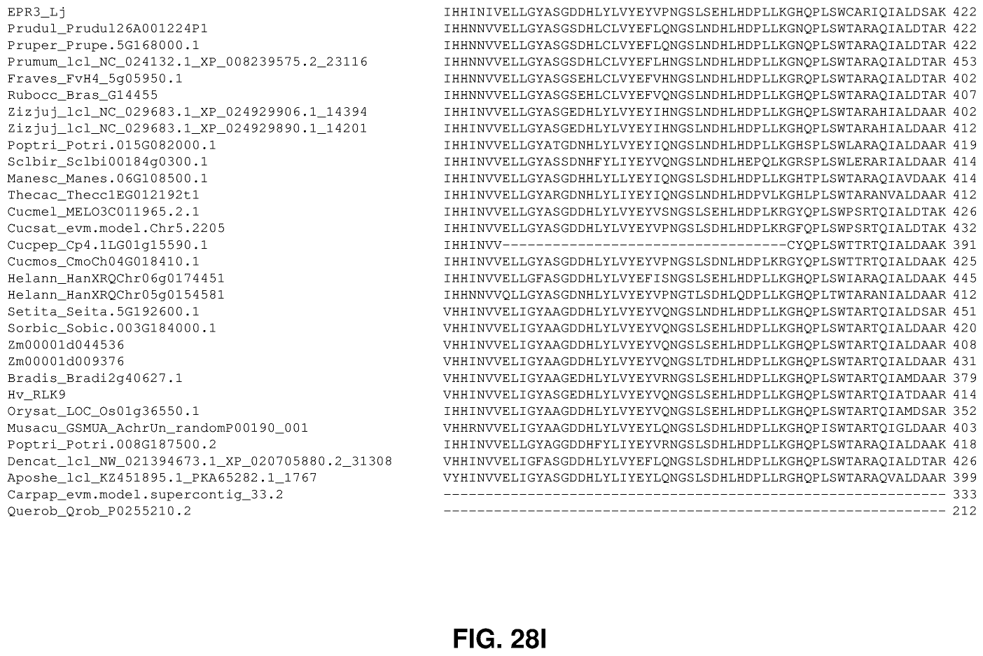

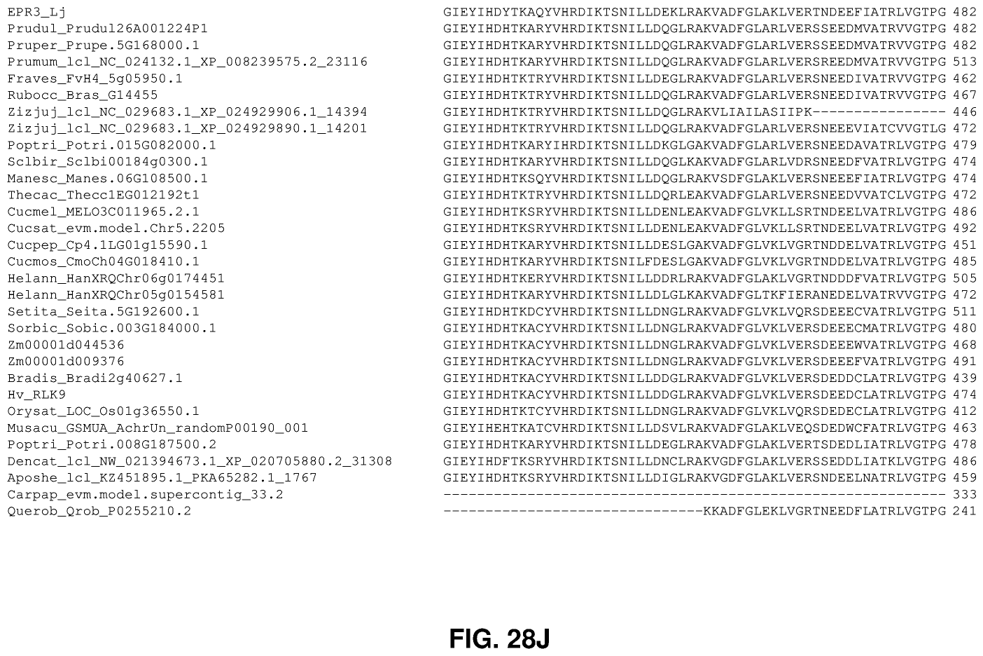

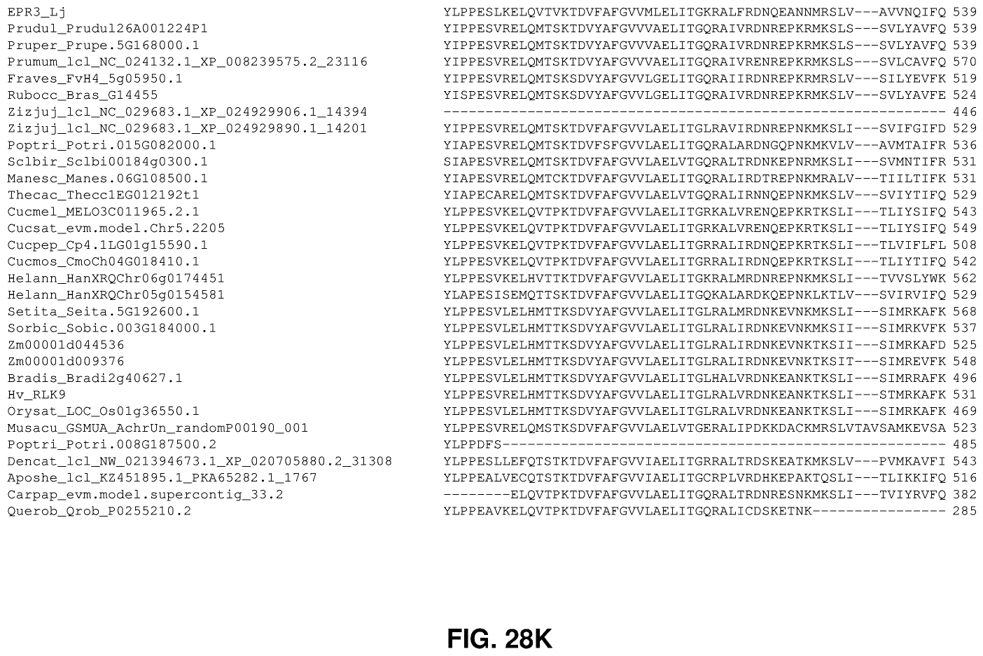

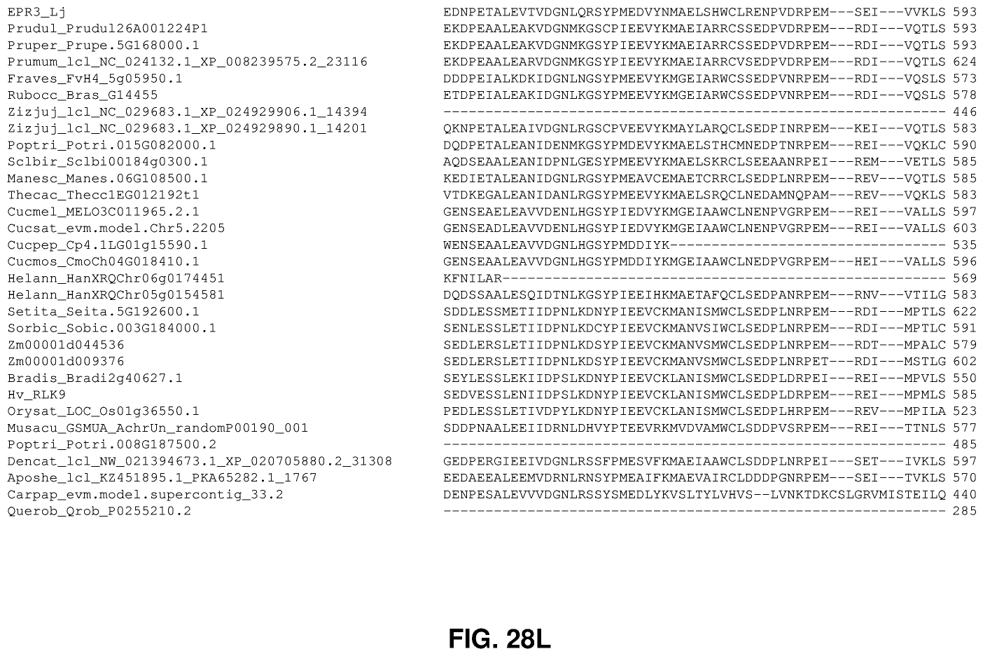

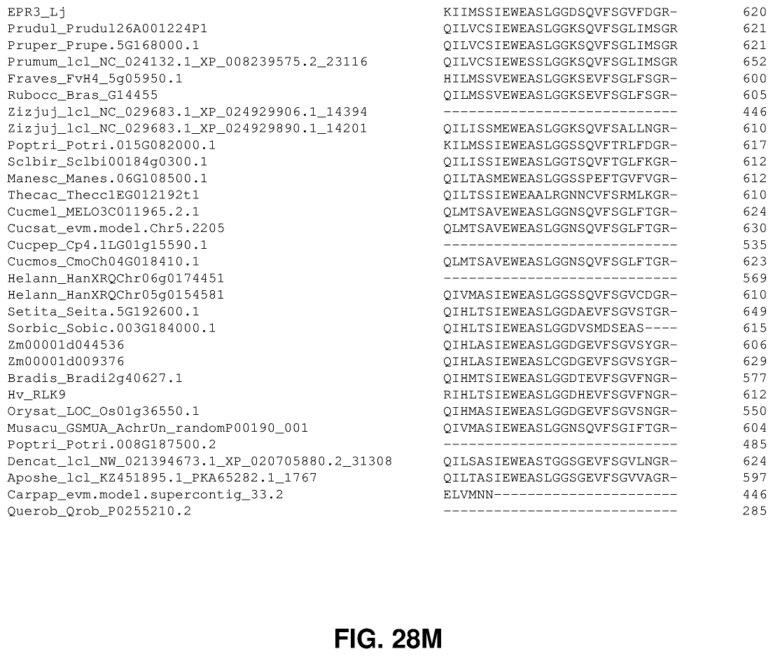

FIGS. 26A-26L, 27A-27L, and 28A-28M show alignments of the L. japonicus EPR3 polypeptide (EPR3_Lj, SEQ ID NO: 61), the L. japonicus EPR3a polypeptide (EPR3A_Lj, SEQ ID NO: 62) and EPR3-like and EPR3a-like polypeptides from a wide variety of other plant species.

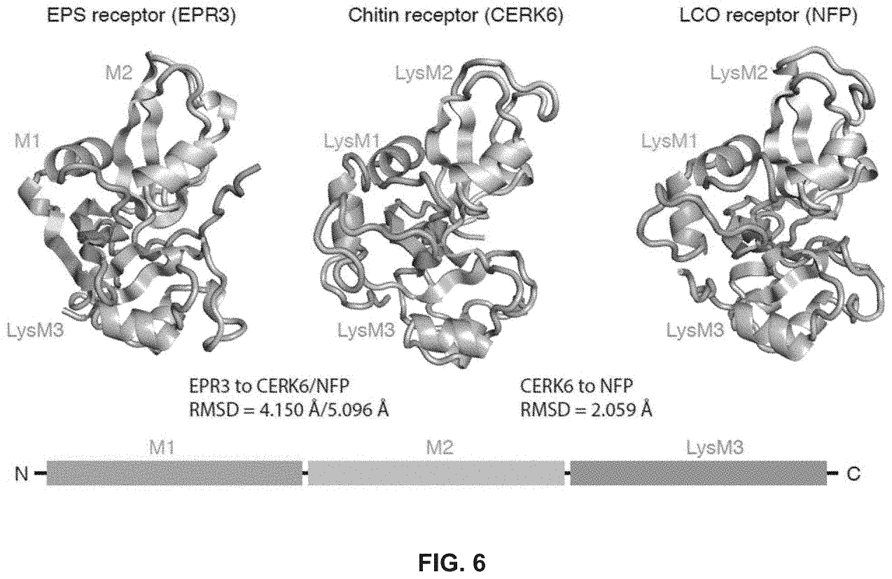

In order to identify EPR3 or EPR3-like polypeptides of the present disclosure, one of skill in the art would apply the teachings of this disclosure. For example, a first step would be to align the amino acid sequence of the potential EPR3 or EPR3-like receptor with one or more known EPR3 or EPR3-like receptor sequences. An exemplary known EPR3 receptor would be L. japonicus EPR3. The alignment would be used to determine the position of the M1 domain, which is at the N-terminal end of the ectodomain, and corresponds to the position of the LysM1 domain in canonical LysM receptors (FIG. 6). A second step would be to use an ab-initio protein structure prediction program such as Quark (Xu and Zhang Proteins 2012 80: 1715-1735) to predict the structure and fold of the new candidate M1 domain. Then, if the modeled M1 domain of the potential EPR3 or EPR3-like receptor shares the same topology, βαββ fold, and superimposes well with the L. japonicus EPR3 M1 domain, it is an EPR3 or EPR3-like polypeptide.

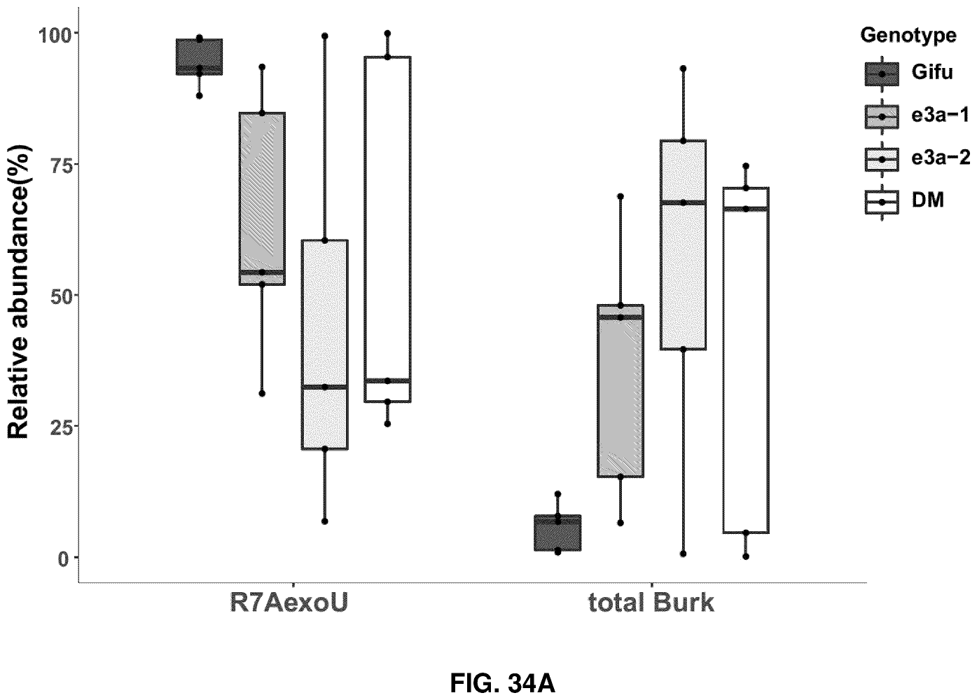

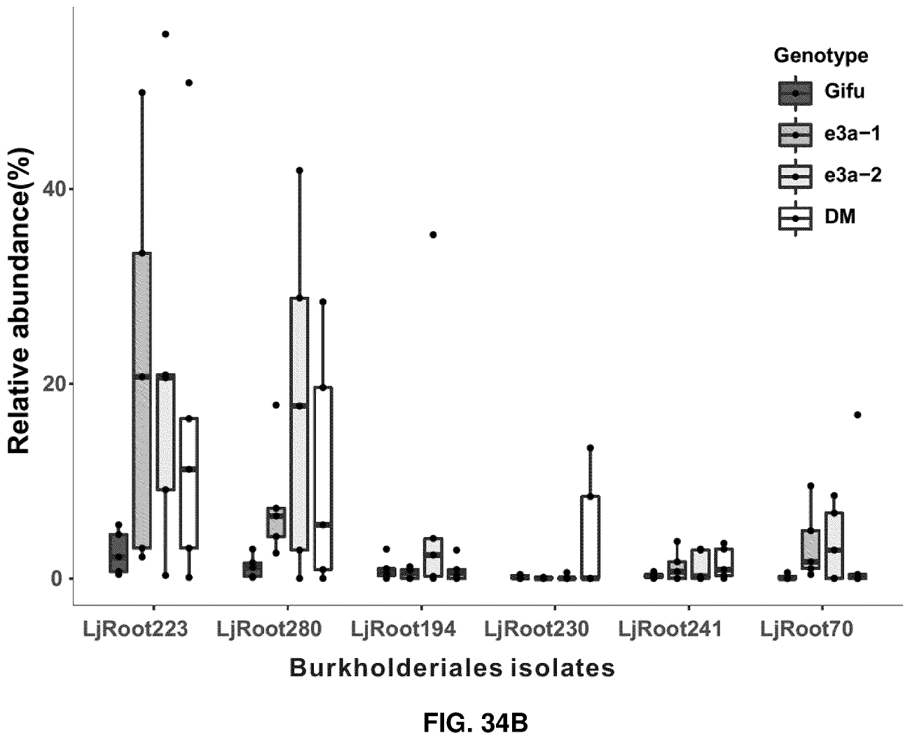

The L. japonicus EPR3 kinase domain has kinase activity (FIG. 11K), as does the L. japonicus EPR3s kinase domain (FIGS. 11L-11M). Without wishing to be bound by theory, EPR3 receptors, EPR3-like receptors, EPR3a receptors, and EPR3a-like receptors therefore may be capable of acting as independent receptors or may act as co-receptors with another protein (see, e.g., FIGS. 19A-19B and 34A-34B). For an EPR3 or EPR3-like receptor, the co-receptor may be the corresponding EPR3a or EPR3a-like receptor. Similarly, for an EPR3a or EPR3a-like receptor, the co-receptor may be the corresponding EPR3 or EPR3-like receptor. The presence or absence of a co-receptor may depend on the type of signal being perceived, and some microbial signals may be transmitted by only an EPR3, EPR3-like, EPR3a, or EPR3a-like receptor.

Transformation and generation of genetically altered monocotyledonous and dicotyledonous plant cells is well known in the art. See, e.g., Weising, et al., Ann. Rev. Genet. 22:421-477 (1988); U.S. Pat. No. 5,679,558; Agrobacterium Protocols, ed: Gartland, Humana Press Inc. (1995); and Wang, et al. Acta Hort. 461:401-408 (1998). The choice of method varies with the type of plant to be transformed, the particular application and/or the desired result. The appropriate transformation technique is readily chosen by the skilled practitioner.

Any methodology known in the art to delete, insert or otherwise modify the cellular DNA (e.g., genomic DNA and organelle DNA) can be used in practicing the inventions disclosed herein. For example, a disarmed Ti plasmid, containing a genetic construct for deletion or insertion of a target gene, in Agrobacterium tumefaciens can be used to transform a plant cell, and thereafter, a transformed plant can be regenerated from the transformed plant cell using procedures described in the art, for example, in EP 0116718, EP 0270822, PCT publication WO 84/02913 and published European Patent application (“EP”) 0242246. Ti-plasmid vectors each contain the gene between the border sequences, or at least located to the left of the right border sequence, of the T-DNA of the Ti-plasmid. Of course, other types of vectors can be used to transform the plant cell, using procedures such as direct gene transfer (as described, for example in EP 0233247), pollen mediated transformation (as described, for example in EP 0270356, PCT publication WO 85/01856, and U.S. Pat. No. 4,684,611), plant RNA virus-mediated transformation (as described, for example in EP 0 067 553 and U.S. Pat. No. 4,407,956), liposome-mediated transformation (as described, for example in U.S. Pat. No. 4,536,475), and other methods such as the methods for transforming certain lines of corn (e.g., U.S. Pat. No. 6,140,553; Fromm et al., Bio/Technology (1990) 8, 833-839); Gordon-Kamm et al., The Plant Cell, (1990) 2, 603-618) and rice (Shimamoto et al., Nature, (1989) 338, 274-276; Datta et al., Bio/Technology, (1990) 8, 736-740) and the method for transforming monocots generally (PCT publication WO 92/09696). For cotton transformation, the method described in PCT patent publication WO 00/71733 can be used. For soybean transformation, reference is made to methods known in the art, e.g., Hinchee et al. (Bio/Technology, (1988) 6, 915) and Christou et al. (Trends Biotech, (1990) 8, 145) or the method of WO 00/42207.

Genetically altered plants of the present invention can be used in a conventional plant breeding scheme to produce more genetically altered plants with the same characteristics, or to introduce the genetic alteration(s) in other varieties of the same or related plant species. Seeds, which are obtained from the altered plants, preferably contain the genetic alteration(s) as a stable insert in nuclear DNA or as modifications to an endogenous gene or promoter. Plants comprising the genetic alteration(s) in accordance with the invention include plants comprising, or derived from, root stocks of plants comprising the genetic alteration(s) of the invention, e.g., fruit trees or ornamental plants. Hence, any non-transgenic grafted plant parts inserted on a transformed plant or plant part are included in the invention.

Introduced genetic elements, whether in an expression vector or expression cassette, which result in the expression of an introduced gene, will typically utilize a plant-expressible promoter. A ‘plant-expressible promoter’ as used herein refers to a promoter that ensures expression of the genetic alteration(s) of the invention in a plant cell. Examples of promoters directing constitutive expression in plants are known in the art and include: the strong constitutive 35S promoters (the “35S promoters”) of the cauliflower mosaic virus (CaMV), e.g., of isolates CM 1841 (Gardner et al., Nucleic Acids Res, (1981) 9, 2871-2887), CabbB S (Franck et al., Cell (1980) 21, 285-294; Kay et al., Science, (1987) 236, 4805) and CabbB JI (Hull and Howell, Virology, (1987) 86, 482-493); cassava vein mosaic virus promoter (CsVMV); promoters from the ubiquitin family (e.g., the maize ubiquitin promoter of Christensen et al., Plant Mol Biol, (1992) 18, 675-689, or the A. thaliana UBQ10 promoter of Norris et al. Plant Mol. Biol. (1993) 21, 895-906), the gos2 promoter (de Pater et al., The Plant J (1992) 2, 834-844), the emu promoter (Last et al., Theor Appl Genet, (1990) 81, 581-588), actin promoters such as the promoter described by An et al. (The Plant J, (1996) 10, 107), the rice actin promoter described by Zhang et al. (The Plant Cell, (1991) 3, 1155-1165); promoters of the Cassava vein mosaic virus (WO 97/48819, Verdaguer et al. (Plant Mol Biol, (1998) 37, 1055-1067), the pPLEX series of promoters from Subterranean Clover Stunt Virus (WO 96/06932, particularly the S4 or S7 promoter), an alcohol dehydrogenase promoter, e.g., pAdh1S (GenBank accession numbers X04049, X00581), and the TR1′ promoter and the TR2′ promoter (the “TR1′ promoter” and “TR2′ promoter”, respectively) which drive the expression of the 1′ and 2′ genes, respectively, of the T DNA (Velten et al., EMBO J, (1984) 3, 2723 2730).

Alternatively, a plant-expressible promoter can be a tissue-specific promoter, i.e., a promoter directing a higher level of expression in some cells or tissues of the plant, e.g., in leaf mesophyll cells. In preferred embodiments, leaf mesophyll specific promoters or leaf guard cell specific promoters will be used. Non-limiting examples include the leaf specific Rbcs1A promoter (A. thaliana RuBisCO small subunit 1A (AT1G67090) promoter), GAPA-1 promoter (A. thaliana Glyceraldehyde 3-phosphate dehydrogenase A subunit 1 (AT3G26650) promoter), and FBA2 promoter (A. thaliana Fructose-bisphosphate aldolase 2 317 (AT4G38970) promoter) (Kromdijk et al., Science, 2016). Further non-limiting examples include the leaf mesophyll specific FBPase promoter (Peleg et al., Plant J, 2007), the maize or rice rbcS promoter (Nomura et al., Plant Mol Biol, 2000), the leaf guard cell specific A. thaliana KATI promoter (Nakamura et al., Plant Phys, 1995), the A. thaliana Myrosinase-Thioglucoside glucohydrolase 1 (TGG1) promoter (Husebye et al., Plant Phys, 2002), the A. thaliana rhal promoter (Terryn et al., Plant Cell, 1993), the A. thaliana AtCHX20 promoter (Padmanaban et al., Plant Phys, 2007), the A. thaliana HIC (High carbon dioxide) promoter (Gray et al., Nature, 2000), the A. thaliana CYTOCHROME P450 86A2 (CYP86A2) mono-oxygenase promoter (pCYP) (Francia et al., Plant Signal & Behav, 2008; Galbiati et al., The Plant Journal, 2008), the potato ADP-glucose pyrophosphorylase (AGPase) promoter (Muller-Rober et al., The Plant Cell 1994), the grape R2R3 MYB60 transcription factor promoter (Galbiati et al., BMC Plant Bio, 2011), the A. thaliana AtMYB60 promoter (Cominelli et al., Current Bio, 2005; Cominelli et al., BMC Plant Bio, 2011), the A. thaliana At1g22690-promoter (pGC1) (Yang et al., Plant Methods, 2008), and the A. thaliana AtMYB 61 promoter (Liang et al., Curr Biol, 2005). These plant promoters can be combined with enhancer elements, they can be combined with minimal promoter elements, or can comprise repeated elements to ensure the expression profile desired.

In some embodiments, genetic elements to increase expression in plant cells can be utilized. For example, an intron at the 5′ end or 3′ end of an introduced gene, or in the coding sequence of the introduced gene, e.g., the hsp70 intron. Other such genetic elements can include, but are not limited to, promoter enhancer elements, duplicated or triplicated promoter regions, 5′ leader sequences different from another transgene or different from an endogenous (plant host) gene leader sequence, 3′ trailer sequences different from another transgene used in the same plant or different from an endogenous (plant host) trailer sequence.

An introduced gene of the present invention can be inserted in host cell DNA so that the inserted gene part is upstream (i.e., 5′) of suitable 3′ end transcription regulation signals (e.g., transcript formation and polyadenylation signals). This is preferably accomplished by inserting the gene in the plant cell genome (nuclear or chloroplast). Preferred polyadenylation and transcript formation signals include those of the A. tumefaciens nopaline synthase gene (Nos terminator; Depicker et al., J. Molec Appl Gen, (1982) 1, 561-573), the octopine synthase gene (OCS terminator; Gielen et al., EMBO J, (1984) 3:835 845), the A. thaliana heat shock protein terminator (HSP terminator); the SCSV or the Malic enzyme terminators (Schunmann et al., Plant Funct Biol, (2003) 30:453-460), and the T DNA gene 7 (Velten and Schell, Nucleic Acids Res, (1985) 13, 6981 6998), which act as 3′ untranslated DNA sequences in transformed plant cells. In some embodiments, one or more of the introduced genes are stably integrated into the nuclear genome. Stable integration is present when the nucleic acid sequence remains integrated into the nuclear genome and continues to be expressed (e.g., detectable mRNA transcript or protein is produced) throughout subsequent plant generations. Stable integration into and/or editing of the nuclear genome can be accomplished by any known method in the art (e.g., microparticle bombardment, Agrobacterium-mediated transformation, CRISPR/Cas9, electroporation of protoplasts, microinjection, etc.).

The term recombinant or modified nucleic acids refers to polynucleotides which are made by the combination of two otherwise separated segments of sequence accomplished by the artificial manipulation of isolated segments of polynucleotides by genetic engineering techniques or by chemical synthesis. In so doing one may join together polynucleotide segments of desired functions to generate a desired combination of functions.

As used herein, the terms “overexpression” and “upregulation” refer to increased expression (e.g., of mRNA, polypeptides, etc.) relative to expression in a wild type organism (e.g., plant) as a result of genetic modification. In some embodiments, the increase in expression is a slight increase of about 10% more than expression in wild type. In some embodiments, the increase in expression is an increase of 50% or more (e.g., 60%, 70%, 80%, 100%, etc.) relative to expression in wild type. In some embodiments, an endogenous gene is overexpressed. In some embodiments, an exogenous gene is overexpressed by virtue of being expressed. Overexpression of a gene in plants can be achieved through any known method in the art, including but not limited to, the use of constitutive promoters, inducible promoters, high expression promoters, enhancers, transcriptional and/or translational regulatory sequences, codon optimization, modified transcription factors, and/or mutant or modified genes that control expression of the gene to be overexpressed.

Where a recombinant nucleic acid is intended for expression, cloning, or replication of a particular sequence, DNA constructs prepared for introduction into a host cell will typically comprise a replication system (e.g. vector) recognized by the host, including the intended DNA fragment encoding a desired polypeptide, and can also include transcription and translational initiation regulatory sequences operably linked to the polypeptide-encoding segment. Additionally, such constructs can include cellular localization signals (e.g., plasma membrane localization signals). In preferred embodiments, such DNA constructs are introduced into a host cell's genomic DNA, chloroplast DNA or mitochondrial DNA.

In some embodiments, a non-integrated expression system can be used to induce expression of one or more introduced genes. Expression systems (expression vectors) can include, for example, an origin of replication or autonomously replicating sequence (ARS) and expression control sequences, a promoter, an enhancer and necessary processing information sites, such as ribosome-binding sites, RNA splice sites, polyadenylation sites, transcriptional terminator sequences, and mRNA stabilizing sequences. Signal peptides can also be included where appropriate from secreted polypeptides of the same or related species, which allow the protein to cross and/or lodge in cell membranes, cell wall, or be secreted from the cell.

Selectable markers useful in practicing the methodologies of the invention disclosed herein can be positive selectable markers. Typically, positive selection refers to the case in which a genetically altered cell can survive in the presence of a toxic substance only if the recombinant polynucleotide of interest is present within the cell. Negative selectable markers and screenable markers are also well known in the art and are contemplated by the present invention. One of skill in the art will recognize that any relevant markers available can be utilized in practicing the inventions disclosed herein.

Screening and molecular analysis of recombinant strains of the present invention can be performed utilizing nucleic acid hybridization techniques. Hybridization procedures are useful for identifying polynucleotides, such as those modified using the techniques described herein, with sufficient homology to the subject regulatory sequences to be useful as taught herein. The particular hybridization techniques are not essential to the subject invention. As improvements are made in hybridization techniques, they can be readily applied by one of skill in the art. Hybridization probes can be labeled with any appropriate label known to those of skill in the art. Hybridization conditions and washing conditions, for example temperature and salt concentration, can be altered to change the stringency of the detection threshold. See, e.g., Sambrook et al. (1989) vide infra or Ausubel et al. (1995) Current Protocols in Molecular Biology, John Wiley & Sons, NY, N.Y., for further guidance on hybridization conditions.

Additionally, screening and molecular analysis of genetically altered strains, as well as creation of desired isolated nucleic acids can be performed using Polymerase Chain Reaction (PCR). PCR is a repetitive, enzymatic, primed synthesis of a nucleic acid sequence. This procedure is well known and commonly used by those skilled in this art (see Mullis, U.S. Pat. Nos. 4,683,195, 4,683,202, and 4,800,159; Saiki et al. (1985) Science 230:1350-1354). PCR is based on the enzymatic amplification of a DNA fragment of interest that is flanked by two oligonucleotide primers that hybridize to opposite strands of the target sequence. The primers are oriented with the 3′ ends pointing towards each other. Repeated cycles of heat denaturation of the template, annealing of the primers to their complementary sequences, and extension of the annealed primers with a DNA polymerase result in the amplification of the segment defined by the 5′ ends of the PCR primers. Because the extension product of each primer can serve as a template for the other primer, each cycle essentially doubles the amount of DNA template produced in the previous cycle. This results in the exponential accumulation of the specific target fragment, up to several million-fold in a few hours. By using a thermostable DNA polymerase such as the Taq polymerase, which is isolated from the thermophilic bacterium Thermus aquaticus, the amplification process can be completely automated. Other enzymes which can be used are known to those skilled in the art.

Nucleic acids and proteins of the present invention can also encompass homologues of the specifically disclosed sequences. Homology (e.g., sequence identity) can be 50%-100%. In some instances, such homology is greater than 80%, greater than 85%, greater than 90%, or greater than 95%. The degree of homology or identity needed for any intended use of the sequence(s) is readily identified by one of skill in the art. As used herein percent sequence identity of two nucleic acids is determined using an algorithm known in the art, such as that disclosed by Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the BLASTN, BLASTP, and BLASTX, programs of Altschul et al. (1990) J. Mol. Biol. 215:402-410. BLAST nucleotide searches are performed with the BLASTN program, score=100, wordlength=12, to obtain nucleotide sequences with the desired percent sequence identity. To obtain gapped alignments for comparison purposes, Gapped BLAST is used as described in Altschul et al. (1997) Nucl. Acids. Res. 25:3389-3402. When utilizing BLAST and Gapped BLAST programs, the default parameters of the respective programs (BLASTN and BLASTX) are used. See www.ncbi.nih.gov. One of skill in the art can readily determine in a sequence of interest where a position corresponding to amino acid or nucleic acid in a reference sequence occurs by aligning the sequence of interest with the reference sequence using the suitable BLAST program with the default settings (e.g., for BLASTP: Gap opening penalty: 11, Gap extension penalty: 1, Expectation value: 10, Word size: 3, Max scores: 25, Max alignments: 15, and Matrix: blosum62; and for BLASTN: Gap opening penalty: 5, Gap extension penalty:2, Nucleic match: 1, Nucleic mismatch—3, Expectation value: 10, Word size: 11, Max scores: 25, and Max alignments: 15).

Preferred host cells are plant cells. Recombinant host cells, in the present context, are those which have been genetically modified to contain an isolated nucleic molecule, contain one or more deleted or otherwise non-functional genes normally present and functional in the host cell, or contain one or more genes to produce at least one recombinant protein. The nucleic acid(s) encoding the protein(s) of the present invention can be introduced by any means known to the art which is appropriate for the particular type of cell, including without limitation, transformation, lipofection, electroporation or any other methodology known by those skilled in the art.

### Plant Breeding Methods

Plant breeding begins with the analysis of the current germplasm, the definition of problems and weaknesses of the current germplasm, the establishment of program goals, and the definition of specific breeding objectives. The next step is the selection of germplasm that possess the traits to meet the program goals. The selected germplasm is crossed in order to recombine the desired traits and through selection, varieties or parent lines are developed. The goal is to combine in a single variety or hybrid an improved combination of desirable traits from the parental germplasm. These important traits may include higher yield, field performance, improved fruit and agronomic quality, resistance to biological stresses, such as diseases and pests, and tolerance to environmental stresses, such as drought and heat.

Each breeding program should include a periodic, objective evaluation of the efficiency of the breeding procedure. Evaluation criteria vary depending on the goal and objectives, but should include gain from selection per year based on comparisons to an appropriate standard, overall value of the advanced breeding lines, and number of successful cultivars produced per unit of input (e.g., per year, per dollar expended, etc.). Promising advanced breeding lines are thoroughly tested and compared to appropriate standards in environments representative of the commercial target area(s) for three years at least. The best lines are candidates for new commercial cultivars; those still deficient in a few traits are used as parents to produce new populations for further selection. These processes, which lead to the final step of marketing and distribution, usually take five to ten years from the time the first cross or selection is made.

The choice of breeding or selection methods depends on the mode of plant reproduction, the heritability of the trait(s) being improved, and the type of cultivar used commercially (e.g., F₁ hybrid cultivar, inbred cultivar, etc.). For highly heritable traits, a choice of superior individual plants evaluated at a single location will be effective, whereas for traits with low heritability, selection should be based on mean values obtained from replicated evaluations of families of related plants. The complexity of inheritance also influences the choice of the breeding method. Backcross breeding is used to transfer one or a few genes for a highly heritable trait into a desirable cultivar (e.g., for breeding disease-resistant cultivars), while recurrent selection techniques are used for quantitatively inherited traits controlled by numerous genes, various recurrent selection techniques are used. Commonly used selection methods include pedigree selection, modified pedigree selection, mass selection, and recurrent selection.

Pedigree selection is generally used for the improvement of self-pollinating crops or inbred lines of cross-pollinating crops. Two parents which possess favorable, complementary traits are crossed to produce an F₁. An F₂ population is produced by selfing one or several F₁s or by intercrossing two F₁s (sib mating). Selection of the best individuals is usually begun in the F₂ population; then, beginning in the F₃, the best individuals in the best families are selected. Replicated testing of families, or hybrid combinations involving individuals of these families, often follows in the F₄ generation to improve the effectiveness of selection for traits with low heritability. At an advanced stage of inbreeding (i.e., F₆ and F₇), the best lines or mixtures of phenotypically similar lines are tested for potential release as new cultivars.

Mass and recurrent selections can be used to improve populations of either self- or cross-pollinating crops. A genetically variable population of heterozygous individuals is either identified or created by intercrossing several different parents. The best plants are selected based on individual superiority, outstanding progeny, or excellent combining ability. The selected plants are intercrossed to produce a new population in which further cycles of selection are continued.

Backcross breeding (i.e., recurrent selection) may be used to transfer genes for a simply inherited, highly heritable trait into a desirable homozygous cultivar or line that is the recurrent parent. The source of the trait to be transferred is called the donor parent. The resulting plant is expected to have the attributes of the recurrent parent (e.g., cultivar) and the desirable trait transferred from the donor parent. After the initial cross, individuals possessing the phenotype of the donor parent are selected and repeatedly crossed (backcrossed) to the recurrent parent. The resulting plant is expected to have the attributes of the recurrent parent (e.g., cultivar) and the desirable trait transferred from the donor parent.

The single-seed descent procedure in the strict sense refers to planting a segregating population, harvesting a sample of one seed per plant, and using the one-seed sample to plant the next generation. When the population has been advanced from the F₂ to the desired level of inbreeding, the plants from which lines are derived will each trace to different F₂ individuals. The number of plants in a population declines each generation due to failure of some seeds to germinate or some plants to produce at least one seed. As a result, not all of the F₂ plants originally sampled in the population will be represented by a progeny when generation advance is completed.

In addition to phenotypic observations, the genotype of a plant can also be examined. There are many laboratory-based techniques available for the analysis, comparison and characterization of plant genotype; among these are Isozyme Electrophoresis, Restriction Fragment Length Polymorphisms (RFLPs), Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length polymorphisms (AFLPs), Simple Sequence Repeats (SSRs—which are also referred to as Microsatellites), and Single Nucleotide Polymorphisms (SNPs).

Molecular markers, or “markers”, can also be used during the breeding process for the selection of qualitative traits. For example, markers closely linked to alleles or markers containing sequences within the actual alleles of interest can be used to select plants that contain the alleles of interest. The use of markers in the selection process is often called genetic marker enhanced selection or marker-assisted selection. Methods of performing marker analysis are generally known to those of skill in the art.

Mutation breeding may also be used to introduce new traits into plant varieties. Mutations that occur spontaneously or are artificially induced can be useful sources of variability for a plant breeder. The goal of artificial mutagenesis is to increase the rate of mutation for a desired characteristic. Mutation rates can be increased by many different means including temperature, long-term seed storage, tissue culture conditions, radiation (such as X-rays, Gamma rays, neutrons, Beta radiation, or ultraviolet radiation), chemical mutagens (such as base analogs like 5-bromo-uracil), antibiotics, alkylating agents (such as sulfur mustards, nitrogen mustards, epoxides, ethyleneamines, sulfates, sulfonates, sulfones, or lactones), azide, hydroxylamine, nitrous acid or acridines. Once a desired trait is observed through mutagenesis the trait may then be incorporated into existing germplasm by traditional breeding techniques. Details of mutation breeding can be found in Principles of Cultivar Development: Theory and Technique, Walter Fehr (1991), Agronomy Books, 1 (https://lib.dr.iastate.edu/agron_books/1).

The production of double haploids can also be used for the development of homozygous lines in a breeding program. Double haploids are produced by the doubling of a set of chromosomes from a heterozygous plant to produce a completely homozygous individual. For example, see Wan, et al., Theor. Appl. Genet., 77:889-892, 1989.

Additional non-limiting examples of breeding methods that may be used include, without limitation, those found in Principles of Plant Breeding, John Wiley and Son, pp. 115-161 (1960); Principles of Cultivar Development: Theory and Technique, Walter Fehr (1991), Agronomy Books, 1 (https://lib.dr.iastate.edu/agron_books/1), which are herewith incorporated by reference.

Having generally described this invention, the same will be better understood by reference to certain specific examples, which are included herein to further illustrate the invention and are not intended to limit the scope of the invention as defined by the claims.

### EXAMPLES

The present disclosure is described in further detail in the following examples which are not in any way intended to limit the scope of the disclosure as claimed. The attached figures are meant to be considered as integral parts of the specification and description of the disclosure. The following examples are offered to illustrate, but not to limit the claimed disclosure.

### Example 1: Structure of a Plant Receptor Perceiving Bacterial Exopolysaccharides

The following example describes the determination of the crystal structure of the Lotus japonicus Exopolysaccharide Receptor 3 (EPR3) ectodomain.

### Materials and Methods

Production of L. japonicus EPR3 Ectodomain Protein

Expression and purification of L. japonicus ecotype Gifu EPR3 ectodomain (ED) was performed as described previously (Kawaharada, Y et al. Nature 2015 523: 308-312). In brief, DNA encoding residues 33-232 of EPR3 (corresponding to the EPR3 ED) containing an N-terminal gp67 secretion signal and a C-terminal 6×His-tag was codon-optimized for insect cell expression (GenScript) and inserted into the pOET2 vector (Oxford Expression Technologies). Baculoviruses, used for infecting Sf9 cells cultured in suspension in serum-free HyClone SFX-Insect medium (FisherScientific), were obtained using the flashBAC GOLD system (OET). Five days post inoculation, the media was dialyzed against buffer containing 50 mM Tris-HCl pH 8.0 and 200 mM NaCl before centrifugation and loaded on a HisTrap excel affinity column (GE Healthcare). The eluted protein was dialyzed against buffer containing 50 mM Tris-HCl pH 8.0 and 200 mM NaCl, and further purified on a HisTrap HP affinity column (GE Healthcare). For crystallization, the EPR3 ED was treated with PNGase F (1:15 w/w ratio) for 1 hour at room temperature and overnight at 4° C. to remove N-linked oligosaccharides. EPR3 ED was then purified on a Mono S 5/50 column (GE Healthcare) and eluted with a linear gradient of 50-300 mM NaCl and 50 mM Tris-HCl, pH 7.0. Both glycosylated and de-glycosylated EPR3 ED were finally purified on a Superdex 75 10/300 column (GE Healthcare) in gel filtration buffer containing 50 mM KH2-PO4 pH 7.8 and 200 mM NaCl (for microscale thermophoresis binding experiments) or 50 mM Tris-HCl pH 8.0 and 200 mM NaCl (for crystallization).

### Nanobody Production

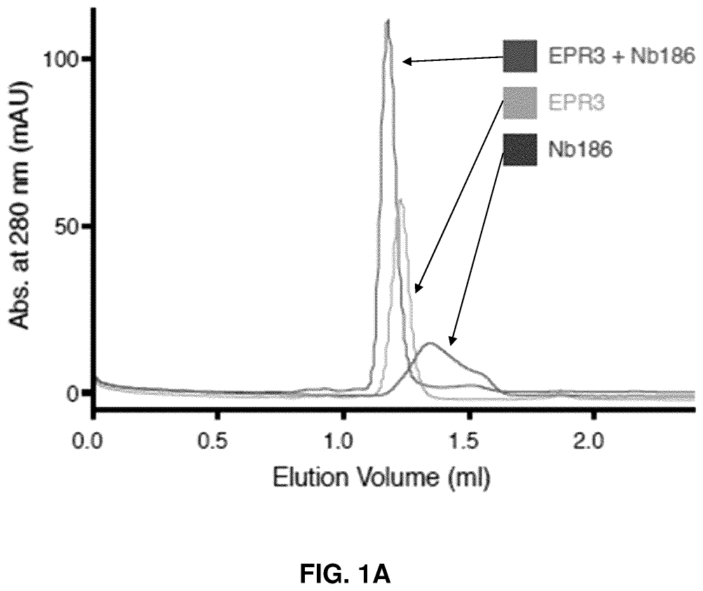

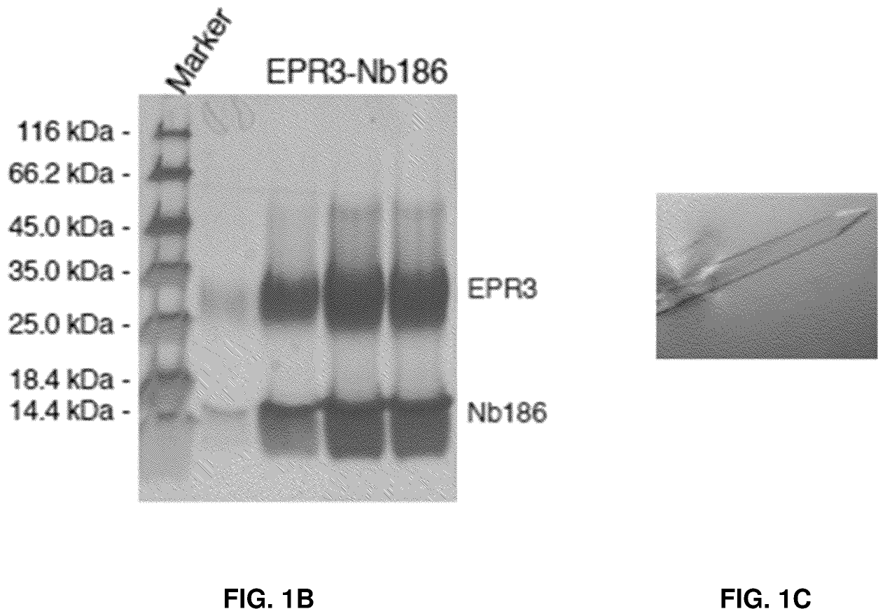

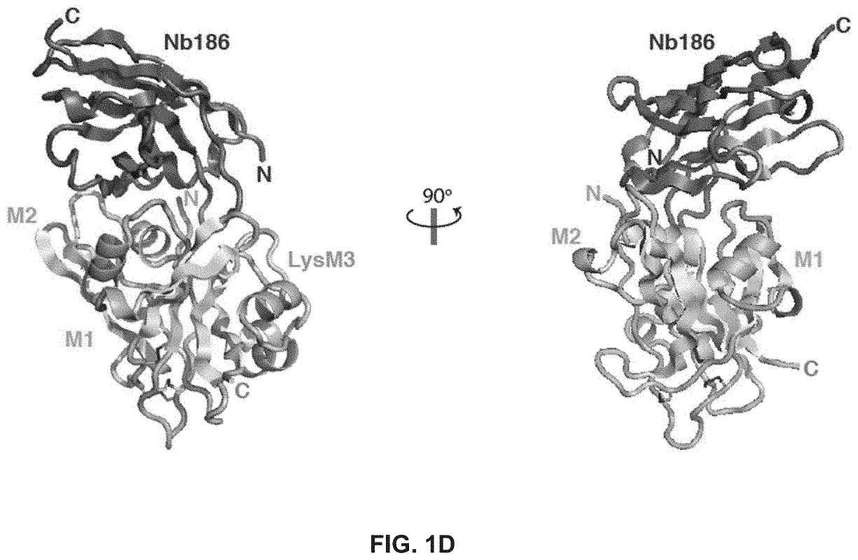

A llama (Lama glama) was immunized four times with 100 μg of purified EPR3 ED. From a blood sample, peripheral blood lymphocytes were isolated and RNA was extracted using RNase Plus Mini Kit (Qiagen). Total cDNA was generated using the Superscript III First-Strand Kit (Invitrogen) with random hexamer primers. The coding regions of the nanobodies (Nbs) were amplified by PCR and inserted into a phagemid vector backbone where the Nbs were C-terminally fused to an E-tag followed by the pIII coat protein. VCSM13 helper phage was used for generating the final M13 phage display Nb library. For selection, EPR3 ED was biotinylated via primary amine coupling using the Chromalink NHS labelling system (Solulink) and 20 μg EPR3 antigen was added to 100 μl MyOne Streptavidin T1 Dynabeads (Thermo Fisher Scientific) in PBS supplemented with 2% BSA. M13 phage particles (2.5×10¹³) were added and incubated with EPR3 coated Dynabeads for 1 hour before 15 wash steps with 1 ml PBS containing 0.1% Tween 20. Phages were eluted by incubating the beads with 0.2 M glycine pH 2.2 for 15 min. The eluted phage particles were amplified and used in a second round of phage display where a reduced amount of EPR3 ED antigen (2 μg) and fewer M13 phage particles (2.5×10¹²) were used. After two rounds of phage display selections, single colonies were picked and grown in LB media in a 96-well plate format for 6 hours before Nb expression was induced with 0.8 mM IPTG overnight at 30° C. The 96-well plate was centrifuged and 50 μl of the supernatant were transferred to an EPR3 ED-coated ELISA plate prepared by coating each well with 0.1 μg EPR3 ED and by blocking with PBS containing 0.1% Tween 20 and 2% BSA. After addition of the supernatant, the EPR3 ED-coated ELISA plate was incubated for 1 hour and then washed six times in PBS with 0.1% Tween 20 before anti-E-tag-HPR antibody (Bethyl) was added at a 1:10,000 dilution. The plate was incubated for 1 hour, washed and developed with 3,3′,5,5′-tetramethylbenzidine. The reaction was quenched with 1 M HCl and the absorbance was measured at 450 nm. Phagemids from positive clones were isolated, sequenced and the encoding DNA were cloned into the pET22b(+)(Novagen) for bacterial expression. Nb186 was expressed in E. coli LOBSTR cells (Andersen, K. R. et al. Proteins 2013 81: 1857-1861) that were grown to an optical density of 0.6 at 600 nm before protein expression was induced with 0.2 mM IPTG at 18° C. overnight. Cells were lysed in buffer containing 50 mM Tris-HCl pH 8.0, 500 mM NaCl, 20 mM imidazole and 1 mM benzamidine, and the cleared supernatant was loaded onto a Ni Sepharose 6 FF affinity column (GE Healthcare) and washed prior to elution in lysis buffer supplemented with 500 mM imidazole. Nb186 was finally purified on a Superdex 75 10/300 gel filtration column (GE Healthcare) in gel filtration buffer containing 50 mM Tris-HCl pH 8.0 and 200 mM NaCl. Complex formation between EPR3 ED and Nb186 was analyzed on an analytic Superdex 75 Increase 3.2/300 column. The high-affinity nanobody Nb186 formed a tight complex with EPR3 ED as demonstrated by a mobility shift in gel filtration (FIG. 1A). FIG. 1B shows the isolation of the co-purified EPR3 ED-Nb186 complex.

### Crystallization and Structure Determination

Purified de-glycosylated EPR3 ED and Nb186 was mixed in a 1:1.1 molar ratio and incubated on ice for 1 hour before purification on a Superdex 75 10/300 column. The peak fractions containing the EPR3 ED-Nb186 complex were pooled and concentrated on a VivaSpin filter (Sartorius) to 5-8 mg/ml and crystallized using the vapor diffusion method by mixing an equal volume of protein and reservoir solution (18% 2-propanol, 0.1 M Sodium Citrate pH 5.5 and 20% PEG 4000). Crystals were cryo-protected in mother liquor with the addition of 20% ethylene glycol before being flash-frozen in liquid nitrogen.

Diffraction data was measured at DESY PX14 beamline at a wavelength of 0.9763 Å and data reduction was performed in XDS (Kabsch, W. XDS. Acta Crystallographica Section D Biological Crystallography 2010 66:125-132). A molecular replacement solution was found with phenix.phaser (McCoy, A. J. et al. Journal of Applied Crystallography 2007 40: 658-674) using a homology model of Nb186 generated with Phyre2 (Kelley, L. A. et al. Nat Protoc 2015 10: 845-858) truncated of its complementarity-determining regions (CDRs). In a second molecular replacement search a homology model of EPR3 generated with Phyre2 and truncated of high b-factor region based on CERKI structure (PDB entry 4EBZ) was placed. The structure of the EPR3-Nb186 complex was built in Coot (Emsley, P. et al. Acta Crystallographica Section D Biological Crystallography 2010 66:486-501) and coordinates and temperature factors were refined using phenix.refine (Adams, P. D. et al. Acta Crystallographica Section D Biological Crystallography 2010 66: 213-221). The final model contained residues 1-119 of Nb186 and residues 36-216 of EPR3 with 98% of the protein residues in the favored region and none in the disallowed region of the Ramachandran plot. The figures were prepared with PyMOL and data and refinement statistics are summarized in Table 1. The co-structure of the EPR3 ED-Nb186 complex was determined from a well-diffracting crystal (FIG. 1C) and refined with data extending to 1.9 Å resolution (FIG. 1D and Table 1).

TABLE 1
Data collection and refinement statistics. Data was
collected from one crystal, and values in parentheses
(denoted with *) are for highest-resolution shell.
EPR3 data collection

| Space group | P 1 21 1 | |
|---|---|---|
| Cell dimensions | 59.82, 36.08, 72.66 | |
| a, b, c (Å) | 90.00, 93.92, 90.00 | |
| α, β, γ (°) | | |
| Resolution (Å) | 47.7-1.87 | (1.94-1.87)* |
| R_{merge} (%) | 8.7 | (82.2) |
| I/σI | 15.96 | (1.94) |
| Completeness (%) | 96.9 | (81.1) |
| Redundancy | 6.3 | (4.3) |

EPR3 refinement

| Resolution (Å) | 47.7-1.87 | (1.94-1.87)* |
|---|---|---|
| No. reflections | 25176 | (2151) |
| R_{work}/R_{free} (%) | 17.42/21.72 | |
| No. atoms | | |
| Protein | 2337 | |
| Glycosylation | 14 | |
| Water | 134 | |
| B-factors (Å²) | | |
| Protein | 42.48 | |
| Glycosylation | 135.82 | |
| Water | 41.35 | |
| R.m.s. deviations | | |

| | |
|---|---|
| Bond lengths (Å) | 0.006 |
| Bond angles (°) | 0.83 |

### Modelling

De novo modelling of the M1 domain of L. japonicus EPR3 and EPR3 homologs (corresponding to residues 56-99 to in L. japonicus EPR3) was performed using atomic-level knowledge-based force field simulations (Xu, D. et al. Proteins 2012 80: 1715-1735).

### Small-Angle X-Ray Scattering (SAXS)

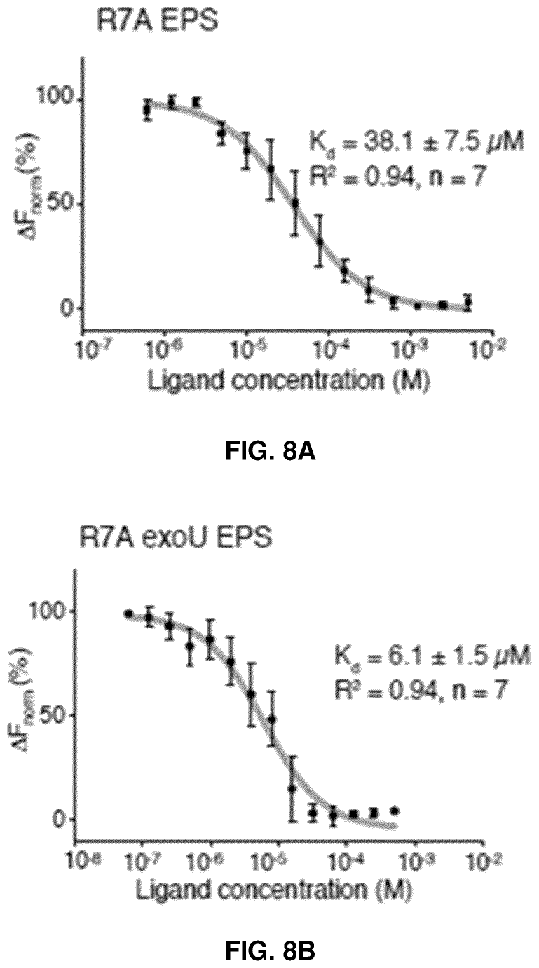

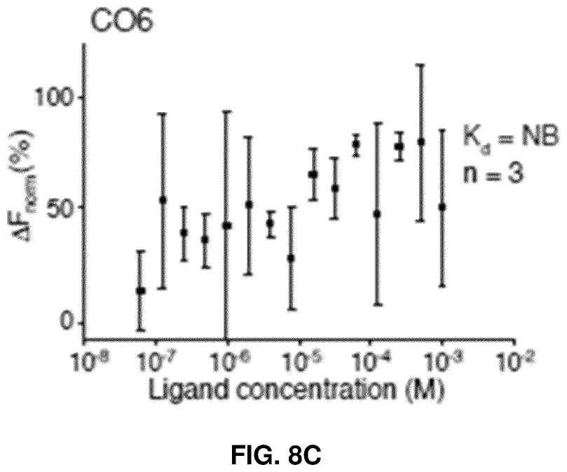

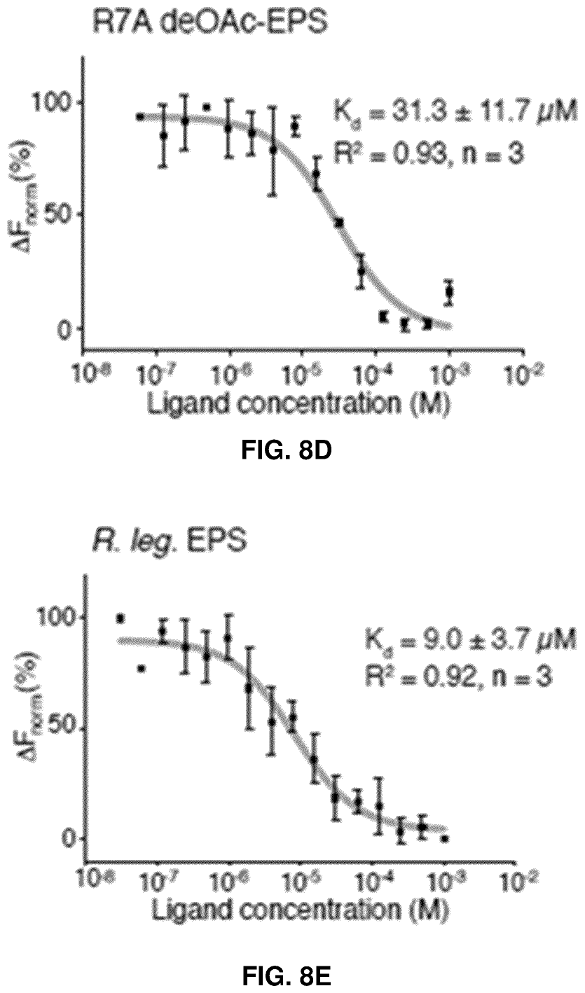

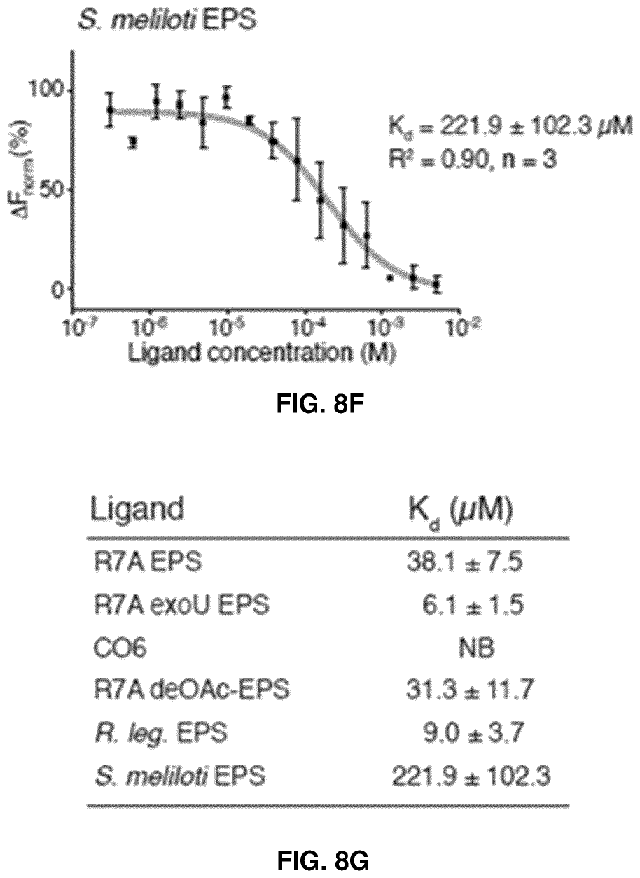

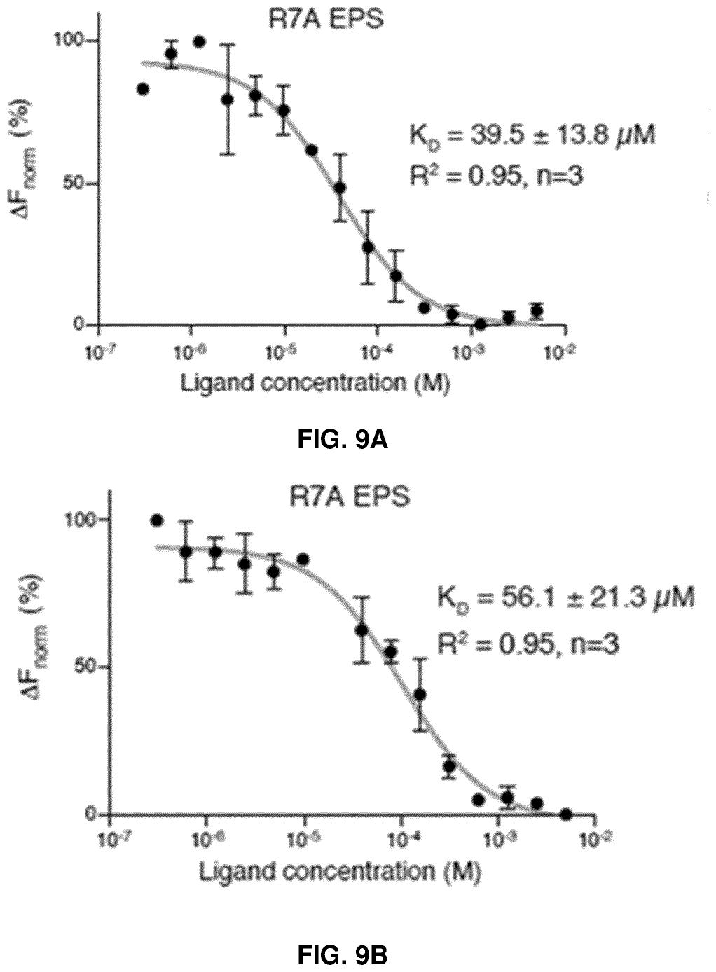

EPR3 ED was purified by gel filtration in gel filtration buffer and a monodisperse peak fraction was collected and used for SAXS measurements. Scattering from EPR3 ED samples (either with no ligand or with R7A EPS (1 mM) or R7A exoU EPS (1 mM) added) at concentrations ranging from 0.6-22.0 mg/ml (multiple technical replicates at different concentrations) were collected at the EMBL P12 beamline PETRA III in a temperature-controlled cell (20° C.) at a wavelength of 1.24 Å. Normalization, radial averaging and buffer subtractions were done at the beamline using the automated pipeline. Data analysis and ab initio low resolution modelling were performed in DAMMIN (Svergun, D. I. Biophysical Journal 1999 76: 2879-2886). The scattering, Guinier plots and pair distance distribution plots were prepared with the GraphPad Prism 7 software (FIGS. 4A-4C, FIGS. 8A-8G).

### Results

### Crystal Structure of the EPR3 Ectodomain

To understand the basis for EPS perception, the L. japonicus EPR3 ectodomain (hereafter referred to as EPR3 ED) was expressed in insect cells, purified and crystallized with the help of llama-derived miniature antibodies (nanobodies) targeting EPR3 ED (Bukowska, M. A. & Grater, M. G. Current Opinion in Structural Biology 2013 23: 409-416; Hansen, S. B. et al. Acta Crystallographica D Structural Biology 2017 73: 804-813). The overall structure of EPR3 ED was found to contain three interconnected domains (M1, M2 and LysM3) arranged in a cloverleaf-shape stabilized by three internal disulfide bridges (FIG. 2A). Interestingly, the crystal structure of the EPR3 ED revealed a novel fold of the M1 domain that was structurally unique. M1 is composed of only one α-helix and three elongated β-strands (NM topology). The exterior β2-strand was stabilized by seven backbone hydrogen bonds to the adjacent β3-strand, which gave M1 an overall βαββ arrangement where the three β-strands formed an extended anti-parallel β-sheet (FIG. 2B). The M2 domain of EPR3 ED was also unusual as it contained βαβ fold and lacked the defined second α-helix compared to a classical LysM domain (FIG. 2C). LysM3 had the LysM βααβ fold, with a root-mean-square deviation (RMSD) of 1.2 Å to the LysM3 domain in the L. japonicus chitin receptor CERK613 (FIG. 2D). Intriguingly, a search in the Protein Data Bank (PDB) revealed that M1 in EPR3 ED had no close structural homologs, while M2 was associated with LysM structures, and LysM3 was classified as a standard LysM motif (Holm, L. & Rosenström, P. Nucleic Acids Research 2010 38:W545-9; Krissinel, E. & Henrick, K. in Computational Life Sciences (Springer Berlin Heidelberg) 2005 3695: 67-78).

### Structure of the EPR3 C-terminus

X-ray scattering (SAXS) experiments were performed to determine the structure of the C-terminus of EPR3 ED and to validate the EPR3 ED structure in solution (FIGS. 3A-3C and Table 2).

TABLE 2
SAXS data collection and scattering-derived parameters

| | | |
|---|---|---|
| | EPR3 + R7A | EPR3 + R7A |
| EPR3 | EPS | exoU EPS |

Data collection

| | | | |
|---|---|---|---|
| Beamline | PETRA III P12 | PETRA III P12 | PETRA III P12 |
| Wavelength (nm) | 1.24 | 1.24 | 1.24 |
| s range (nm⁻¹) | 0.0531-3.5526 | 0.1693-3.5996 | 0.0780-3.6134 |
| Exposure time (s) | 0.045 | 0.045 | 0.045 |
| Concentration range (mg/ml) | 0.6-22 | 0.6-22 | 0.6-22 |
| Ligand concentration (M) | N/A | 0.001 | 0.001 |
| Temperature (° C.) | 20 | 20 | 20 |

Structural parameters

| | | | |
|---|---|---|---|
| Rg (nm) from Guinier | 2.25 ± 0.6 | 2.21 ± 0.14 | 2.22 ± 0.25 |
| Rg (nm) from P(r) plot | 2.25 ± 0.02 | 2.18 ± 0.02 | 2.21 ± 0.02 |
| Dₘₐₓ (nm) | 7.34 | 6.73 | 7.27 |
| Porod volume estimate (Å³) | 52.6 | 49.8 | 50.7 |
| Estimated molecular mass from | 31.0 | 29.3 | 29.8 |
| Porod volume (kDa) | | | |
| Estimated molecular mass from | 24.7 | 24.7 | 24.7 |
| forward scattering I(0) (kDa) | | | |
| Calculated molecular mass from | 23.6 | 23.6 | 23.6 |
| amino acid sequence (kDa)* | | | |
| Estimated mass of glycosylated | 25-35 | 25-35 | 25-35 |
| EPR3 from SDS-PAGE (kDa) | | | |

Software employed

| | |
|---|---|
| Primary data reduction | Primus |
| Ab initio analysis | DAMMIF/DAMMIN |
| Rigid body modeling | Situs/colours |
| Visualization | PyMOL |

Surprisingly, the distance distribution showed that EPR3 ED in solution was almost twice the length of the crystal structure, but maintained the same molecular weight (FIG. 3C). The low-resolution SAXS envelope revealed a globular volume with a protruding stem-like structure at one end (FIG. 3D). The crystal structure of EPR3 ED accommodated into the globular part of the envelope and the missing C-terminal residues were modelled with good molecular fits into the stem-like structure (FIG. 3A, FIG. 3D). Interestingly, the EPR3 ED stem-like structure was well-defined in solution, similar to what was observed for NFP (unpublished data). The stem region shows conservation among EPR3 homologues both in terms of length and composition, which is dominated by glycine and positively charged lysine and arginine residues (FIG. 3F). Without wishing to be bound by theory, these results suggested that these receptors were positioned with a spacer to the plasma membrane potential important for efficient signaling (FIG. 3E).

### Structurally Unique EPR3 ED M1 Domain Defines a New Class of Receptors

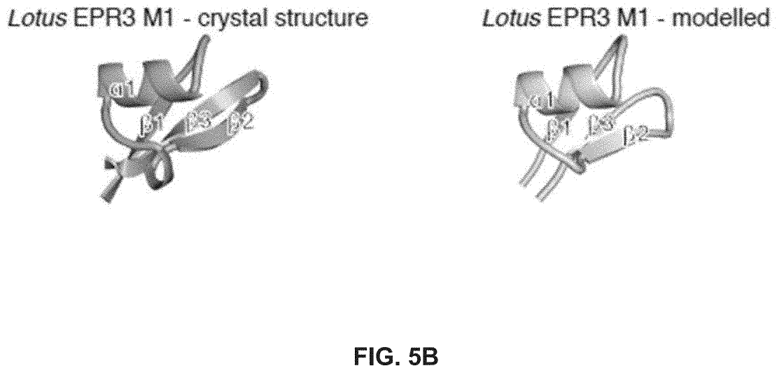

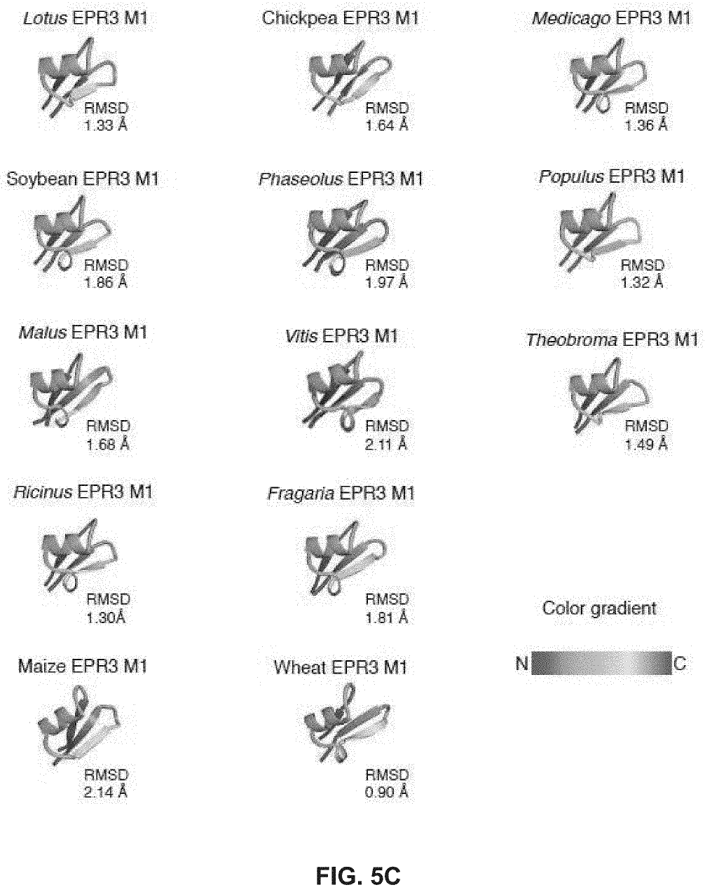

The primary sequence and secondary structure of EPR3 ED, with unique N-terminal M1 (βαβ) and atypical M2 (βαβ) folds, followed by a classical LysM3 domain (βααβ) was highly conserved and defined a novel class of receptors (FIGS. 4A-4C, FIGS. 5A-5C). This class of receptors was not only restricted to legumes, but was also present in non-legume and monocot plants (FIGS. 4A-4C), suggesting that surveillance of EPS, or other microbial surface polysaccharides, was a widely conserved plant trait. To solidify this observation, the small (˜43 residue) M1 domain was modelled in the EPR3 homologs using atomic-level force field simulations. The related receptors shared the same topology and βαββ fold, and all modelled domains superpositioned extraordinarily well with the M1 domain in L. japonicus EPR3 (FIGS. 5A-5C). M1 of these receptors formed a surface exposed β-sheet structurally different from all known carbohydrate-binding domains identified in nature so far (Hashimoto, H. Cell. Mol. Lift. Sci. 2006 63: 2954-2967). As shown in FIG. 6, the comparison to the chitin receptor CERK6 ectodomain and the LCO receptor NFP ectodomain clearly indicated that EPR3 ED defined a distinct class of receptors.

Together, these results demonstrated that the M1-M2-LysM3 configuration of EPR3 ED defined a conserved class of new receptors that ware evolutionarily distinct from the chitin and LCO LysM receptors.

### Example 2: Determining the Specificity of EPR3

The following example describes microscale thermophoresis (MST) experiments measuring the ability of EPR3 to bind and distinguish between EPS of different structure and composition in solution.

### Materials and Methods

### Characterization of Exopolysaccharides (EPS) Ligands

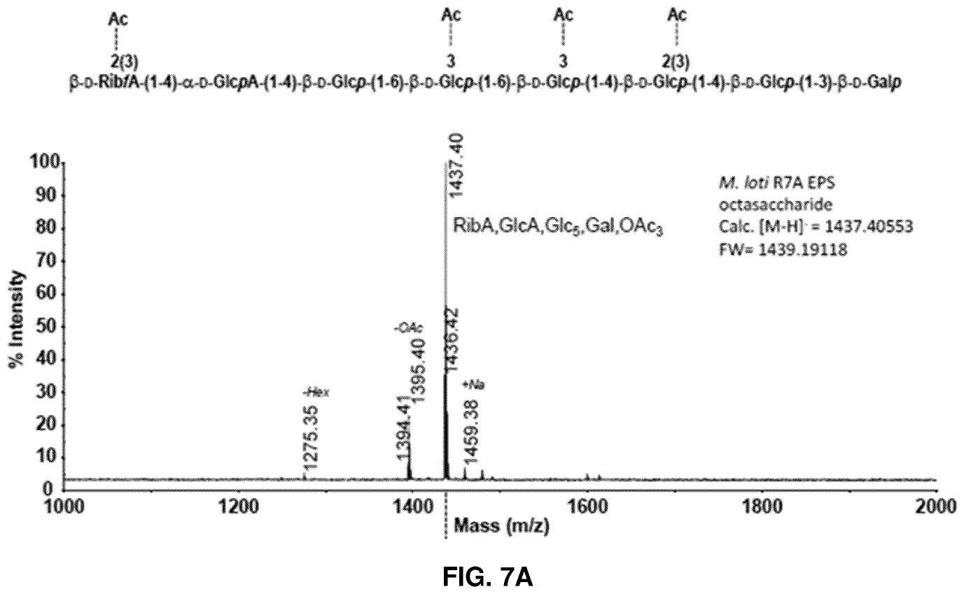

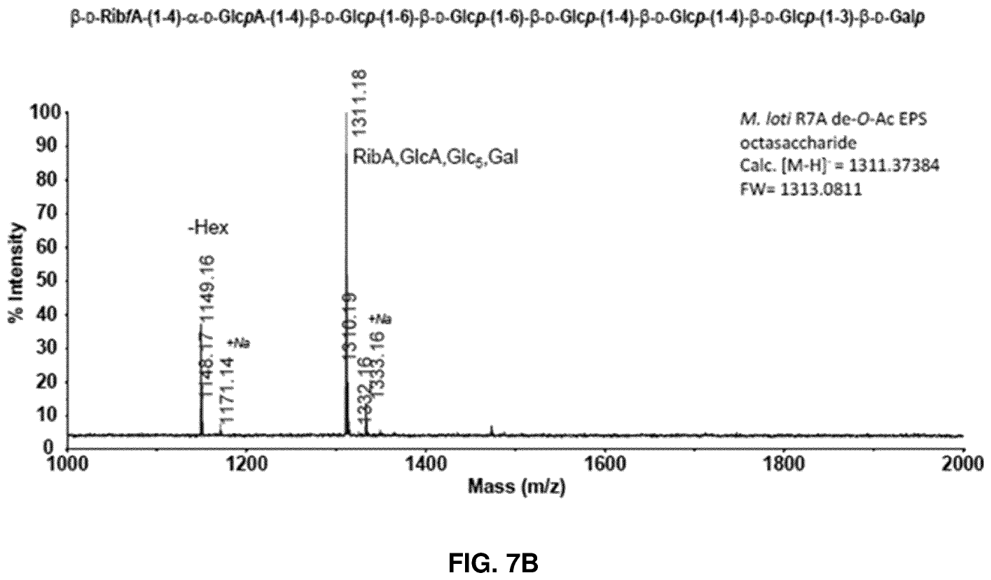

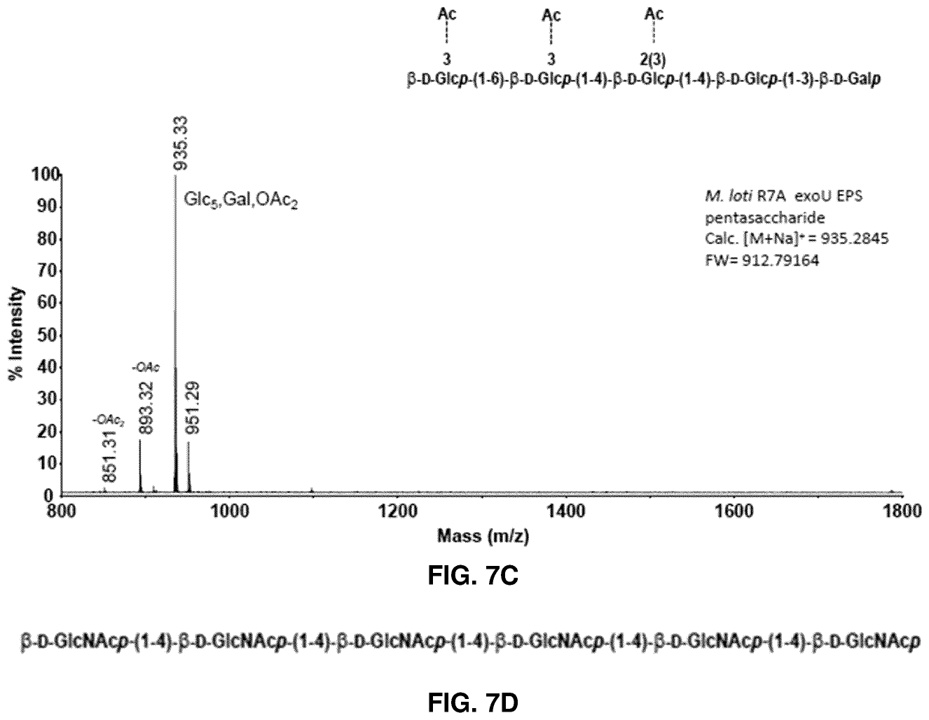

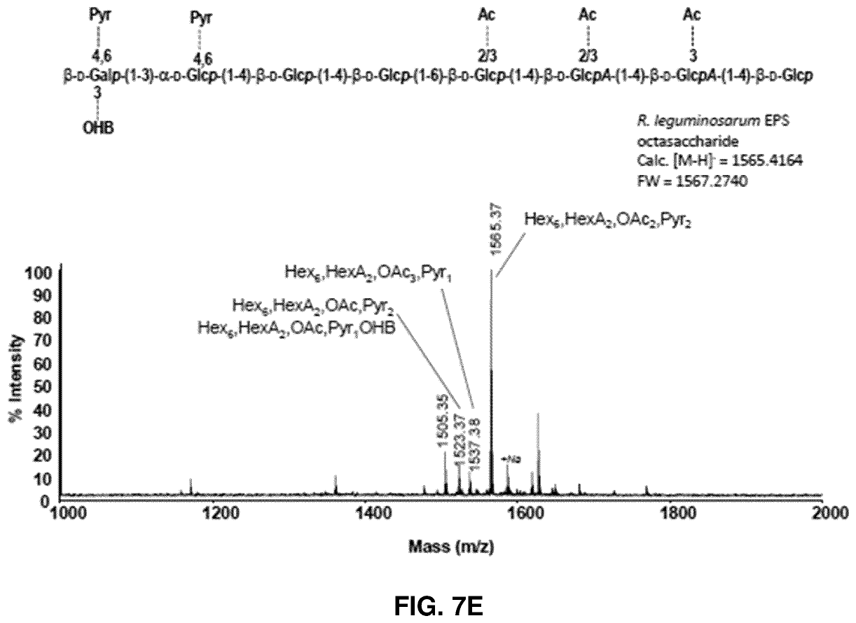

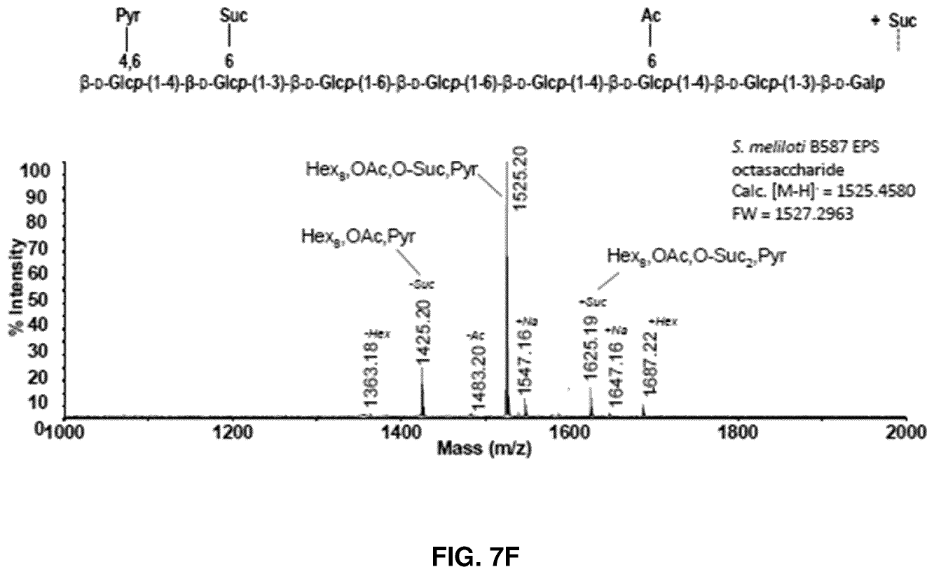

Low molecular mass (LMM) exopolysaccharides (EPS) were isolated from various rhizobial strains including Mesorhizobium loti strain R7A ndvB6, R. leguminosarum bv. viciae 3855 (this work) and Sinorhizobium meliloti B578 (Griffins, J. S. et al. Mol. Microbiol. 2008 69: 479-490) that were deficient in cyclic glucan production were grown on minimal media with glucose as the sole source of carbon. The LMM EPS was isolated from the bacterial culture supernatants and purified via sequential precipitation with 6 volumes of 99.8% EtOH (v/v), followed by 9 volumes EtOH (v/v) and purified by size exclusion chromatography (SEC) as previously described (Muszyński, A. et al. J. Biol. Chem. 2016 291: 20946-20961). O-acetyl groups were removed chemically by mild overnight treatment of native EPS samples with 12.5% NH₄OH (Muszyński, A. et al. J. Biol. Chem. 2016 291: 20946-20961). Native and de-O-acetylated samples were verified via matrix-assisted laser desorption/ionization time of flight mass spectrometry (MALDI-TOF MS) analysis on Applied Biosystems AB SCIEX TOF/TOF 5800 system in either negative or positive reflector ionization modes. The glycosyl composition and linkage was determined as previously described (Muszyński, A. et al. J. Biol. Chem. 2016 291: 20946-20961). Proposed structures of the EPS ligands and results of MALDI-TOF MS are shown in FIGS. 7A-7F.

Native and de-O-acetylated R7A EPS

It was previously demonstrated that M. loti strain R7A produces a LMM EPS that is structurally similar to high-molecular mass EPS polymer, and is an O-acetylated octasaccharide with the structure (2,3/3-OAc)β-D-RibfA-(1→4)-a-D-GlcpA-(1→4)-β-D-Glcp-(1→6)-(3OAc)β-D-Glcp-(1→6)-(2OAc)β-D-Glcp-(1→4)-(2/3OAc)β-D-Glcp-(1→4)-β-D-Glcp-(1→3)-β-D-Galp, and the average molecule is substituted with three O-acetyl groups at four glycosyl residues in a non-stoichiometric ratio (Muszyński, A. et al. J. Biol. Chem. 2016 291: 20946-20961). The experiment was repeated, showing similar structural properties of the isolated LMM EPS. In particular, MALDI TOF-MS analysis confirmed that the average molecular [M-H]⁻ mass of the R7ΔAndvB EPS was m/z 1437.40, consistent with RibAGlcAGlc₅GalOAc₃ (FIG. 7A). De-O-acetylation of the wild-type native R7AAndvB EPS resulted in a shift of molecular mass from m/z 1437.40 to m/z 1311.18. This is consistent with loss of all 3 O-acetyl groups from RibAGlcAGlc5Gal octasaccharide (FIG. 7B).

R7A exoU EPS

In addition, low molecular mass oligosaccharides from the supernatant of a minimal media culture of the exoU mutant of M. loti strain R7A were isolated. This strain was described as defective in the expression of the putative Glc transferase involved in the addition of sixth hexose in the biosynthesis of the R7A EPS precursor (Kelly, S. J. et al. Mol. Plant Microbe Interact. 2013 26: 319-329). Composition and glycosyl linkage analysis showed the presence of 3-linked Galp, 4-linked Glcp, 6-linked GlcA, and terminally linked Glcp. Positive ionization mode MALDI-TOF MS analysis demonstrated major [M+Na]⁺ ion at m/z 935.33 that likely corresponds to the Glc₄Gal pentasaccharide substituted non-stoichiometrically with two O-acetyl groups out of three possible acetylation sites (FIG. 7C).

### Chitohexose (CO6)

CO6 were Obtained from Megazyme (FIG. 7D).

R. leguminosarum EPS

An ndvB mutant of Rhizobium leguminosarum bv. viciae 3855 was constructed by insertion of a suicide vector into the ndvB gene as previously described (Kelly, S. J. et al. Mol. Plant Microbe Interact. 2013 26: 319-329). Native R. leguminosarum 3855 ndvB EPS SEC purification of 9 volumes EtOH precipitated EPS yielded one major low molecular mass fraction (LMM EPS). R. leguminosarum 3855 (this work) R. leguminosarum bv. viciae 3855, produces EPS in an octasaccharide polymer consisting of five D-glucose, two D-glucuronic acid, and one D-galactose residues substituted with three 2-O-acetyl (or 3-O-acetyl), two 4,6-pyruvyl and one hydroxybutanoyl group (Philip-Hollingsworth, S. et al. J. Biol. Chem. 1989 264: 5710-5714; Robertsen, B. K. et al. Plant Physiol. 1981 67: 5710-5714; O'Neil, M. A. et al. J. Biol. Chem 1991 266: 9549-9555). Composition and glycosyl linkage analysis indicated the presence of 4-linked Glcp, 6-linked Glcp, 4-linked GlcpA, 4,6-linked Glcp, 4,6-linked Galp 3,4,6-linked Glcp (all branching sugars likely due to 4,6 substitution with pyruvate), and terminally linked Glcp. Negative ionization mode MALDI-TOF MS analysis demonstrated a heterogeneous mixture of Hex₆HexA₂ octasaccharide with a different number of non-carbohydrate substituents, and major [M-H]⁻ ion at m/z 1656.37, likely due to the fact that octasaccharide was substituted with two O-acetyl and two 4,6-pyruvyl groups. The structures substituted with hydroxybutanoate were also detected, but these are not major moieties (FIG. 7E).

S. meliloti EPS

SEC purification of precipitated S. meliloti EPS yielded one major low molecular mass fraction (LMM EPS). S. meliloti B587 is an ndvB mutant of Rm1021 that is proposed to be deficient in cyclic glucan production while producing normal EPS (Griffitts, J. S. et al. Mol. Microbiol. 2008 69: 479-490). The Rm1021 EPS (succinoglycan) or EPS I is an octasaccharide polymer consisting of seven D-glucose and one D-galactose residues substituted with 6-O-succinyl, 6-O-acetyl, and 4,6-puryvyl groups (Reinhold, B. B. et al. Journal of Bacteriology 1994 176: 1997-2002; Choulry, C. et al. Int. J Bio. Macromol. 1995 17:357-363; Wang, L. X. et al. Journal of Bacteriology 1999 181: 6788-6796). Composition and glycosyl linkage analysis indicated the presence of 3-linked Galp; 4-linked Glcp; 6-linked Glcp; 3-linked Glcp, 4.6-linked Glcp (likely due to 4,6 substitution with pyruvate). Consistent with early reports (Griffitts, J. S. et al. Mol. Microbiol. 2008 69: 479-490), no 2-linked glucose was detected, confirming there was no cyclic glucan production. Negative ionization mode matrix-assisted laser desorption/ionization time of flight mass spectrometry (MALDI-TOF MS) analysis indicated major [M-H]⁻ ion at m/z 1525.20. This ion corresponds to octasaccharide composed of eight hexose residues substituted with O-acetatyl, 4,6-pyruvyl and succinyl groups (Hex8OAcOSucPyr) (FIG. 7F).

### Microscale Thermophoresis (MST) Binding Experiments

Purified EPR3 was fluorescently labeled using the Monolith NT.115TM Protein Labelling Kit Blue NHS (NanoTemper Technologies) according to the manufacturer's instructions. All experiments were performed in MST buffer (50 mM K2PO4, pH 7.8, 500 mM NaCl, and 0.05% Tween-20) with a constant concentration of EPR3 ED (100 nM and ˜50% labelling efficiency) and dilution series of the various ligands. The samples were incubated for 30 minutes at room temperature before loaded into standard capillaries for measurements on a Monolith NT.115 TM instrument (NanoTemper Technologies) at 25° C., with blue LED power of 50% and MST power of 20%. To accurately measure the experimental errors and ensure data reproducibility, all MST binding experiments were performed with at least three independently purified samples of EPR3. At the highest ligand concentrations, weak ligand binding to the fluorescent label itself was occasionally observed. To accurately account for this unspecific binding ligand binding to 50 nM free fluorescent label was measured and this small background was subtracted contribution from all the respective MST binding measurements. The competition experiments were performed by pre-incubating labeled EPR3 ED with 250 μM R7A EPS or R7A exoU EPS and assessing the binding of EPR3 ED to titrated R7A exoU EPS or R7A EPS, respectively. Binding data were processed with the GraphPad Prism 7 software (GraphPad Software, Inc.) and the equilibrium dissociation constants (K_{d}) values (95% confidence interval) were calculated using the sigmoidal dose-response equations.

### Small-Angle X-Ray Scattering (SAXS)

EPR3 ED was purified by gel filtration in gel filtration buffer and a monodisperse peak fraction was collected and used for SAXS measurements. Scattering from EPR3 ED samples (either with no ligand or with R7A EPS (1 mM) or R7A exoU EPS (1 mM) added) at concentrations ranging from 0.6-22.0 mg/ml were collected at the EMBL P12 beamline PETRA III in a temperature-controlled cell (20° C.) at a wavelength of 1.24 Å. Normalization, radial averaging and buffer subtractions were done at the beamline using the automated pipeline. Data analysis and ab initio low resolution modelling were performed in DAMMIN (Svergun, D. I. Biophysical Journal 1999 76: 2879-2886). The scattering, Guinier plots and P(r) distance distribution plots were prepared with the GraphPad Prism 7 software. To detect if ligand binding affected EPR3 ED oligomerization as a potential signaling mechanism, the solution SAXS of EPR3 ED in the R7A EPS or R7A exoU EPS-loaded state were solved.

### Results

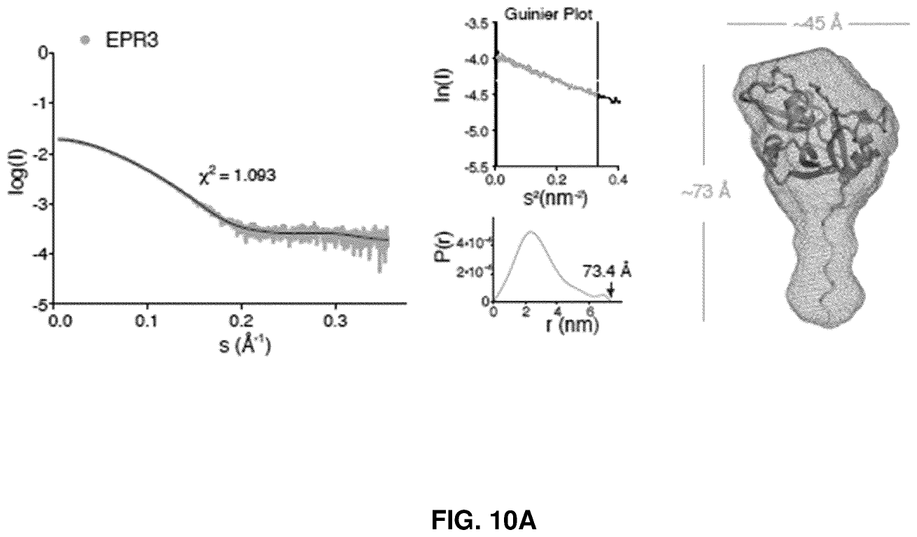

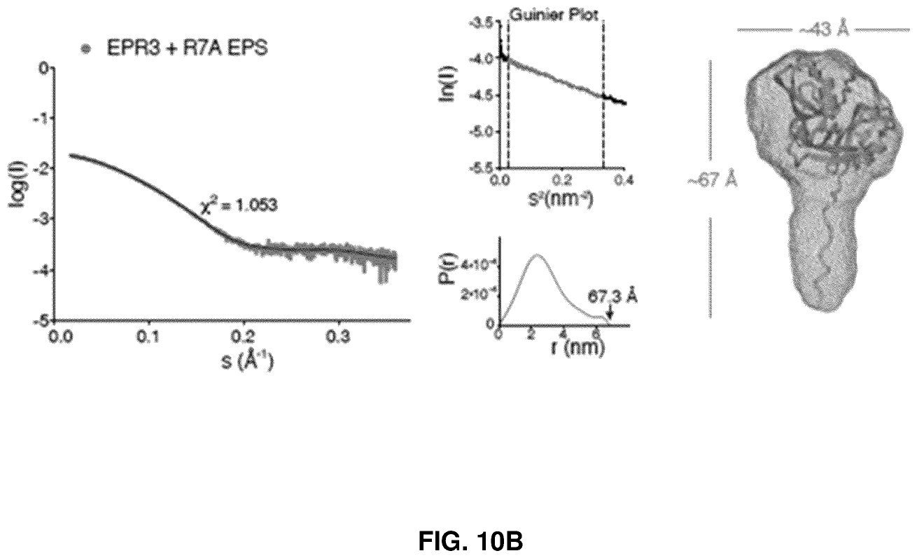

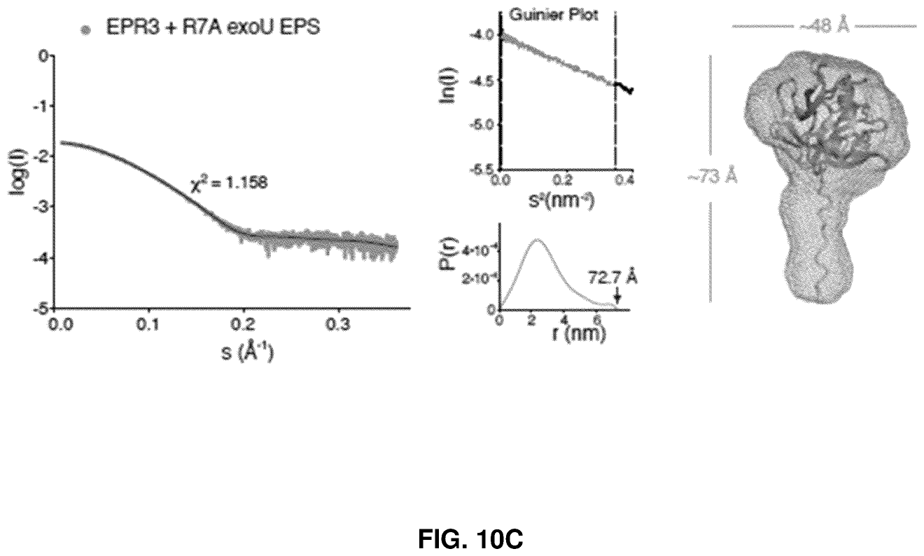

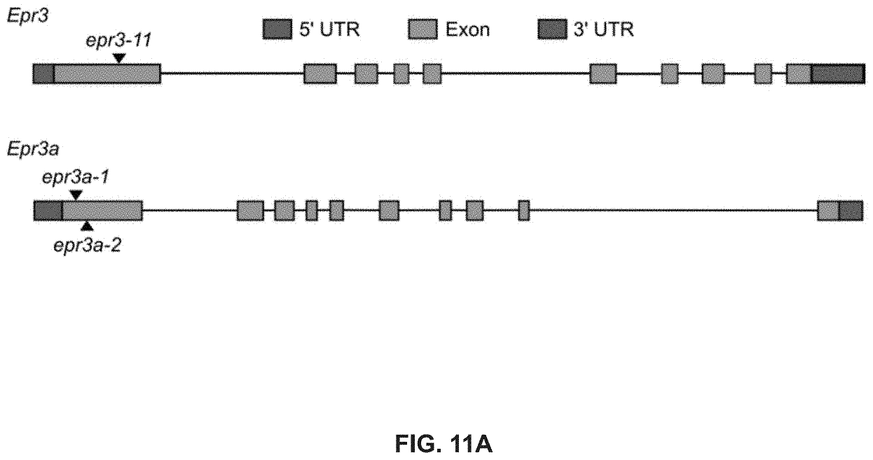

EPR3 ED bound the monomeric octasaccharide R7A EPS (from the rhizobium species M. loti, a species compatible with L. japonicus for root nodule formation) with an equilibrium dissociation constant (K_{d}) of 38.1±7.5 μM (FIGS. 8A and 10A). Interestingly, EPR3 ED also bound the incompatible truncated R7A exoU EPS pentasaccharide with even 6-fold higher affinity (K_{d} of 6.1±1.5 μM) compared to R7A EPS (FIG. 8B).

To detect if ligand binding affected EPR3 ED oligomerization as a potential signaling mechanism, the solution SAXS of EPR3 ED in the presence of R7A EPS or R7A exoU EPS-loaded state was solved. The scattering measurements and ab-initio reconstructions showed that the ligand-bound receptor retained its monomeric state with the same overall structure, dimensions and stem arrangement as the ligand-free state (FIGS. 10A-10C and Table 2). Together, the data showed that EPR3 ED was able to bind both compatible and incompatible EPS while maintaining a monomeric structure. Without wishing to be bound by theory, this suggested that the observed phenotype of impaired infections in L. japonicus, alfalfa, and pea inoculated with EPS-defective bacterial symbionts related to the ligand-induced ability of the receptor to activate downstream signaling.

To investigate ligand-selectivity, it was assessed whether EPR3 ED bound the immune-response-inducing chitin polymers (C06) known to be perceived by canonical LysM receptors (Bozsoki, Z. et al. Proc. Natl. Acad. Sci. U.S.A. 2017 114: E8118-E8127; Liu, T. et al. Science 2012 336: 1160-1164; Liu, S. et al. Structure 2016 24: 1192-1200). Results showed that EPR3 ED was unable to bind C06 (FIG. 8C), supporting the structural data that EPR3 belonged to a unique class of receptors. The N-acetyl groups of chitin polymers have been demonstrated to be important contact points for LysM proteins (Liu, T. et al. Science 2012 336: 1160-1164; Hayafune, M. et al. Proc. Natl. Acad. Sci. U.S.A. 2014 111: E404-13; Wong, J. E. M. M. et al. FEBSJ 2014 281:1196-1208; Sanchez-Vallet, A. et al. Elife 2013 2: e00790) and therefore whether corresponding O-acetyl groups in EPS were important moieties recognized by EPR3 ED was investigated. However, chemical removal of the O-acetyl groups in R7A EPS (deOAc-EPS) did not affect binding, as EPR3 ED bound R7A deOAc-EPS with a K_{d} of 31.3±11.7 μM, similar to that of fully O-acetylated R7A EPS (FIG. 8D). This suggested a difference in the ligand perception mechanism between EPR3 ED and classical LysM receptors binding chitinous ligands, e.g. AtCERK1 (Liu, T. et al. Science 2012 336: 1160-1164). In the crystal structure of AtCERK1, the position of chitin in the LysM2 binding pocket allowed the carbonyl oxygen of the N-acetyl moieties to form hydrogen bonds with backbone-amide-nitrogens of the main chain (Liu, T. et al. Science 2012 336: 1160-1164). Such rigorous recognition was unlikely for the O-acetyl groups in EPS as EPS is non-stoichiometrically O-acetylated, in contrast to chitin, which has a uniform distribution of N-acetyl groups.

Supporting this notion, monomeric octasaccharide EPS from both R. leguminosarum and S. meliloti that have different O-acetylation patterns were purified and characterized (FIGS. 7A-7F). These rhizobia do not normally infect L. japonicus but interestingly, EPR3 ED still bound R. leguminosarum octasaccharide EPS with a K_{d} of 9.0±3.7 μM (FIG. 8E) and S. meliloti EPS (succinoglycan) with a K_{d} of 221.9±102.3 μM (FIG. 8F-8G), which showed that EPR3 was a promiscuous receptor capable of binding EPS from different bacterial species. Perception of compatible EPS in legumes is believed to promote infection of bacteria and to deny root entry of incompatible strains (Kawaharada, Y. et al. Nature 2015 523: 308-213; Kawaharada, Y. et al. Nat Commun. 2017 8: 14534; Kelly, S. J. et al. Mol. Plant Microbe Interact. 2013 26: 319-329). The data suggested that ligand binding per se was not the sole discriminating factor for eliciting a response to compatible or incompatible bacteria.

In summary, EPR3 was a defining member of a large and conserved new class of plant receptors able to directly perceive EPS from different bacterial species. The evolutionary conservation observed highlighted a widespread requirement for recognition of EPS or other microbial surface polysaccharides in plants.

### Example 3: Identification and Characterization of Epr3a, a Homolog of Epr3

The following example describes the identification of Epr3a, a homolog of Epr3 in L. japonicus that shares 65% amino acid identity with Epr3a. Further, the following example describes the generation of mutations in both Epr3a and Epr3 in L. japonicus, and functional and phenotypic studies of the two genes.

### Materials and Methods

### Identification of Epr3a

L. japonicus Epr3a was identified based on its 65% amino acid identity to Epr3 (FIG. 11B).

### Mutant Alleles of Epr3 and Epr3a and Plant Lines

Lotus retrotransposon 1 element LORE1 mutant alleles of Epr3 and Epr3a were isolated from the LORE1 mutant resource (https://lotus.au.dk/). FIG. 11A shows a schematic of the LORE1 locations in the Epr3 and Epr3a genes. Homozygous mutants were identified through PCR-based genotyping.

A double mutant was isolated from crosses of epr3-11 and epr3a-2 mutants. Homozygous double mutants were identified through PCR-based genotyping.

### EPR3a ED Purification

The EPR3a ED was expressed in insect cells, and the protein was purified as described in Example 1 (FIG. 11D).

### EPR3 and EPR3a Kinase Domain Purification

The EPR3 and EPR3a kinase domains were expressed in E. coli, and the proteins were purified. EPR3 and EPR3a kinases were expressed in E. coli LOBSTR cells that were grown to an optical density of 0.6 at 600 nm before protein expression was induced with 0.2 mM IPTG at 18° C. overnight. Cells were lysed in buffer containing 50 mM Tris-HCl pH 8.0, 500 mM NaCl, 20 mM imidazole, and 1 mM benzamidine, and the cleared supernatant was loaded onto a Ni Sepharose 6 FF affinity column (GE Healthcare) and washed prior to elution in lysis buffer supplemented with 500 mM imidazole. Purification tags on EPR3 and EPR3a kinases were enzymatically removed with 3C protease and the proteins were finally purified on a Superdex 75 10/300 gel filtration column (GE Healthcare) in gel filtration buffer containing 50 mM Tris-HCl pH 8.0 and 200 mM NaCl.

### EPR3 and EPR3a Kinase Activity Assay

Proteins were incubated with 100 nCi [γ32-P]ATP (PerkinElmer) in 50 mM Tris.HCl, pH 8, buffer containing 10 mM MgCl2, 5 mM MnCl2, and 20 μM cold ATP at room temperature for 1 h. The samples were then separated on SDS/PAGE gels, which were exposed overnight on phosphor plates (Molecular Dynamics). The phosphor plates were scanned with the typhoon TRIO scanner (Amersham Biosciences). The kinase activity assay results are shown in FIGS. 11K-11M.

### Microscale Thermophoresis (MST) Binding Experiments

MST experiments were performed as described in Example 2 (FIG. 11C, FIGS. 11E-11J).

L. japonicus Gene Expression Profiling by RNA-Seq

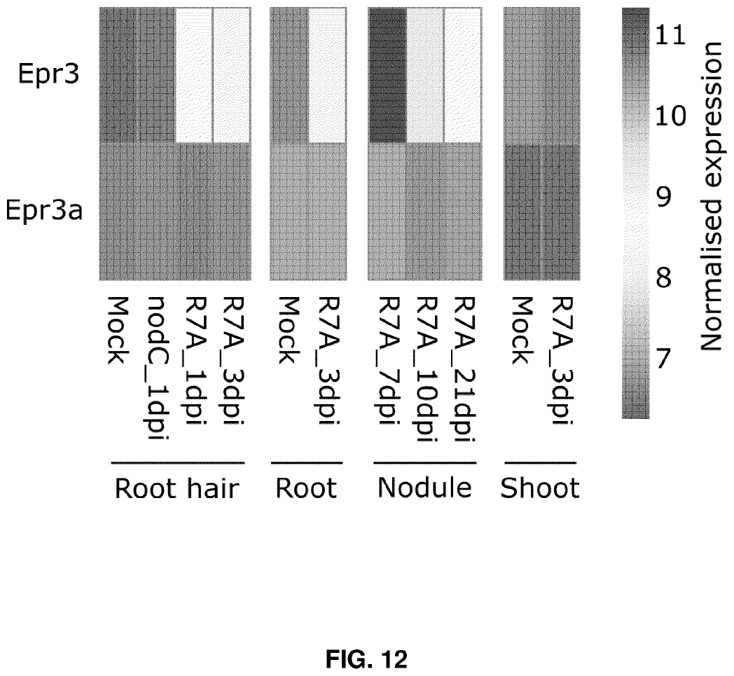

Epr3 and Epr3a expression was measured across L. japonicus tissue types using RNA-seq. Wild-type L. japonicus was treated with water or the symbiotic bacteria M. loti strain R7A. For tissues treated with M. loti strain R7A, RNA was collected either 1, 3, 7, or 21 days following treatment with M. loti strain R7A (FIG. 12). Total RNA was extracted using a NucleoSpin® RNA Plant kit (Macherey-Nagel) according to the manufacturer's instructions. RNA quality was assessed on an Agilent 2100 Bioanalyser and samples were sent to GATC-Biotech for library preparation and sequencing. Reads were mapped to the L. japonicus v. 3.0 genome and differential gene expression was analyzed using CLC Genomics Workbench 9.5.3 (Qiagen). For each sample, a minimum of 30 million reads were obtained with >90% of reads mapped to the reference genome. Relative expression levels of Epr3 and Epr3a was calculated in shoots, nodules, nodule primordia, root hairs, and roots.

### Plate Nodulation Assay

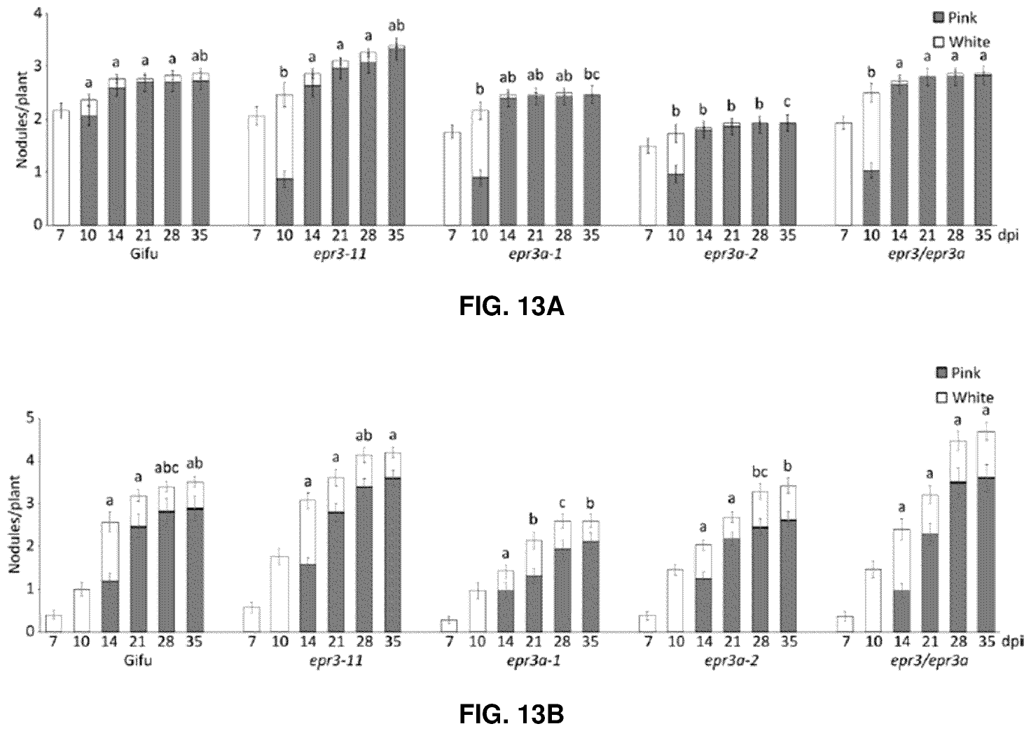

L. japonicus plants were grown on agar with or without the addition of M. loti strain R7A, and the number of nodules per plant was counted (FIGS. 13A-13B). Seeds were scarified with sandpaper, surface sterilized in 0.5% bleach, and germinated on wet filter paper for 3 days. Seeds with emerging radicles were transferred to square Petri dishes (Sigma-Aldrich) with agar slopes containing 0.25× B&D medium. The plate medium was solidified with 1.4% Agar Noble (Difco) and the surface of the slope was covered with filter paper (AGF 651, Frisenette ApS). A metal bar with 3-mm holes for roots was inserted at the top of the agar slope. Plates were incubated in an upright position in specially fabricated boxes, excluding light from the roots below the metal bar. Plants were incubated at 21° C. under a 16 h light/8 h dark cycle. Plates containing ten plants each were inoculated with 750 μl of M. loti at an optical density of OD₆₀₀=0.02.

### Nodules Per Plant

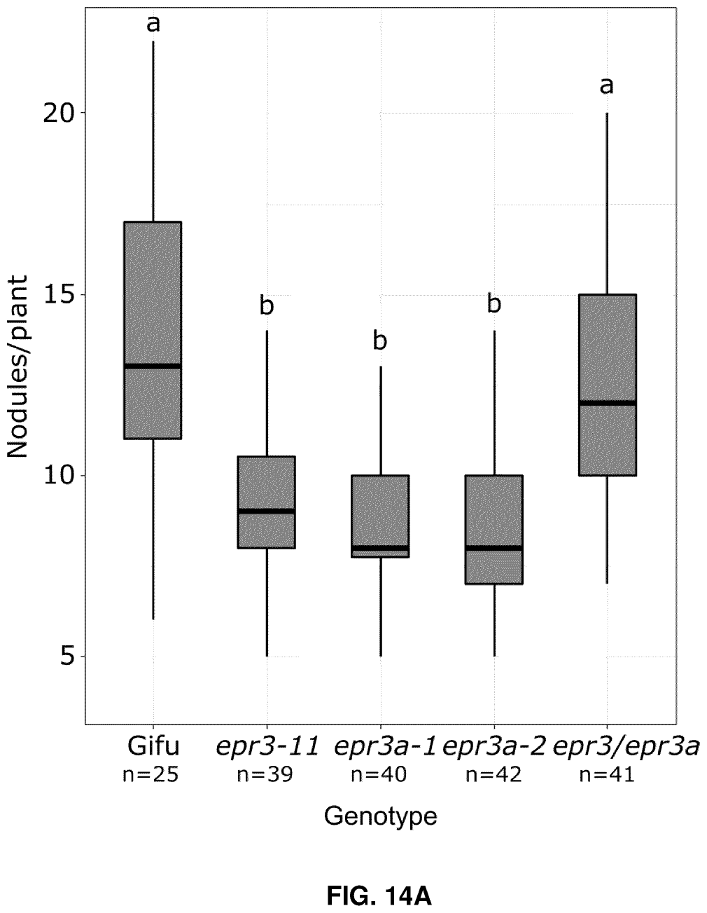

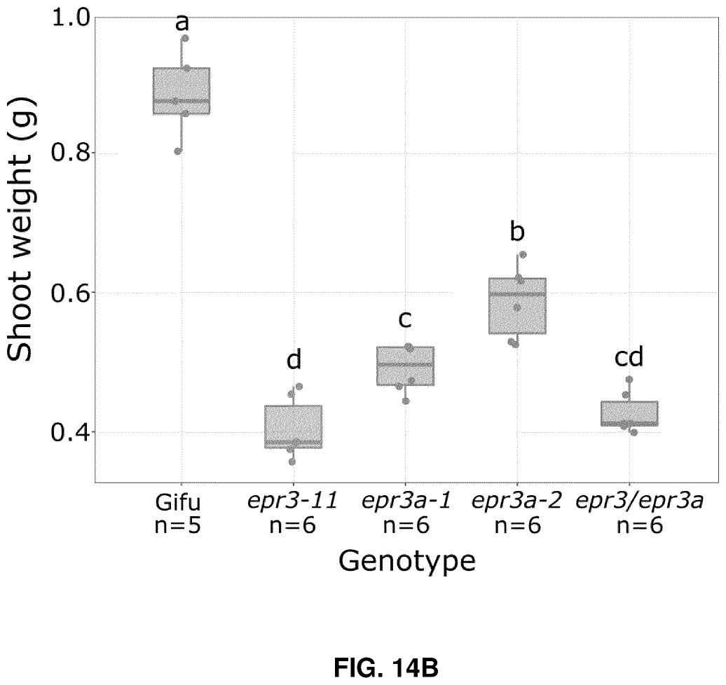

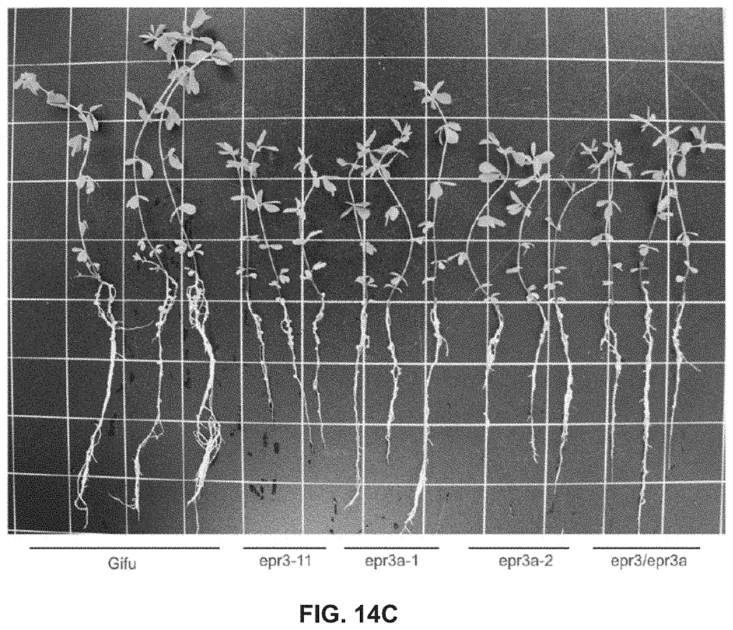

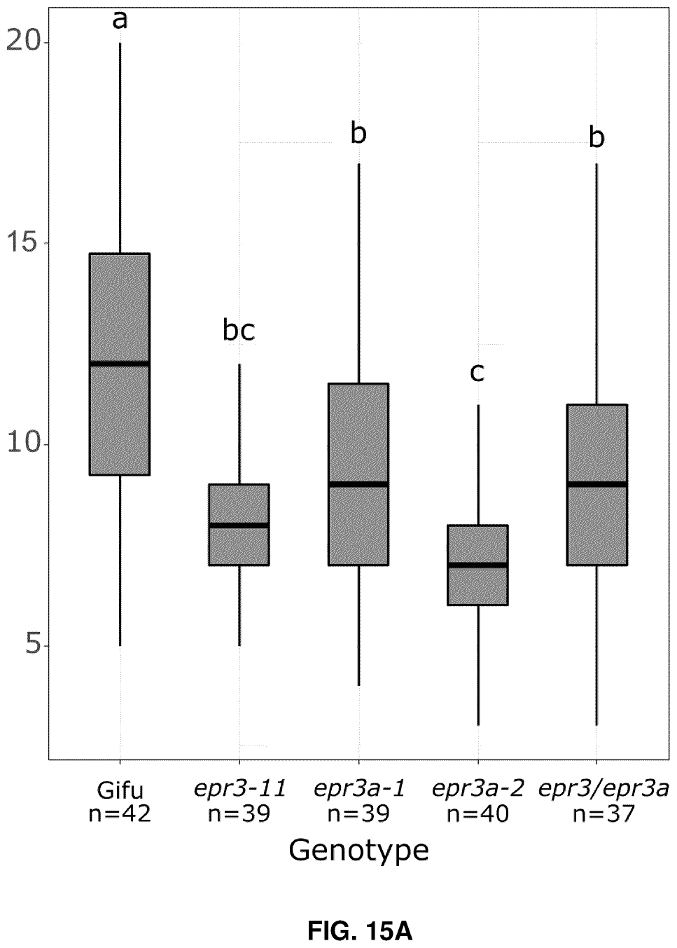

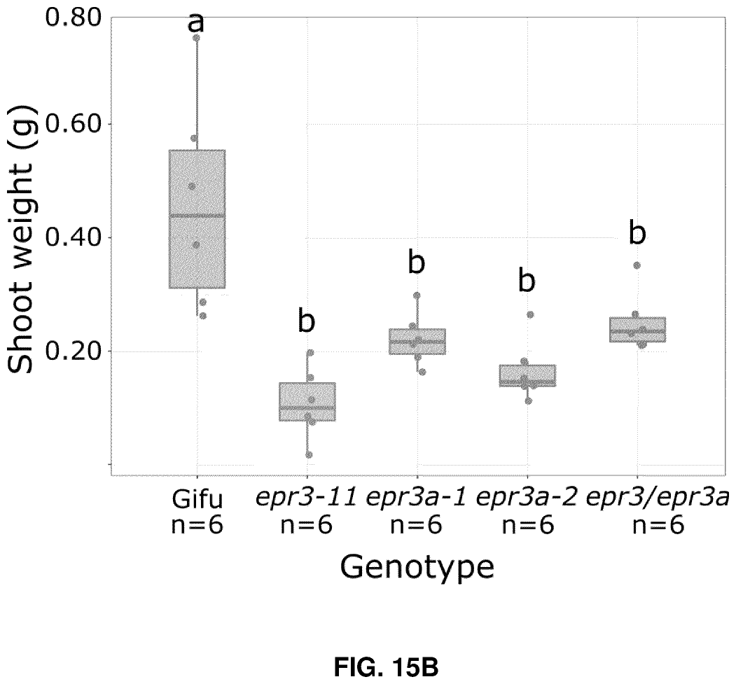

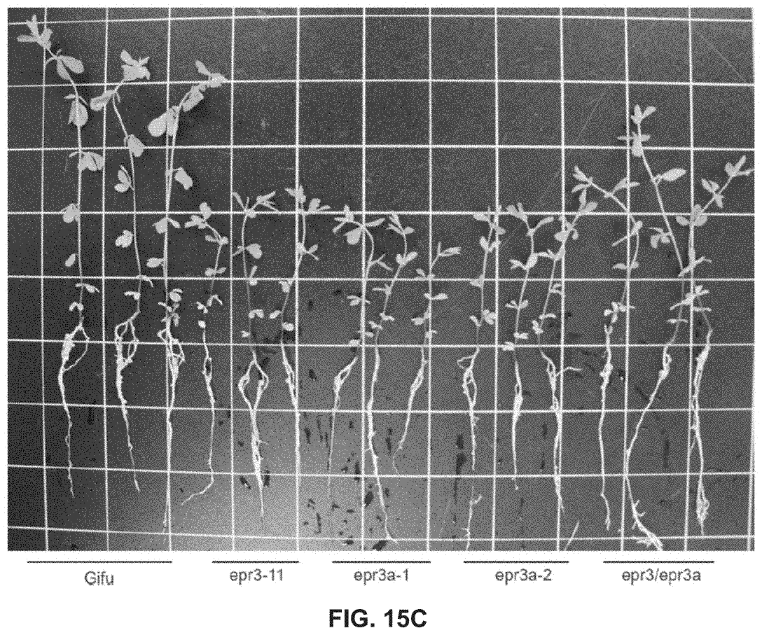

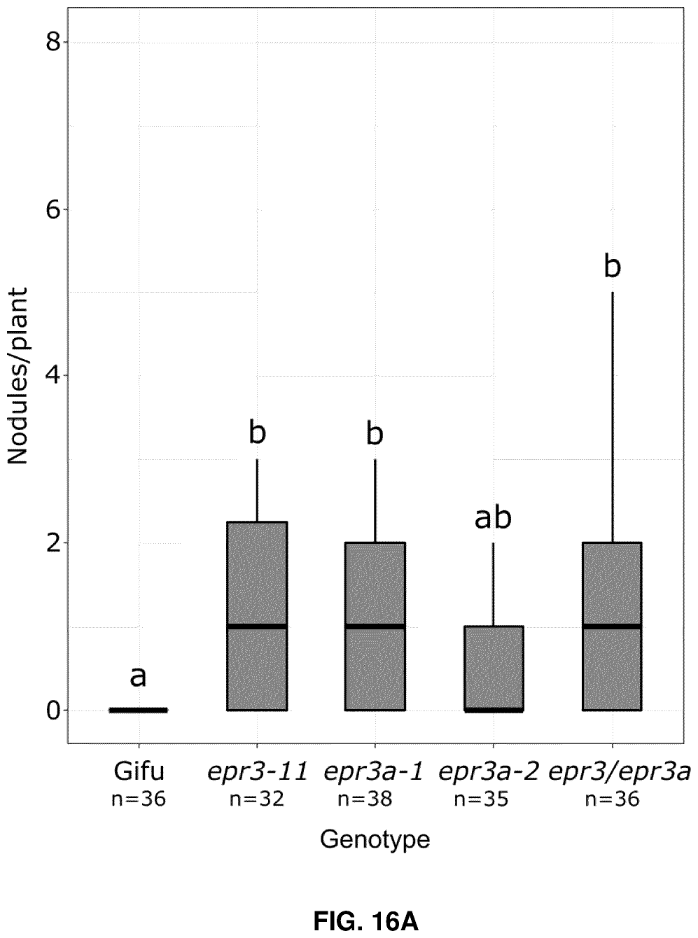

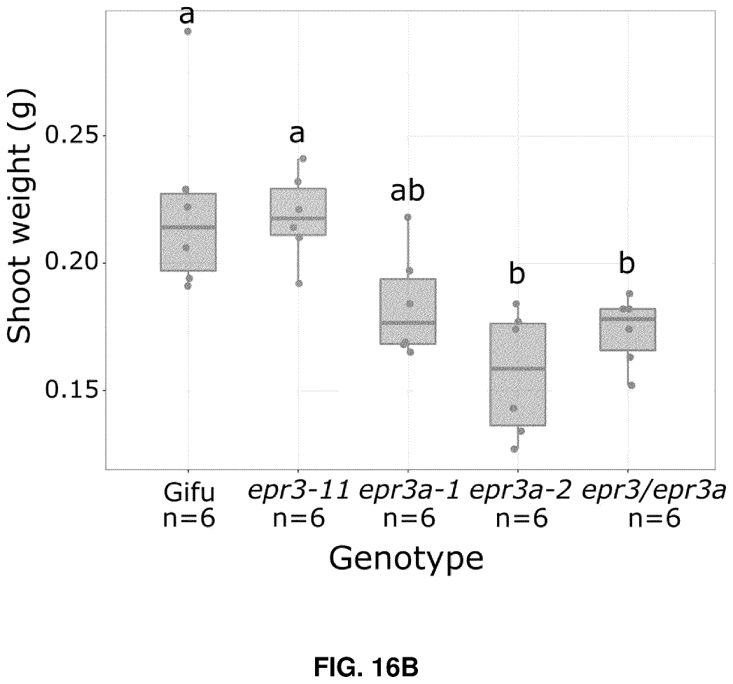

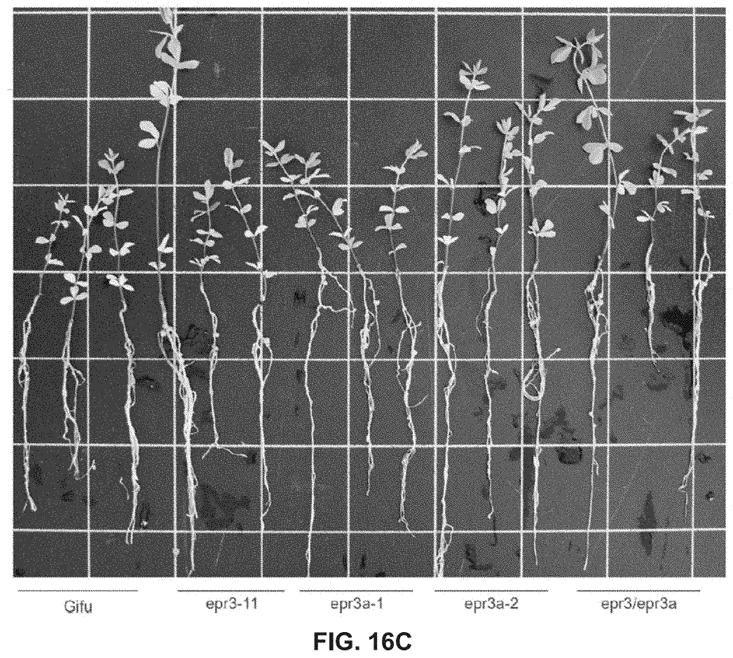

The number of root nodules formed per L. japonicus plant was counted (FIG. 14A, FIG. 15A, FIG. 16A).

### Plant Shoot Weight

Plant shoot weight was measured at the conclusion of nodulation assays by separating the shoot from the root and weighing on a fine-balance scale (FIG. 14B, FIG. 15B, FIG. 16B).

### Infection Threads

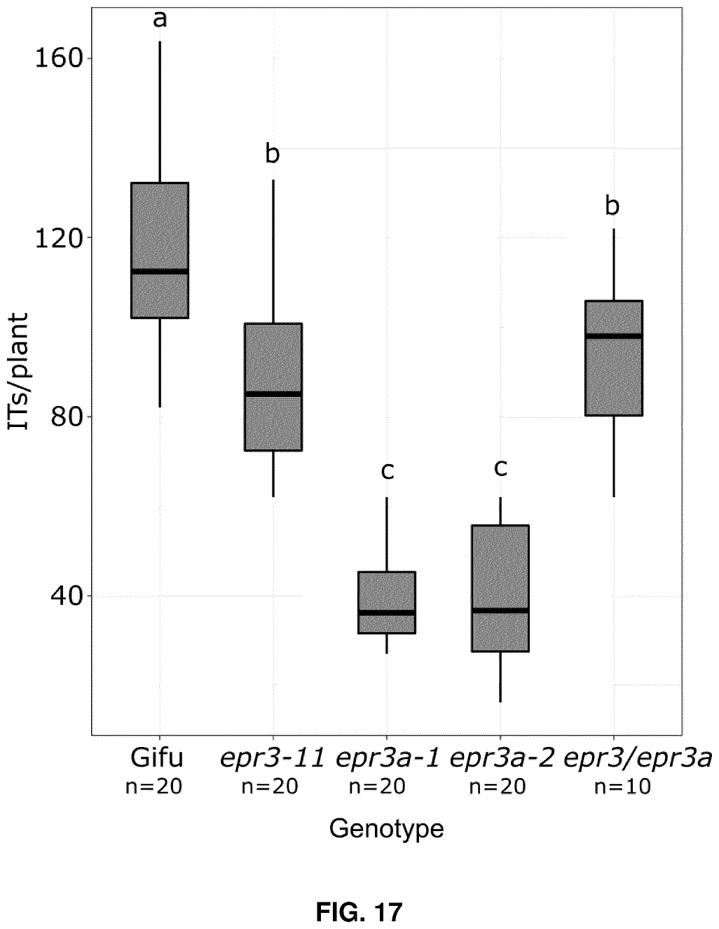

The number of infection threads formed during the development of L. japonicus root nodules was counted (FIG. 17). Roots used for counting infection threads were inspected using a Zeiss Axioplan 2 image fluorescence microscope.

qRT-PCR

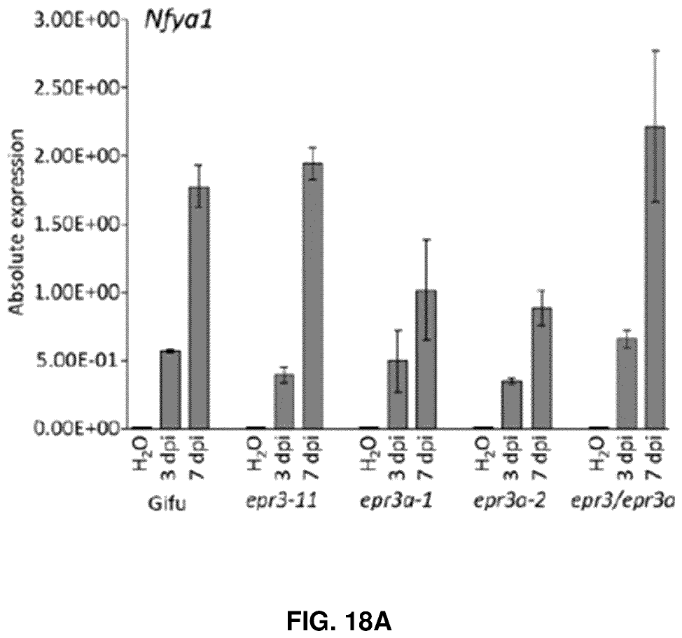

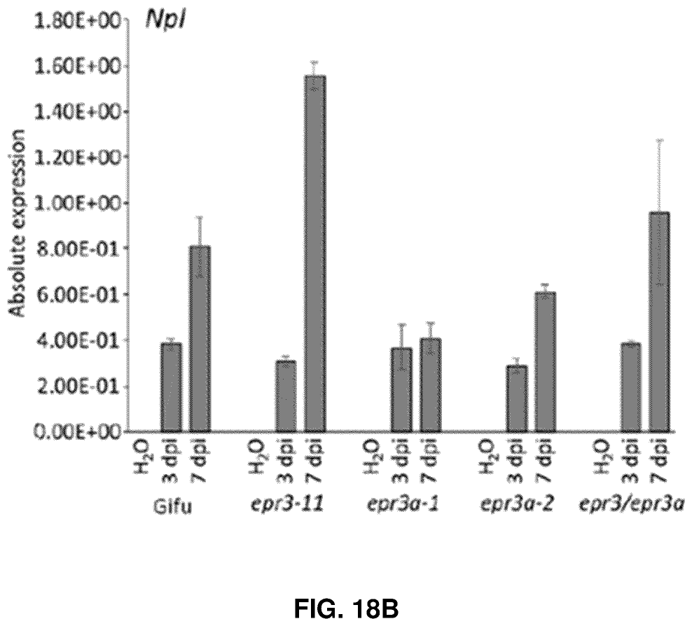

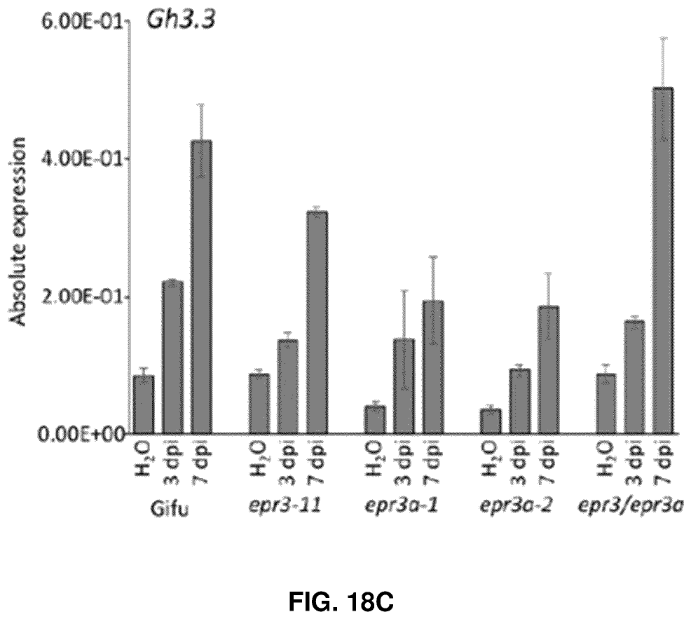

qRT-PCR was performed to measure the absolute expression of the symbiotic genes Gh3. 3, Nfyal, and NpI. Wild-type L. japonicus (Gifu) or L. japonicus with mutations in epr3 and/or epr3a were treated with water or M. loci strain R7A, and symbiotic gene expression was measured at 3 and 7 days post treatment (FIGS. 18A-18C). RNA was isolated using the NucleoSpin RNA Plant kit (Macherey-Nagel). RevertAid Reverse Transcriptase (Thermo) was used for cDNA synthesis according to the manufacturer's protocols. All cDNA samples were tested for genomic DNA contamination using primers specific for the NIN gene promoter. A LightCycler480 instrument and LightCycler480 SYBR Green I master (Roche Diagnostics) were used for the real-time quantitative PCR. ATP synthase and ubiquitin-conjugating enzyme were used as reference genes. The cDNA starting concentration for each gene was calculated using per amplicon PCR efficiency calculations calculated using LinRegPCR. Target genes were compared with the geometric mean of the housekeeping genes for each of three biological repetitions (each consisting of 10 plants). At least two technical replicates were performed in each analysis.

### Results

### Characterization of Epr3a

The EPR3a ED was purified and tested for binding M. loti EPS in a MST experiment (FIG. 11C). EPR3a ED bound M. loti EPS with an equilibrium dissociation constant of Kd=25.6 μM, indicating that, like EPR3, EPR3a is an EPS-binding proteins.

Further, the EPR3a ED was purified, resulting in a pure and monodisperse preparation suitable for biochemical studies, as shown in FIG. 11D. MST experiments were performed to test the ability of the EPR3a ED preparations shown in FIG. 11D to bind M. loti EPS, M. loti de-O-acetylated EPS, S. meliloti EPS, R. leguminosarum EPS, chitin (chitotetraose), or maltodextrin (FIGS. 11E-11J). The EPR3a ED bound to M. loti EPS with an equilibrium dissociation constant of K_{d}=44.4±11.2 04 (FIG. 11E), to M. loti de-O-acetylated EPS with an equilibrium dissociation constant of K_{d}=57.2±15.6 04 (FIG. 11F), to S. meliloti EPS with an equilibrium dissociation constant of K_{d}=936.7±389.4 04 (FIG. 11G), and to R. leguminosarum EPS with an equilibrium dissociation constant of K_{d}=12.5±4.0 04 (FIG. 11H). As shown in FIGS. 11I-11J, the EPR3a ED did not bind to chitin or maltodextrin.

### Comparison of Kinase Activity of Epr3 and Epr3a

EPR3 and EPR3a kinase domains were purified from E. coli, and their kinase activities was measured. As shown in FIGS. 11K-11M, the kinase domains of both EPR3 and EPR3a showed robust kinase activity, including both auto-phosphorylation activity and phosphorylation of an acceptor protein (MBP).

### Comparison of Expression of Epr3 and Epr3a

Analysis of L. japonicus RNA-seq data and qRT-PCR results showed that in roots, Epr3 expression was induced by M. loti strain R7A, whereas Epr3a was expressed in un-inoculated (i.e., H₂O-treated) roots. Epr3 and Epr3a ware both expressed in M. loti strain R7A-treated nodule primordia. Epr3a was expressed at a low level in root tissues and its transcription did not respond to rhizobial inoculation (note that FIG. 12 shows relative expression, so the expression appears high in the root relative to the shoot), whereas Epr3 expression was upregulated upon treatment with M. loti strain R7A (FIG. 12). Expression of Epr3a was highest in nodule primordia and mature nodules compared to other root tissues (FIG. 12).

Phenotypic Comparison of Wild-Type L. japonicus to L. japonicus with Mutant Epr3 and/or epr3a

Symbiotic phenotyping of epr3a single mutants shows a reduction in nitrogen-fixing nodule formation and a severe reduction in the number of infection threads formed with the compatible symbiont M. loti strain R7A (FIG. 14A). The epr3/epr3a double mutant showed a reduction in infection thread formation/symbiotic phenotype compared to wild-type, but the reduction was not as severe as the reduction in infection thread formation in the single mutants epr3a-1 and epr3a-2 (FIG. 17). Comparable effects on symbiotic phenotypes were observed with the EPS-deficient M. loti strain R7AexoY/F mutant (FIG. 15A). epr3, epr3a, and epr3/epr3a double mutants were able to form nodules in association with M. loti strain R7exoU that produces a truncated EPS, while the wild-type L. japonicus Gifu was not (FIG. 16A).

### Induction of Symbiotic Genes

qRT-PCR analysis indicated that symbiotic gene induction in response to M. loti strain R7A was reduced in the epr3a single mutants, while in epr3 and epr3/epr3a double mutants the level of symbiotic gene induction was comparable to wild-type (FIGS. 13A-13B).

Epr3a and Epr3 exhibited genetic epistasis, as observed in the infection thread phenotype and symbiotic gene induction phenotypes in which the double mutation of both genes removed the defects observed in plants with Epr3a alone mutated (FIG. 17, FIGS. 18A-18C).

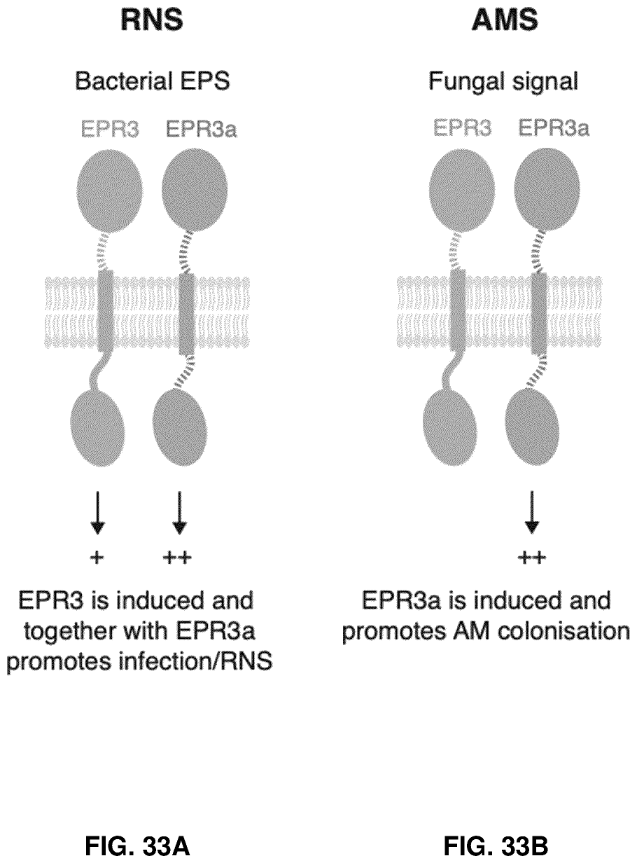

Overall, the results indicated that EPR3a and EPR3 were both EPS-receptors that were functionally important for interaction of L. japonicus with symbiotic bacteria. FIGS. 19A-19B and FIGS. 33A-33B each provide models for symbiosis signalling with EPR3a and EPR3.

### Example 4: L. japonicus EPR3 Functions in Shaping Soil Microbiota Diversity

The following example describes a comparison of the root microbiota from soil-grown L. japonicus wild-type (Gifu) and mutant plants impaired for exopolysaccharide perception (epr3).

Materials and Methods

16S rRNA Sequencing of Bacterial Communities

Wild-type (ecotype Gifu) L. japonicus and mutant L. japonicus in Epr3 (epr3-1 1, epr3-10 and epr3-13) were cultivated in parallel, in natural soil. Bacterial communities of unplanted soil, rhizosphere, and endosphere/root compartments of wild-type and epr3-13 genotypes at bolting stage were characterized following the established protocol (Zgadzaj, R. et al. P Natl Acad Sci USA 2016 113: E7996-E8005). Visible nodules and root primordia were removed from the roots prior to sample processing for community profiling. The V5-V7 hypervariable region of the bacterial 16S rRNA gene was amplified from the aforementioned compartments and genotypes and sequenced using Illumina technology. Low-quality reads were removed, and chimeras and sequences were assigned to plant-derived organellar DNA. Three biological and three technical replicates were sequenced.

### Operational Taxonomic Unit (OTU) Clustering

The identified V5-V7 amplicons were clustered into Operational Taxonomic Units (OTUs) at 97% sequence similarity. In order to determine the effect of EPR3 mutation on the composition of bacterial communities, the observed community shifts were dissected by arranging OTUs according to their taxonomic assignment.

### Calculation of α- and β-Diversity and OTU Enrichment

Information on the number and relative abundance of operational taxonomic units (OTUs) in each compartment was used to calculate α-diversity (Shannon index; within sample diversity) and β-diversity (Bray-Curtis distances; between samples diversity), OTU enrichment, and taxonomic composition in different compartments and genotypes. Rarefaction was conducted for each sample to calculate the Shannon index. According to the minimum read number in all of the samples, 14,041 reads rarefied from each sample. The script “alpha_diversity.py” in QIIME1 was used to calculate the Shannon index. Normalization of the OTU table was conducted to calculate the Bray-Curtis distances. The relative abundance of each OTU in each sample was employed as the normalization method. According to the OTU relative abundance, OTUs that were lower than 0.01% abundance were deleted before calculating Bray-Curtis distances. The script “beta_diversity.py” in QIIME1 was used to calculate Bray-Curtis distances. R script was used for the visualization of α-diversity and β-diversity.

### Canonical Analysis of Principle Components Coordinates

In order to assess the impact of the different host compartments and genotypes in community composition, a Canonical Analysis of Principle Components Coordinates (CAP) was performed. The function “capscale” in the R package “vegan” was employed to run the CAP analysis. The effect of biological replicates and technical replicates was partialled out.

### Results

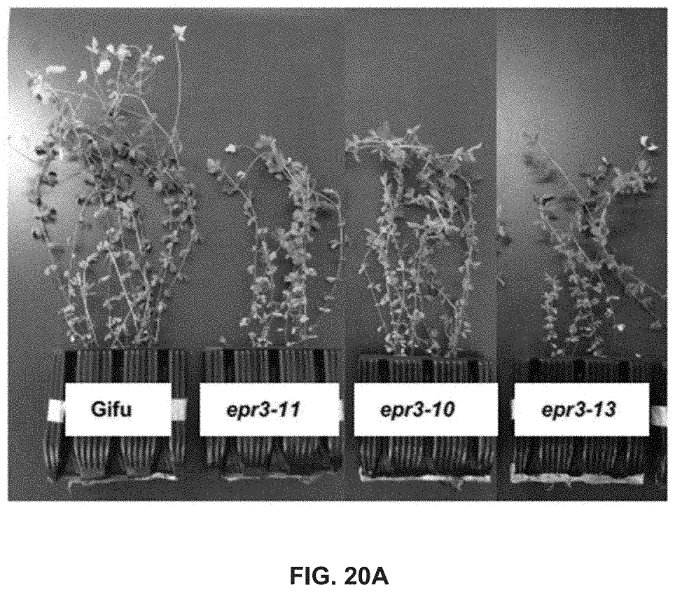

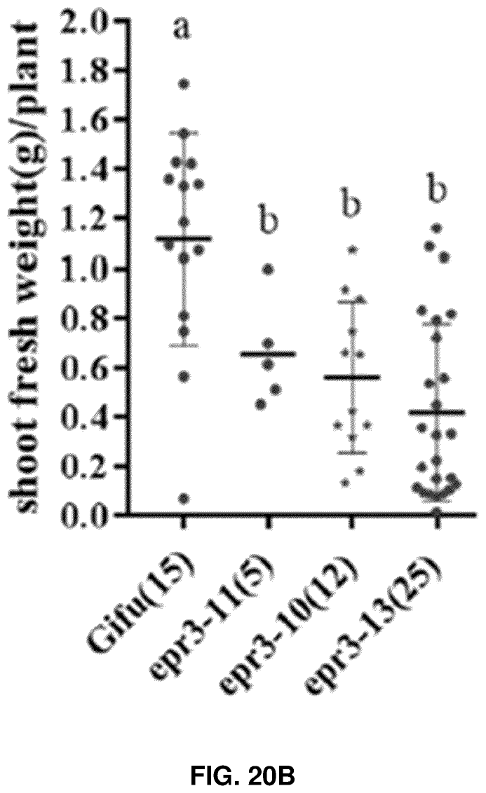

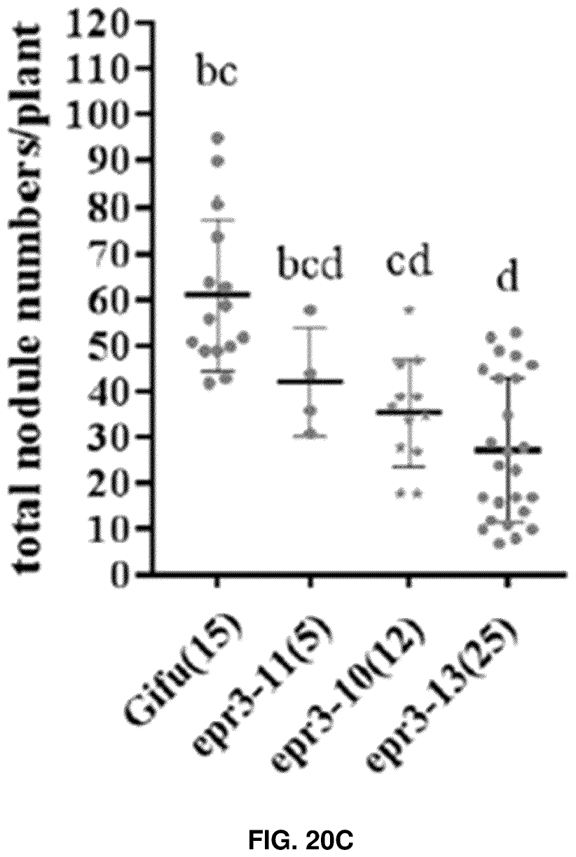

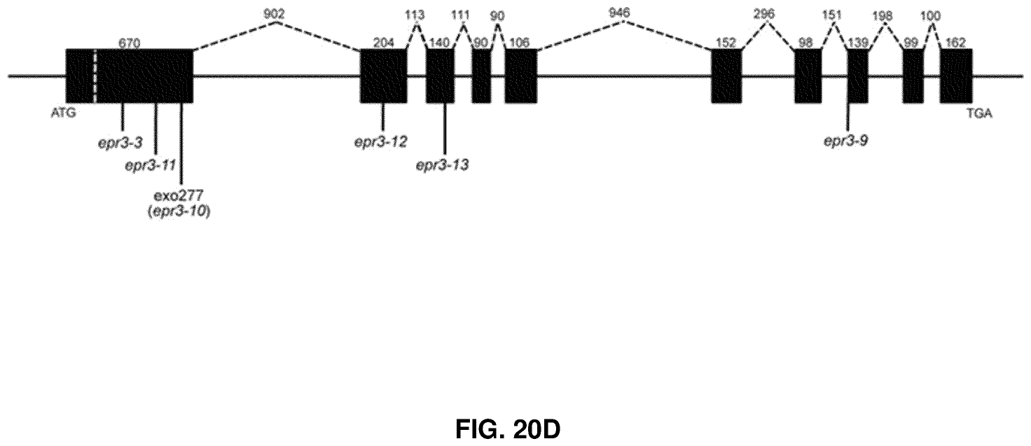

Studies of microbial communities associated with healthy plants led to the paradigm that plant roots have an intrinsic capacity to attract and accommodate a selection of microbial taxa from the rich environment present in the soil for their own benefit (Bulgarelli, D. et al. Annu Rev Plant Biol 2013 64: 807-838). In order to determine the impact of EPR3 on root microbiota composition wild-type (ecotype Gifu) L. japonicus and L. japonicus mutant in Epr3 (epr3-11, epr3-10 and epr3-13; schematic shown in FIG. 20D) were cultivated in parallel, in natural soil. All plant genotypes appeared healthy and formed a large number of symbiotic root nodules (FIG. 20A). In spite of this apparent well-developed phenotype, the shoot length, shoot fresh weight and the number of nodules/plants of the epr3 mutant plants were significantly reduced in comparison to wild-type (FIG. 20B, FIG. 20C), suggesting that genetic disruption of EPR3 was detrimental for the fitness of plants grown in natural soil.

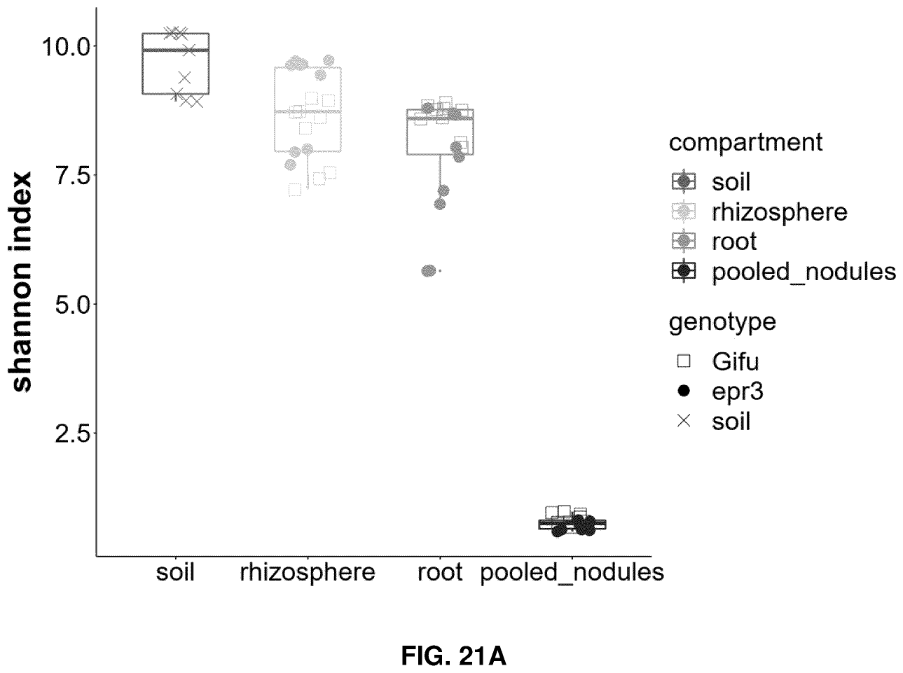

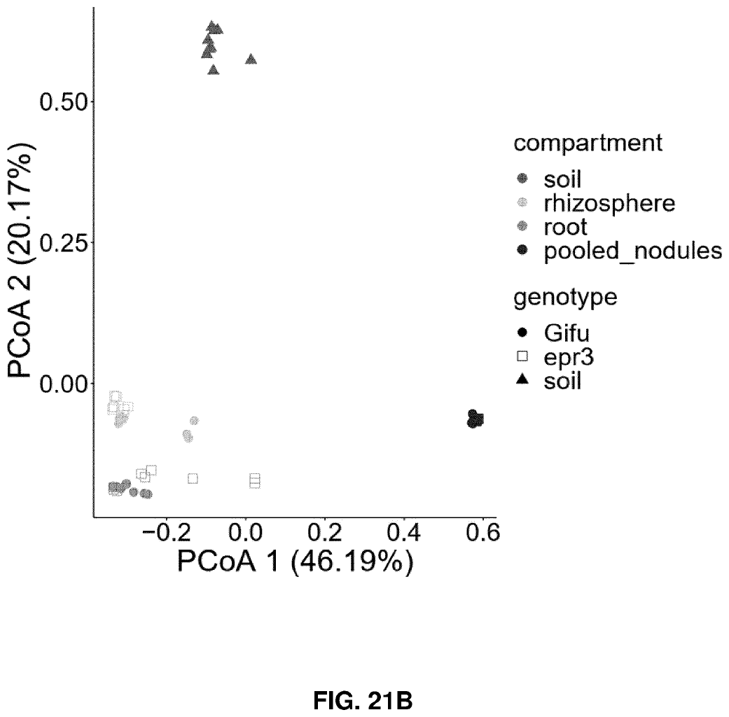

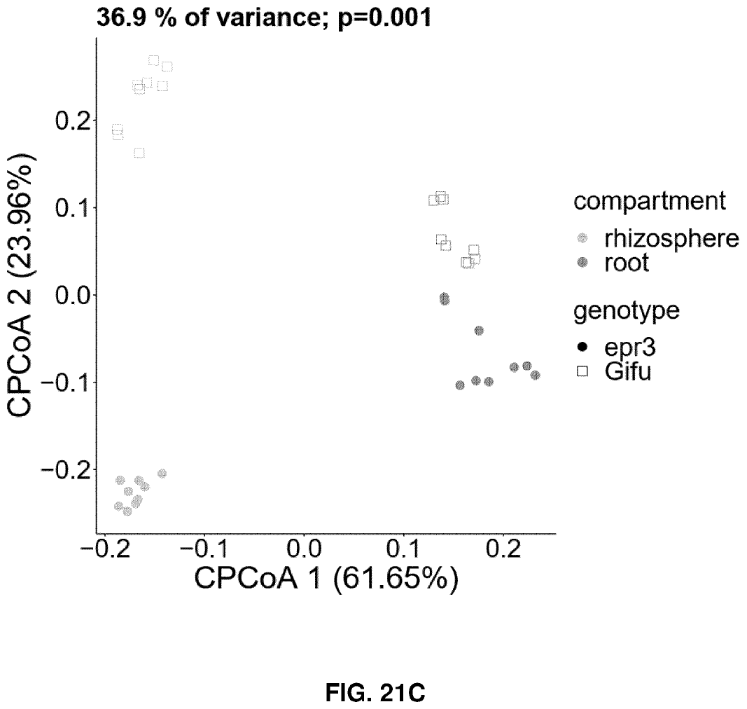

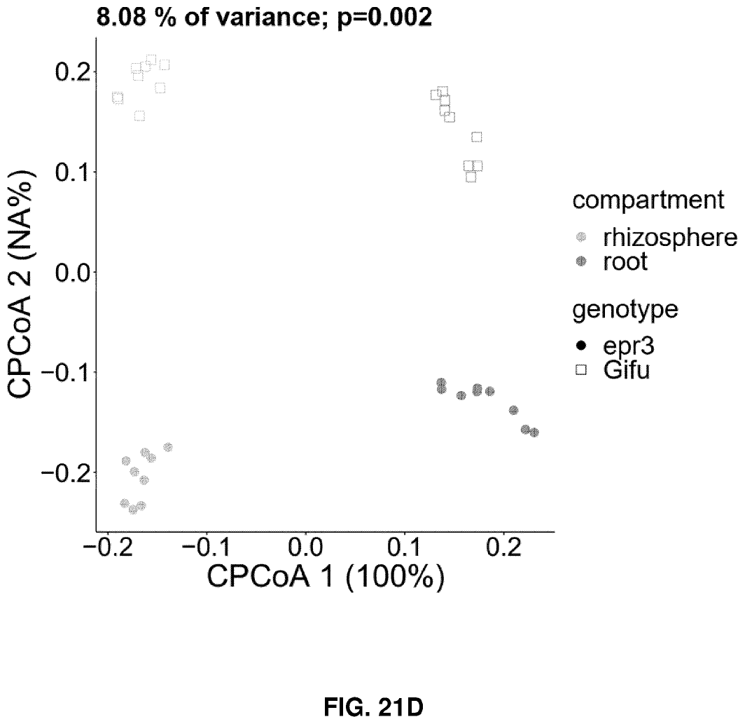

Analysis of α-diversity revealed a general reduction of complexity from unplanted soil to rhizosphere, endosphere/root and lastly the nodule compartments for bacterial communities which is consistent with previous studies from L. japonicus and A. thaliana microbial communities (Bulgarelli, D. et al. Nature 2012 488: 91-95; Lundberg, D. S. et al. Nature 2012 488: 86; Zgadzaj, R. et al. P Natl Acad Sci USA 2016 113: E7996-E8005). No significant difference was observed between mutant and wild-type, indicating no significant changes in diversity within samples (FIG. 21A).

In order to assess the impact of the different host compartment and genotypes in community composition, a Canonical Analysis of Principle Components Coordinates (CAP) was performed (FIGS. 21B-21C). This revealed a clear differentiation of bacterial communities in the tested plant genotypes in both endosphere/root and rhizosphere compartments, with the host genotype explaining as much as 8% of the overall variance of the 16S rRNA data (FIG. 21D; P<0.002). The rhizosphere and endosphere compartments of wild-type plants were found to harbor different bacterial communities that were separate from those of epr3 (FIG. 21C, FIG. 21D) illustrating a pronounced host genotype effect in addition to the compartment effect (36.9% of variance in the dataset is explained by compartment and genotype) for the root-associated bacterial communities. Interestingly, the analysis of β-diversity in the nodule compartment, revealed no significant differences between wild-type and mutant, indicating that a similar bacterial composition is hosted inside the symbiotic root organ (FIG. 21B).

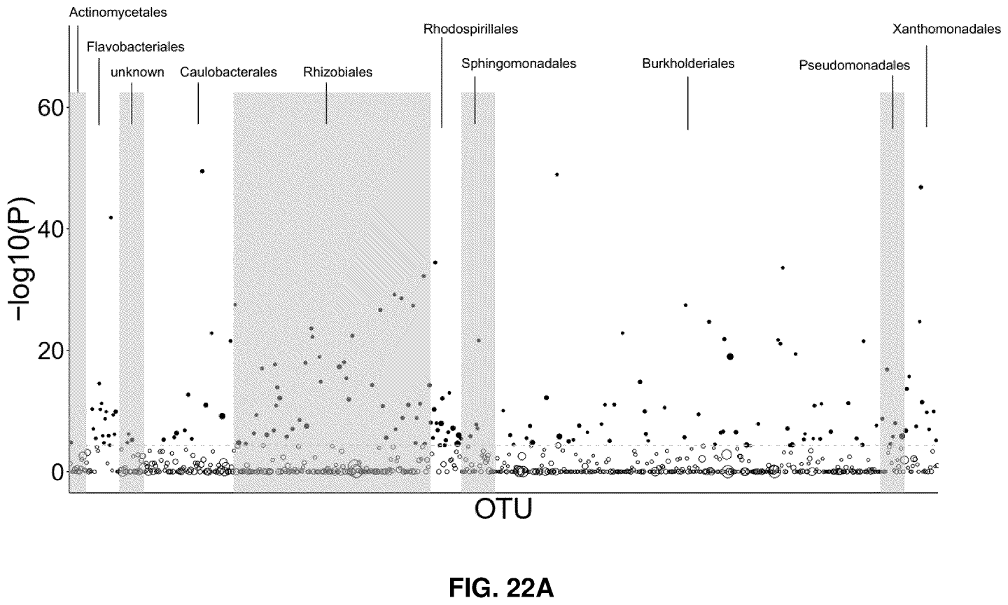

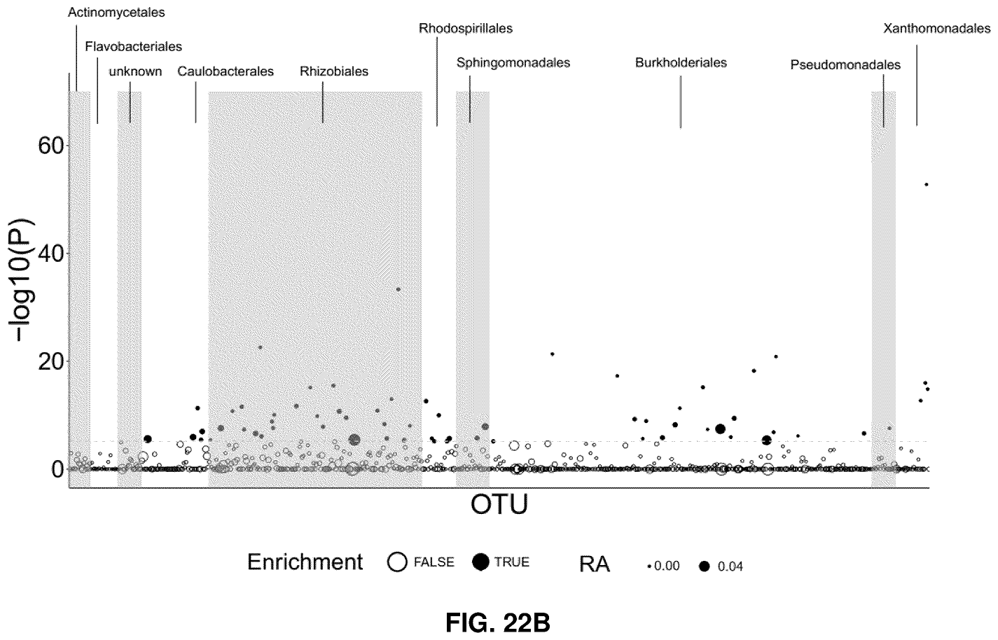

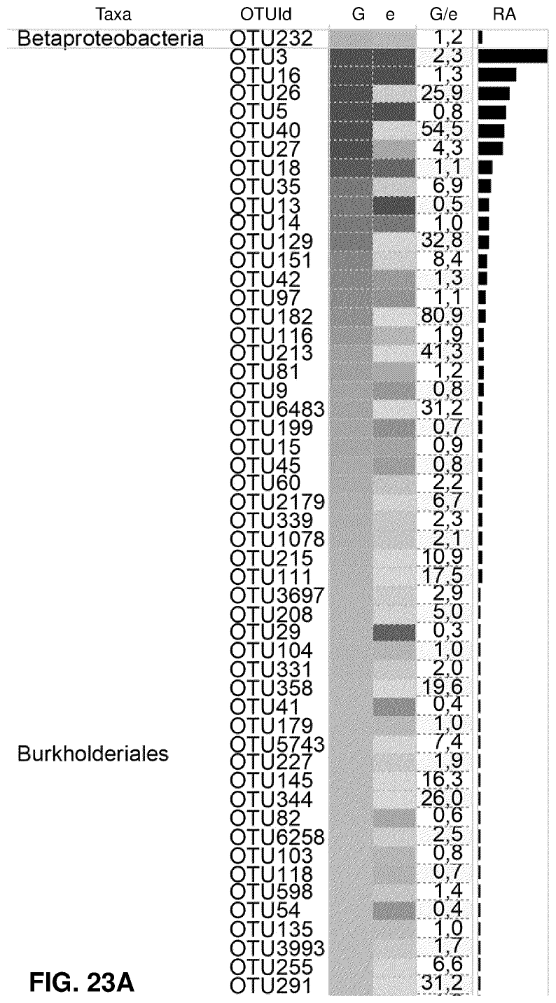

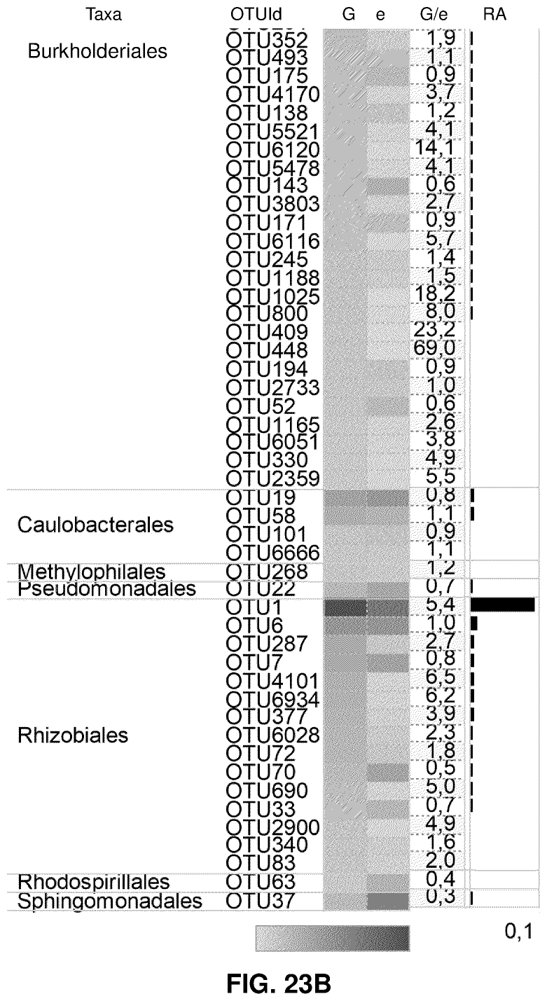

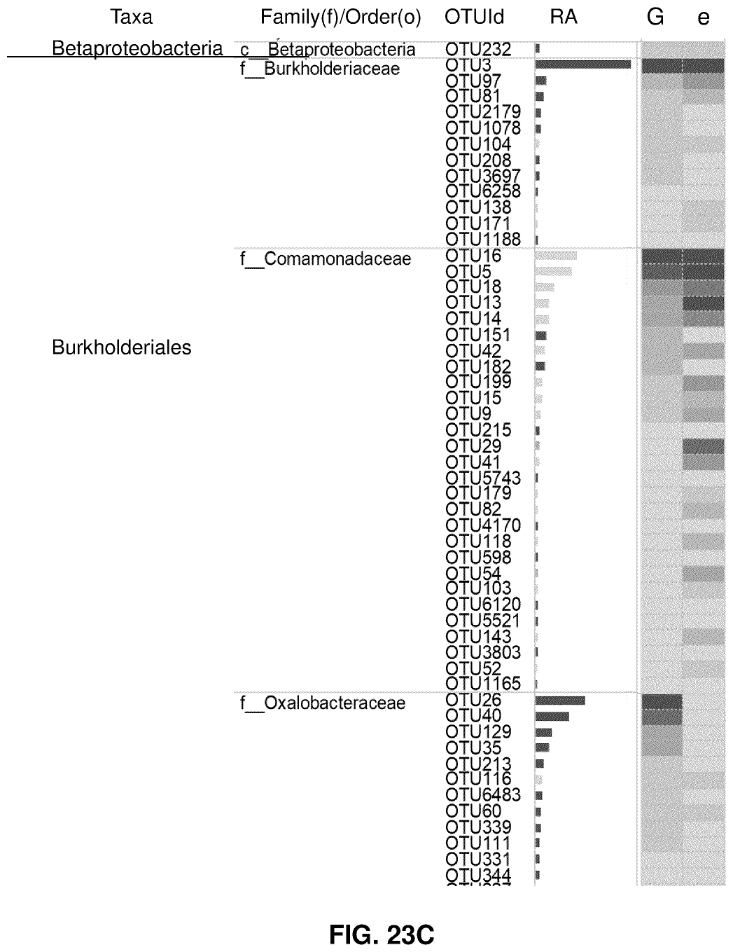

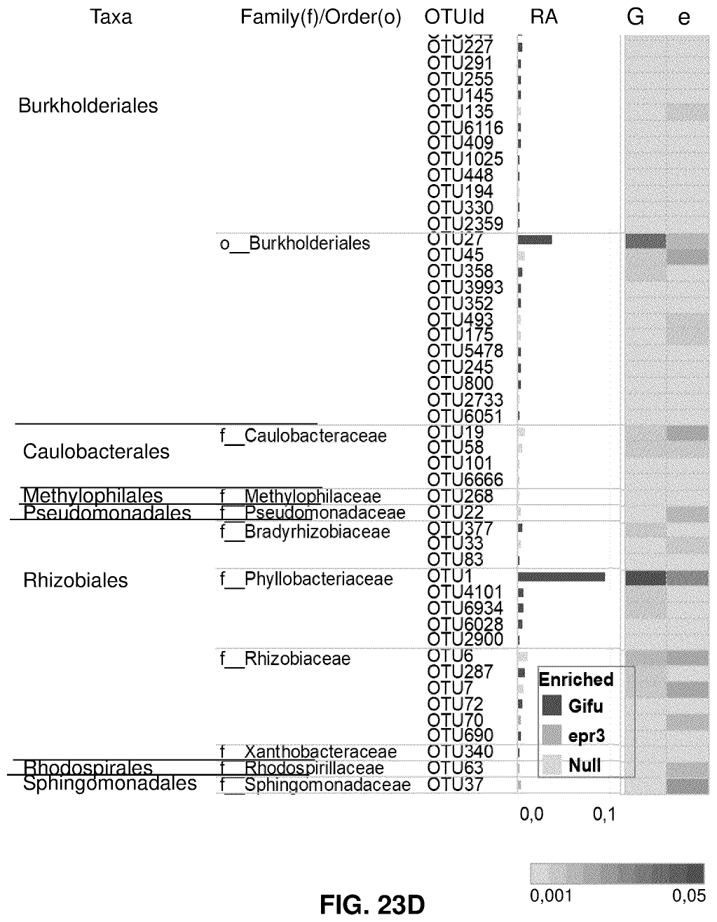

In order to determine the effect of EPR3 mutation on the composition of bacterial communities, the observed community shifts were dissected by arranging OTUs according to their taxonomic assignment. This revealed that, with the exception of OTUs assigned to Burkholderiales or Rhizobiales, in both rhizosphere and endosphere only few OTUs assigned to other orders had a significant difference in their relative abundance between wild-type and epr3 mutant plants (FIGS. 22A-22B). Remarkably, a large number of OTUs belonging to Burkholderiales and Rhizobiales had a significantly reduced abundance in the endosphere/root and rhizosphere compartments of the epr3 mutant compared to wild-type, indicating a taxonomically selective depletion (FIGS. 22A-22B). In the rhizosphere compartment, 75 out of all 328 OTUs assigned to Burkholderiales and 42 out of all 199 OTUs assigned to Rhizobiales had a significantly larger abundance in wild-type compared to the epr3 mutant (FIGS. 22A-22B). Among the top 100 OTUs identified in the wild-type rhizosphere, 48 of 76 Burkholderiales OTUs and 11 of 15 Rhizobiales OTUs, together accounting for 0.66 total aggregated abundance, were depleted in the epr3-13 rhizosphere (FIGS. 23A-23D). A less pronounced, but still significant effect was identified in the endosphere/root compartment, where 12 of 38 Burkholderiales OTUs and 7 of 39 Rhizobiales OTUs from the top 100 most abundant OTUs in wild-type, had a reduced abundance in the mutant endosphere/root. In epr3 mutant endosphere/root and rhizosphere, the depletion of a large number of Burkholderiaceae and Oxalobacteraceae taxa was accompanied by a concomitant enrichment of members from Comamonadaceae, suggesting compensatory niche replacement within the Burkholderiales community.

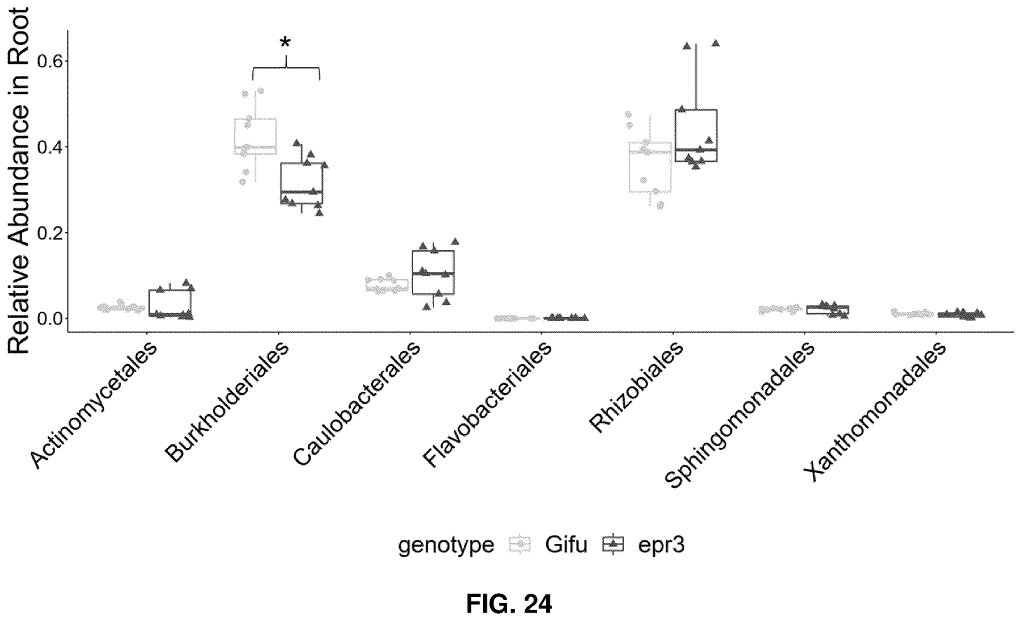

The Rhizobiales OTU1, which is the most abundant bacterial taxa detected in wild-type and epr3 nodules (93.9% RA) was found more abundant in the epr3 mutant endosphere/root (RA=0.11) when compared to wild-type (RA=0.04) (FIG. 24). Detailed microscopic studies of the infection pattern manifested by compatible symbionts in L. japonicus epr3 mutants revealed an impaired progress of infection from the epidermal into the nodule cortex (Kawaharada, Y. et al. Nature 2015 523: 308-312). The observed increased abundance of OTU1 in the epr3 endosphere/roots could reflect a similar pattern for the compatible symbiont present in this native soil. Its ability to initiate root infection seems not to be restricted by mutation of EPR3, but, like M. loti R7A model strain, it may remain blocked during infection of the root compartment leading to reduced nodulation.

Members of Burkholderiales and Rhizobiales have been designated keystone taxa due to their large prevalence, and abundance in different environments (Thompson, L. R. et al. Nature 2017 551: 457-463). However, there is only detailed knowledge on particular members of these two orders: the symbiotic diazotrophs and the pathogenic isolates (Angus, A. A. et al. PLoS One 2014 9: e83779; Chen, W. M. et al. J Bacteriol 2003 185: 7266-7272; Denny, T. in Plant-Associated Bacteria 2007). These represent only a small fraction of the overall Burkholderiales and Rhizobiales taxonomic units found to be habitual colonizers (endophytes) of healthy plant roots (Banerjee, S. et al. Nat Rev Microbiol 2018 16: 567-576; Garrido-Oter, R. et al. Cell Host Microbe 2018 24: 155-167). Currently, the molecular bases accounting for their prevalence and abundance across diverse range of environments are not known, nor their contribution to plant growth and development.

The results provided the first genetic evidence that a plant host was able to enrich for specific members of two of bacterial orders from the soil biome. EPS perception by the EPR3 receptor in L. japonicus enabled colonization of endosphere/root and rhizosphere by distinct members of Burkholderiales and Rhizobiales leading to increased plant fitness.

### Example 5: Engineering Modifications of Epr3 and Epr3a

The following example describes the genetic engineering of Epr3 in plants such as cowpea, soybean, cassava, rice, soy, wheat, and tobacco.

### Materials and Methods

### Materials and Methods Relevant for Engineering Modifications of Epr3 and Epr3a

The EPS-binding specificity of engineered alleles of EPR3 and EPR3a are characterized as described in Example 2.

The soil microbiota of the transformed plant lines are characterized as described in Example 4.

### Results

### Genetic Engineering Endogenous Epr3 or Epr3a in Plants

A genetically altered allele of Epr3 or Epr3a is introduced into a crop plant, replacing one or more endogenous copies of Epr3 or Epr3a. The composition of the rhizosphere and root bacterial communities are measured by 16S rRNA sequencing. Crop plants with altered Epr3 or Epr3a will affect the composition of the rhizosphere and/or root bacterial community differently than plants with wild-type Epr3 or Epr3a.

### Generating Chimeric Alleles of Epr3 in Plants

A chimeric allele of Epr3 with an M1 domain from a homologous Epr3 gene, or an ectodomain sequence from a homologous Epr3 gene is introduced into a crop plant. The composition of the rhizosphere and root bacterial communities are measured by 16S rRNA sequencing. Crop plants with chimeric Epr3 will affect the composition of the rhizosphere and/or root bacterial community differently than plants with wild-type Epr3.

### Insertion of an Extra Copy Epr3 or Epr3a in Plants

An extra, exogenous copy of Epr3 or Epr3a is inserted into a crop plant. The composition of the rhizosphere and root bacterial communities are measured by 16S rRNA sequencing. Crop plants with an extra copy of Epr3 or Epr3a will affect the composition of the rhizosphere and/or root bacterial community differently than plants with a wild-type number of Epr3 or Epr3a genes.

### Enriching for Commensal Bacteria in Soil

Crop plants with genetically altered Epr3 alleles or copy numbers are grown. The composition of the soil microbiota is measured by 16S sequencing. Crop plants with genetically altered Epr3 genes will affect the soil microbiota such that compatible bacteria are enriched in the plant's local environment.

### Screening for Compatible Commensal Bacteria

Binding to the EPR3 ED is used as a means of recognizing EPS produced by the commensal bacteria M. loti.

### Example 6: Exemplary Structural Alignment to Identify Novel EPR3 Receptors

One of skill in the art would have no difficulty applying the teachings of this disclosure to identify novel EPR3 receptors. Exemplary steps would be:

1. Align the amino acid sequence of the potential EPR3 receptor with one or more known EPR3 receptor sequences (as in FIGS. 4A-4C). The alignment is used to determine the position of the M1 domain, which is at the N-terminal end of the ectodomain. The position of the M1 domain corresponds to the position of the LysM1 domain in canonical LysM receptors (FIG. 6).

The M1 domain in L. japonicus EPR3 is 43 residues (EPR3 amino acid residues 55-97, NSLLYHISIGLKVEEIARFYSVNLSRIKPITRGTKQDYLVSVP), and can be aligned to identify new candidate M1 sequences.

2. An ab-initio protein structure prediction program such as Quark is used to predict the structure and fold of the new candidate M1 domain (Xu and Zhang Proteins 2012 80: 1715-1735).

The structure generated by the ab-initio protein structure prediction program is highly accurate, as shown in FIG. 5B. The output structure from Quark (Lotus EPR3 M1-modelled) shares the same topology, βαββ fold, and superpositions extraordinarily well with the M1 domain of Lotus EPR3 crystal structure (RMSD of 1.33 Å).

3. If the modeled M1 domain of the potential EPR3 receptor shares the same topology, βαββ fold, and superimposes well with the L. japonicus EPR3 M1 domain, it is a new EPR3 homolog.

### Example 7: Identification of EPR3 Receptors

The following example describes the identification of homologs of Epr3 and Epr3a genes in various plant species.

### Materials and Methods

### Identification of EPR3 Receptors

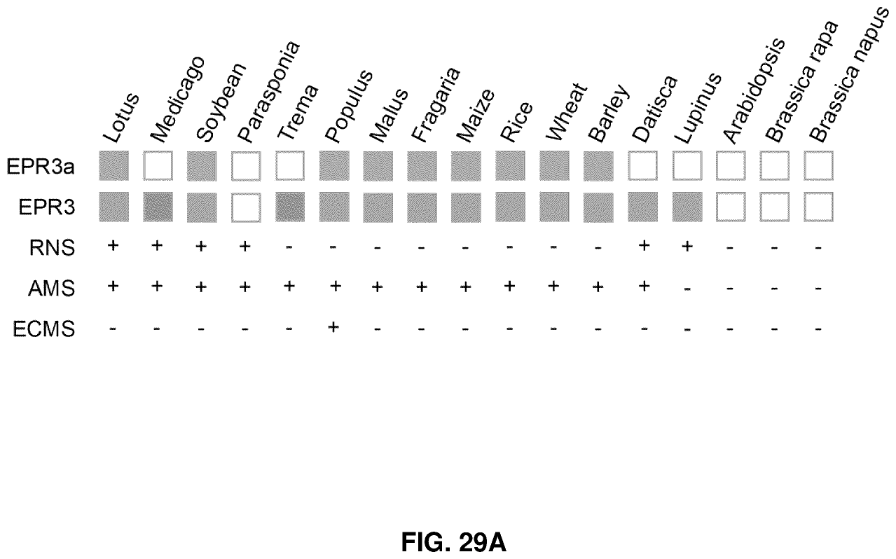

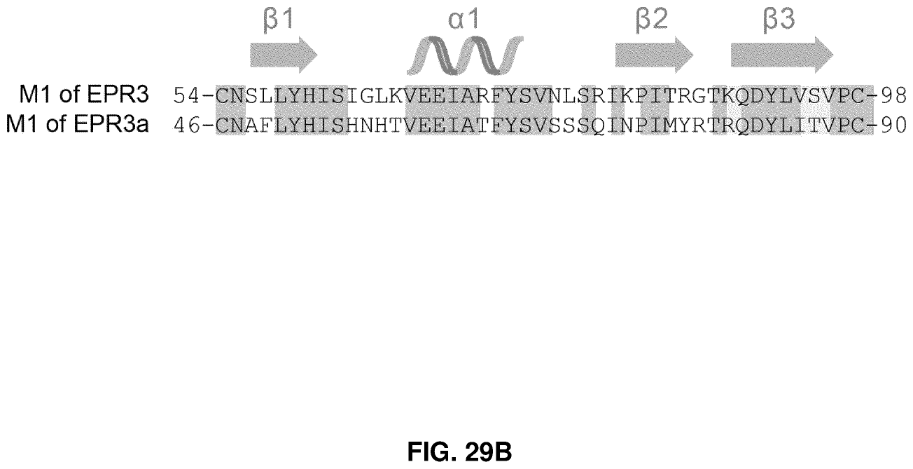

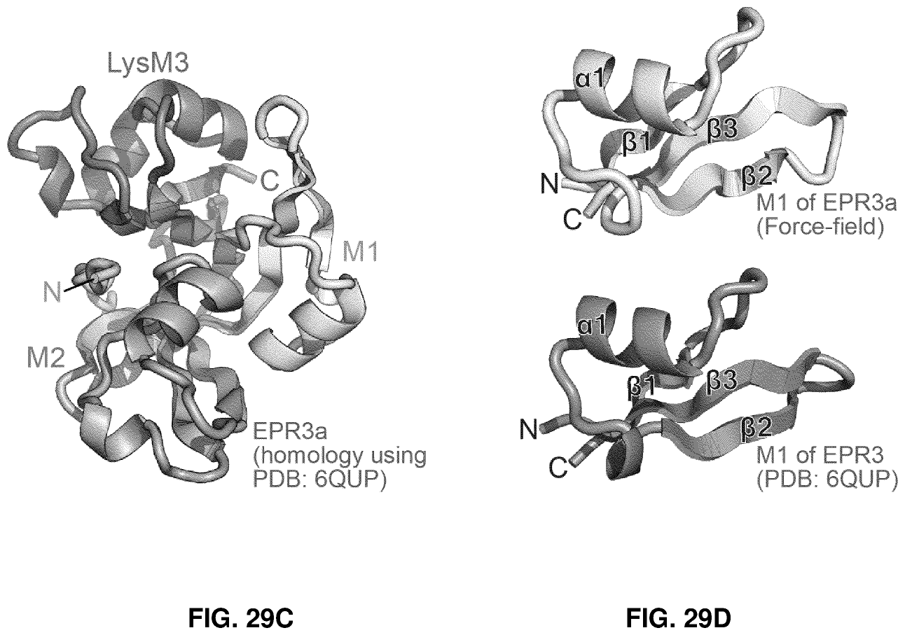

To identify homologs of EPR3 receptors, amino acid sequence alignments, ab-initio protein structure predictions, and structural modeling of the M1 domain were performed as described in Example 6. Lotus, Medicago, soybean, Parasponia, Trema, Populus, Malus, Fragaria, maize, rice, wheat, barley, Datisca, Lupinus, Arabidopsis, Brassica rapa, and Brassica napus genomes were analyzed, as shown in FIG. 29A.

### Results

Analysis of available plant genome sequences identified EPR3 and EPR3a paralogs in most plant species that form mutualistic associations (symbiosis) with arbuscular mycorrhizal fungi, ectomycorrhizal fungi, and/or plants engaging in symbiosis with rhizobia (FIG. 29A). Many plants, including both dicots and monocot, had two copies of the EPR3 class of receptors (FIG. 29A). This was true for important crops, including maize, rice, wheat and barley (FIG. 29A). Arabidopsis thaliana, Brassica rapa and Brassica napus, which do not form mutualistic associations, had neither EPR3 nor EPR3a genes (FIG. 29A).

Force field modelling of the M1 domain as a signature for this class of unique receptors revealed that all contained the 13413 fold. The conserved structure of the 13413 fold is shown for Lotus EPR3 and EPR3a in FIGS. 29B-29D.

### Example 8: Epr3a Functions in Arbuscular Mycorrhizal Symbiosis

The following example describes gene expression and phenotypic studies of Epr3a and Epr3 in L. japonicus. Further, a gene expression analysis of a M. truncatula EPR3/EPR3a-like gene is described.

### Materials and Methods

qRT-PCR

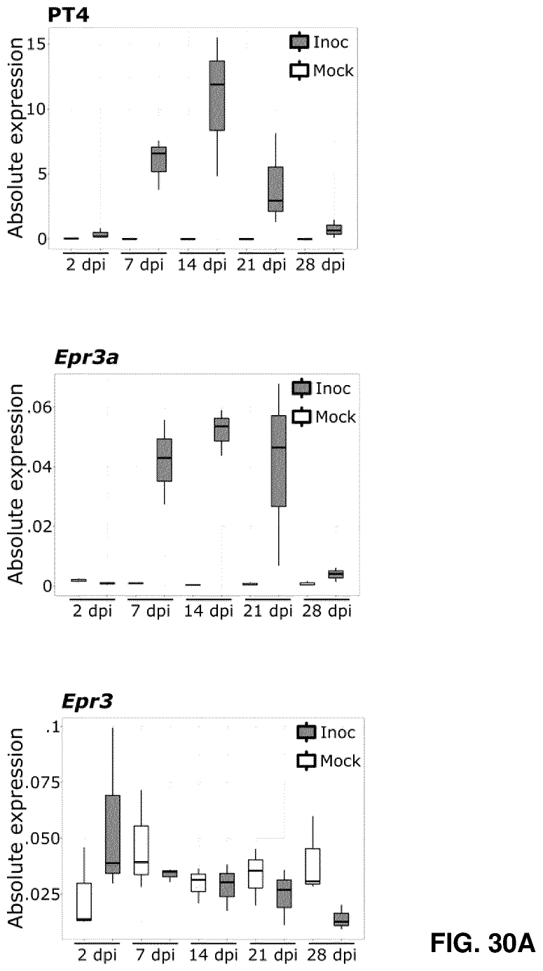

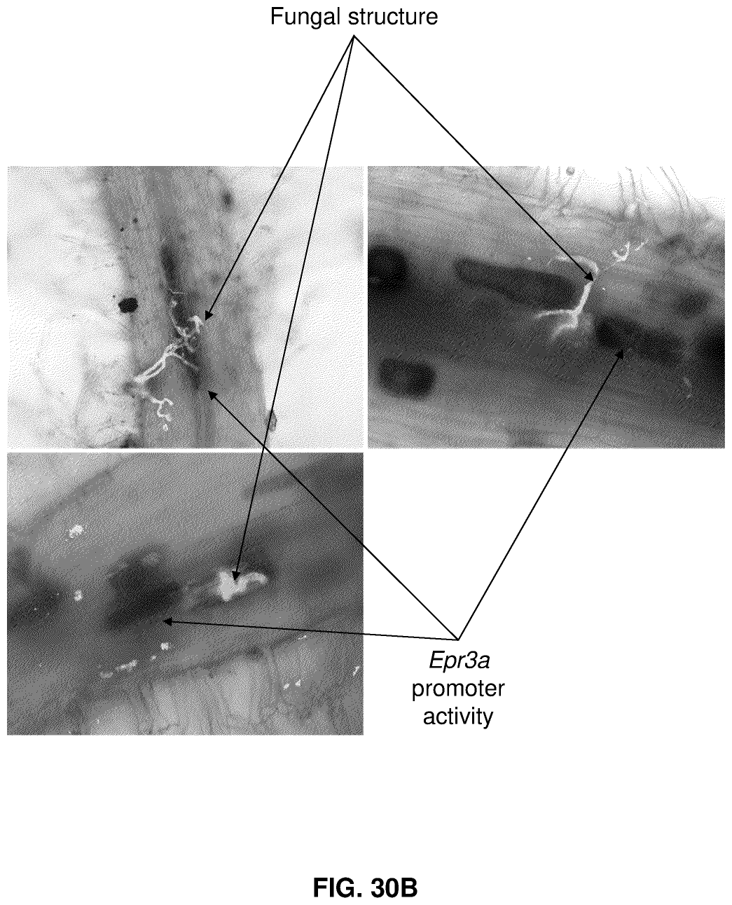

qRT-PCR was performed to measure the absolute expression of the phosphate transporter PT4 (a gene expression marker of arbuscular mycorrhizal symbiosis), Epr3a, and Epr3. Wild-type L. japonicus (Gifu) was inoculated with arbuscular mycorrhiza or a mock inoculation control, and gene expression was measured at 2, 7, 14, 21, or 28 days post inoculation (FIG. 30A). qRT-PCR was performed as described in Example 3, above.

### Epr3a Promoter Activity in Plant Roots

The Epr3a promoter was placed upstream of GUS and transformed into L. japonicus. Hairy root plants expressing the pEpr3a-GUS construct were inoculated with arbuscular mycorrhizal spores, and promoter activity was measured by measuring GUS activity (FIG. 30B). Further, the arbuscular mycorrhizae were labeled with fluorescently labeled wheat germ agglutinin (WGA) for visualization.

### Mutant Alleles of Epr3 and Epr3a and Plant Lines

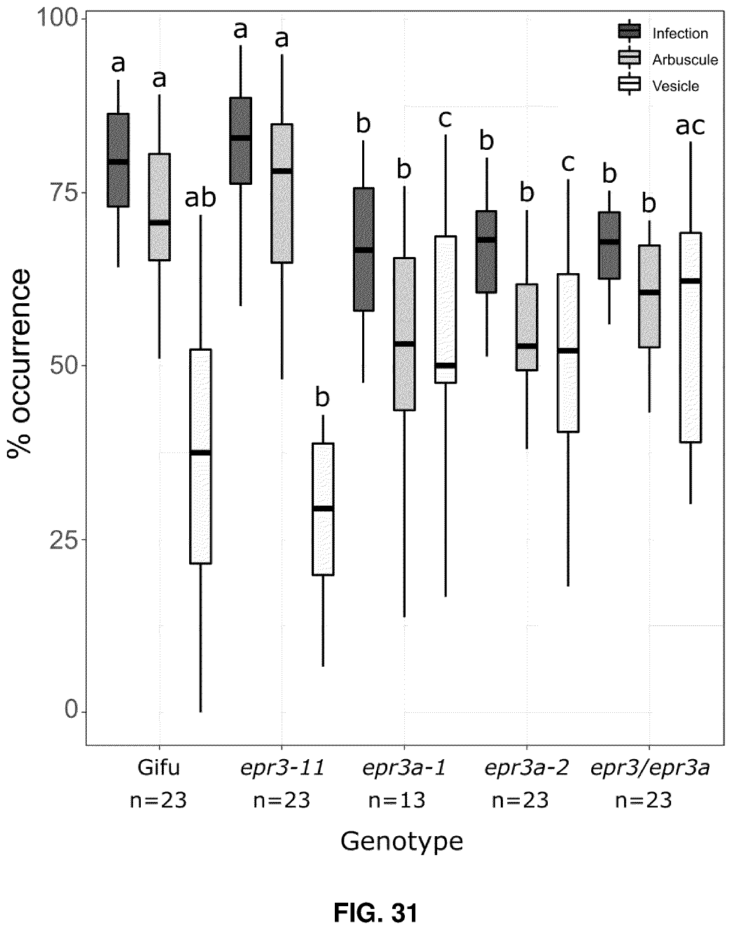

Lines of L. japonicus with epr3 and/or epr3a mutations were used, as described in Example 3 (FIG. 31).

### Arbuscular Mycorrhizae Symbiosis Phenotypes

Wild type L. japonicus Gifu and L. japonicus with mutations in epr3 and/or epr3a were inoculated with arbuscular mycorrhizae. 6 weeks post inoculation, roots were ink-stained and observed under the 20× objective lens on a Zeiss Axioplan II light microscope. For each field of view observed, the occurrence of (% occurrence) fungal hyphae, arbuscules, and vesicles within plant cells was measured (FIG. 31).

### Results

qRT-PCR analysis indicated that expression of Epr3a was induced during arbuscular mycorrhizal symbiosis, while Epr3 showed no induction (FIG. 30A). The expression pattern of Epr3a mirrored that of the PT4 phosphate transporter, a gene expression marker of arbuscular mycorrhizae symbiosis (Harrison et al., Plant Cell 2002 14: 2413-2429). This expression pattern suggested that EPR3a was involved in arbuscular mycorrhiza symbiosis. Therefore, phenotypes related to symbiosis with arbuscular mycorrhiza were measured in L. japonicus lines with epr3 and/or epr3a mutations. epr3a single mutants and the epr3/epr3a double mutant showed a comparable, statistically significant reduction in fungal infection and arbuscule formation and an increase in the presence of arbuscular mycorrhizal vesicles (FIG. 31). No significant difference in arbuscular mycorrhizal symbiosis was observed in the epr3 mutant. Further, an analysis of the activity of the Epr3a promoter showed that it was expressed in L. japonicus roots during colonization with arbuscular mycorrhizae in the cell layer where arbuscules form (FIG. 30B).

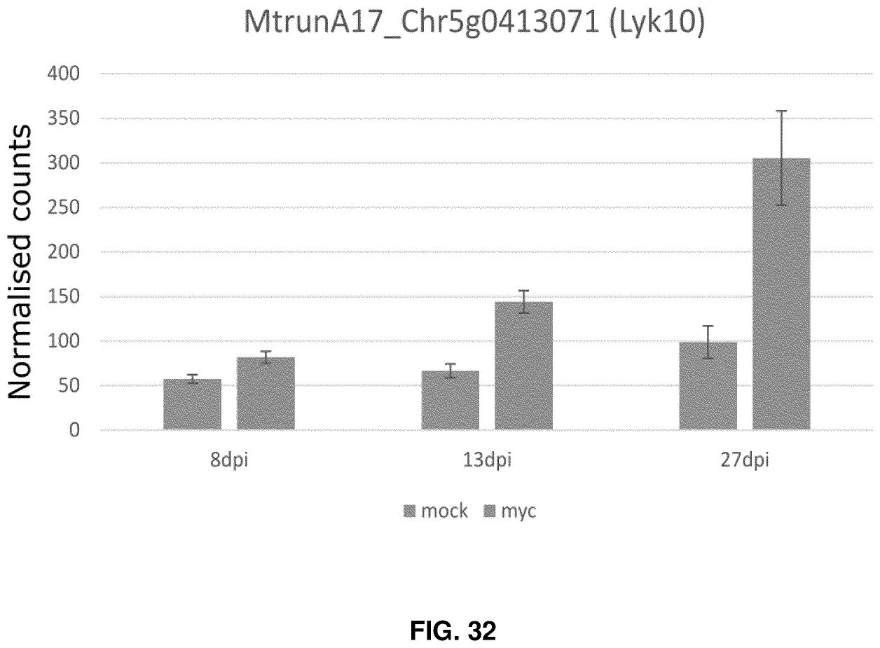

Further, expression of the M. truncatula A17 EPR3/EPR3a-like gene MtrunA17_Chr5g0413071 (Lyk10) was measured during arbuscular mycorrhizal symbiosis by mining and analyzing RNA-seq data presented in Gobbato, E. et al. (Curr Biol 2012 22(23):2236-41). As shown in FIG. 32, expression of MtrunA17_Chr5g0413071 was elevated during arbuscular mycorrhizal symbiosis relative to a mock inoculation control.

Finally, a model for EPR3 and EPR3a signaling in root nodule symbiosis (RNS) and arbuscular mycorrhizal symbiosis (AMS) is presented (FIGS. 33A-33B). Symbiotic rhizobia bacteria induces EPR3, whereas arbuscular mycorrhizal fungi induces EPR3a. Both receptors contribute positive signaling to promote infection by compatible rhizobia in root nodule symbiosis, whereas only EPR3a contributes positive signaling for arbuscular mycorrhiza colonization in mycorrhizal symbiosis.

### Example 9: Analysis of Epr3a Function in Colonization by Non-Symbiotic Burkholderiales

The following example describes a phenotypic study of Epr3a in L. japonicus. Specifically, the ability of wild-type and epr3a mutant L. japonicus to support colonization by co-inoculated M. loti and Burkholderiales was tested.

### Materials and Methods

### Mutant Alleles of Epr3 and Epr3a and Plant Lines

The ability of wild-type L. japonicus (Gifu), and L. japonicus with epr3a (alleles epr3a-1 and epr3a-2), or epr3/epr3a double mutations (“DM” in FIGS. 34A-34B) to support colonization by various Burkholderiales bacteria was tested. Lines of L. japonicus with epr3a or epr3a and epr3 mutations were used, as described in Example 3 (FIG. 31).

Inoculation with M. loti and Burkholderiales Bacteria

25 isolates of bacteria from Burkholderiales orders were co-inoculated with M. loti R7A exoU bacteria. The co-inoculated Burkholderiales belonged to the following genera: LjRoot223—Burkholderia; LjRoot280—Duganella; LjRoot194—Acidovorax; LjRoot230—Burkholderia; LjRoot241—Burkholderia; LjRoot70—Pseudoduganella; LjRoot29—Burkholderia; LjRoot1—Achromobacter; LjRoot131—Polaromonas; LjRoot296—Cupriavidus; LjRoot122—Massilia; LjRoot39—Pseudorhodoferax; and LjRoot294—Variovorax. M. loti R7A exoU bacteria were used because of their ability to induce infection threads, which allows other bacteria (e.g., Burkholderiales) to access the root endosphere. The number of infection threads induced by M. loti R7A exoU bacteria is variable between plants.

A gnotobiotic system was used that was made up of autoclaved magenta boxes filled with well-washed light expanded clay aggregate (LECA) substrate. Burkholderiales isolates were grown in liquid 10% TSB media in a 28° C. shaking incubator until the growth reached exponential stage. To limit the effect of secondary metabolites produced by bacteria, the liquid cultures of bacteria were washed twice and resuspended into ¼ B&D media. Then, OD₆₀₀ of each isolate was measured and adjusted to equal concentrations, and all bacteria were mixed together. The final OD₆₀₀ used for the inoculation was 0.02. For each genotype (Gifu, epr3a-1, epr3a-2, and the epr3epr3A double mutant), 5 biological replicates were used, and plants were allowed to grow with the inoculated bacteria for 4 weeks. At 28 days post inoculation all genotypes had formed nodule primordia, indicating that the M. loti symbiont was active and able to initiate the symbiotic process in the presence of the Burkholderiales isolates. Plants grown in the same magenta boxes were collected and washed with sterile water (two 30 second washes), 80% ethanol (one 30 second wash), and bleach (one 30 second wash) to remove bacteria from rhizoplane, then washed three times using sterile water to remove the ethanol and bleach.

### Measurement of Relative Abundance of Bacteria

The root and nodule primordia tissues from each plant were collected and homogenized by mortar grinding with liquid nitrogen. DNA was extracted using the FastDNA Spin kit for Soil (MP Bioproducts) according to the manufacturer's protocol. DNA concentrations were measured fluorometrically (Quant-iT™425 PicoGreen dsDNA assay kit, Life Technologies, Darmstadt, Germany) and adjusted to 3.5 ng/μl. The variable v5-v7 regions of the 16S rRNA gene were amplified based on MAUI-seq approach. Nextera XT barcode primers were used to distinguished samples. PCR products were purified, pooled and sequenced using an Illumina Iseq instrument. The reads were mapped to the 16S sequence of the input bacteria isolates, and relative abundances were calculated (FIG. 34A).

### Results

In order to investigate the role of EPR3a in the detection of associative, non-symbiotic bacteria, the ability of wild-type L. japonicus (Gifu), epr3a mutant (alleles epr3a-1 and epr3a-2), and epr3epr3A double mutant plants to support colonization by different members of Burkholderiales was tested (FIGS. 34A-34B). It was hypothesized that if EPR3a is important for root and nodule colonization by Burkholderiales isolates, then there would be differences in the abundance of Burkholderiales between wild-type and mutant plants.

As shown in FIGS. 34A-34B, in wild-type plants, the root and nodules were occupied almost exclusively by the symbiont M. loti. In contrast, in the epr3a and epr3/epr3a mutant plants, the relative abundance of M. loti was greatly reduced, with a corresponding increase in the abundance of LjRoot223, LjRoot280, LjRoot194, LjRoot230, LjRoot241 and LjRoot70 Burkholderiales isolates. This suggested that in wild-type plants, EPR3a prevents colonization of infection threads by associative, non-symbiotic bacteria belonging to Burkholderiales. Burkholderiales isolates LjRoot29, LjRoot1, LjRoot131, LjRoot296, LjRoot122, LjRoot39, and LjRoot294 did not show changes in abundance; their individual relative abundances are not shown in FIGS. 34A-34B, but their cumulative relative abundances were included in the values for total Burkholderiales in FIGS. 34A-34B.

Colonization of mutants varied among Burkholderiales isolates, and, without wishing to be bound by theory, it is predicted this was based on the capacity of the Burkholderiales isolates to produce compatible EPS. A great deal of variation was observed between the biological replicates, and the increase in Burkholderiales abundance in epr3a mutants was not found to be statistically significant. Without wishing to be bound by theory, it is believed that this large variability was due to the highly variable ability of M. loti R7A exoU bacteria to induce infection threads, thereby enabling other bacteria to access the root endosphere. The differences observed between wild-type and mutant plants, particularly for the total Burkholderiales, indicate that EPR3a acts to selectively modulate the root microbiota.

1. A genetically altered plant or part thereof comprising a first nucleic acid sequence encoding a heterologous EPR3a or EPR3a-like polypeptide or a modified EPR3a or EPR3a-like polypeptide, wherein the heterologous EPR3a or EPR3a-like polypeptide or the modified EPR3a or EPR3a-like polypeptide provides increased selectivity for a beneficial commensal microbe as compared to a wild-type plant under the same conditions.

2. The genetically altered plant or part thereof of claim 1, wherein the beneficial commensal microbe is a mycorrhizal fungi.

3. The genetically altered plant or part thereof of claim 2, wherein the plant or part thereof further comprises a second nucleic acid sequence encoding a heterologous EPR3 or EPR3-like polypeptide or a modified EPR3 or EPR3-like polypeptide, wherein the heterologous EPR3 or EPR3-like polypeptide or the modified EPR3 or EPR3-like polypeptide provides increased selectivity for a beneficial commensal microbe as compared to a wild-type plant under the same conditions.

4. The genetically altered plant or part thereof of claim 3, wherein the modified EPR3a or EPR3a-like polypeptide comprises a modified ectodomain that has been replaced with all or a portion of an ectodomain of the heterologous EPR3a or EPR3a-like polypeptide, optionally all or a part of the M1 domain, the M2 domain, the LysM3 domain, or all three; and wherein the modified EPR3 or EPR3-like polypeptide comprises a modified ectodomain that has been replaced with all or a portion of an ectodomain of the heterologous EPR3 or EPR3-like polypeptide, optionally all or a part of the M1 domain, the M2 domain, the LysM3 domain, or all three.

5. The genetically altered plant or part thereof of claim 3, wherein the expression of the heterologous EPR3a or EPR3a-like polypeptide, the modified EPR3a or EPR3a-like polypeptide, the heterologous EPR3 or EPR3-like polypeptide, the modified EPR3 or EPR3-like polypeptide, or a combination thereof allows the plant or part thereof to recognize an EPS, a beta-glucan, a cyclic beta-glucan, a LPS, or a surface carbohydrate produced by the microbe, and wherein the microbe is a commensal bacteria, optionally a nitrogen-fixing bacteria, or a mycorrhizal fungi.

6. The genetically altered plant of claim 5, wherein the heterologous EPR3a or EPR3a-like polypeptide, the modified EPR3a or EPR3a-like polypeptide, the heterologous EPR3 or EPR3-like polypeptide, or the modified EPR3 or EPR3-like polypeptide is localized to a plant cell plasma membrane, or both the EPR3 or EPR3-like polypeptide and the EPR3a or EPR3a-like polypeptide are localized to a plant cell plasma membrane, and wherein the plant cell is a root cell.

7. A method of producing the genetically altered plant of claim 3, comprising introducing a genetic alteration to the plant comprising the first nucleic acid sequence encoding the heterologous EPR3a or EPR3a-like polypeptide, and optionally further comprising introducing a genetic alteration to the plant comprising the second nucleic acid sequence encoding the heterologous EPR3 or EPR3-like polypeptide.

8. A method of producing the genetically altered plant of claim 3, comprising genetically editing a gene encoding an endogenous LysM receptor polypeptide in the plant to comprise the modified ectodomain,
wherein the endogenous LysM receptor polypeptide is an endogenous EPR3a or EPR3a-like polypeptide, and wherein the modified EPR3a or EPR3a-like polypeptide was generated by:
(a) providing a heterologous EPR3a or EPR3a-like polypeptide model comprising a structural model, a molecular model, a surface characteristics model, and/or an electrostatic potential model of a M1 domain, a M2 domain, a LysM3 domain, any combination thereof, or the ectodomain of the heterologous EPR3a or EPR3a-like polypeptide having selectivity for the beneficial commensal microbe and an unmodified EPR3a or EPR3a-like polypeptide;
(b) identifying one or more amino acid residues for modification in the unmodified EPRa3 polypeptide by comparing amino acid residues of a oligosaccharide binding feature in the unmodified EPR3a or EPR3a-like polypeptide with the corresponding amino acid residues in the heterologous EPR3a or EPR3a-like polypeptide model; and
(c) generating the unmodified EPR3a or EPR3a-like polypeptide wherein the one or more amino acid residues in the oligosaccharide binding feature of the unmodified EPR3a or EPR3a-like polypeptide have been substituted with corresponding amino acid residues from the heterologous EPR3a or EPR3a-like polypeptide; or
wherein the endogenous LysM receptor polypeptide is an endogenous EPR3 or EPR3-like polypeptide, and wherein the modified EPR3 or EPR3-like polypeptide was generated by:
(a) providing a heterologous EPR3 or EPR3-like polypeptide model comprising a structural model, a molecular model, a surface characteristics model, and/or an electrostatic potential model of a M1 domain, a M2 domain, a LysM3 domain, any combination thereof, or the ectodomain of the heterologous EPR3 or EPR3-like polypeptide having selectivity for the beneficial commensal microbe and an unmodified EPR3 or EPR3-like polypeptide;
(b) identifying one or more amino acid residues for modification in the unmodified EPR3 or EPR3-like polypeptide by comparing amino acid residues of a oligosaccharide binding feature in the unmodified EPR3 or EPR3-like polypeptide with the corresponding amino acid residues in the heterologous EPR3 or EPR3-like polypeptide model; and
(c) generating the unmodified EPR3 or EPR3-like polypeptide wherein the one or more amino acid residues in the oligosaccharide binding feature of the unmodified EPR3 or EPR3-like polypeptide have been substituted with corresponding amino acid residues from the heterologous EPR3 or EPR3-like polypeptide.

9. A genetically altered plant or part thereof comprising a first nucleic acid sequence encoding a heterologous EPR3 or EPR3-like polypeptide or a modified EPR3 or EPR3-like polypeptide, wherein the heterologous EPR3 or EPR3-like polypeptide or the modified EPR3 or EPR3-like polypeptide provides increased selectivity for a beneficial commensal microbe as compared to a wild-type plant under the same conditions.

10. The genetically altered plant or part thereof of claim 9, wherein the plant or part thereof further comprises a second nucleic acid sequence encoding a heterologous EPR3a or EPR3a-like polypeptide or a modified EPR3a or EPR3a-like polypeptide, wherein the heterologous EPR3a or EPR3a-like polypeptide or the modified EPR3a or EPR3a-like polypeptide provides increased selectivity for a beneficial commensal microbe as compared to a wild-type plant under the same conditions.

11. The genetically altered plant or part thereof of claim 10, wherein the modified EPR3 or EPR3-like polypeptide comprises a modified ectodomain that has been replaced with all or a portion of an ectodomain of the heterologous EPR3 or EPR3-like polypeptide, optionally all or a part of the M1 domain, the M2 domain, the LysM3 domain, or all three; and wherein the modified EPR3a or EPR3a-like polypeptide comprises a modified ectodomain that has been replaced with all or a portion of an ectodomain of the heterologous EPR3a or EPR3a-like polypeptide, optionally all or a part of the M1 domain, the M2 domain, the LysM3 domain, or all three.

12. The genetically altered plant or part thereof of claim 10, wherein the expression of the heterologous EPR3 or EPR3-like polypeptide, the modified EPR3 or EPR3-like polypeptide, the heterologous EPR3a or EPR3a-like polypeptide, the modified EPR3a or EPR3a-like polypeptide, or a combination thereof allows the plant or part thereof to recognize an exopolysaccharide (EPS), a beta-glucan, a cyclic beta-glucan, a LPS, or a surface carbohydrate produced by the microbe, and wherein the microbe is a commensal bacteria, optionally a nitrogen-fixing bacteria, or a mycorrhizal fungi.

13. The genetically altered plant or part thereof of claim 12, wherein the heterologous EPR3 or EPR3-like polypeptide, the modified EPR3 or EPR3-like polypeptide, the heterologous EPR3a or EPR3a-like polypeptide, or the modified EPR3a or EPR3a-like polypeptide is localized to a plant cell plasma membrane, or both the EPR3 or EPR3-like polypeptide and the EPR3a or EPR3a-like polypeptide are localized to a plant cell plasma membrane, and wherein the plant cell is a root cell.

14. A method of producing the genetically altered plant of claim 10, comprising introducing a genetic alteration to the plant comprising the first nucleic acid sequence encoding the heterologous EPR3 or EPR3-like polypeptide, and optionally further comprising introducing a genetic alteration to the plant comprising the second nucleic acid sequence encoding the heterologous EPR3a or EPR3a-like polypeptide.

15. A method of producing the genetically altered plant of claim 10, comprising genetically editing a gene encoding an endogenous LysM receptor polypeptide in the plant to comprise the modified ectodomain,
wherein the endogenous LysM receptor polypeptide is an endogenous EPR3 or EPR3-like polypeptide, and wherein the modified EPR3 or EPR3-like polypeptide was generated by:
(a) providing a heterologous EPR3 or EPR3-like polypeptide model comprising a structural model, a molecular model, a surface characteristics model, and/or an electrostatic potential model of a M1 domain, a M2 domain, a LysM3 domain, any combination thereof, or the ectodomain of the heterologous EPR3 or EPR3-like polypeptide having selectivity for the beneficial commensal microbe and an unmodified EPR3 or EPR3-like polypeptide;
(b) identifying one or more amino acid residues for modification in the unmodified EPR3 or EPR3-like polypeptide by comparing amino acid residues of a oligosaccharide binding feature in the unmodified EPR3 or EPR3-like polypeptide with the corresponding amino acid residues in the heterologous EPR3 or EPR3-like polypeptide model; and
(c) generating the unmodified EPR3 or EPR3-like polypeptide wherein the one or more amino acid residues in the oligosaccharide binding feature of the unmodified EPR3 or EPR3-like polypeptide have been substituted with corresponding amino acid residues from the heterologous EPR3 or EPR3-like polypeptide; or
wherein the endogenous LysM receptor polypeptide is an endogenous EPR3a or EPR3a-like polypeptide, and wherein the modified EPR3a or EPR3a-like polypeptide was generated by:
(a) providing a heterologous EPR3a or EPR3a-like polypeptide model comprising a structural model, a molecular model, a surface characteristics model, and/or an electrostatic potential model of a M1 domain, a M2 domain, a LysM3 domain, any combination thereof, or the ectodomain of the heterologous EPR3a or EPR3a-like polypeptide having selectivity for the beneficial commensal microbe and an unmodified EPR3a or EPR3a-like polypeptide;
(b) identifying one or more amino acid residues for modification in the unmodified EPR3a or EPR3a-like polypeptide by comparing amino acid residues of a oligosaccharide binding feature in the unmodified EPR3a or EPR3a-like polypeptide with the corresponding amino acid residues in the heterologous EPR3a or EPR3a-like polypeptide model; and
(c) generating the unmodified EPR3a or EPR3a-like polypeptide wherein the one or more amino acid residues in the oligosaccharide binding feature of the unmodified EPR3a or EPR3a-like polypeptide have been substituted with corresponding amino acid residues from the heterologous EPR3a or EPR3a-like polypeptide.

16. A method of identifying a beneficial commensal microbe capable of participating in a plant root microbiota comprising:
a) providing a first polypeptide comprising an EPR3a or EPR3a-like polypeptide, an ectodomain of an EPR3a or EPR3a-like polypeptide, a M1 domain of an EPR3a or EPR3a-like polypeptide, a M2 domain of an EPR3a or EPR3a-like polypeptide, or a LysM3 domain of an EPR3a or EPR3a-like polypeptide of the plant;
b) contacting the first polypeptide with a sample comprising a microbe or an EPS, a beta-glucan, a cyclic beta-glucan, a LPS, or a surface carbohydrate produced by the microbe;
c) detecting binding of the EPS, the beta-glucan, the cyclic beta-glucan, the LPS, or the surface carbohydrate produced by the microbe to the polypeptide, wherein binding of the EPS, the beta-glucan, the cyclic beta-glucan, the LPS, or the surface carbohydrate to the polypeptide indicates that the microbe is a beneficial commensal microbe capable of participating in the plant root microbiota; optionally, the detecting is by a functional assay optionally selected from (i) detecting enrichment of taxa in Burkholderiales and/or Rhizobiales in a plant rhizosphere or endosphere, wherein enrichment of taxa in Burkholderiales and/or Rhizobiales in the plant rhizosphere or endosphere indicates that the microbe is a beneficial commensal microbe capable of participating in a plant root microbiota; optionally, (ii) detecting nodulation in a plant root system, wherein nodulation indicates that the microbe is a beneficial commensal microbe capable of participating in a plant root microbiota; and/or (iii) detecting mycorrhization in a plant root system, wherein mycorrhization indicates that the microbe is a beneficial commensal microbe capable of participating in a plant root microbiota, or optionally the detecting is by a direct binding assay optionally selected from (1) a competition assay optionally with a known signaling saccharide, or (2) an affinity assay optionally wherein the detected affinity is compared to the affinity for the known signaling saccharide; and
optionally further comprising:
d) culturing the beneficial commensal microbe if binding is detected in step (c); and
e) applying the beneficial commensal microbe to the plant or a part thereof or applying the beneficial commensal microbe, optionally in admixture with a soil-compatible carrier, a fungal carrier, or a growth medium, optionally soil, where the plant is growing or is to be grown.

17. The method of claim 16, further comprising providing a second polypeptide comprising an EPR3 or EPR3-like polypeptide, an ectodomain of an EPR3 or EPR3-like polypeptide, a M1 domain of an EPR3 or EPR3-like polypeptide, a M2 domain of an EPR3 or EPR3-like polypeptide, or a LysM3 domain of an EPR3 or EPR3-like polypeptide of the plant in step (a), wherein the second polypeptide is in contact with the first polypeptide.

18. A method of identifying a beneficial commensal microbe capable of participating in a plant root microbiota comprising:
a) providing a first polypeptide comprising an EPR3 or EPR3-like polypeptide, an ectodomain of an EPR3 or EPR3-like polypeptide, a M1 domain of an EPR3 or EPR3-like polypeptide, a M2 domain of an EPR3 or EPR3-like polypeptide, or a LysM3 domain of an EPR3 or EPR3-like polypeptide of the plant;
b) contacting the first polypeptide with a sample comprising a microbe or an EPS, a beta-glucan, a cyclic beta-glucan, a LPS, or a surface carbohydrate produced by the microbe;
c) detecting binding of the EPS, the beta-glucan, the cyclic beta-glucan, the LPS, or the surface carbohydrate produced by the microbe to the polypeptide, wherein binding of the EPS, the beta-glucan, the cyclic beta-glucan, the LPS, or the surface carbohydrate to the polypeptide indicates that the microbe is a beneficial commensal microbe capable of participating in a plant root microbiota; optionally, the detecting is by a functional assay optionally selected from (i) detecting enrichment of taxa in Burkholderiales and/or Rhizobiales in a plant rhizosphere or endosphere, wherein enrichment of taxa in Burkholderiales and/or Rhizobiales in the plant rhizosphere or endosphere indicates that the microbe is a beneficial commensal microbe capable of participating in a plant root microbiota; optionally, (ii) detecting nodulation in a plant root system, wherein nodulation indicates that the microbe is a beneficial commensal microbe capable of participating in a plant root microbiota; and/or (iii) detecting mycorrhization in a plant root system, wherein mycorrhization indicates that the microbe is a beneficial commensal microbe capable of participating in a plant root microbiota, or optionally the detecting is by a direct binding assay optionally selected from (1) a competition assay optionally with a known signaling saccharide, or (2) an affinity assay optionally wherein the detected affinity is compared to the affinity for the known signaling saccharide; and
optionally further comprising:
d) culturing the beneficial commensal microbe if binding is detected in step (c); and
e) applying the beneficial commensal microbe to the plant or a part thereof or applying the beneficial commensal microbe, optionally in admixture with a soil-compatible carrier, a fungal carrier, or a growth medium, optionally soil, where the plant is growing or is to be grown.

19. The method of claim 18, further comprising providing a second polypeptide comprising an EPR3a or EPR3a-like polypeptide, an ectodomain of an EPR3a or EPR3a-like polypeptide, a M1 domain of an EPR3a or EPR3a-like polypeptide, a M2 domain of an EPR3a or EPR3a-like polypeptide, or a LysM3 domain of an EPR3a or EPR3a-like polypeptide of the plant in step (a), wherein the second polypeptide is in contact with the first polypeptide.
